SEQUENCE LISTING (389 sequences)

SEQ ID NO: 1 - protein, 304 aa, homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 2 - protein, 216 aa, homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 3 - protein, 120 aa, Mus musculus
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 4 - protein, 107 aa, Mus musculus
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 5 - protein, 122 aa, Mus musculus
EVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSA

SEQ ID NO: 6 - protein, 107 aa, Mus musculus
DIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 7 - protein, 116 aa, Mus musculus
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 8 - protein, 107 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 9 - protein, 117 aa, Mus musculus
QVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSA

SEQ ID NO: 10 - protein, 107 aa, Mus musculus
DIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 11 - protein, 115 aa, Mus musculus
QVQLQQPGSVLVRPGASVKLSCKASGYTFTSSWMHWAKQRPGQGLEWIGHIHPNSGISNYNEKFKGKATLTVDTSSSTAYVDLSSLTSEDSAVYYCARGGRFDDWGAGTTVTVSS

SEQ ID NO: 12 - protein, 107 aa, Mus musculus
DIQMTQSPSSLSASLGERVSLTCRASQDIGSSLNWLQQEPDGTIKRLIYATSSLDSGVPKRFSGSRSGSDYSLTISSLESEDFVDYYCLQYASSPWTFGGGTKLEIK

SEQ ID NO: 13 - protein, 120 aa, Mus musculus
DVQLQESGPGLVKPSQSLSLTCTVTGYSITSDYAWNWIRQFPGNKLEWMGYITYSGSTNYNPSLKSRISITRDTSKNQFFLQLNSVTTEDTATYYCARCWDYALYAMDCWGQGTSVTVSS

SEQ ID NO: 14 - protein, 107 aa, Mus musculus
DIQMTQSPASLSASVGETVTITCRTSENIYSYLAWCQQKQGKSPQLLVYNAKTLAEGVPSRFSGSGSGTHFSLKINSLQPEDFGIYYCQHHYDTPLTFGAGTKLELK

SEQ ID NO: 15 - protein, 5 aa, mus musculus
DYVIN

SEQ ID NO: 16 - protein, 7 aa, mus musculus
GYTFTDY

SEQ ID NO: 17 - protein, 8 aa, mus musculus
GYTFTDYV

SEQ ID NO: 18 - protein, 17 aa, mus musculus
EIYPGSGTNYYNEKFKA

SEQ ID NO: 19 - protein, 4 aa, mus musculus
PGSG

SEQ ID NO: 20 - protein, 16 aa, mus musculus
GYTFTDYVIYPGSGTN

SEQ ID NO: 21 - protein, 11 aa, mus musculus
RGRYGLYAMDY

SEQ ID NO: 22 - protein, 9 aa, mus musculus
GRYGLYAMD

SEQ ID NO: 23 - protein, 13 aa, mus musculus
ARRGRYGLYAMDY

SEQ ID NO: 24 - protein, 11 aa, mus musculus
RASQDISNYLN

SEQ ID NO: 25 - protein, 7 aa, Mus musculus
SQDISNY

SEQ ID NO: 26 - protein, 6 aa, mus musculus
QDISNY

SEQ ID NO: 27 - protein, 7 aa, Mus musculus
YTSRLHS

SEQ ID NO: 28 - protein, 9 aa, mus musculus
QQGNTRPWT

SEQ ID NO: 29 - protein, 9 aa, Mus musculus
YTSGNTRPW

SEQ ID NO: 30 - protein, 12 aa, Mus musculus
YTSQQGNTRPWT

SEQ ID NO: 31 - protein, 6 aa, Mus musculus
SDYAWN

SEQ ID NO: 32 - protein, 8 aa, Mus musculus
GYSITSDY

SEQ ID NO: 33 - protein, 9 aa, Mus musculus
GYSITSDYA

SEQ ID NO: 34 - protein, 16 aa, Mus musculus
YITYSGSTSYNPSLES

SEQ ID NO: 35 - protein, 7 aa, Mus musculus
ITYSGST

SEQ ID NO: 36 - protein, 13 aa, Mus musculus
GGYYGSSWGVFAY

SEQ ID NO: 37 - protein, 11 aa, Mus musculus
GYYGSSWGVFA

SEQ ID NO: 38 - protein, 15 aa, Mus musculus
ARGGYYGSSWGVFAY

SEQ ID NO: 39 - protein, 11 aa, Mus musculus
RVSENIYSYLA

SEQ ID NO: 40 - protein, 7 aa, Mus musculus
SENIYSY

SEQ ID NO: 41 - protein, 6 aa, Mus musculus
ENIYSY

SEQ ID NO: 42 - protein, 7 aa, Mus musculus
NAKTLAE

SEQ ID NO: 43 - protein, 9 aa, Mus musculus
QHHYGTPWT

SEQ ID NO: 44 - protein, 6 aa, Mus musculus
HYGTPW

SEQ ID NO: 45 - protein, 9 aa, mus musculus
QHHYGTPWT

SEQ ID NO: 46 - protein, 5 aa, Mus musculus
EYTMH

SEQ ID NO: 47 - protein, 7 aa, Mus musculus
GYTFTEY

SEQ ID NO: 48 - protein, 8 aa, Mus musculus
GYTFTEYT

SEQ ID NO: 49 - protein, 17 aa, Mus musculus
GISPNIGGTSYNQKFKG

SEQ ID NO: 50 - protein, 8 aa, Mus musculus
ISPNIGGT

SEQ ID NO: 51 - protein, 7 aa, Mus musculus
RGGSFDY

SEQ ID NO: 52 - protein, 5 aa, Mus musculus
GGSFD

SEQ ID NO: 53 - protein, 9 aa, Mus musculus
ARRGGSFDY

SEQ ID NO: 54 - protein, 11 aa, Mus musculus
RASQSISDYLH

SEQ ID NO: 55 - protein, 7 aa, Mus musculus
SQSISDY

SEQ ID NO: 56 - protein, 6 aa, Mus musculus
QSISDY

SEQ ID NO: 57 - protein, 7 aa, Mus musculus
YASQSIS

SEQ ID NO: 58 - protein, 9 aa, Mus musculus
QNGHSFPLT

SEQ ID NO: 59 - protein, 6 aa, Mus musculus
GHSFPL

SEQ ID NO: 60 - protein, 5 aa, Mus musculus
SFTMH

SEQ ID NO: 61 - protein, 7 aa, Mus musculus
GYTFTSF

SEQ ID NO: 62 - protein, 8 aa, Mus musculus
GYTFTSFT

SEQ ID NO: 63 - protein, 17 aa, Mus musculus
YINPSSGYTEYNQKFKD

SEQ ID NO: 64 - protein, 8 aa, Mus musculus
INPSSGYT

SEQ ID NO: 65 - protein, 8 aa, Mus musculus
GSSRGFDY

SEQ ID NO: 66 - protein, 6 aa, Mus musculus
SSRGFD

SEQ ID NO: 67 - protein, 10 aa, Mus musculus
VRGSSRGFDY

SEQ ID NO: 68 - protein, 11 aa, Mus musculus
RASENIYSNLA

SEQ ID NO: 69 - protein, 6 aa, Mus musculur
ENIYSN

SEQ ID NO: 70 - protein, 7 aa, mus musculur
AATNLAD

SEQ ID NO: 71 - protein, 9 aa, Mus musculus
QHFWGTPRT

SEQ ID NO: 72 - protein, 6 aa, Mus musculus
FWGTPR

SEQ ID NO: 73 - protein, 5 aa, Mus musculus
SSWMH

SEQ ID NO: 74 - protein, 7 aa, Mus musculus
GYTFTSS

SEQ ID NO: 75 - protein, 8 aa, Mus musculus
GYTFTSSW

SEQ ID NO: 76 - protein, 17 aa, Mus musculus
HIHPNSGISNYNEKFKG

SEQ ID NO: 77 - protein, 8 aa, Mus musculus
IHPNSGIS

SEQ ID NO: 78 - protein, 6 aa, Mus musculus
GGRFDD

SEQ ID NO: 79 - protein, 8 aa, Mus musculus
ARGGRFDD

SEQ ID NO: 80 - protein, 11 aa, Mus musculus
RASQSISDYLH

SEQ ID NO: 81 - protein, 11 aa, Mus musculus
GRFDSQSISDY

SEQ ID NO: 82 - protein, 10 aa, Mus musculus
QNGHSFLMYT

SEQ ID NO: 83 - protein, 7 aa, Mus musculus
GHSFLMY

SEQ ID NO: 84 - protein, 13 aa, Mus musculus
YASQNGHSFLMYT

SEQ ID NO: 85 - protein, 6 aa, mus musculus
SDYAWN

SEQ ID NO: 86 - protein, 8 aa, Mus musculus
GYSITSDY

SEQ ID NO: 87 - protein, 9 aa, Mus musculus
GYSITSDYA

SEQ ID NO: 88 - protein, 16 aa, Mus musculus
YITYSGSTNYNPSLKS

SEQ ID NO: 89 - protein, 11 aa, Mus musculus
CWDYALYAMDC

SEQ ID NO: 90 - protein, 9 aa, mus musculus
WDYALYAMD

SEQ ID NO: 91 - protein, 13 aa, Mus musculus
ARCWDYALYAMDC

SEQ ID NO: 92 - protein, 11 aa, Mus musculus
RTSENIYSYLA

SEQ ID NO: 93 - protein, 7 aa, Mus musculus
NAKTLAE

SEQ ID NO: 94 - protein, 9 aa, Mus musculus
QHHYDTPLT

SEQ ID NO: 95 - protein, 9 aa, mus musculus
NAKHYDTPL

SEQ ID NO: 96 - protein, 9 aa, Mus musculus
QHHYDTPLT

SEQ ID NO: 97 - protein, 5 aa, Mus musculus
NYGMN

SEQ ID NO: 98 - protein, 7 aa, mus musculus
GYTFTNY

SEQ ID NO: 99 - protein, 8 aa, mus musculus
GYTFTNYG

SEQ ID NO: 100 - protein, 17 aa, mus musculus
WINTNTGEPTYAEEFKG

SEQ ID NO: 101 - protein, 8 aa, Mus musculus
INTNTGEP

SEQ ID NO: 102 - protein, 8 aa, Mus musculus
DYLYYFDY

SEQ ID NO: 103 - protein, 6 aa, mus musculus
YLYYFD

SEQ ID NO: 104 - protein, 10 aa, Mus musculus
ARDYLYYFDY

SEQ ID NO: 105 - protein, 11 aa, Mus musculus
KASENVVTYVS

SEQ ID NO: 106 - protein, 7 aa, Mus musculus
SENVVTY

SEQ ID NO: 107 - protein, 6 aa, Mus musculus
ENVVTY

SEQ ID NO: 108 - protein, 7 aa, Mus musculus
GASNRYT

SEQ ID NO: 109 - protein, 9 aa, mus musculus
GQGYSYPYT

SEQ ID NO: 110 - protein, 6 aa, mus musculus
GYSYPY

SEQ ID NO: 111 - protein, 9 aa, mus musculus
GQGYSYPYT

SEQ ID NO: 112 - protein, 120 aa, Artificial
  Chimeric
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSS

SEQ ID NO: 113 - protein, 120 aa, Artificial
  Chimeric
QVQLVQSGAEVKKPGSSVKVSCKASGYTFTDYVINWGRQAPGQGLEWIGEIYPGSGTNYYNEKFKAKATITADKSTSTAYMELSSLRSEDTAVYFCARRGRYGLYAMDYWGQGTTVTVSS

SEQ ID NO: 114 - protein, 107 aa, Artificial
  Chimeric
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 115 - protein, 122 aa, Artificial
  Chimeric
QVQLQESGPGLVKPSQTLSLTCTVSGYSISSDYAWNWIRQPPGKGLEWIGYITYSGSTSYNPSLESRVTISRDTSKNQFSLKLSSVTAADTAVYYCARGGYYGSSWGVFAYWGQGTLVTVSS

SEQ ID NO: 116 - protein, 122 aa, Artificial
  Chimeric
QVQLQESGPGLVKPSQTLSLTCTVSGYSISSDYAWNWIRQPPGKGLEWMGYITYSGSTSYNPSLESRITISRDTSKNQFSLKLSSVTAADTAVYYCARGGYYGSSWGVFAYWGQGTLVTVSS

SEQ ID NO: 117 - protein, 122 aa, Artificial
  Chimeric
QVQLQESGPGLVKPSQTLSLTCTVSGYSITSDYAWNWIRQPPGKGLEWMGYITYSGSTSYNPSLESRITISRDTSKNQFSLKLSSVTAADTAVYYCARGGYYGSSWGVFAYWGQGTLVTVSS

SEQ ID NO: 118 - protein, 107 aa, Artificial
  Chimeric
DIQMTQSPSSLSASVGDRVTITCRVSENIYSYLAWYQQKPGKAPKLLVYNAKTLAEGVPSRFSGSGSGTDFTLTISSLQPEDFATYYCQHHYGTPWTFGGGTKVEIK

SEQ ID NO: 119 - protein, 116 aa, Artificial
  Chimeric
QVQLVQSGAEVKKPGSSVKVSCKASGYTFSEYTMHWVRQAPGQGLEWMGGISPNIGGTSYNQKFKGRVTITADKSTSTAYMELSSLRSEDTAVYYCARRGGSFDYWGQGTTVTVSS

SEQ ID NO: 120 - protein, 116 aa, Artificial
  Chimeric
QVQLVQSGAEVKKPGSSVKVSCKASGYTFSEYTMHWVRQAPGQGLEWIGGISPNIGGTSYNQKFKGRATITADKSTSTAYMELSSLRSEDTAVYYCARRGGSFDYWGQGTTVTVSS

SEQ ID NO: 121 - protein, 116 aa, Artificial
  Chimeric
QVQLVQSGAEVKKPGSSVKVSCKASGYTFSEYTMHWVRQAPGQGLEWIGGISPNIGGTSYNQKFKGRATLTADKSTSTAYMELSSLRSEDTAVYYCARRGGSFDYWGQGTTVTVSS

SEQ ID NO: 122 - protein, 107 aa, Artificial
  Chimeric
EIVMTQSPATLSVSPGERATLSCRASQSISDYLHWYQQKPGQAPRLLIKYASQSISGIPARFSGSGSGTDFTLTISSLEPEDFAVYYCQNGHSFPLTFGQGTKLEIK

SEQ ID NO: 123 - protein, 117 aa, Artificial
  Chimeric
QVQLVQSGAEVKKPGASVKVSCKASGYTFTSFTMHWVRQAPGQGLEWIGYINPSSGYTEYNQKFKDRVTITADKSTSTAYMELSSLRSEDTAVYYCVRGSSRGFDYWGQGTLVTVSS

SEQ ID NO: 124 - protein, 117 aa, Artificial
  Chimeric
QVQLVQSGAEVKKPGASVKVSCKASGYTFTSFTMHWVRQAPGQGLEWIGYINPSSGYTEYNQKFKDRTTITADKSTSTAYMELSSLRSEDTAVYYCVRGSSRGFDYWGQGTLVTVSS

SEQ ID NO: 125 - protein, 117 aa, Artificial
  Chimeric
QVQLVQSGAEVKKPGASVKVSCKASGYTFTSFTMHWVRQAPGQGLEWIGYINPSSGYTEYNQKFKDRTTLTADKSTSTAYMELSSLRSEDTAVYYCVRGSSRGFDYWGQGTLVTVSS

SEQ ID NO: 126 - protein, 107 aa, Artificial
  Chimeric
DIQMTQSPSSLSASVGDRVTITCRASENIYSNLAWFQQKPGKAPKLLVYAATNLADGVPSRFSGSGSGTDYTLTISSLQPEDFATYYCQHFWGTPRTFGGGTKVEIK

SEQ ID NO: 127 - protein, 120 aa, Artificial
  Chimeric
QVQLQESGPGLVKPSQTLSLTCTVSGGSISSDYAWNWIRQPPGKGLEWIGYITYSGSTNYNPSLKSRVTISRDTSKNQFSLKLSSVTAADTAVYYCARCWDYALYAMDCWGQGTTVTVSS

SEQ ID NO: 128 - protein, 120 aa, Artificial
  Chimeric
QVQLQESGPGLVKPSQTLSLTCTVSGYSISSDYAWNWIRQPPGKGLEWIGYITYSGSTNYNPSLKSRVTISRDTSKNQFSLKLSSVTAADTAVYYCARCWDYALYAMDCWGQGTTVTVSS

SEQ ID NO: 129 - protein, 120 aa, Artificial
  Chimeric
QVQLQESGPGLVKPSQTLSLTCTVSGYSISSDYAWNWIRQPPGKGLEWMGYITYSGSTNYNPSLKSRITISRDTSKNQFSLKLSSVTAADTAVYYCARCWDYALYAMDCWGQGTTVTVSS

SEQ ID NO: 130 - protein, 107 aa, Artificial
  Chimeric
DIQMTQSPSSLSASVGDRVTITCRTSENIYSYLAWCQQKPGKAPKLLIYNAKTLAEGVPSRFSGSGSGTDFTLTISSLQPEDFATYYCQHHYDTPLTFGQGTKLEIK

SEQ ID NO: 131 - protein, 107 aa, Artificial
  Chimeric
DIQMTQSPSSLSASVGDRVTITCRTSENIYSYLAWCQQKPGKAPKLLVYNAKTLAEGVPSRFSGSGSGTDFTLTISSLQPEDFATYYCQHHYDTPLTFGQGTKLEIK

SEQ ID NO: 132 - protein, 120 aa, artificial [sequence omitted]

SEQ ID NO: 133 - protein, 107 aa, artificial [sequence omitted]

SEQ ID NO: 134 - protein, 120 aa, artificial
  synthetic
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 135 - protein, 107 aa, artificial
  synthetic
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 136 - protein, 124 aa, artificial
  synthetic
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 137 - protein, 111 aa, artificial
  synthetic
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 138 - protein, 121 aa, artificial [sequence omitted]

SEQ ID NO: 139 - protein, 106 aa, artificial [sequence omitted]

SEQ ID NO: 140 - protein, 119 aa, artificial
  synthetic
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 141 - protein, 112 aa, artificial
  synthetic
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 142 - protein, 119 aa, artificial
  synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 143 - protein, 107 aa, artificial
  synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 144 - protein, 119 aa, artificial [sequence omitted]

SEQ ID NO: 145 - protein, 107 aa, artificial [sequence omitted]

SEQ ID NO: 146 - protein, 123 aa, artificial [sequence omitted]

SEQ ID NO: 147 - protein, 111 aa, artificial
  synthetic
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQI

SEQ ID NO: 148 - protein, 121 aa, artificial [sequence omitted]

SEQ ID NO: 149 - protein, 107 aa, artificial [sequence omitted]

SEQ ID NO: 150 - protein, 116 aa, artificial
  synthetic
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 151 - protein, 107 aa, artificial
  synthetic
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 152 - protein, 121 aa, artificial
  synthetic
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 153 - protein, 107 aa, artificial
  synthetic
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 154 - protein, 121 aa, artificial
  synthetic
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 155 - protein, 107 aa, artificial
  synthetic
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIK

SEQ ID NO: 156 - protein, 98 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 157 - protein, 108 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 158 - protein, 108 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 159 - protein, 106 aa, homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 216 aa, homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 325 aa, artificial
  synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 162 - protein, 329 aa, artificial
  synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 329 aa, artificial
  synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 329 aa, artificial
  synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 329 aa, artificial
  synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 13 aa, homo sapiens
THTCPPCPAPELL

SEQ ID NO: 167 - protein, 15 aa, homo sapiens
NASSMCVPSPAPELL

SEQ ID NO: 168 - protein, 13 aa, homo sapiens
THTSPPSPAPELL

SEQ ID NO: 169 - protein, 13 aa, artificial
  synthetic
THTCSSCPAPELL

SEQ ID NO: 170 - protein, 13 aa, artificial
  synethetic
THTCSSCPAPELL

SEQ ID NO: 171 - protein, 5 aa, artificial
  synthetic
GSSSS

SEQ ID NO: 172 - protein, 10 aa, artificial
  synthetic
GSSSSGSSSS

SEQ ID NO: 173 - protein, 12 aa, artificial
  synthetic
GSSSSGSSSSGS

SEQ ID NO: 174 - protein, 15 aa, artificial
  synthetic
GSSSSGSSSSGSSSS

SEQ ID NO: 175 - protein, 449 aa, artificial
  synthetic
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 176 - protein, 612 aa, artificial
  synthetic
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSVEGGSGGSGGSGGSGGVDDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGSSSSGSSSSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 177 - protein, 219 aa, artificial
  synthtic
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 449 aa, artificial; identical to SEQ ID NO: 175
  synthetic

SEQ ID NO: 179 - protein, 607 aa, artificial
  synthetic
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSVEGGSGGSGGSGGSGGVDDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGSSSSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 180 - protein, 219 aa, artificial; identical to SEQ ID NO: 177
  synthetic

SEQ ID NO: 181 - protein, 449 aa, artificial; identical to SEQ ID NO: 175
  synthetic

SEQ ID NO: 182 - protein, 616 aa, artificial
  synthetic
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSVEGGSGGSGGSGGSGGVDDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGSSSSGSSSSGSSSSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 183 - protein, 219 aa, artificial; identical to SEQ ID NO: 177
  synthetic

SEQ ID NO: 184 - protein, 98 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 185 - protein, 98 aa, Homo sapiens
QVQLVQSGAEVKKPGASVKVSCKASGYTFTGYYMHWVRQAPGQGLEWMGRINPNSGGTNYAQKFQGRVTSTRDTSISTAYMELSRLRSDDTVVYYCAR

SEQ ID NO: 186 - protein, 98 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 187 - protein, 98 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 188 - protein, 98 aa, Homo sapiens
QMQLVQSGAEVKKTGSSVKVSCKASGYTFTYRYLHWVRQAPGQALEWMGWITPFNGNTNYAQKFQDRVTITRDRSMSTAYMELSSLRSEDTAMYYCAR

SEQ ID NO: 189 - protein, 98 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 190 - protein, 98 aa, Homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 191 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 192 - protein, 98 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 193 - protein, 98 aa, Homo sapiens; identical to SEQ ID NO: 191

SEQ ID NO: 194 - protein, 98 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 195 - protein, 100 aa, Homo sapiens
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARI

SEQ ID NO: 196 - protein, 100 aa, Homo sapiens
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHR

SEQ ID NO: 197 - protein, 100 aa, Homo sapiens
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARI

SEQ ID NO: 198 - protein, 100 aa, Homo sapiens
QVTLKESGPALVKPTQTLTLTCTFSGFSLSTSGMRVSWIRQPPGKALEWLARIDWDDDKFYSTSLKTRLTISKDTSKNQVVLTMTNMDPVDTATYYCARI

SEQ ID NO: 199 - protein, 98 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 200 - protein, 97 aa, Homo sapiens
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCAR

SEQ ID NO: 201 - protein, 100 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 202 - protein, 98 aa, Homo sapiens
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCAR

SEQ ID NO: 203 - protein, 98 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 204 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 205 - protein, 98 aa, Homo sapiens; identical to SEQ ID NO: 204

SEQ ID NO: 206 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 207 - protein, 98 aa, Homo sapiens; identical to SEQ ID NO: 206

SEQ ID NO: 208 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 209 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 210 - protein, 99 aa, Homo sapiens
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAKD

SEQ ID NO: 211 - protein, 99 aa, Homo sapiens
EVQLVESGGVVVQPGGSLRLSCAASGFTFDDYAMHWVRQAPGKGLEWVSLISWDGGSTYYADSVKGRFTISRDNSKNSLYLQMNSLRAEDTALYYCAKD

SEQ ID NO: 212 - protein, 98 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAR

SEQ ID NO: 213 - protein, 100 aa, Homo sapiens
EVQLVESGGGLVQPGRSLRLSCTASGFTFGDYAMSWFRQAPGKGLEWVGFIRSKAYGGTTEYTASVKGRFTISRDGSKSIAYLQMNSLKTEDTAVYYCTR

SEQ ID NO: 214 - protein, 97 aa, Homo sapiens
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 215 - protein, 98 aa, Homo sapiens
  X (46)..(46): X may be any amino acid
EVQLVESGEGLVQPGGSLRLSCAASGFTFSSSAMHWVRQAPRKGLXWVSVISTSGDTVLYTDSVKGRFTISRDNAQNSLSLQMNSLRAEGTVVYYCVK

SEQ ID NO: 216 - protein, 98 aa, Homo sapiens
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCAR

SEQ ID NO: 217 - protein, 98 aa, Homo sapiens
EVQLVESGGGLVQPGGSLRLSCSASGFTFSSYAMHWVRQAPGKGLEYVSAISSNGGSTYYADSVKGRFTISRDNSKNTLYLQMSSLRAEDTAVYYCVK

SEQ ID NO: 218 - protein, 97 aa, Homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 219 - protein, 98 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 220 - protein, 100 aa, Homo sapiens
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAR

SEQ ID NO: 221 - protein, 100 aa, Homo sapiens
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTR

SEQ ID NO: 222 - protein, 98 aa, Homo sapiens
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAR

SEQ ID NO: 223 - protein, 99 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKD

SEQ ID NO: 224 - protein, 98 aa, Homo sapiens
QVQLVESGGGVVQPGGSLRLSCAASGFTFSSYGMHWVRQAPGKGLEWVSVIYSGGSSTYYADSVKGRFTISRDNSKNTLYLQMNSLRAEDTAVYYCAK

SEQ ID NO: 225 - protein, 98 aa, Homo sapiens
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCAR

SEQ ID NO: 226 - protein, 99 aa, Homo sapiens
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCAR

SEQ ID NO: 227 - protein, 99 aa, Homo sapiens
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAR

SEQ ID NO: 228 - protein, 99 aa, Homo sapiens
QVQLQESGPGLVKPSQTLSLTCTVSGGSISSGGYYWSWIRQHPGKGLEWIGYIYYSGSTYYNPSLKSLVTISVDTSKNQFSLKLSSVTAADTAVYYCAR

SEQ ID NO: 229 - protein, 97 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 230 - protein, 98 aa, Homo sapiens
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAR

SEQ ID NO: 231 - protein, 99 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 232 - protein, 98 aa, Homo sapiens
QVQLQESGPGLVKPPGTLSLTCAVSGGSISSSNWWSWVRQPPGKGLEWIGEIYHSGSTNYNPSLKSRVTISVDKSKNQFSLKLSSVTAADTAVYCCAR

SEQ ID NO: 233 - protein, 97 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 234 - protein, 99 aa, Homo sapiens
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAR

SEQ ID NO: 235 - protein, 98 aa, Homo sapiens
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCAR

SEQ ID NO: 236 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 237 - protein, 101 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAR

SEQ ID NO: 238 - protein, 98 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQICSLKAEDTAVYYCAR

SEQ ID NO: 239 - protein, 98 aa, Homo sapiens
QVQLVQSWAEVRKSGASVKVSCSFSGFTITSYGIHWVQQSPGQGLEWMGWINPGNGSPSYAKKFQGRFTMTRDMSTTTAYTDLSSLTSEDMAVYYYAR

SEQ ID NO: 240 - protein, 98 aa, Homo sapiens
QVQLVQSGAEVKKPGASVKVSCKASGYIFTDYYMHWVRQAPGQELGWMGRINPNSGGTNYAQKFQGRVTMTRDTSISTAYTELSSLRSEDTATYYCAR

SEQ ID NO: 241 - protein, 86 aa, Homo sapiens
KPGASVKVSCKASGYTFTSYCMHWVHQVHAQGLEWMGLVCPSDGSTSYAQKFQARVTITRDTSMSTAYMELSSLRSEDTAMYYCVR

SEQ ID NO: 242 - protein, 98 aa, Homo sapiens
QVQLMQSGAEVKKPGASVRISCKASGYTFTSYCMHWVCQAHAQGLEWMGLVCPSDGSTSYAQKFQGRVTITRDTSMGTAYMELSSLRSEDTAMYYCVR

SEQ ID NO: 243 - protein, 98 aa, Homo sapiens
QVQLVQSGAEVKKPGASVKVSCKASGYTITSYCMHWVHQVHAQGLEWMGLVCPSDGSTSYAQKFQARVTITRDTSMSTAYMELSSLRSEDTAMYYCVR

SEQ ID NO: 244 - protein, 100 aa, Homo sapiens
QVTLKESGPALVKPTETLTLTCTLSGFSLSTSGMGMSWIRQPPGKALEWLAHIFLNDKKSYSTSLKNRLIISKDTSKSQVVLTMTNMDPVDTATYYCAWR

SEQ ID NO: 245 - protein, 98 aa, Homo sapiens
EVQLVESGGGLVQPGGSLRLSCAASGFTFSNSDMNWARKAPGKGLEWVSGVSWNGSRTHYVDSVKRRFIISRDNSRNSLYLQKNRRRAEDMAVYYCVR

SEQ ID NO: 246 - protein, 98 aa, Homo sapiens
  X (49)..(49): X may be any amino acid
EMQLVESGGGLQKPAWSPRLSCAASQFTFSSYYMNCVRQAPGNGLELVXQVNPNGGSTYLIDSGKDRFNTSRDNAKNTLHLQMNSLKTEDTALYYCTR

SEQ ID NO: 247 - protein, 98 aa, Homo sapiens
EVQLVESGGGLVQPGGSLRLSCAASGFTFSNSDMNWVHQAPGKGLEWVSGVSWNGSRTHYADSVKGRFIISRDNSRNTLYLQTNSLRAEDTAVYYCVR

SEQ ID NO: 248 - protein, 97 aa, Homo sapiens
EVQLVESGGGLVQPRGSLRLSCAASGFTVSSNEMSWIRQAPGKGLEWVSSISGGSTYYADSRKGRFTISRDNSKNTLYLQMNNLRAEGTAAYYCARY

SEQ ID NO: 249 - protein, 96 aa, Homo sapiens
EVQLVESRGVLVQPGGSLRLSCAASGFTVSSNEMSWVRQAPGKGLEWVSSISGGSTYYADSRKGRFTISRDNSKNTLHLQMNSLRAEDTAVYYCKK

SEQ ID NO: 250 - protein, 100 aa, Homo sapiens
EVQLVESGGGLVQPGGSLRLSCAASGFTFSDHYMSWVRQAQGKGLELVGLIRNKANSYTTEYAASVKGRLTISREDSKNTMYLQMSNLKTEDLAVYYCAR

SEQ ID NO: 251 - protein, 97 aa, Homo sapiens
EVQLVQSGGGLVHPGGSLRLSCAGSGFTFSSYAMHWVRQAPGKGLEWVSAIGTGGGTYYADSVKGRFTISRDNAKNSLYLQMNSLRAEDMAVYYCAR

SEQ ID NO: 252 - protein, 98 aa, Homo sapiens
EVQLVESGRGLAQPGGYLKLSGAASGFTVGSWYMSWIHQAPGKGLEWVSYISSSGCSTNYADSVKGRFTISTDNSKNTLYLQMNSLRVEDTAVYYCAR

SEQ ID NO: 253 - protein, 98 aa, Homo sapiens
EVQLVESGGGLVQPGGSLRLSCAASGFTFSSYWMHWVRQAPGKGLVWVSRINSDGSSTSYADSMKGQFTISRDNAKNTLYLQMNSLRAEDMAVYYCTR

SEQ ID NO: 254 - protein, 98 aa, Homo sapiens
EVQLVESGGGLVQPGGSLRLSCPDSGFTFSNHYMSWVRQAPGKGLEWISYISGDSGYTNYADSVKGRFTISRDNANNSPYLQMNSLRAEDTAVYYCVK

SEQ ID NO: 255 - protein, 97 aa, Homo sapiens
EVQLVQSGGGLVQPGGSLRLSCAGSGFTFSSYAMHWVRQAPGKGLEWVSAIGTGGGTYYADSVKGRFTISRDNAKNSLYLQMNSLRAEDMAVYYCAR

SEQ ID NO: 256 - protein, 100 aa, Homo sapiens
EVQLVESAGGLGTAWGSLRLSCAASGFTCSNAWMSWVRQAPGKGLEWVGCIKSKANGGTTDYAAPVKGRFTISRDDSKNTLYLQMISLKTEDTAVYYCTT

SEQ ID NO: 257 - protein, 98 aa, Homo sapiens
EVQLVESGGGLVQPGGSLRLSCPASGFTFSNHYMSWVRQAPGKGLEWVSYISGDSGYTNYADSVKGRFTISRDNANNSPYLQMNSLRAEDTAVYYCVK

SEQ ID NO: 258 - protein, 98 aa, Homo sapiens
EVQLVESGGGLVQPGGSLRLSCAASGFTFSNHYTSWVRQAPGKGLEWVSYSSGNSGYTNYADSVKGRFTISRDNAKNSLYLQMNSLRAEDTAVYYCVK

SEQ ID NO: 259 - protein, 98 aa, Homo sapiens
QVQLQESGPGLVKPSETLSLTCVVSGGSISSSNWWSWVRQPPGKGLEWIGEIYHSGSPNYNPSLKSRVTISVDKSKNQFSLKLSSVTAADTAVYYCAR

SEQ ID NO: 260 - protein, 98 aa, Homo sapiens
QVQLVQSGHEVKQPGASVKVSCKASGYSFTTYGMNWVPQAPGQGLEWMGWFNTYTGNPTYAQGFTGRFVFSMDTSASTAYLQISSLKAEDMAMYYCAR

SEQ ID NO: 261 - protein, 98 aa, Homo sapiens
  X (14)..(14): X can be any amion acid
EAQLTESGGDLVHXEGPLRLSCAASWFTFSIYEIHWVCQASGKGLEWVAVIWRSESHQYNADYVRGRLTTSRDNTKYMLYMQMNSLRTQNMAAFNCAG

SEQ ID NO: 262 - protein, 95 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 263 - protein, 95 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNNYP

SEQ ID NO: 264 - protein, 95 aa, Homo sapiens
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

SEQ ID NO: 265 - protein, 95 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYP

SEQ ID NO: 266 - protein, 95 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAP

SEQ ID NO: 267 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 268 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 269 - protein, 95 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYCQQYNSYS

SEQ ID NO: 270 - protein, 95 aa, Homo sapiens
AIQMTQSPSSLSASVGDRVTITCRASQGIRNDLGWYQQKPGKAPKLLIYAASSLQSGVPSRFSGSGSGTDFTLTISSLQPEDFATYYCLQDYNYP

SEQ ID NO: 271 - protein, 95 aa, Homo sapiens
AIRMTQSPSSFSASTGDRVTITCRASQGISSYLAWYQQKPGKAPKLLIYAASTLQSGVPSRFSGSGSGTDFTLTISCLQSEDFATYYCQQYYSYP

SEQ ID NO: 272 - protein, 95 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYP

SEQ ID NO: 273 - protein, 95 aa, Homo sapiens
DIQMTQSPSSLSASVGDRVTITCRASQGISNSLAWYQQKPGKAPKLLLYAASRLESGVPSRFSGSGSGTDYTLTISSLQPEDFATYYCQQYYSTP

SEQ ID NO: 274 - protein, 95 aa, Homo sapiens; identical to SEQ ID NO: 262

SEQ ID NO: 275 - protein, 95 aa, Homo sapiens; identical to SEQ ID NO: 263

SEQ ID NO: 276 - protein, 95 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 277 - protein, 95 aa, Homo sapiens
NIQMTQSPSAMSASVGDRVTITCRARQGISNYLAWFQQKPGKVPKHLIYAASSLQSGVPSRFSGSGSGTEFTLTISSLQPEDFATYYCLQHNSYP

SEQ ID NO: 278 - protein, 95 aa, Homo sapiens; identical to SEQ ID NO: 267

SEQ ID NO: 279 - protein, 95 aa, Homo sapiens; identical to SEQ ID NO: 268

SEQ ID NO: 280 - protein, 95 aa, Homo sapiens
AIRMTQSPFSLSASVGDRVTITCWASQGISSYLAWYQQKPAKAPKLFIYYASSLQSGVPSRFSGSGSGTDYTLTISSLQPEDFATYYCQQYYSTP

SEQ ID NO: 281 - protein, 95 aa, Homo sapiens
VIWMTQSPSLLSASTGDRVTISCRMSQGISSYLAWYQQKPGKAPELLIYAASTLQSGVPSRFSGSGSGTDFTLTISCLQSEDFATYYCQQYYSFP

SEQ ID NO: 282 - protein, 100 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFP

SEQ ID NO: 283 - protein, 100 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 284 - protein, 100 aa, Homo sapiens
  X (93)..(93): X may be any amino acid
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYXMQGIHLP

SEQ ID NO: 285 - protein, 100 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWP

SEQ ID NO: 286 - protein, 101 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFP

SEQ ID NO: 287 - protein, 100 aa, Homo sapiens
EIVMTQTPLSLSITPGEQASISCRSSQSLLHSDGYTYLYWFLQKARPVSTLLIYEVSNRFSGVPDRFSGSGSGTDFTLKISRVEAEDFGVYYCMQDAQDP

SEQ ID NO: 288 - protein, 100 aa, Homo sapiens; identical to SEQ ID NO: 283

SEQ ID NO: 289 - protein, 100 aa, Homo sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLP

SEQ ID NO: 290 - protein, 100 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWP

SEQ ID NO: 291 - protein, 101 aa, Homo sapiens; identical to SEQ ID NO: 286

SEQ ID NO: 292 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 293 - protein, 95 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 294 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 295 - protein, 95 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWH

SEQ ID NO: 296 - protein, 95 aa, Homo sapiens; identical to SEQ ID NO: 293

SEQ ID NO: 297 - protein, 96 aa, Homo sapiens
EIVLTQSPATLSLSPGERATLSCGASQSVSSSYLAWYQQKPGLAPRLLIYDASSRATGIPDRFSGSGSGTDFTLTISRLEPEDFAVYYCQQYGSSP

SEQ ID NO: 298 - protein, 96 aa, Homo sapiens
EIVMTQSPATLSLSPGERATLSCRASQSVSSSYLSWYQQKPGQAPRLLIYGASTRATGIPARFSGSGSGTDFTLTISSLQPEDFAVYYCQQDYNLP

SEQ ID NO: 299 - protein, 101 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 300 - protein, 95 aa, Homo sapiens
ETTLTQSPAFMSATPGDKVNISCKASQDIDDDMNWYQQKPGEAAIFIIQEATTLVPGIPPRFSGSGYGTDFTLTINNIESEDAAYYFCLQHDNFP

SEQ ID NO: 301 - protein, 95 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 302 - protein, 95 aa, Homo sapiens; identical to SEQ ID NO: 301

SEQ ID NO: 303 - protein, 95 aa, Homo sapiens
DIQLTQSPSSLSASVGDRVTITCRVSQGISSYLNWYRQKPGKVPKLLIYSASNLQSGVPSRFSGSGSGTDFTLTISSLQPEDVATYYGQRTYNAP

SEQ ID NO: 304 - protein, 95 aa, Homo sapiens
DIQMTQSPSSLSASVGDRVTITCRASQGISNNLNWYQQKPGKTPKLLIYAAPSLQSGIPSRFSDSGSGADYTLTIRSLQPEDFATYYCQQSDSTP

SEQ ID NO: 305 - protein, 95 aa, Homo sapiens
DIQVTQSPSSLSASVGDRVTITCRASQGISNGLSWYQQKPGQAPTLLIYAASSLQSGVPSRFSGSGSGTDFTLTISSLQPEDVATYYCLQDYTTP

SEQ ID NO: 306 - protein, 95 aa, Homo sapiens; identical to SEQ ID NO: 303

SEQ ID NO: 307 - protein, 95 aa, Homo sapiens
DIQMIQSPSFLSASVGDRVSIICWASEGISSNLAWYLQKPGKSPKLFLYDAKDLHPGVSSRFSGRGSGTDFTLTIISLKPEDFAAYYCKQDFSYP

SEQ ID NO: 308 - protein, 100 aa, Homo sapiens
DIVMTQTPLSSPVTLGQPASISFRSSQSLVHSDGNTYLSWLQQRPGQPPRLLIYKVSNRFSGVPDRFSGSGAGTDFTLKISRVEAEDVGVYYCTQATQFP

SEQ ID NO: 309 - protein, 96 aa, Homo sapiens
EIVMTQSPPTLSLSPGERVTLSCRASQSVSSSYLTWYQQKPGQAPRLLIYGASTRATSIPARFSGSGSGTDFTLTISSLQPEDFAVYYCQQDHNLP

SEQ ID NO: 310 - protein, 96 aa, Homo sapiens; identical to SEQ ID NO: 298

SEQ ID NO: 311 - protein, 95 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHP

SEQ ID NO: 312 - protein, 98 aa, Homo sapiens
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNG

SEQ ID NO: 313 - protein, 99 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 314 - protein, 98 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNG

SEQ ID NO: 315 - protein, 98 aa, Homo sapiens
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSG

SEQ ID NO: 316 - protein, 98 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSA

SEQ ID NO: 317 - protein, 98 aa, Homo sapiens
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLSA

SEQ ID NO: 318 - protein, 99 aa, Homo sapiens
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTF

SEQ ID NO: 319 - protein, 99 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTL

SEQ ID NO: 320 - protein, 99 aa, Homo sapiens
QSALTQPPSVSGSPGQSVTISCTGTSSDVGSYNRVSWYQQPPGTAPKLMIYEVSNRPSGVPDRFSGSKSGNTASLTISGLQAEDEADYYCSLYTSSSTF

SEQ ID NO: 321 - protein, 99 aa, Homo sapiens
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTL

SEQ ID NO: 322 - protein, 99 aa, Homo sapiens
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNF

SEQ ID NO: 323 - protein, 95 aa, Homo sapiens
SYELTQPPSVSVSPGQTASITCSGDKLGDKYACWYQQKPGQSPVLVIYQDSKRPSGIPERFSGSNSGNTATLTISGTQAMDEADYYCQAWDSSTA

SEQ ID NO: 324 - protein, 96 aa, Homo sapiens
SYELTQPPSVSVSPGQTARITCSGDALPKKYAYWYQQKSGQAPVLVIYEDSKRPSGIPERFSGSSSGTMATLTISGAQVEDEADYYCYSTDSSGNH

SEQ ID NO: 325 - protein, 96 aa, Homo sapiens
SYELTQPHSVSVATAQMARITCGGNNIGSKAVHWYQQKPGQDPVLVIYSDSNRPSGIPERFSGSNPGNTTTLTISRIEAGDEADYYCQVWDSSSDH

SEQ ID NO: 326 - protein, 96 aa, Homo sapiens
SYELTQPPSVSVSLGQMARITCSGEALPKKYAYWYQQKPGQFPVLVIYKDSERPSGIPERFSGSSSGTIVTLTISGVQAEDEADYYCLSADSSGTY

SEQ ID NO: 327 - protein, 96 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNH

SEQ ID NO: 328 - protein, 96 aa, Homo sapiens
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDH

SEQ ID NO: 329 - protein, 94 aa, Homo sapiens
SYELTQLPSVSVSPGQTARITCSGDVLGENYADWYQQKPGQAPELVIYEDSERYPGIPERFSGSTSGNTTTLTISRVLTEDEADYYCLSGDEDN

SEQ ID NO: 330 - protein, 96 aa, Homo sapiens
SYELMQPPSVSVSPGQTARITCSGDALPKQYAYWYQQKPGQAPVLVIYKDSERPSGIPERFSGSSSGTTVTLTISGVQAEDEADYYCQSADSSGTY

SEQ ID NO: 331 - protein, 94 aa, Homo sapiens
SYELTQPSSVSVSPGQTARITCSGDVLAKKYARWFQQKPGQAPVLVIYKDSERPSGIPERFSGSSSGTTVTLTISGAQVEDEADYYCYSAADNN

SEQ ID NO: 332 - protein, 95 aa, Homo sapiens
SYELTQPLSVSVALGQTARITCGGNNIGSKNVHWYQQKPGQAPVLVIYRDSNRPSGIPERFSGSNSGNTATLTISRAQAGDEADYYCQVWDSSTA

SEQ ID NO: 333 - protein, 103 aa, Homo sapiens
LPVLTQPPSASALLGASIKLTCTLSSEHSTYTIEWYQQRPGRSPQYIMKVKSDGSHSKGDGIPDRFMGSSSGADRYLTFSNLQSDDEAEYHCGESHTIDGQVG

SEQ ID NO: 334 - protein, 99 aa, Homo sapiens
QPVLTQSSSASASLGSSVKLTCTLSSGHSSYIIAWHQQQPGKAPRYLMKLEGSGSYNKGSGVPDRFSGSSSGADRYLTISNLQLEDEADYYCETWDSNT

SEQ ID NO: 335 - protein, 99 aa, Homo sapiens
QLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGI

SEQ ID NO: 336 - protein, 104 aa, Homo sapiens
QPVLTQPPSSSASPGESARLTCTLPSDINVGSYNIYWYQQKPGSPPRYLLYYYSDSDKGQGSGVPSRFSGSKDASANTGILLISGLQSEDEADYYCMIWPSNAS

SEQ ID NO: 337 - protein, 104 aa, Homo sapiens
QPVLTQPTSLSASPGASARFTCTLRSGINVGTYRIYWYQQKPGSLPRYLLRYKSDSDKQQGSGVPSRFSGSKDASTNAGLLLISGLQSEDEADYYCAIWYSSTS

SEQ ID NO: 338 - protein, 104 aa, Homo sapiens
QAVLTQPASLSASPGASASLTCTLRSGINVGTYRIYWYQQKPGSPPQYLLRYKSDSDKQQGSGVPSRFSGSKDASANAGILLISGLQSEDEADYYCMIWHSSAS

SEQ ID NO: 339 - protein, 105 aa, Homo sapiens
QPVLTQPSSHSASSGASVRLTCMLSSGFSVGDFWIRWYQQKPGNPPRYLLYYHSDSNKGQGSGVPSRFSGSNDASANAGILRISGLQPEDEADYYCGTWHSNSKT

SEQ ID NO: 340 - protein, 98 aa, Homo sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSN

SEQ ID NO: 341 - protein, 98 aa, Homo sapiens
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQ

SEQ ID NO: 342 - protein, 98 aa, Homo sapiens
QAVVTQEPSLTVSPGGTVTLTCGSSTGAVTSGHYPYWFQQKPGQAPRTLIYDTSNKHSWTPARFSGSLLGGKAALTLSGAQPEDEAEYYCLLSYSGAR

SEQ ID NO: 343 - protein, 98 aa, Homo sapiens
QTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGI

SEQ ID NO: 344 - protein, 104 aa, Homo sapiens
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFV

SEQ ID NO: 345 - protein, 98 aa, Homo sapiens
QSVLTQPPSVSAAPGQKVTISCSGSSSDMGNYAVSWYQQLPGTAPKLLIYENNKRPSGIPDRFSGSKSGTSATLGITGLWPEDEADYYCLAWDTSPRA

SEQ ID NO: 346 - protein, 99 aa, Homo sapiens
QSVLTQPPSVSGAPGQRVTISCTGSSSNIGAGYVVHWYQQLPGTAPKLLIYGNSNRPSGVPDQFSGSKSGTSASLAITGLQSEDEADYYCKAWDNSLNA

SEQ ID NO: 347 - protein, 104 aa, Homo sapiens
RPVLTQPPSLSASPGATARLPCTLSSDLSVGGKNMFWYQQKPGSSPRLFLYHYSDSDKQLGPGVPSRVSGSKETSSNTAFLLISGLQPEDEADYYCQVYESSAN

SEQ ID NO: 348 - protein, 99 aa, Homo sapiens
QSALTQPPFVSGAPGQSVTISCTGTSSDVGDYDHVFWYQKRLSTTSRLLIYNVNTRPSGISDLFSGSKSGNMASLTISGLKSEVEANYHCSLYSSSYTF

SEQ ID NO: 349 - protein, 96 aa, Homo sapiens
SSGPTQVPAVSVALGQMARITCQGDSMEGSYEHWYQQKPGQAPVLVIYDSSDRPSRIPERFSGSKSGNTTTLTITGAQAEDEADYYYQLIDNHATQ

SEQ ID NO: 350 - protein, 104 aa, Homo sapiens
QPVLTQPTSLSASPGASARLTCTLRSGINLGSYRIFWYQQKPESPPRYLLSYYSDSSKHQGSGVPSRFSGSKDASSNAGILVISGLQSEDEADYYCMIWHSSAS

SEQ ID NO: 351 - protein, 98 aa, Homo sapiens
QSVVTQEPSLSGSPGGTVTLTCALSSGSVSTSHYPRWYQQTPGQAPHMLICSPNTCPSGVPGRFSGSILGNKAALTITGTQVDDDSDHYCVLYMGSGN

SEQ ID NO: 352 - protein, 133 aa, homo sapiens [sequence omitted]

SEQ ID NO: 353 - protein, 130 aa, homo sapiens
SSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 354 - protein, 133 aa, artificial
  synthetic
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 355 - protein, 130 aa, artificial
  synthetic
SSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 356 - protein, 130 aa, artificial
  synthetic
SSSTKKTQLQLEHLLLDLQMILNGINNYKNPKLTAMLAKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 357 - protein, 8 aa, artificial
  synthetic
EHALYDAL

SEQ ID NO: 358 - protein, 8 aa, artificial
  synthetic
YAFNFELI

SEQ ID NO: 359 - protein, 9 aa, artificial
  synthetic
ITILQSWIF

SEQ ID NO: 360 - protein, 22 aa, artificial
  synthetic
PKKKIQLHAEHALYDALMILNI

SEQ ID NO: 361 - protein, 22 aa, artificial
  synthetic
LEDYAFNFELILEEIARLFESG

SEQ ID NO: 362 - protein, 21 aa, artificial
  synthetic
EDEQEEMANAIITILQSWIFS

SEQ ID NO: 363 - protein, 100 aa, artificial
  synthetic
PKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 364 - protein, 115 aa, artificial
  synthetic
GSSSSGSSSSGSSSSPKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 365 - protein, 302 aa, artificial
  synthetic
SKNFHLRPRDLISNINVIVLELKGSETTFMCEYADETATIVEFLNRWITFSQSIISTLTGGSSSTKKTQLQLEHLLLDLQMILNGINNYKNPKLTRMLTFKFYMPKKATELKHLQCLEEELKPLEEVLNLAQGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFWGQMVYYQCVQGYRALHRGPAESVCKMTHGKTRWTQPQLICTG

SEQ ID NO: 366 - protein, 114 aa, homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 367 - protein, 65 aa, homo sapiens
ITCPPPMSVEHADIWVKSYSLYSRERYICNSGFKRKAGTSSLTECVLNKATNVAHWTTPSLKCIR

SEQ ID NO: 368 - protein, 138 aa, homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 369 - protein, 133 aa, artificial
  synthetic
  X (5)..(5): X may be any amino acid
  X (8)..(9): X may be any amino acid
  X (11)..(16): X may be any amino acid
  X (19)..(19): X may be any amino acid
  X (23)..(23): X may be any amino acid
  X (65)..(66): X may be any amino acid
  X (68)..(73): X may be any amino acid
  X (75)..(80): X may be any amino acid
  X (109)..(110): X may be any amino acid
  X (112)..(113): X may be any amino acid
  X (116)..(117): X may be any amino acid
  X (119)..(120): X may be any amino acid
  X (123)..(123): X may be any amino acid
QGQDXHMXXMXXXXXXVDXLKNXVNDLVPEFLPAPEDVETNCEWSAFSCFQKAQLKSANTGNNEXXIXXXXXXLXXXXXXTNAGRRQKHRLTCPSCDSYEKKPPKEFLXXFXXLLXXMXXQHXSSRTHGSEDS

SEQ ID NO: 370 - protein, 157 aa, homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 371 - protein, 165 aa, homo sapiens [sequence omitted]

SEQ ID NO: 372 - protein, 166 aa, homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 373 - protein, 166 aa, homo sapiens
CDLPQTHSLGNRRALILLAQMGRISHFSCLKDRHDFGFPEEEFDGHQFQKTQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 374 - protein, 166 aa, homo sapiens
CDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 375 - protein, 166 aa, homo sapiens
CDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 376 - protein, 166 aa, homo sapiens
CDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTNLKKGLRRKD

SEQ ID NO: 377 - protein, 166 aa, homo sapiens
CDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 378 - protein, 166 aa, homo sapiens
CDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 379 - protein, 166 aa, homo sapiens
CNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 380 - protein, 166 aa, homo sapiens
CDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 381 - protein, 166 aa, homo sapiens
CDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 382 - protein, 166 aa, homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 383 - protein, 152 aa, homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 384 - protein, 215 aa, homo sapiens
FPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 385 - protein, 209 aa, homo sapiens
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 386 - protein, 197 aa, homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 387 - protein, 306 aa, homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 388 - protein, 18 aa, artificial
  synthetic
GEGTSTGSGGSGGSGGAD

SEQ ID NO: 389 - protein, 10 aa, homo sapiens
EPKSCDKTHS

(19) United States
(12) Patent Application Publication
(10) Pub. No.: US 2024/0392008 A1
(43) Pub. Date: Nov. 28, 2024

(54) MULTISPECIFIC PROTEINS BINDING TO NKP46, A CYTOKINE RECEPTOR, A TUMOUR ANTIGEN AND CD16A

(71) Applicant: INNATE PHARMA, Marseille (FR)

(72) Inventors: Laurent GAUTHIER, Marseille (FR); Yannis MOREL, Marseille (FR); Olivier DEMARIA, Marseille (FR)

(21) Appl. No.: 18/568,310

(22) PCT Filed: Jun. 8, 2022
(86) PCT No.: PCT/EP2022/065493

§ 371 Date: Dec. 8, 2023

Related U.S. Application Data
(60) Provisional application No. 63/208,511, filed on Jun. 9, 2021.

(51) Int. Cl. C07K 16/28; A61K 38/00; C07K 14/55

(52) U.S. Cl. CPC C07K 16/283; C07K 14/55; A61K 38/00; C07K 2317/31; C07K 2317/522; C07K 2317/524; C07K 2317/526; C07K 2317/53; C07K 2317/55; C07K 2317/622; C07K 2317/73; C07K 2319/30

(57) ABSTRACT

Multispecific proteins that bind NKp46 and specifically redirect effector cells to lyse a target cell of interest via multiple receptors are provided. The proteins have utility in the treatment of disease, notably cancer or infectious disease.

### CROSS-REFERENCE TO RELATED APPLICATIONS

This application claims the benefit of U.S. Provisional Application No. 63/208,511 filed 9 Jun. 2021, the disclosure of which is incorporated herein by reference in its entirety; including any drawings and sequence listings.

### REFERENCE TO THE SEQUENCE LISTING

The present application is being filed along with a Sequence Listing in electronic format. The Sequence Listing is provided as a file entitled “NKp46-13 PCT_ST25 txt”, created May 6, 2022, which is 326 KB in size. The information in the electronic format of the Sequence Listing is incorporated herein by reference in its entirety.

### FIELD OF THE INVENTION

Multispecific proteins that bind and specifically redirect effector cells to lyse a target cell of interest via multiple receptors are provided. The proteins have utility in the treatment of disease, notably cancer or infectious disease.

### BACKGROUND

Interleukin 2 (IL2 or IL-2) is one example of a pluripotent cytokine that acts on a cytokine receptor expressed by NK cells. IL-2 is mainly produced by activated T cells, especially CD4+T helper cells, and functions in aiding the proliferation and differentiation of B cells, T cells and NK cells. IL-2 is also essential for Treg function and survival. In eukaryotic cells, human IL-2 (uniprot: P60568) is synthesized as a precursor peptide of 153 amino acids with a 20 residue signal sequence, that gives rise to a mature secreted IL-2 having the amino acid sequence of SEQ ID NO: 352. Interleukin 2 has four antiparallel, amphiphilic alpha helices. These four alpha helices form a quaternary structure that is essential for its function. In most cases, IL-2 works through three different receptors: interleukin 2 receptor alpha (IL-2Rα; CD25), interleukin 2 receptor beta (IL-2Rβ; CD122), and interleukin 2 receptor gamma (IL-2Rγ; CD132). IL-2Rβ and IL-2Rγ are essential for IL-2 signaling, while IL-2Rα (CD25) is not necessary for signaling, but can confer high affinity binding of IL-2 to receptors. The trimer receptor (IL-2αβγ) formed by the combination of IL-2Rα, β, and γ is the IL-2 high affinity receptor (KD about 10 pM), and the dimer receptor (IL-2βγ) is an intermediate affinity receptor (KD about 1 nM).

Immune cells express dimer or trimer IL-2 receptors. Dimer receptors are expressed on cytotoxic CD8+T cells and natural killer cells (NK), while trimer receptors are mainly expressed on activated lymphocytes and CD4+CD25+FoxP3+inhibitory regulatory T cells (Treg). Because resting effector T cells and NK cells do not have CD25 on the cell surface, they are relatively insensitive to IL-2. Treg cells consistently express the highest level of CD25 in the body. Due to the low concentrations of IL-2 that typically exists in tissues, IL-2 preferentially activates cells that express the high affinity receptor complex (CD25:CD122:CD132), and therefore under normal circumstances, IL-2 will preferentially stimulate Treg cell proliferation.

IL-15, IL-12, IL-7, IL-27, IL-18, IL-21, and IFN-α share many aspects of receptor binding, complex assembly and signaling with IL-2. For example IL-15, IL-21 and IL-7 like IL-2 both act on NK cells via the common-γ chain receptor (CD132). IL-15 binds to the IL-15 receptor (IL-15R) which is composed of three subunits: IL-15Rα, CD122, and CD132. Two of these subunits, CD122 and CD132, are shared with the receptor for IL-2, but IL-2 receptor has an additional subunit (CD25). IL-15Rα (CD215) specifically binds IL15 with very high affinity, and is capable of binding IL-15 independently of other subunits. IL-21 is another example of a type I cytokine, and its IL-21 receptor (IL-21R) has been shown to form a heterodimeric receptor complex with the IL-2/IL-15 receptor common gamma chain (CD132).

NK cells have the potential to mediate anti-tumor immunity. However, NK cells have been shown to cause toxicity in mice through their hyper-activation and secretion of multiple inflammatory cytokines when IL-2 was administered together with IFN-α (Rothschilds et al, Oncoimmunology. 2019; 8(5):). In addition, NK cells were also shown to cause toxicity of the cytokine IL-15 that also signals through IL-2Rβγ (see WO2020247843 citing Guo et al, J Immunol. 2015; 195(5):2353-64).

One potential solution to the immune toxicity mediated by cytokines such as IL-2 was to fuse it to or associate it with a tumor-specific antibody. However, it was found that that while IL-2 indeed synergized with antitumor antibody in anti-tumor effect in vivo, the inclusion of IL-2 and anti-tumor antigen antibody in the same molecule presented no efficacy or toxicity advantage. The IL-2 moiety entirely governed biodistribution, explaining the observation that immunocytokines recognizing irrelevant antigen performed equivalently to tumor-specific immunocytokines when combined with antibody (Tzeng et al. Proc Natl Acad Sci USA. 2015 Mar. 17; 112(11): 3320-332).

Studies focusing on the effect of cytokines on NK cells have generally focused on single cytokines or simple combinations. More recently, it has been reported that IL-15, IL-18, IL-21, and IFN-α, alone and in combination, and their potential to synergize with IL-2, and that very low concentrations of both innate and adaptive common γ chain cytokines synergize with equally low concentrations of IL-18 to drive rapid and potent NK cell CD25 and IFN-γ expression (Nielsen et al. Front Immunol. 2016; 7: 101). However, administration of cytokines to humans has involved toxicity, which makes combination treatment with cytokines challenging. Furthermore, little remains known on potential synergies or interaction between cytokine receptor signaling pathways and other activating receptors in NK cells. There is therefore a need for new ways to mobilize NK cells in the treatment of disease, particularly cancer.

### SUMMARY OF THE INVENTION

The present invention arises from the discovery of functional multi-specific proteins that bind to NKp46 and a cytokine receptor (e.g. CD122 and/or CD132) on NK cells, and optionally that further bind CD16A on NK cells, and that also bind to an antigen of interest (e.g. a cancer antigen) on a target cell, where in the multi-specific proteins are capable of increasing NK cell cytotoxicity toward a target cell that expresses the antigen of interest (e.g., a cell that contributes to disease, a cancer cell). The advantages observed with these proteins are believed to result from their ability to co-engage NKp46, cytokine receptor (e.g. CD122 and/or CD132), and optionally further CD16A, on an NK cell. The examples made use of a variant IL-2 cytokine (IL-2v) which was modified to reduce affinity for its receptor(s) on T cells (CD25) but retained substantially full affinity of wild-type IL-2 for its receptor on NK cells (CD122 and/or CD132). The configurations of multispecific proteins were designed to present the respective antigen binding domains so as to permit co-engagement of NKp46 and cytokine receptor on the same cell surface plane (i.e. in NKp46, cytokine receptor (and further CD16A) are bound in cis). Further, the examples used a protein with a wild-type Fc domain that binds CD16A placed between the NKp46 binding domain and the cytokine, showing that binding to CD16A does not negatively affect the tumor and NK-targeted biodistribution and instead led to triple co-engagement of NKp46, CD16A and cytokine receptor, and in turn permitted the incorporation of a cytokine that retained binding affinity for its receptor on NK cells. By incorporating into the multispecific protein anti-NKp46 VH/VL domains that conferred a binding affinity for NKp46 in the low nanomolar range for the KD (KD of about 15 nM), cytokines could be used that retained substantially full binding affinity for their receptor on NK cells; the cytokines generally have a KD for binding to their receptor on NK cells that is no lower than that of the affinity of the multispecific protein for NKp46.

It is believed, in view of the results herein, that targeting the cytokine, e.g. a type 1 cytokine such as an IL-2, IL-15, IL-21, IL-7, IL-27 or IL-12 cytokine, an IL-18 cytokine or a type 1 interferon (e.g. IFN-α, IFN-β), to NKp46 promotes cis-presentation to the cytokine's receptor (e.g. IL2/15βγ, IL-21R, IL-7Ra, IL-27Ra, IL-12R, IL-18R, IFNAR), as shown in FIG. 1 for the cytokine IL2 and cytokine receptor complex IL2βγ). As shown herein, IL2v placed immediately adjacent to (and on the C-terminal side of) an Fc domain permitted the triple receptor cis-presentation to occur.

The multispecific proteins directed to NKp46 on NK cells have the advantage that they permit a range of cytokines to be used without a requirement for reduced binding affinity for their receptor on NK cells (e.g. CD122). While cytokokines can optionally be modified to have reduced binding affinity for their receptor on NK cells compared to their wild-type counterpart, it will also be possible to use a range of cytokines in the multispecific proteins that do not incorporate such modification or attenuation. The multispecific proteins directed to NKp46 on NK cells can thus make use of several cytokines in their wild-type form, particularly where the cytokine does not have substantially reduced activity at its receptor on NK cells, and/or where the cytokine's affinity for its receptor is no stronger than the affinity of the NKp46 ABD for NKp46. Accordingly, in any embodiment, the cytokine ABD (e.g. cytokine moiety within the multispecific protein) can be specified as having a binding affinity and/or an activity (e.g. induction of signalling) on its receptor on NK cells that is not substantially reduced compared to the wild-type form of the cytokine. Optionally, the cytokine moiety induces signalling at its receptor on NK cells (e.g. CD122) that is at least 70% or 80% of that observed with the wild-type form of the cytokine. Accordingly, in any embodiment, the cytokine ABD (e.g. cytokine moiety within the multispecific protein) can be specified as having an affinity for its receptor on NK cells that is not substantially reduced compared to the wild-type form of the cytokine. Optionally, the cytokine moiety has a binding affinity for its receptor on NK cells (e.g. CD122) that is within 3-log, 2-log or 1-log of that of the wild-type form of the cytokine (e.g. the cytokine moiety has a KD for binding to the cytokine receptor that is not more than 3-, 2- or 1-log higher that of the wild-type form of the cytokine). Affinity can be KD for binding to recombinant receptor protein, as determined using SPR. Signaling or receptor binding affinity of cytokines can be specified as being when incorporated into an otherwise equivalent multispecific protein.

There is therefore a particular advantage of a therapeutic molecule that combines the ability to bind each of NKp46 and cytokine receptor (e.g. CD122), and further CD16A, on an individual NK cell, particularly for a therapeutic agent having a long in vivo half-life. Combining these binding features in a single multispecific protein provided for greater in vivo anti-tumor activity compared to administering the agents (NKp46 multispecific protein and IL-2) separately.

The multispecific proteins are particularly advantageous due to high potency in enhancing NK cell activity (e.g. NK cell proliferation, activation, cytotoxicity and/or cytokine release, including by tumor-infiltrating NK cells), yet with low immune toxicity, e.g., low systemic increase or release of cytokines IL-6 and TNF-α. The present disclosure provides examples using protein formats that permit sufficient distance between NKp46 and cytokine receptor (e.g. CD122) and CD16A binding domains to permit all three receptors to be bound by a single NK cell, thereby providing combined NK cell receptor activation. Importantly, the combined binding on a single cell may account for the minimal off-target immune toxicity and lack of fratricidal killing of NKp46-expressing and/or CD16-expressing cells (e.g., NK cells) because the multispecific protein is bound by at least one activating receptor in addition to cytokine receptor (e.g. CD122) at the surface of the NKp46 and/or CD16+ effector cell.

The multispecific proteins can be useful to potentiate the activity and/or proliferation of both NKp46⁺CD16⁺ and NKp46⁺CD16A⁻ NK cells. Combined dual binding to NKp46 and CD122, even in the absence of binding to CD16A, demonstrates strong potentiation of NK cell activity. In healthy individuals, the CD16⁻ population represents 5-15% of the total NK cell population, while in some cancer patients the proportion of CD16⁻ NK cells is greatly increased, making up as much as 50% of the total NK cell population. Further, the tumor micro-environment has been shown to affect the phenotype of CD16A⁺ NK cells by either inducing shedding of CD16A from the surface of the cells or promoting conversion from CD16A⁺ to CD16⁻ NK cells. In addition, due to CD16A polymorphism, some individuals have mutations in CD16A (e.g. at residue 158 of CD16A) that result in reduced ability to mediate ADCC. Overcoming CD16A deficiencies, particular as may occur in the tumor environment, while increasing both the number of activated NKp46⁺ NK cells in the tumor, is particularly advantageous. Yet further, multispecific proteins do not require binding or signaling via NKG2D and can be used to potentiate NK cell activity in patients having NK and/or T cells characterized by relatively low levels of surface expression of the activating receptor NKG2D, for example as is known to be a general or common feature in gastric and prostate cancer.

Provided, inter alia, is a multispecific protein (e.g. an Fc-containing protein) comprising an Fc domain (e.g. an Fc dimer), a NKp46-binding domain that binds to a human NKp46 polypeptide, a binding domain that binds an antigen of interest (e.g. a tumor-associated or cancer antigen; an antigen of interest present expressed by a target cell), and an antigen binding domain that binds to a human cytokine receptor polypeptide expressed on NK cells. The Fc domain (e.g. the Fc dimer) can be specified as being interposed between the NKp46-binding domain and the antigen binding domain that binds to a human cytokine receptor polypeptide. In one embodiment, the ABD that binds to a human NKp46 polypeptide is connected at its C-terminus to the N-terminus of Fc domain, optionally via an Ig-derived (e.g. hinge domain or portion thereof) or non-Ig-derived polypeptide linker, and the Fc domain is connected at its C-terminus to the N-terminus of the the ABD that binds a human cytokine receptor is connected, via a polypeptide linker.

The Fc domain (e.g. an Fc monomer part of the Fc dimer), the NKp46-binding domain (or a part thereof, e.g. a variable region or variable region-CH1/CK unit) and the ABD that binds to a human cytokine receptor can optionally be specified as being placed on the same polypeptide chain (the first polypeptide chain), for example a NKp46-binding domain portion is fused at its C-terminus to the N-terminus of an Fc monomer via a hinge domain sequence or any domain linker, in turn fused at its C-terminus to the N-terminus of the ABD that binds to a human cytokine receptor via a domain linker. The remaining elements of the protein (complementary part of the NKp46 binding domain, complementary Fc monomer, binding domain that binds an antigen of interest) can be placed on one or more additional polypeptide chains that dimerize with the first polypeptide chain. The NKp46-binding domain, the Fc domain and the cytokine can thus be able to adopt a membrane-planar binding configuration.

In one embodiment, the Fc domain is fused at its C-terminus to the ABD that binds a human cytokine receptor by a linker peptide having 20 or less than 20 amino acid residues, optionally less than 15 amino acid residues, optionally 10 or less than 10 amino acid residues, optionally between 5 and 15 residues, optimally between 5 and 10 residues, optionally between 3 and 5 residues.

In one embodiment, protein has only one ABD that binds to a human NKp46 polypeptide, only one ABD that binds an antigen of interest, only one dimeric Fc domain, and only one ABD that binds a human cytokine receptor such that the protein exhibits monovalent binding to each of NKp46, CD16A, antigen of interest and the human cytokine receptor. A multispecific protein can optionally be characterized as having the NKp46-binding domain and the binding domain that binds an antigen of interest positioned N-terminal to the Fc dimer within the topology of the multispecific protein, and the human cytokine receptor polypeptide positioned C-terminal to the Fc dimer within the topology of the multispecific protein.

The Fc domain can be specified as binding to a human FcRn polypeptide optionally with or optionally without binding to a human CD16A polypeptide.

Provided, for example, is a multispecific protein (e.g. an Fc-containing protein) comprising an Fc domain (e.g. an Fc dimer), a NKp46-binding domain that binds to a human NKp46 polypeptide positioned N-terminal to the Fc domain, a binding domain that binds an antigen of interest (e.g. a tumor-associated or cancer antigen; an antigen of interest present expressed by a target cell) positioned N-terminal to the Fc domain, and an antigen binding domain that binds to a human cytokine receptor polypeptide expressed on NK cells positioned C-terminal to the Fc domain. The cytokine receptor can be for example CD122 (IL2/15Rβ), IL-21R, IL-7Ra, IL-27Ra, IL-12R, IL-18R, IFNAR (IFNAR1 and/or IFNAR2). The Fc domain can be specified as binding to a human FcRn polypeptide optionally with or optionally without binding to a human CD16A polypeptide.

The antigen binding domain that binds a cytokine receptor can be a variant cytokine having a modification that reduces binding to a receptor counterpart found on non-NK cells (e.g. T cells, Treg cells) compared to its wild-type form, positioned at the C-terminal of the polypeptide chain on which it is found. The cytokine can be specified as being positioned at the C-terminus topologically within the protein and/or at the C-terminus of the polypeptide chain(s) on which it is placed.

The antigen binding domain that binds a cytokine receptor can be a human cytokine polypeptide (e.g. IL-2, IL-15, IL-21) that is modified (e.g. by introducing amino acid modifications) to reduce the binding affinity for a cytokine receptor to which it binds, optionally wherein binding affinity is selectively reduced for a receptor not expressed at the surface of NK cells. As further described herein, where a cytokine has more than one receptor as its natural binding partner and one of the receptors is expressed on non-NK cells, the cytokine polypeptide can be modified to reduce binding to such receptor that is expressed on non-NK cells (e.g. Treg cells, T cells) compared to its wild-type cytokine counterpart.

In one embodiment, exemplified by the protein incorporating the CDRs of the NKp46-1 VH/VL pair, the NKp46-binding domain binds to the D1/D2 junction of the NKp46 polypeptide. Based on x-ray crystallography studies of NKp46, it is believed that the D1/D2 junction of the NKp46 polypeptide is positioned at about 70 Angstroms from the cell surface, which corresponds to the predicted distance from the cell surface for the cytokine binding site of CD122. Binding to the D1/D2 junction of the NKp46 polypeptide and/or to the region or epitope bound by NKp46-1 can provide a positioning of the NKp46 ABD at a distance from the NK cell surface that permits optimal engagement of a cytokine receptor such as CD122. In turn, domain linkers of reduced length (e.g. between 2 and 5 residues, between 2 and 10 residues; 3, 4, 5, 6, 7, 8, 9 or 10 residues) can be used between the cytokine and the NKp46 ABD or rest of the multispecific protein without any decrease in potency. When other domains on NKp46 are bound, longer domain linkers can be used, e.g. between 5 and 15 residues, between 10 and 15 residues, or more.

In one embodiment, the multispecific protein comprises an Fc domain or portion thereof fused, optionally via a domain linker, to a cytokine receptor-binding domain, e.g. a cytokine that binds a receptor expressed at the surface of an NK cell.

In one embodiment, the multispecific protein is a trimer (e.g. a heterotimer) comprising:
(i) a first polypeptide chain comprising, from N- to C-terminal, an scFv that binds an NKp46 polypeptide, optionally a domain linker or a hinge polypeptide, a CH2 domain, a CH3 domain, optionally a domain linker, and a wild-type or variant IL-2, IL-15, IL-21, IL-7, IL-27, IL-12, IL-18, IFN-α or IFN-β polypeptide, and
(ii) a second polypeptide chain comprising, from N- to C-terminal, a variable domain of a binding domain that binds an antigen of interest, a human CH1 or CL constant domain, optionally a domain linker or hinge polypeptide, a CH2 domain, and a CH3 domain; and
(iii) a third polypeptide chain comprising, from N- to C-terminal, a variable domain that associates with the variable domain of (ii) to form a binding domain that binds an antigen of interest, and a human CH1 or CL constant domain,
wherein one of the variable domains of (ii) and (iii) is a VH and the other is a VL wherein one of the constant domains of (ii) and (iii) is a CH1 and the other is a CL such that the constants domains of (ii) and (iii) associate by CH1-CL dimerization. Accordingly, chains (ii) and (iii) can dimerize to form a Fab structure and chains (i) and (ii) dimerize via CH3-CH3 interactions to form a dimeric Fc domain.

In one embodiment, the multispecific protein is a trimer (e.g. a heterotimer) comprising:
(i) a first polypeptide chain comprising, from N- to C-terminal, a variable domain of an NKp46-binding domain, a human CH1 or CL constant domain, optionally a domain linker or hinge polypeptide, a CH2 domain, a CH3 domain, optionally a domain linker, and a wild-type or variant IL-2, IL-15, IL-21, IL-7, IL-27, IL-12, IL-18, IFN-α or IFN-β polypeptide, and
(ii) a second polypeptide chain comprising, from N- to C-terminal, a variable domain that associates with the variable domain of chain (i) to form a NKp46-binding domain, and a human CH1 or CL constant domain;
wherein one of the variable domains of (i) and (ii) is a VH and the other is a VL wherein one of the constant domains of chains (i) and (ii) is a CH1 and the other is a CL such that the constants domains of chains (i) and (ii) associate by CH1-CL dimerization; and
(iii) a third polypeptide chain comprising, from N- to C-terminal, an svFc that binds an antigen of interest, a domain linker or hinge polypeptide, a CH2 domain, and a CH3 domain. Accordingly, chains (i) and (ii) can dimerize to form a Fab structure and chains (i) and (iii) dimerize via CH3-CH3 interactions to form a dimeric Fc domain.

In one embodiment, the multispecific protein is a tetramer (e.g. a heterotetramer) comprising:
(i) a first polypeptide chain comprising, from N- to C-terminal, a variable domain of an NKp46-binding domain, a human CH1 or CL constant domain, optionally a domain linker or hinge polypeptide, a CH2 domain, a CH3 domain, optionally a domain linker, and a wild-type or variant IL-2, IL-15, IL-21, IL-7, IL-27, IL-12, IL-18, IFN-α or IFN-β polypeptide, and
(ii) a second polypeptide chain comprising, from N- to C-terminal, a variable domain that associates with the variable domain of (i) to form a NKp46-binding domain, and a human CH1 or CL constant domain;
wherein one of the variable domains of (i) and (ii) is a VH and the other is a VL wherein one of the constant domains of (i) and (ii) is a CH1 and the other is a CL such that the constants domains of (i) and (ii) associate by CH1-CL dimerization; and
(iii) a third polypeptide chain comprising, from N- to C-terminal, a variable domain of a binding domain that binds an antigen of interest, a human CH1 or CL constant domain, optionally a domain linker or hinge polypeptide, a CH2 domain, and a CH3 domain; and
(iv) a fourth polypeptide chain comprising, from N- to C-terminal, a variable domain that associates with the variable domain of (iii) to form a binding domain that binds an antigen of interest, and a human CH1 or CL constant domain,
wherein one of the variable domains of (iii) and (iv) is a VH and the other is a VL wherein one of the constant domains of (iii) and (iv) is a CH1 and the other is a CL such that the constants domains of (iii) and (iv) associate by CH1-CL dimerization. Accordingly, chains (i) and (ii) can dimerize to form a Fab structure, chains (iii) and (iv) can dimerize to form a Fab structure, and chains (i) and (ili) dimerize via CH3-CH3 interactions to form a dimeric Fc domain.

In one embodiment, the multispecific protein is a tetramer e.g. a heterotetramer) comprising:
(i) a first polypeptide chain comprising, from N- to C-terminal, a variable domain of an NKp46-binding domain, a human CH1 or CL constant domain, optionally a domain linker or hinge polypeptide, a CH2 domain, a CH3 domain, and
(ii) a second polypeptide chain comprising, from N- to C-terminal, a variable domain that associates with the variable domain of (i) to form a NKp46-binding domain, and a human CH1 or CL constant domain;
wherein one of the variable domains of (i) and (ii) is a VH and the other is a VL wherein one of the constant domains of (i) and (ii) is a CH1 and the other is a CL such that the constants domains of (i) and (ii) associate by CH1-CL dimerization; and
(iii) a third polypeptide chain comprising, from N- to C-terminal, a variable domain of a binding domain that binds an antigen of interest, a human CH1 or CL constant domain, optionally a domain linker or hinge polypeptide, a CH2 domain, a CH3 domain, optionally a domain linker, and a wild-type or variant IL-2, IL-15, IL-21, IL-7, IL-27, IL-12, IL-18, IFN-α or IFN-β polypeptide;
(iv) a fourth polypeptide chain comprising, from N- to C-terminal, a variable domain that associates with the variable domain of (iii) to form a binding domain that binds an antigen of interest, and a human CH1 or CL constant domain, wherein one of the variable domains of (iii) and (iv) is a VH and the other is a VL wherein one of the constant domains of (iii) and (iv) is a CH1 and the other is a CL such that the constants domains of (iii) and (iv) associate by CH1-CL dimerization. Accordingly, chains (i) and (ii) can dimerize to form a Fab structure, chains (iii) and (iv) can dimerize to form a Fab structure, and chains (i) and (ili) dimerize via CH3-CH3 interactions to form a dimeric Fc domain.

In one embodiment, the multispecific protein is a dimer (e.g. a heterodimer) comprising:
(i) a first polypeptide chain comprising, from N- to C-terminal, an scFv that binds an NKp46 polypeptide, optionally a domain linker or hinge polypeptide, a CH2 domain, a CH3 domain, optionally a domain linker, and a wild-type or variant IL-2, IL-15, IL-21, IL-7, IL-27, IL-12, IL-18, IFN-α or IFN-β polypeptide, and
(ii) a second polypeptide chain comprising, from N- to C-terminal, an scFv that binds an antigen of interest, a domain linker or hinge polypeptide, a CH2 domain, and a CH3 domain. Accordingly, chains (i) and (ii) dimerize via CH3-CH3 interactions to form a dimeric Fc domain.

In one embodiment, the multispecific protein is a dimer (e.g. a heterodimer) comprising:
(i) a first polypeptide chain comprising, from N- to C-terminal, an scFv that binds an antigen of interest, optionally a domain linker or hinge polypeptide, a CH2 domain, a CH3 domain, optionally a domain linker, and a wild-type or variant IL-2, IL-15, IL-21, IL-7, IL-27, IL-12, IL-18, IFN-α or IFN-β polypeptide, and
(ii) a second polypeptide chain comprising, from N- to C-terminal, an svFc that binds an NKp46 polypeptide, a domain linker or hinge polypeptide, a CH2 domain, and a CH3 domain. Accordingly, chains (i) and (ii) dimerize via CH3-CH3 interactions to form a dimeric Fc domain.

In one aspect of any embodiment herein, the cytokine or cytokine receptor ABD binds its receptor, as determined by SPR, with a binding affinity (KD) of 1 μM or lower, 200 nM or lower, 100 nM or lower, 50 nM or lower, or 25 nM or lower, as tested either as free cytokine or as incorporated into the multispecific protein). In one embodiment, the cytokine or cytokine receptor ABD binds its receptor, as determined by SPR, with a binding affinity (KD) that is 1 nM or higher than 1 nM, optionally that is higher than 10 nM optionally that is higher than 15 nM. In one embodiment, the cytokine or cytokine receptor ABD binds its receptor, as determined by SPR, with a binding affinity (KD) between about 1 nm and about 200 nm, optionally between about 1 nm and about 100 nm, optionally between about 10 nM and about 1 μM, optionally between about 10 nM and about 200 μM, optionally between about 10 nM and about 100 nM, optionally between about 15 nM and about 1 μM, or optionally between about 15 nM and about 200 nM.

In one embodiment, the cytokine is a wild-type cytokine or a fragment or variant thereof that has at least 80% of the ability of a wild-type cytokine counterpart to induce signaling in NK cells, optionally wherein signaling is assessed by bringing the isolated cytokine moiety into contact with an NK cell and measuring STAT phosphorylation in the NK cells. In one embodiment, the cytokine is a wild-type cytokine or fragment thereof that retains at least 50%, 60%, 70%, 80% or 90% of the affinity for its cytokine receptor present on NK cells, compared to the wild-type cytokine counterpart. In one embodiment, the cytokine is a variant cytokine, wherein the cytokine retains at least 50%, 60%, 70%, 80% or 90% of the affinity for its cytokine receptor present on NK cells, compared to the wild-type cytokine counterpart. In one embodiment, the cytokine does not comprise mutations that substantially reduce the affinity of the cytokine for the cytokine receptor present on NK cells. In one embodiment, the multispecific protein (or the cytokine when included in the multispecific protein) exhibits an EC50 for cytokine pathway signaling in NK cells that is lower than that observed with its wild-type cytokine counterpart alone. In one embodiment, the multispecific protein (or the cytokine when included in the multispecific protein) exhibits an EC50 for cytokine pathway signaling in NK cells that is lower than that observed with the cytokine alone or in a protein of comparable structure but lacking a NKp46 ABD and/or a CD16 ABD. Optionally the EC50 for cytokine pathway signaling in NK cells is at least 10-fold or 100-fold lower, optionally wherein cytokine pathway signaling is assessed by bringing the respective cytokine or multispecific protein into contact with an NK cell and measuring STAT phosphorylation in the NK cells.

In one embodiment, the multispecific protein is configured such that an Fc domain (or CD16-binding domain), the NKp46-binding domain and the cytokine receptor-binding domain are each capable of binding to their respective NKp46, CD16A or cytokine receptor binding partner when such binding partners are present together at the surface of a cell (e.g. an NK cell). In some embodiments, the multispecific protein can be characterized by monovalent binding to NKp46 (e.g. the multispecific protein comprises only one NKp46 ABD), monovalent (or optionally bivalent) binding to antigen of interest, monovalent binding to CD16A (e.g. the multispecific protein comprises only one Fc domain dimer), and monovalent binding to cytokine receptor (e.g., the multispecific protein comprises only one cytokine receptor ABD).

In one embodiment, the multispecific protein is configured e.g., through placement or configuration of the domain within a multispecific protein, optionally through use of one or more using domain linkers having a maximal potential length of 18 Angstroms (5 amino acid residues), 36 Angstroms (10 residues) or 54 Angstroms (15 residues) when in a stretched configuration such that the NKp46-binding domain and the cytokine receptor-binding domain, and the CD16-binding domain when present and capable of binding CD16, can assume a membrane planar binding conformation such that each of NKp46, CD16A and cytokine receptor are bound at the surface of an NK cell.

For example, the multispecific protein comprises a Fc domain dimer comprised of a first and second Fc domain monomer positioned on different polypeptide chains (that dimerize via CH3-CH3 association). The first Fc domain monomer can be fused at its N-terminus to an anti-NKp46 ABD (or portion thereof), and at its C-terminus to a cytokine. The portion of an anti-NKp46 ABD can be for example a ((VH or VL)-CH1) unit or ((VH or VL)-CL) unit where the ABD is a Fab. The second Fc domain monomer can be fused at its N-terminus to an ABD that binds an antigen of interest (or portion thereof). The portion of ABD that binds an antigen of interest can be for example a ((VH or VL)-CH1) unit or ((VH or VL)-CL) unit where the ABD is a Fab. FIGS. 2-4 show exemplary domain configurations.

In any embodiment, the cytokine receptor-binding domain (cytokine receptor ABD), the NKp46-binding domain (NKp46 ABD) and the CD16-binding domain (CD16 ABD) can be specified as being placed within the one or more polypeptide chains that make up the multispecific protein so that the domains are oriented in a configuration in which they are adjacent to one another on the multimeric (e.g. heteromultimeric) protein. Domains can be optionally separated by a domain linker e.g. a linking peptide of 5-20 residues that does not itself bind to a predetermined antigen.

In any embodiment, the multispecific protein can be specified as being configured e.g., through placement or configuration of the domain within a multispecific protein, such that the NKp46 ABD and the cytokine receptor ABD (e.g. the cytokine moiety) have the ability to assume a position where they are on the same side or face of the Fc domain dimer within the multispecific protein molecule, so as to enhance the ability to bind NKp46, CD16A and cytokine receptor in a membrane planar binding conformation. Optionally, in the heterodimeric, heterotrimeric or heterotetrameric proteins of the disclosure, the NKp46 ABD (or a part thereof, if the ABD is formed from association of two polypeptide chains) and the cytokine receptor ABD (or a part thereof, if the ABD is formed from association of two polypeptide chains) are positioned on the same polypeptide chain, together with one of the Fc domain monomers.

In one embodiment, the NKp46-binding domain binds NKp46 such that the NKp46 binding domain of the multispecific protein, when bound to NKp46 at the surface of a cell, is about 70 Angstroms from the cell membrane. In one such embodiment, exemplified by the protein incorporating the CDRs of the NKp46-1 VH/VL pair, the NKp46-binding domain binds to the D1/D2 junction of the NKp46 polypeptide. Optionality, the NKp46-binding domain exhibits decreased binding to the NKp46 mutant 2 (having a mutation at residues K41, E42 and E119) and mutant Supp7 (having a mutation at residues Y121 and Y194) compared to the wild-type NKp46 polypeptide. In one embodiment, an NKp46 antigen binding domain can be characterized as displaying decreased binding to a NKp46 mutant polypeptide having one, two, three, four or five of the mutations: K41, E42, E119, Y121 and Y194 compared to a wild-type NKp46 polypeptide.

In another embodiment, exemplified by the protein incorporating the CDRs of the NKp46-3 VH/VL pair, the NKp46-binding domain binds to the D2 domain of the NKp46 polypeptide which is positioned more proximal to the NK cell membrane compared to the D1/D2 junction. Optionality, the NKp46-binding domain exhibits decreased binding to the NKp46 mutant 19 (having a mutation at residues 1135, and S136) and mutant Supp8 (having a mutation at residues P132 and E133) compared to the wild-type NKp46 polypeptide. In one embodiment, an NKp46 antigen binding domain can be characterized as displaying decreased binding to a NKp46 mutant polypeptide having one, two, three or four of the mutations: 1135, S136, P132 and E133 compared to a wild-type NKp46 polypeptide. In one embodiment, the multispecific protein comprises a domain linker of at least 10 amino acid residues between an NKp46 binding domain that binds within the D2 domain and the cytokine.

In any embodiment herein, the multispecific protein the multispecific protein can be characterized by having only one (a single) cytokine receptor binding domain.

In any embodiment herein, the multispecific protein the multispecific protein can be characterized by having only one (a single) NKp46 binding domain.

The NKp46 ABD can conveniently be a Fab, a single domain antibody or an scFv. The Fc domain monomer or dimer can be of human IgG1, IgG2, IgG3 or IgG4 subtype, optionally comprising one or more (e.g. 1-5, 1-10) amino acid substitutions or other modifications). The cytokine (Cyt) can be for example an IL-2, IL-15, IL-21, IL-7, IL-27, IL-12, IL-18, IFN-α or IFN-β) polypeptide, optionally wherein the polypeptide is a variant cytokine that differs by at least one amino acid residue from the wild-type human cytokine counterpart.

In one aspect the protein comprises an ABD that binds an antigen of interest on a target cell (Antigen ABD) and the ABD that binds NKp46 both placed topologically N-terminal to the Fc domain dimer, as in a heteromultimeric protein having the structure below (topological N-terminus on left and C-terminus on right):
(NKp46 ABD)
   (Fc domain dimer) (Cyt)
(Antigen ABD)
wherein the Antigen ABD and the Fc domain dimer are connected by a linker or immunoglobulin hinge polypeptide, wherein the NKp46 ABD and the Fc domain dimer are connected by a linker or immunoglobulin hinge polypeptide, and wherein the Fc domain dimer and Cyt are connected by a linker.

In one embodiment, the multispecific protein binds monovalently to each of the NKp46 polypeptide and the cytokine receptor, and the multispecific protein is capable of directing an NKp46-expressing NK cell to lyse a target cell expressing the antigen of interest.

Advantageously, in on embodiment, the presence of NK cells and target cells, the multi-specific protein can bind (i) to antigen of interest on target cells, (ii) to NKp46 on NK cells, (iii) to CD16A on NK cells and (iv) to the cytokine receptor on NK cells (e.g. CD122, IL-21R, IL-7Ra, IL-27Ra, IL-12R, IL-18R, IFNAR), and when bound to such proteins on the target cell and NK cells, can induce signaling in and/or activation of the NK cells through NKp46 (the protein acts as an NKp46 agonist) and the cytokine receptor (the protein acts as a cytokine receptor agonist), thereby promoting activation of NK cells and/or lysis of target cells, notably via the activating signal transmitted by NKp46.

In one embodiment, in the presence of NK cells and target cells, the multi-specific protein can induce the cytotoxicity of, cytokine receptor pathway signaling in (as assessed by STAT signaling) and/or activation of the NK cells, wherein such cytotoxicity, activation and/or signaling is greater (e.g. at least 100-fold or 1000-fold lower EC₅₀ value) than that observed when the multi-specific protein is contacted with NK cells in the absence of target cells.

Optionally, the multi-specific protein can bind NKp46 and CD122 on NK cells (e.g. the protein comprises an IL2 or IL15 moiety, optionally an modified or variant IL2 or IL15 with decreased binding to CD25), and, when bound to both NKp46 and CD122, can induce signaling in the NK cells through both NKp46 and CD122. Optionally, the multi-specific protein can bind NKp46, CD16A and CD122 on NK cells, and, when bound to NKp46, CD16 and CD122, can induce signaling in the NK cells through NKp46, CD16A and CD122. Signalling via NKp46 and/or CD16A can be assessed by a marker of NK cell activation (e.g. a marker used in the Examples, CD69 expression, etc.). Optionally, cytokine signaling is assessed by measuring STAT5, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-21R on NK cells (e.g. the protein comprises an IL21 moiety), and, when bound to both NKp46 and IL-21R, can induce signaling in the NK cells through both NKp46 and IL-21R. Optionally, the multi-specific protein can bind NKp46, CD16A and IL-21R on NK cells, and, when bound to NKp46, CD16A and IL-21R, can induce signaling in the NK cells through NKp46, CD16A and IL-21R. Signalling via NKp46 and/or CD16A can be assessed by a marker of NK cell activation (e.g. a marker used in the Examples, CD69 expression, etc.). Optionally, cytokine signaling is assessed by measuring STAT3, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-18R on NK cells (e.g. the protein comprises an IL18 moiety), and, when bound to both NKp46 and IL-18R (IL-18Ra and/or IL-18Rβ), can induce signaling in the NK cells through both NKp46 and IL-18R. Optionally, the multi-specific protein can bind NKp46, CD16A and IL-18R on NK cells, and, when bound to NKp46, CD16A and IL-18R, can induce signaling in the NK cells through both NKp46, CD16A and IL-18R. Signalling via NKp46 and/or CD16A can be assessed by a marker of NK cell activation (e.g. a marker used in the Examples, CD69 expression, etc.). Optionally, cytokine signaling is assessed by measuring STAT3, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-7R (e.g. IL-7Rα (CD127) and/or CD132) on NK cells (e.g. the protein comprises an IL-7 moiety), and, when bound to both NKp46 and IL-7R, can induce signaling in the NK cells through both NKp46 and IL-7Ra. Optionally, the multi-specific protein can bind NKp46, CD16A and IL-7R on NK cells, and, when bound to NKp46, CD16A and IL-7R, can induce signaling in the NK cells through both NKp46, CD16A and IL-7R. Signalling via NKp46 and/or CD16A can be assessed by a marker of NK cell activation (e.g. a marker used in the Examples, CD69 expression, etc.). Optionally, cytokine signaling is assessed by measuring STAT5, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-27R (e.g. IL-27Rα and/or GP130) on NK cells (e.g. the protein comprises an IL-27 moiety), and, when bound to both NKp46 and IL-27R, can induce signaling in the NK cells through both NKp46 and IL-27R. Optionally, the multi-specific protein can bind NKp46, CD16A and IL-27R on NK cells, and, when bound to NKp46, CD16A and IL-27R, can induce signaling in the NK cells through both NKp46, CD16A and IL-27R. Signalling via NKp46 and/or CD16A can be assessed by a marker of NK cell activation (e.g. a marker used in the Examples, CD69 expression, etc.). Optionally, cytokine signaling is assessed by measuring STAT1, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-12R (e.g., IL-12Rβ1 and/or IL-12Rβ2) on NK cells (e.g. the protein comprises an IL-27 moiety), and, when bound to both NKp46 and IL-12R, can induce signaling in the NK cells through both NKp46 and IL-12R. Optionally, the multi-specific protein can bind NKp46, CD16A and IL-12R on NK cells, and, when bound to NKp46, CD16A and IL-12R, can induce signaling in the NK cells through both NKp46, CD16A and IL-12R. Signalling via NKp46 and/or CD16A can be assessed by a marker of NK cell activation (e.g. a marker used in the Examples, CD69 expression, etc.). Optionally, cytokine signaling is assessed by measuring STAT4, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IFNAR on NK cells, and, when bound to both NKp46 and IFNAR (IFNAR1 and/or IFNAR2), can induce signaling in the NK cells through both NKp46 and IFNAR. For example, the multi-specific protein can comprise an IFN-α or IFN-β moiety) Optionally, the multi-specific protein can bind NKp46, CD16A and IFNAR on NK cells, and, when bound to both NKp46, CD16A and IFNAR, can induce signaling in the NK cells through NKp46, CD16A and IFNAR. Signalling via NKp46 and/or CD16A can be assessed by a marker of NK cell activation (e.g. a marker used in the Examples, CD69 expression, etc.). Optionally, cytokine signaling is assessed by measuring STAT (e.g., STAT1, STAT2 or IFN regulatory factor (IRF)-9), wherein the observed signaling is greater than that observed with a comparator protein in which the NKp46 binding domain is replaced with a control ABD (e.g. that does not bind to any protein present in the assay system).

In some embodiment, the multispecific protein comprises a full-length Fc domain or at least a portion of a human Fc domain sufficient such that the Fc domain is bound by a human FcRn polypeptide, optionally wherein said FcRn binding affinity as assessed by SPR is within 1-log of that of a conventional human IgG1 antibody.

The multispecific proteins advantageously are able to potently mobilize both CD16⁺ and CD16⁻ NK cells (all NK cells are NKp46⁺).

In one aspect, the multispecific protein comprises two or more polypeptide chains, i.e. it comprises a multi-chain protein (also referred as a multimeric protein). For example, the multispecific protein or multi-chain protein can be a hetero-dimer, hetero-trimer or hetero-tetramer or may comprise more than four polypeptide chains.

Any antigen binding domain (e.g. the ABD that binds the antigen of interest (e.g. tumor antigen), NKp46, or cytokine receptor) can be contained entirely on a single polypeptide chain, for example as an scFv or single antigen binding domain such as a sdAb or nanobody, a V_{NAR} or VHH domain or a DARPin® protein module). Alternatively, an antigen binding domain can be made of two or more protein domains placed on separate polypeptide chains, such that the antigen binding domain binds its target when two or more complementary protein domains (e.g. as VH/VL pairs) are associated in the multimeric protein.

An ABD can be connected to an Fc domain monomer (or CH2 or CH3 domain thereof) via a flexible domain linker (optionally via intervening sequences such as constant region domains or portions thereof, e.g. CH1 or CK). The linker can be a polypeptide linker, for example peptide linkers comprising a length of at least 5 residues, at least 10 residues, at least 15 residues, at least 20 residues, or more. In other embodiments, the linkers comprises a length of between 2-4 residues, between 2-5 residues, between 2-6 residues, between 2-8 residues, between 5-10 residues, between 2-15 residues, between 4-15 residues, between 5-15 residues, between 10-15 residues, between 4-20 residues, between 5-20 residues, between 2-20 residues, between 10-30 residues, or between 10-50 residues. Optionally a linker comprises an amino acid sequence derived from an antibody constant region, e.g., an N-terminal CH1 or a hinge sequence. Optionally a linker comprises the amino acid sequence RTVA. Optionally a linker is a flexible linker predominantly or exclusively comprised of glycine and/or serine residues, e.g., comprising an amino acid sequence (GₓS)ₙ where G is 1, 2, 3 or 4 and n is an integer from 1-10, from 1-6 or from 1-4. Optionally the linker comprises 1-20 or 1-10 further amino acid residues, for example the amino acid sequence GEGTSTGS(G₂S)₂GGAD.

In one embodiment, provided is a heterotrimer having a polypeptide chain 1, 2 and 3:

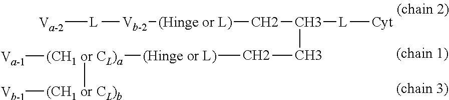

wherein:
Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD) that binds an antigen of interest, wherein one of Vₐ₋₂ and V_{b-2} is a V_{H} and the other is a V_{L} and wherein Vₐ₋₂ and V_{b-2} form a second ABD that binds NKp46;
CH1 is a human immunoglobulin CH1 domain and CL is a light chain constant domain;
one of (CH1 or CL)ₐ and (CH1 or CL)_{b} is a CH1 and the other is a CL such that a (CH1/CL) pair is formed;
Hinge is an immunoglobulin hinge region or portion thereof;
L is an amino acid domain linker, wherein each L can be different or the same;
CH2 and CH3 are human immunoglobulin CH2 and CH3 domains, respectively; and
Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

In one embodiment, provided is a heterotrimer having a polypeptide chain 1, 2 and 3:

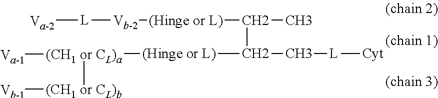

wherein:
Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD) that binds NKp46, wherein one of Vₐ₋₂ and V_{b-2} is a V_{H} and the other is a V_{L} and wherein Vₐ₋₂ and V_{b-2} form a second ABD that an binds antigen of interest;
CH1 is a human immunoglobulin CH1 domain and CL is a light chain constant domain;
one of (CH1 or CL)ₐ and (CH1 or CL)_{b} is a CH1 and the other is a CL such that a (CH1/CL) pair is formed;
Hinge is an immunoglobulin hinge region or portion thereof;
L is an amino acid domain linker, wherein each L can be different or the same;
CH2 and CH3 are human immunoglobulin CH2 and CH3 domains, respectively; and
Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

In one embodiment, provided is a heterotetramer having a polypeptide chain 1, 2, 3 and 4:

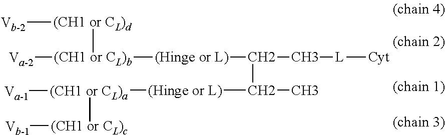

wherein:
Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD) that binds an antigen of interest, wherein one of Vₐ₋₂ and V_{b-2} is a V_{H} and the other is a V_{L} and wherein Vₐ₋₂ and V_{b-2} form a second ABD that binds NKp46;
CH1 is a human immunoglobulin CH1 domain and CL is a light chain constant domain;
one of (CH1 or CL)ₐ and (CH1 or CL)_{c} is a CH1 and the other is a CL such that a (CH1/CL) pair is formed;
one of (CH1 or CL)_{b} and (CH1 or CL)_{d} is a CH1 and the other is a CL such that a (CH1/CL) pair is formed;
Hinge is an immunoglobulin hinge region or portion thereof;
L is an amino acid domain linker, wherein each L can be different or the same;
CH2 and CH3 are human immunoglobulin CH2 and CH3 domains, respectively; and
Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

In proteins herein, when a hinge polypeptide is present in chain 1, a hinge polypeptide will also be present in chain 2. Polypeptides chains 1 and 2 can thus form and be bound to one another by interchain disulfide bonds.

The disclosure further provides further heterodimer, heterotrimer and heterotetramer multispecific molecules and domain arrangements as further described herein. In one aspect, a multispecific protein is a heteromultimer, heterodimer, heterotrimer, heterotetramer having a structure or domain arrangement as shown in any of FIGS. 2-4.

In one aspect of any of the embodiments described herein, an ABD (e.g., the anti-NKp46 ABD, the ABD that binds the antigen of interest or tumor antigen) can be specified as comprising an immunoglobulin heavy chain variable domain (VH) and an immunoglobulin light chain variable domain (VL), wherein each VH and VL comprises three complementary determining regions (CDR-1, CDR-2 and CDR-3). Optionally, CDRs are derived from a non-human mammal, e.g. a mouse or rat. In one aspect of any of the embodiments described herein, a VH can be specified as having the amino acid sequence of a human VH domain (e.g. frameworks and optionally further CDRs derived or originating from a human IGHV gene). In one aspect of any of the embodiments described herein, a VL can be specified as having the amino acid sequence of a human VL domain (e.g. frameworks and optionally further CDRs derived or originating from a human IGKV or IGLV gene).

In one aspect of any of the embodiments, a VH region comprises an amino acid sequence having at least about 80%, 85%, 90%, 95%, 97%, 98% or 99% identity, to the amino acid sequence encoded by a gene of a human V gene group selected from the group consisting of IGHV1-18, IGHV1-2, IGHV1-24, IGHV1-3, IGHV1-45, IGHV1-46, IGHV1-58, IGHV1-69, IGHV1-69-2, IGHV1-69D, IGHV1-8, IGHV2-26, IGHV2-5, IGHV2-70, IGHV2-70D, IGHV3-11, IGHV3-13, IGHV3-15, IGHV3-20, IGHV3-21, IGHV3-23, IGHV3-23D, IGHV3-30, IGHV3-30-3, IGHV3-30-5, IGHV3-33, IGHV3-43, IGHV3-43D, IGHV3-48, IGHV3-49, IGHV3-53, IGHV3-62, IGHV3-64, IGHV3-64D, IGHV3-66, IGHV3-7, IGHV3-72, IGHV3-73, IGHV3-74, IGHV3-9, IGHV3-NL1, IGHV4-28, IGHV4-30-2, IGHV4-30-4, IGHV4-31, IGHV4-34, IGHV4-38-2, IGHV4-39, IGHV4-4, IGHV4-59, IGHV4-61, IGHV5-10-1, IGHV5-51, IGHV6-1, IGHV7-4-1, IGHV1-38-4, IGHV1/OR15-1, IGHV1/OR15-5, IGHV1/OR15-9, IGHV1/OR21-1, IGHV2-70, IGHV2/OR16-5, IGHV3-16, IGHV3-20, IGHV3-25, IGHV3-35, IGHV3-38, IGHV3-38-3, IGHV3/OR15-7, IGHV3/OR16-10, IGHV3/OR16-12, IGHV3/OR16-13, IGHV3/OR16-17, IGHV3/OR16-20, IGHV3/OR16-6, IGHV3/OR16-8, IGHV3/OR16-9, IGHV4-61, IGHV4/OR15-8, IGHV7-81, and IGHV8-51-1. Optionally, a VH region comprises a VH comprising an amino acid sequence (e.g. CDR(s) and/or a human framework region(s), for example according to Kabat numbering) from said gene. In one aspect of any of the embodiments, a VH region comprises an amino acid sequence having at least about 80%, 85%, 90%, 95%, 97%, 98% or 99% identity, to the amino acid sequence of SEQ ID NOS: 184-261.

In one aspect of any of the embodiments, a VL region comprises an amino acid sequence having at least about 80%, 85%, 90%, 95%, 97%, 98% or 99% identity, to the amino acid sequence encoded by a gene of a human V gene group selected from the group consisting of IGKV1-12, IGKV1-13, IGKV1-16, IGKV1-17, IGKV1-27, IGKV1-33, IGKV1-39, IGKV1-5, IGKV1-6, IGKV1-8, IGKV1-9, IGKV1-NL1, IGKV1D-12, IGKV1D-13, IGKV1D-16, IGKV1D-17, IGKV1D-33, IGKV1D-43, IGKV1D-8, IGKV2-24, IGKV2-28, IGKV2-29, IGKV2-30, IGKV2-40, IGKV2D-26, IGKV2D-28, IGKV2D-29, IGKV2D-30, IGKV2D-40, IGKV3-11, IGKV3-15, IGKV3-20, IGKV3D-11, IGKV3D-15, IGKV3D-20, IGKV3D-7, IGKV4-1, IGKV5-2, IGKV6-21, IGKV6D-21, IGKV1-37, IGKV1/OR2-0, IGKV1/OR2-108, IGKV1D-37, IGKV1D-42, IGKV2D-24, IGKV3-7, IGKV3/OR2-268, IGKV3D-20, IGKV6D-41, IGLV1-36, IGLV1-40, IGLV1-44, IGLV1-47, IGLV1-51, IGLV10-54, IGLV2-11, IGLV2-14, IGLV2-18, IGLV2-23, IGLV2-8, IGLV3-1, IGLV3-10, IGLV3-12, IGLV3-16, IGLV3-19, IGLV3-21, IGLV3-22, IGLV3-25, IGLV3-27, IGLV3-9, IGLV4-3, IGLV4-60, IGLV4-69, IGLV5-37, IGLV5-39, IGLV5-45, IGLV5-52, IGLV6-57, IGLV7-43, IGLV7-46, IGLV8-61, IGLV9-49, IGLV1-41, IGLV1-50, IGLV11-55, IGLV2-33, IGLV3-32, IGLV5-48 and IGLV8/OR8-1. Optionally, a VL region comprises a VL comprising an amino acid sequence (e.g. CDR(s) and/or a human framework region(s), for example according to Kabat numbering) from said gene. In one aspect of any of the embodiments, a VL region comprises an amino acid sequence having at least about 80%, 85%, 90%, 95%, 97%, 98% or 99% identity, to the amino acid sequence of SEQ ID NOS: 262-351.

In one aspect of any of the embodiments described herein, an ABD comprises an scFv or Fab, wherein the scFv comprises a VH comprising an amino acid sequence at least 90% identical to a sequence selected from SEQ ID NOS: 3, 5, 7, 9, 11, 13, 112, 113, 115, 116, 117, 119, 120, 121, 123, 124, 125, 127, 128, 129, 132, 134, 136, 138, 140, 142, 144, 146, 148, 150, 152, 154 and any of 184-261, a domain linker, and a VL comprising an amino acid sequence at least 90% identical to a sequence selected from SEQ ID NOS: 4, 6, 8, 10, 12, 14, 114, 118, 122, 126, 130, 133, 135, 137, 139, 141, 143, 145, 147, 149, 151, 153, 155 and any of 262-351; and wherein the Fab comprises one VH comprising an amino acid sequence at least 90% identical to a selected from SEQ ID NOS: 3, 5, 7, 9, 11, 13, 112, 113, 115, 116, 117, 119, 120, 121, 123, 124, 125, 127, 128, 129, 132, 134, 136, 138, 140, 142, 144, 146, 148, 150, 152, 154 and any of 184-261 one VL comprising an amino acid sequence at least 90% identical to a sequence selected from SEQ ID NOS: 4, 6, 8, 10, 12, 14, 114, 118, 122, 126, 130, 133, 135, 137, 139, 141, 143, 145, 147, 149, 151, 153, 155 and any of 262-351; one human CH1 domain comprising an amino acid sequence at least 90% identical to SEQ ID NO: 156 and one human CL domain comprising an amino acid sequence at least 90% identical to SEQ ID NO: 159, wherein the VH is fused to one of the CH1 or CL domains, and the VL is fused to the other of the CH1 or CL domains.

In one aspect of any of the embodiments described herein, an IL2 comprises an amino acid sequence having at least about 80%, 85%, 90%, 95%, 97%, 98% or 99% identity to the IL-2 polypeptide of any of SEQ ID NOS: 354-365, or to a contiguous sequence of at least 40, 50, 60, 70, 80 or 100 amino acid residues thereof. Optionally the IL2 further comprises 2, 3, 4, 5 or more amino acid substitutions that reduce binding to CD25, e.g. substitutions at any of the residues disclosed herein.

In one aspect of any of the embodiments described herein, an IL15 comprises an amino acid sequence having at least about 80%, 85%, 90%, 95%, 97%, 98% or 99% identity to the IL-15 polypeptide of any of SEQ ID NO: 366, or to a contiguous sequence of at least 40, 50, 60, 70, 80 or 100 amino acid residues thereof.

In one aspect of any of the embodiments described herein, an IL12 comprises an amino acid sequence having at least about 80%, 85%, 90%, 95%, 97%, 98% or 99% identity to the IL-12 polypeptide of any of SEQ ID NOS: 386 and/or 387, or to a contiguous sequence of at least 40, 50, 60, 70, 80 or 100 amino acid residues thereof.

In one aspect of any of the embodiments described herein, an IL7 comprises an amino acid sequence having at least about 80%, 85%, 90%, 95%, 97%, 98% or 99% identity to the IL-7 polypeptide of any of SEQ ID NO: 383, or to a contiguous sequence of at least 40, 50, 60, 70, 80 or 100 amino acid residues thereof.

In one aspect of any of the embodiments described herein, an IL27 comprises an amino acid sequence having at least about 80%, 85%, 90%, 95%, 97%, 98% or 99% identity to the IL-21 polypeptide of any of SEQ ID NOS: 384 and/or 385, or to a contiguous sequence of at least 40, 50, 60, 70, 80 or 100 amino acid residues thereof.

In one aspect of any of the embodiments described herein, an IL21 comprises an amino acid sequence having at least about 80%, 85%, 90%, 95%, 97%, 98% or 99% identity to the IL-27 polypeptide of any of SEQ ID NOS: 368 or 369, or to a contiguous sequence of at least 40, 50, 60, 70, 80 or 100 amino acid residues thereof.

In one aspect of any of the embodiments described herein, an IL18 comprises an amino acid sequence having at least about 80%, 85%, 90%, 95%, 97%, 98% or 99% identity to the IL-18 polypeptide of any of SEQ ID NO: 370, or to a contiguous sequence of at least 40, 50, 60, 70, 80 or 100 amino acid residues thereof.

In one aspect of any of the embodiments described herein, an IFN-α comprises an amino acid sequence having at least about 80%, 85%, 90%, 95%, 97%, 98% or 99% identity to the IFN-α polypeptide of any of SEQ ID NOS: 371-381, or to a contiguous sequence of at least 40, 50, 60, 70, 80 or 100 amino acid residues thereof.

In one aspect of any of the embodiments described herein, an IFN-β comprises an amino acid sequence having at least about 80%, 85%, 90%, 95%, 97%, 98% or 99% identity to the IFN-α polypeptide of any of SEQ ID NO: 382, or to a contiguous sequence of at least 40, 50, 60, 70, 80 or 100 amino acid residues thereof.

In one aspect of any of the embodiments described herein, an Fc domain comprises an amino acid sequence having at least about 80%, 85%, 90%, 95%, 97%, 98% or 99% identity to the Fc polypeptide of any of SEQ ID NOS: 160-165, or to a contiguous sequence of at least 40, 50, 60, 70, 80 or 100 amino acid residues thereof.

In one aspect of any of the embodiments described herein, a CH1, CH2 and CH3 domain respectively comprise an amino acid sequence having at least about 80%, 85%, 90%, 95%, 97%, 98% or 99% identity to the CH1, CH2 or CH3 polypeptide of SEQ ID NO: 156,157 or 158, or to a contiguous sequence of at least 40, 50, 60, 70, 80 or 100 amino acid residues thereof.

In one aspect of any of the embodiments described herein, a CK or CL domain comprises an amino acid sequence having at least about 80%, 85%, 90%, 95%, 97%, 98% or 99% identity to the CK polypeptide of any of SEQ ID NO: 159, or to a contiguous sequence of at least 40, 50, 60, 70, 80 or 100 amino acid residues thereof.

In one aspect of any of the embodiments described herein, a hinge domain comprises an amino acid sequence having at least about 80%, 85%, 90%, 95%, 97%, 98% or 99% identity to the CK polypeptide of any of SEQ ID NO: 166-170.

In one embodiment, a multispecific protein comprises: a polypeptide comprising an amino acid sequence having at least 80%, 90% or 95% sequence identity to the amino acid sequence of a first chain of a heterotrimeric protein described herein, a polypeptide comprising an amino acid sequence having at least 80%, 90% or 95% sequence identity to the amino acid sequence of a second chain of a heterotrimeric protein described herein and a polypeptide comprising an amino acid sequence having at least 80%, 90% or 95% sequence identity to the amino acid sequence of a third chain of a heterotrimeric protein described herein. In one embodiment, a multispecific protein comprises: a polypeptide comprising an amino acid sequence having at least 80%, 90% or 95% sequence identity to the amino acid sequence of a first chain of a heterodimeric protein described herein, and a polypeptide comprising an amino acid sequence having at least 80%, 90% or 95% sequence identity to the amino acid sequence of a second chain of a heterodimeric protein described herein.

In one embodiment, a multispecific protein comprises: a polypeptide comprising an amino acid sequence of a first chain of a heterotrimeric protein described herein, a polypeptide comprising an amino acid sequence of a heterotrimeric protein described herein and a polypeptide comprising an amino acid sequence of a third chain of a heterotrimeric protein described herein.

In one embodiment, a multispecific protein comprises: a polypeptide comprising an amino acid sequence having at least 80%, 90% or 95% sequence identity to the amino acid sequence of SEQ ID NO: 175, a polypeptide comprising an amino acid sequence having at least 80%, 90% or 95% sequence identity to the amino acid sequence of SEQ ID NO: 176 and a polypeptide comprising an amino acid sequence having at least 80%, 90% or 95% sequence identity to the amino acid sequence of SEQ ID NO: 177.

In one embodiment, a multispecific protein comprises: a polypeptide comprising an amino acid sequence having at least 80%, 90% or 95% sequence identity to the amino acid sequence of SEQ ID NO: 178, a polypeptide comprising an amino acid sequence having at least 80%, 90% or 95% sequence identity to the amino acid sequence of SEQ ID NO: 179 and a polypeptide comprising an amino acid sequence having at least 80%, 90% or 95% sequence identity to the amino acid sequence of SEQ ID NO: 180.

In one embodiment, a multispecific protein comprises: a polypeptide comprising an amino acid sequence having at least 80%, 90% or 95% sequence identity to the amino acid sequence of SEQ ID NO: 181, a polypeptide comprising an amino acid sequence having at least 80%, 90% or 95% sequence identity to the amino acid sequence of SEQ ID NO: 182 and a polypeptide comprising an amino acid sequence having at least 80%, 90% or 95% sequence identity to the amino acid sequence of SEQ ID NO: 183.

In one aspect the invention provides an isolated multispecific heterotrimeric protein comprising a first polypeptide chain comprising an amino acid sequence which is at least 50%, 60%, 70%, 80%, 85%, 90%, 95%, 98 or 99% identical to the sequence of a first polypeptide chain of a T53A protein disclosed herein; a second polypeptide chain comprising an amino acid sequence which is at least 50%, 60%, 70%, 80%, 85%, 90%, 95%, 98 or 99% identical to the sequence of a second polypeptide chain of the respective T53A protein disclosed herein; and optionally a third polypeptide chain comprising an amino acid sequence which is at least 50%, 60%, 70%, 80%, 85%, 90%, 95%, 98 or 99% identical to the sequence of a third polypeptide chain of a T53A protein disclosed herein. In one embodiment, CDRs are excluded from the sequences that are considered for computing sequence identity. In one embodiment, VH and/or VL variable regions are excluded from the sequences that are considered for computing sequence identity of a polypeptide chain. Optionally each VH region comprises an amino acid sequence having at least about 80%, 85%, 90%, 95%, 97%, 98% or 99% identity, to the amino acid sequence of SEQ ID NOS: 3, 5, 7, 9, 11, 13, 112, 113, 115, 116, 117, 119, 120, 121, 123, 124, 125,127, 128, 129, 132, 134, 136, 138, 140, 142, 144, 146, 148, 150, 152, 154 and any of 184-261. Optionally each VL region comprises an amino acid sequence having at least about 80%, 85%, 90%, 95%, 97%, 98% or 99% identity, to the amino acid sequence of SEQ ID NOS: 4, 6, 8, 10, 12, 14, 114, 118, 122, 126, 130, 133, 135, 137, 139, 141, 143, 145, 147, 149, 151, 153, 155 and any of 262-351.

In one aspect of any of the embodiments described herein, provided is a recombinant nucleic acid encoding a first polypeptide chain, and/or a second polypeptide chain, and/or a third polypeptide chain and/or a fourth polypeptide. In one aspect of any of the embodiments described herein, the invention provides a recombinant host cell comprising a nucleic acid encoding a first polypeptide chain, and/or a second polypeptide chain and/or a third polypeptide chain, optionally wherein the host cell produces a multimeric or other protein according to the invention with a yield (final productivity or concentration before or after purification) of at least 1, 2, 3 or 4 mg/L. Also provided is a kit or set of nucleic acids comprising a recombinant nucleic acid encoding a first polypeptide chain of the according to the invention, a recombinant nucleic acid encoding a second polypeptide chain according to the invention, and, optionally, a recombinant nucleic acid encoding a third polypeptide chain according to the invention. Also provided are methods of making dimeric, trimeric and tetrameric proteins according to the invention.

Any of the methods can further be characterized as comprising any step described in the application, including notably in the “Detailed Description of the Invention”). The invention further relates to methods of identifying, testing and/or making proteins described herein. The invention further relates to a multispecific protein obtainable by any of present methods. The disclosure further relates to pharmaceutical or diagnostic formulations containing at least one of the multispecific proteins disclosed herein. The disclosure further relates to methods of using the subject multispecific proteins in methods of treatment or diagnosis.

These and additional advantageous aspects and features of the invention may be further described elsewhere herein.

### BRIEF DESCRIPTION OF THE FIGURES

FIG. 1 shows the topology of the multispecific NK cell engager (NKCE) protein that binds on one face to a tumor antigen on a tumor cell, and on another face to an NK cell via a triple receptor cis-presentation of IL2βγ complex, NKp46 and CD16A. IL2v capture on NK cells may improve binding to CD122 and mimic CD25-mediated IL-2 presentation.

FIGS. 2A and 2B show an exemplary multispecific protein in T53A format that binds to NKp46, CD16A and cytokiner receptor (e.g. CD122) on an NK cell, and to tumor antigen (e.g. TA, Tag, CD20) on a tumor cell. In FIG. 2A, the star in the CH3 domain indicates mutations H435R and Y436F (Kabat EU numbering).

FIG. 3 shows an exemplary multispecific protein in heterotetramer format that binds to NKp46, CD16A and cytokine receptor (e.g. CD122) on an NK cell, and to tumor antigen (TA) on a tumor cell, where the NKp46 binding domain and the TA binding domain are both Fabs positioned topologically N-terminally in the protein (and N-terminal with respect to the Fc domain), and the cytokine is placed topologically at the C-terminus. The dimeric Fc domain is interposed between the TA ABD and the NKp46 ABD which are on the N-terminal side of the Fc domain dimer and the cytokine which is on the C-terminal side of the Fc domain dimer. The proteins have one NKp46 ABD, one TA ABD, one dimeric Fc domain and one cytokine moiety, thereby having a 1:1:1 format for TA, NKp46 and cytokine receptor binding.

FIG. 4A shows exemplary multispecific proteins that have two TA binding domains and one NKp46 binding domain positioned topologically at the N-terminus of the protein, a dimeric Fc domain, and a cytokine placed topologically at the C-terminus. The proteins have one dimeric Fc domain and one cytokine, thereby having a 2:1:1 format for TA, NKp46 and cytokine receptor binding. Shown in FIG. 4B are exemplary heterotetramer protein structures for the proteins of FIG. 4A made from three different chains. Shown in FIG. 4C are exemplary heteropentamer protein structures for the proteins of FIG. 4A made from four different chains. In FIGS. 4B and 4C, the star in the CH3 domain indicates mutations H435R and Y436F (Kabat EU numbering).

FIG. 5 shows activation of TReg cells by heterotrimer proteins that contained either a wild-type IL-2 or a variant IL2, and that lacked binding to NKp46, CD16A and antigen of interest. The protein containing the variant IL2 showed a strongly decreased ability to activate Treg cells compared to wild-type IL-2 and to the heterotrimer protein containing wild-type IL-2.

FIG. 6 shows ability to direct purified NK cells to lyse CD20-positive RAJI tumor target cells by CD20×NKp46 binding proteins. GA101-T53A-NKp46-IL2v proteins having different linkers between the Fc domain and the IL2v were highly potent in mediating NK cell lysis of tumor target cells.

FIGS. 7, 8, 9 and 10 show % of pSTAT5 cells among NK cells, CD4 T cells CD8 T cells and Treg cells, respectively. GA101-T53A-NKp46-IL2v having 5, 10 or 15 residue linkers displayed comparable activation of each cell type. The GA101-T53A-NKp46-IL2v resulted in a strong increase in potency in the ability to cause an increase in percent of pSTAT5+ cells among the NK cells, compared to wild-type human IL-2 that did not bind NKp46 or CD16A. Yet further, the GA101-T53A-NKp46-IL2v resulted in a decrease in potency in the ability to cause an increase in percent of pSTAT5+ cells among the CD4 T cells CD8 T cells and especially the Treg cells, compared to wild-type human IL-2 that did not bind NKp46 or CD16A, combined with a strong decrease. The GA101-T5-NKp46-IL2v protein therefore permitted a selective activation of NK cells over Treg cells, CD4 T cells and CD8 T cells.

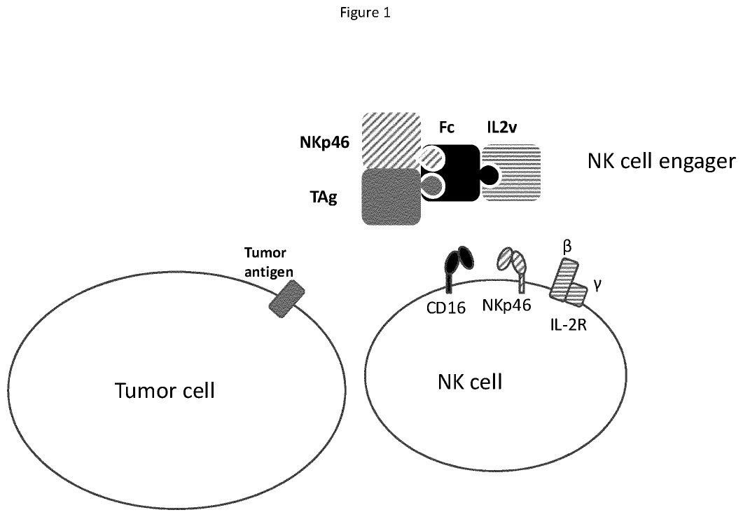

### DETAILED DESCRIPTION OF THE INVENTION

### Definitions

As used in the specification, “a” or “an” may mean one or more. As used in the claim(s), when used in conjunction with the word “comprising”, the words “a” or “an” may mean one or more than one.

Where “comprising” is used, this can optionally be replaced by “consisting essentially of”, or optionally by “consisting of”.

As used herein, the term “antigen binding domain” or “ABD” refers to a domain comprising a three-dimensional structure capable of immunospecifically binding to an epitope. Thus, in one embodiment, said domain can comprise a hypervariable region, optionally a V_{H} and/or V_{L} domain of an antibody chain, optionally at least a V_{H} domain. In another embodiment, the binding domain may comprise at least one complementarity determining region (CDR) of an antibody chain. In another embodiment, the binding domain may comprise a polypeptide domain from a non-immunoglobulin scaffold.

The term “antibody” herein is used in the broadest sense and specifically includes full-length monoclonal antibodies, polyclonal antibodies, multispecific antibodies (e.g., bispecific antibodies), and antibody fragments and derivatives, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988). An “antibody fragment” comprises a portion of a full-length antibody, e.g. antigen-binding or variable regions thereof. Examples of antibody fragments include Fab, Fab′, F(ab)₂, F(ab′)₂, F(ab)₃, Fv (typically the V_{L} and V_{H} domains of a single arm of an antibody), single-chain Fv (scFv), dsFv, Fd fragments (typically the V_{H} and CH1 domain), and dAb (typically a V_{H} domain) fragments; V_{H}, V_{L}, VhH, and V-NAR domains; minibodies, diabodies, triabodies, tetrabodies, and kappa bodies (see, e.g., III et al., Protein Eng 1997; 10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219, and published U.S. Patent Applications 20050238646 and 20020161201.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

By “constant region” as defined herein is meant an antibody-derived constant region that is encoded by one of the light or heavy chain immunoglobulin constant region genes.

By “constant light chain” or “light chain constant region” or “CL” as used herein is meant the region of an antibody encoded by the kappa (CK) or lambda (CA) light chains. The constant light chain typically comprises a single domain, and as defined herein refers to positions 108-214 of CK, or CA, wherein numbering is according to the EU index (Kabat et al., 1991, Sequences of Proteins of Immunological Interest, 5th Ed., United States Public Health Service, National Institutes of Health, Bethesda).

By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fab” or “Fab region” as used herein is meant a unit that comprises the V_{H}, CH1, V_{L}, and CL immunoglobulin domains. The term Fab includes a unit that comprises a V_{H}-CH1 moiety that associates with a V_{L}—CL moiety, as well as crossover Fab structures in which there is crossing over or interchange between light- and heavy-chain domains. For example a Fab may have a V_{H}-CL unit that associates with a V_{L}—CH1 unit. Fab may refer to this region in isolation, or this region in the context of a protein, multispecific protein or ABD, or any other embodiments as outlined herein.

By “single-chain Fv” or “scFv” as used herein are meant antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994).

By “Fv” or “Fv fragment” or “Fv region” as used herein is meant a polypeptide that comprises the V_{L} and V_{H} domains of a single antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ3 (CH3) and optionally the hinge between Cγ1 and Cγ2. Although the boundaries of the Fc region may vary, the human IgG heavy chain Fc region is usually defined to comprise residues C226, P230 or A231 to its carboxyl-terminus, wherein the numbering is according to the EU index. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides herein include but are not limited to antibodies, Fc fusions and Fc fragments. Also, Fc regions according to the invention include variants containing at least one modification that alters (enhances or diminishes) an Fc associated effector function. Also, Fc regions according to the invention include chimeric Fc regions comprising different portions or domains of different Fc regions, e.g., derived from antibodies of different isotype or species.

By “variable region” as used herein is meant the region of an antibody that comprises one or more Ig domains substantially encoded by any of the V_{L} (including Vκ (Vκ) and Vλ) and/or V_{H} genes that make up the light chain (including κ and λ) and heavy chain immunoglobulin genetic loci respectively. A light or heavy chain variable region (V_{L} or V_{H}) consists of a “framework” or “FR” region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

The term “specifically binds to” means that an antibody or polypeptide can bind preferably in a competitive binding assay to the binding partner, e.g. NKp46, as assessed using either recombinant forms of the proteins, epitopes therein, or native proteins present on the surface of isolated target cells. Competitive binding assays and other methods for determining specific binding are further described below and are well known in the art.

When an antibody or polypeptide is said to “compete with” a particular multispecific protein or a particular monoclonal antibody (e.g. NKp46-1, -2, -4, -6 or -9 in the context of an anti-NKp46 mono-specific antibody or a multi-specific protein), it means that the antibody or polypeptide competes with the particular multispecific protein or monoclonal antibody in a binding assay using either recombinant target (e.g. NKp46) molecules or surface expressed target (e.g. NKp46) molecules. For example, if a test antibody reduces the binding of NKp46-1, -2, -4, -6 or -9 to a NKp46 polypeptide or NKp46-expressing cell in a binding assay, the antibody is said to “compete” respectively with NKp46-1, -2, -4, -6 or -9.

The term “affinity”, as used herein, means the strength of the binding of an antibody or protein to an epitope. The affinity of an antibody is given by the dissociation constant K_{D}, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Preferred methods for determining the affinity of proteins can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of proteins is the use of surface plasmon resonance (SPR) screening (such as by analysis with a BIAcore™ SPR analytical device).

Within the context of this invention a “determinant” designates a site of interaction or binding on a polypeptide.

The term “epitope” refers to an antigenic determinant, and is the area or region on an antigen to which an antibody or protein binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’. Epitopes may be identified by different methods known in the art including but not limited to alanine scanning, phage display, X-ray crystallography, array-based oligo-peptide scanning or pepscan analysis, site-directed mutagenesis, high throughput mutagenesis mapping, H/D-Ex Mass Spectroscopy, homology modeling, docking, hydrogen-deuterium exchange, among others. (See e.g., Tong et al., Methods and Protocols for prediction of immunogenic epitopes”, Briefings in Bioinformatics 8(2):96-108; Gershoni, Jonathan M; Roitburd-Berman, Anna; Siman-Tov, Dror D; Tarnovitski Freund, Natalia; Weiss, Yael (2007). “Epitope Mapping”. BioDrugs 21 (3): 145-56; and Flanagan, Nina (May 15, 2011); “Mapping Epitopes with H/D-Ex Mass Spec: ExSAR Expands Repertoire of Technology Platform Beyond Protein Characterization”, Genetic Engineering & Biotechnology News 31 (10).

“Valent” or “valency” denotes the presence of a determined number of antigen-binding moieties in the antigen-binding protein. A natural IgG has two antigen-binding moieties and is bivalent. A molecule having one binding moiety for a particular antigen is monovalent for that antigen.

By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. Amino acid substitutions are indicated by listing the residue present in wild-type protein/position of residue/residue present in mutant protein. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a polypeptide will exhibit 98%, 98%, or 99% homogeneity for polypeptides in the context of all present peptide species in the composition or at least with respect to substantially active peptide species in the context of proposed use.

In the context herein, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

As used herein, the phrase “NK cells” refers to a sub-population of lymphocytes that is involved in non-conventional immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell K S and Colonna M). Humana Press. pp. 219-238 (2000).

As used herein, an agent that has “agonist” activity at NKp46 is an agent that can cause or increase “NKp46 signaling”. “NKp46 signaling” refers to an ability of an NKp46 polypeptide to activate or transduce an intracellular signaling pathway. Changes in NKp46 signaling activity can be measured, for example, by assays designed to measure changes in NKp46 signaling pathways, e.g. by monitoring phosphorylation of signal transduction components, assays to measure the association of certain signal transduction components with other proteins or intracellular structures, or in the biochemical activity of components such as kinases, or assays designed to measure expression of reporter genes under control of NKp46-sensitive promoters and enhancers, or indirectly by a downstream effect mediated by the NKp46 polypeptide (e.g. activation of specific cytolytic machinery in NK cells). Reporter genes can be naturally occurring genes (e.g. monitoring cytokine production) or they can be genes artificially introduced into a cell. Other genes can be placed under the control of such regulatory elements and thus serve to report the level of NKp46 signaling.

“NKp46” refers to a protein or polypeptide encoded by the Ncr1 gene or by a cDNA prepared from such a gene. Any naturally occurring isoform, allele, ortholog or variant is encompassed by the term NKp46 polypeptide (e.g., an NKp46 polypeptide 90%, 95%, 98% or 99% identical to SEQ ID NO 1, or a contiguous sequence of at least 20, 30, 50, 100 or 200 amino acid residues thereof). The 304 amino acid residue sequence of human NKp46 (isoform a) is shown below:

(SEQ ID NO: 1)
MSSTLPALLC VGLCLSQRIS AQQQTLPKPF IWAEPHFMVP
KEKQVTICCQ GNYGAVEYQL HFEGSLFAVD RPKPPERINK
VKFYIPDMNS RMAGQYSCIY RVGELWSEPS NLLDLVVTEM
YDTPTLSVHP GPEVISGEKV TFYCRLDTAT SMFLLLKEGR
SSHVQRGYGK VQAEFPLGPV TTAHRGTYRC FGSYNNHAWS
FPSEPVKLLV TGDIENTSLA PEDPTFPADT WGTYLLTTET
GLQKDHALWD HTAQNLLRMG LAFLVLVALV WFLVEDWLSR
KRTRERASRA STWEGRRRLN TQTL.

SEQ ID NO: 1 corresponds to NCBI accession number NP_004820, the disclosure of which is incorporated herein by reference. The human NKp46 mRNA sequence is described in NCBI accession number NM_004829, the disclosure of which is incorporated herein by reference.

### Producing Polypeptides

The proteins described herein can be conveniently configured and produced using well known immunoglobulin-derived domains, notably heavy and light chain variable domains, hinge regions, CH1, CL, CH2 and CH3 constant domains, and wild-type or variant cytokine polypeptides. Domains placed on a common polypeptide chain can be fused to one another either directly or connected via linkers, depending on the particular domains concerned. The immunoglobulin-derived domains will preferably be humanized or of human origin, thereby providing decreased risk of immunogenicity when administered to humans. As shown herein, advantageous protein formats are described that use minimal non-immunoglobulin linking amino acid sequences (e.g. not more than 4 or 5 domain linkers, in some cases as few as 1 or 2 domain linkers, and use of domains linkers of short length), thereby further reducing risk of immunogenicity.

Immunoglobulin variable domains are commonly derived from antibodies (immunoglobulin chains), for example in the form of associated V_{L} and V_{H} domains found on two polypeptide chains, or a single chain antigen binding domain such as an scFv, a V_{H} domain, a V_{L} domain, a dAb, a V-NAR domain or a V_{H}H domain. In certain advantageous proteins formats disclosed herein that directly enable the use of a wide range of variable regions from Fab or scFv without substantial further requirements for pairing and/or folding, the an antigen binding domain (e.g., ABD₁ and ABD₂) can also be readily derived from antibodies as a Fab or scFv.

The term “antigen-binding protein” can be used to refer to an immunoglobulin derivative with antigen binding properties. The binding protein comprises an immunologically functional immunoglobulin portion capable of binding to a target antigen. The immunologically functional immunoglobulin portion may comprise immunoglobulins, or portions thereof, fusion peptides derived from immunoglobulin portions or conjugates combining immunoglobulin portions that form an antigen binding site. An antigen binding moiety can thus comprise at least the necessarily one, two or three CDRs of the immunoglobulin heavy and/or light chains from which the antigen binding moiety was derived. In some aspects, an antigen-binding protein can consist of a single polypeptide chain (a monomer). In other embodiments the antigen-binding protein comprises at least two polypeptide chains. Such an antigen-binding protein is a multimer, e.g., dimer, trimer or tetramer.

Examples of antigen binding proteins includes antibody fragments, antibody derivatives or antibody-like binding proteins that retain specificity and affinity for their antigen.

Typically, antibodies are initially obtained by immunization of a non-human animal, e.g., a mouse, rat, guinea pig or rabbit, with an immunogen comprising a polypeptide, or a fragment or derivative thereof, typically an immunogenic fragment, for which it is desired to obtain antibodies (e.g. a human polypeptide). The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, NY (1988), the entire disclosure of which is herein incorporated by reference). Human antibodies may also be produced by using, for immunization, transgenic animals that have been engineered to express a human antibody repertoire (Jakobovitz et al. Nature 362 (1993) 255), or by selection of antibody repertoires using phage display methods. For example, a XenoMouse (Abgenix, Fremont, CA) can be used for immunization. A XenoMouse is a murine host that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference. Antibodies may also be produced by selection of combinatorial libraries of immunoglobulins, as disclosed for instance in (Ward et al. Nature, 341 (1989) p. 544, the entire disclosure of which is herein incorporated by reference). Phage display technology (McCafferty et al (1990) Nature 348:552-553) can be used to produce antibodies from immunoglobulin variable (V) domain gene repertoires from unimmunized donors. See, e.g., Griffith et al (1993) EMBO J. 12:725-734; U.S. Pat. Nos. 5,565,332; 5,573,905; 5,567,610; and 5,229,275). When combinatorial libraries comprise variable (V) domain gene repertoires of human origin, selection from combinatorial libraries will yield human antibodies.

In any embodiment, an antigen binding domain can be obtained from a humanized antibody in which residues from a complementary-determining region (CDR) of a human antibody are replaced by residues from a CDR of the original antibody (the parent or donor antibody, e.g. a murine or rat antibody) while maintaining the desired specificity, affinity, and capacity of the original antibody. The CDRs of the parent antibody, some or all of which are encoded by nucleic acids originating in a non-human organism, are grafted in whole or in part into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. An antigen binding domain can thus have non-human hypervariable regions or CDRs and human frameworks region sequences (optionally with back mutations).

Additionally, a wide range of antibodies are available in the scientific and patent literature, including DNA and/or amino acid sequences, or from commercial suppliers. Antibodies will typically be directed to a pre-determined antigen. Examples of antibodies include antibodies that recognize an antigen expressed by a target cell that is to be eliminated, for example a proliferating cell or a cell contributing to a disease pathology. Examples include antibodies that recognize tumor antigens, microbial (e.g. bacterial or parasite) antigens or viral antigens.

Alternatively, antigen binding domains used in the proteins described herein can be readily derived from any of a variety of non-immunoglobulin scaffolds, for example affibodies based on the Z-domain of staphylococcal protein A, engineered Kunitz domains, monobodies or adnectins based on the 10th extracellular domain of human fibronectin III, anticalins derived from lipocalins, DARPins® (designed ankyrin repeat domains, multimerized LDLR-A module, avimers or cysteine-rich knottin peptides. See, e.g., Gebauer and Skerra (2009) Current Opinion in Chemical Biology 13:245-255, the disclosure of which is incorporated herein by reference.

As further exemplified herein, an antigen binding domain can conveniently comprise a VH and a VL (a VH/VL pair). In some embodiments, the VH/VL pair can be integrated in a Fab structure further comprising a CH1 and CL domain (a CH1/CL pair). A VH/VL pair refers to one VH and one VL domain that associate with one another to form an antigen binding domain. A CH1/CL pair refers to one CH1 and one CL domain bound to one another by covalent or non-covalent bonds, preferably non-covalent bonds, thus forming a heterodimer (e.g., within a protein such as a heterotrimer, heterotetramer, heteropentamer that can comprise one or more further polypeptide chains). The constant chain domains forming the pair can be present on the same or on different polypeptide chain, in any suitable combination.

Exemplary CDRs or VH and VL domains that bind NKp46 can be derived from the anti-NKp46 antibodies provided herein (see section “NKp46 variable region and CDR sequences”), or can be selected from any of the CDRs, VH and VL domains of PCT publication nos. WO2016/207278 and WO2017/114694, the disclosures of which are incorporated herein by reference. Variable regions can be used directly, or can be modified by selecting hypervariable or CDR regions from the NKp46 antibodies and placing them into the desired V_{L} or V_{H} framework, for example human frameworks. Antigen binding domains that bind NKp46 can also be derived de novo using methods for generating antibodies. Antibodies can be tested for binding to NKp46 polypeptides. In one aspect of any embodiment herein, a polypeptide (e.g. multispecific protein) that binds to NKp46 will be capable of binding NKp46 expressed on the surface of a cell, e.g. native NKp46 expressed by a NK cell.

Antigen binding domains (ABDs) that bind an antigen of interest can be selected based on the desired predetermined antigen of interest (e.g. an antigen other than NKp46), and may include for example cancer antigens such as antigens present on tumor cells and/or on immune cells capable of mediating a pro-tumoral effect, e.g. a monocyte or a macrophage, optionally a suppressor T cell, regulatory T cell, or myeloid-derived suppressor cell (for the treatment of cancer); bacterial or viral antigens (for the treatment of infectious disease); or antigens present on pro-inflammatory immune cells, e.g. T cells, neutrophils, macrophages, etc. (for the treatment of inflammatory and/or autoimmune disorder).

As used herein, the term “bacterial antigen” includes, but is not limited to, intact, attenuated or killed bacteria, any structural or functional bacterial protein or carbohydrate, or any peptide portion of a bacterial protein of sufficient length (typically about 8 amino acids or longer) to be antigenic. Examples include gram-positive bacterial antigens and gram-negative bacterial antigens. In some embodiments the bacterial antigen is derived from a bacterium selected from the group consisting of Helicobacter species, in particular Helicobacter pyloris; Borrelia species, in particular Borrelia burgdorferi; Legionella species, in particular Legionella pneumophilia; Mycobacteria s species, in particular M. tuberculosis, M. avium, M. intracellulare, M. kansasii, M. gordonae; Staphylococcus species, in particular Staphylococcus aureus; Neisseria species, in particular N. gonorrhoeae, N. meningitidis; Listeria species, in particular Listeria monocytogenes; Streptococcus species, in particular S. pyogenes, S. agalactiae; S. faecalis; S. bovis, S. pneumoniae; anaerobic Streptococcus species; pathogenic Campylobacter species; Enterococcus species; Haemophilus species, in particular Haemophilus influenzae; Bacillus species, in particular Bacillus anthracis; Corynebacterium species, in particular Corynebacterium diphtheriae; Erysipelothrix species, in particular Erysipelothrix rhusiopathiae; Clostridium species, in particular C. perfringens, C. tetani; Enterobacter species, in particular Enterobacter aerogenes, Klebsiella species, in particular Klebsiella 1S pneumoniae, Pasteurella species, in particular Pasteurella multocida, Bacteroides species; Fusobacterium species, in particular Fusobacterium nucleatum; Streptobacillus species, in particular Streptobacillus moniliformis; Treponema species, in particular Treponema pertenue; Leptospira; pathogenic Escherichia species; and Actinomyces species, in particular Actinomyces israeli.

As used herein, the term “viral antigen” includes, but is not limited to, intact, attenuated or killed whole virus, any structural or functional viral protein, or any peptide portion of a viral protein of sufficient length (typically about 8 amino acids or longer) to be antigenic. Sources of a viral antigen include, but are not limited to viruses from the families: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., Ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Bornaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), Hepatitis C; Norwalk and related viruses, and astroviruses). Alternatively, a viral antigen may be produced recombinantly.

As used herein, the terms “cancer antigen” and “tumor antigen” are used interchangeably and refer to antigens (other than the cytokine receptor expressed on NK cells, NKp46, and CD16) that are differentially expressed by cancer cells or are expressed by non-tumoral cells (e.g. immune cells) having a pro-tumoral effect (e.g. an immunosuppressive effect), and can thereby be exploited in order to target cancer cells. Cancer antigens can be antigens which can potentially stimulate apparently tumor-specific immune responses. Some of these antigens are encoded, although not necessarily expressed, or expressed at lower levels or less frequently, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Still other cancer antigens can be expressed on immune cells capable of contributing to or mediating a pro-tumoral effect, e.g. cell that contributes to immune evasion, a monocyte or a macrophage, optionally a suppressor T cell, regulatory T cell, or myeloid-derived suppressor cell.

The cancer antigens are usually normal cell surface antigens which are either over-expressed or expressed at abnormal times, or are expressed by a targeted population of cells. Ideally the target antigen is expressed only on proliferative cells (e.g., tumor cells) or pro-tumoral cells (e.g. immune cells having an immunosuppressive effect), however this is rarely observed in practice. As a result, target antigens are in many cases selected on the basis of differential expression between proliferative/disease tissue and healthy tissue. Example of cancer antigens include: Receptor Tyrosine Kinase-like Orphan Receptor 1 (ROR1), Crypto, CD4, CD19, CD20, CD30, CD38, CD47, Glycoprotein NMB, CanAg, Her2 (ErbB2/Neu), a Siglec family member, for example CD22 (Siglec2) or CD33 (Siglec3), CD79, CD123, CD138, CD171, PSCA, L1-CAM, PSMA (prostate specific membrane antigen), BCMA, CD52, CD56, CD80, CD70, E-selectin, EphB2, Melanotransferrin, Mud 6 and TMEFF2. Examples of cancer antigens also include Immunoglobulin superfamily (IgSF) such as cytokine receptors, Killer-Ig Like Receptor, CD28 family proteins, for example, Killer-Ig Like Receptor 3DL2 (KIR3DL2), B7-H3, B7-H4, B7-H6, PD-L1. Examples also include MAGE, MART-1/Melan-A, gp100, major histocompatibility complex class I-related chain A and B polypeptides (MICA and MICB), adenosine deaminase-binding protein (ADAbp), cyclophilin b, colorectal associated antigen (CRC)-C017-1A/GA733, protein tyrosine kinase 7(PTK7), receptor protein tyrosine kinase 3 (TYRO-3), nectins (e.g. nectin-4), major histocompatibility complex class I-related chain A and B polypeptides (MICA and MICB), proteins of the UL16-binding protein (ULBP) family, proteins of the retinoic acid early transcript-1 (RAET1) family, carcinoembryonic antigen (CEA) and its immunogenic epitopes CAP-1 and CAP-2, etv6, aml1, prostate specific antigen (PSA), T-cell receptor/CD3-zeta chain, MAGE-family of tumor antigens, GAGE-family of tumor antigens, anti-Müllerian hormone Type II receptor, delta-like ligand 4 (DLL4), DR5, ROR1 (also known as Receptor Tyrosine Kinase-Like Orphan Receptor 1 or NTRKR1 (EC 2.7.10.1), BAGE, RAGE, LAGE-1, NAG, GnT-V, MUM-1, CDK4, MUC family, VEGF, VEGF receptors, Angiopoietin-2, PDGF, TGF-alpha, EGF, EGF receptor, members of the human EGF-like receptor family, e.g., HER-2/neu, HER-3, HER-4 or a heterodimeric receptor comprised of at least one HER subunit, gastrin releasing peptide receptor antigen, Muc-1, CA125, integrin receptors, αvß3 integrins, α5ß1 integrins, αIIbß3-integrins, PDGF beta receptor, SVE-cadherin, hCG, CSF1R (tumor-associated monocytes and macrophages), α-fetoprotein, E-cadherin, α-catenin, ß-catenin and γ-catenin, p120ctn, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2, although this is not intended to be exhaustive.

Optionally, a multispecific protein can be specified as excluding or not requiring a stromal modifying moiety, e.g., a moiety capable of altering or degrading a component of, the stroma such as an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin. For example, the stromal modifying moiety can be a hyaluronan degrading enzyme, an agent that inhibits hyaluronan synthesis, or an antibody molecule against hyaluronic acid. Optionally, a multispecific protein can be specified as excluding a mesothelin targeting moiety or mesothelin-binding ABD. Optionally, a multispecific protein can be specified as excluding a PD-L1 targeting moiety, a HER3 targeting moiety, an IGFIR targeting moiety or a hyaluronidase 1 targeting moiety, or a combination a stroma targeting moiety or ABD and a cancer-antigen targeting moiety. Optionally a cancer antigen or antigen of interest can be specified as being other than a PD-L1, a HER3, an IGFIR or hyaluronidase 1.

By way of examples, when the ABD that binds an antigen of interest binds to a HER2 polypeptide, exemplary VH and VL pairs can be selected from antibodies trastuzumab, pertuzumab or margetuximab:

Trastuzumab heavy chain variable region
(SEQ ID NO: 132)
EVQLVESGGG LVQPGGSLRL SCAASGFNIK
DTYIHWVRQA PGKGLEWVAR IYPTNGYTRY
ADSVKGRFTI SADTSKNTAY LQMNSLRAED
TAVYYCSRWG GDGFYAMDYW GQGTLVTVSS.
Trastuzumab light chain variable region
(SEQ ID NO: 133)
DIQMTQSPSS LSASVGDRVT ITCRASQDVN
TAVAWYQQKP GKAPKLLIYS ASFLYSGVPS
RFSGSRSGTD FTLTISSLQP EDFATYYCQQ
HYTTPPTFGQ GTKVEIK.
Margetuximab VH:
(SEQ ID NO: 134)
QVQLQQSGPE LVKPGASLKL SCTASGFNIK
DTYIHWVKQR PEQGLEWIGRIYPTNGYTRY
DPKFQDKATI TADTSSNTAY LQVSRLTSED
TAVYYCSRWG GDGFYAMDYW GQGASVTVSS.
Margetuximab VL:
(SEQ ID NO: 135)
DIVMTQSHKF MSTSVGDRVS ITCKASQDVN
TAVAWYQQKP GHSPKLLIYS ASFRYTGVPD
RFTGSRSGTD FTFTISSVQA EDLAVYYCQQ
HYTTPPTFGG GTKVEIK.

In another example, when the ABD that binds an antigen of interest binds to a CD19 polypeptide, exemplary VH and VL pairs can be selected from the VL and VL pair from blinatumomab.

Blinatumomab VH:
(SEQ ID NO: 136)
QVQLQQSGAELVRPGSSVKISCKASGYAFSSYWMNWVKQRP
GQGLEWIGQIWPGDGDTNYNGKFKGKATLTADESSSTAYMQ
LSSLASEDSAVYFCARRETTTVGRYYYAMDYWGQGTTVTVS
S.
Blinatumomab VL:
(SEQ ID NO: 137)
DIQLTQSPASLAVSLGQRATISCKASQSVDYDGDSYLNWYQ
QIPGQPPKLLIYDASNLVSGIPPRFSGSGSGTDFTLNIHPV
EKVDAATYHCQQSTEDPWTFGGGTKLEIK.

In another example, when the ABD that binds an antigen of interest binds to a CD20 polypeptide, exemplary VH and VL pairs can be selected from VL and VL pair from rituximab and obinutuzumab:

Rituximab VH:
(SEQ ID NO: 138)
QVQLQQPGAELVKPGASVKMSCKASGYTFTSYNMHWVKQTPGRGLEWIG
AIYPGNGDTSYNQKFKGKATLTADKSSSTAYMQLSSLTSEDSAVYYCAR
STYYGGDWYFNVWGAGTTVTVSA.
Rituximab VL:
(SEQ ID NO: 139)
QIVLSQSPAILSASPGEKVTMTCRASSSVSYIHWFQQKPGSSPKPWIYA
TSNLASGVPVRFSGSGSGTSYSLTISRVEAEDAATYYCQQWTSNPPTFG
GGTKLEIK.
Obinutuzumab VH:
(SEQ ID NO: 140)
QVQLVQSGAEVKKPGSSVKVSCKASGYAFSYSWINWVRQAPGQGLEWMG
RIFPGDGDTDYNGKFKGRVTITADKSTSTAYMELSSLRSEDTAVYYCAR
NVFDGYWLVYWGQGTLVTVSS.
Obinutuzumab VL:
(SEQ ID NO: 141)
DIVMTQTPLSLPVTPGEPASISCRSSKSLLHSNGITYLYWYLQKPGQSP
QLLIYQMSNLVSGVPDRFSGSGSGTDFTLKISRVEAEDVGVYYCAQNLE
LPYTFGGGTKVEIK.

In another example, when the ABD that binds an antigen of interest binds to a EGFR polypeptide, exemplary VH and VL pairs can be selected from the EGFR-binding VL and VL pair from cetuximab, panitumumab, nimotuzumab, depatuxizumab and necitumumab:

Cetuximab VH:
(SEQ ID NO: 142)
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSA.
Cetuximab VL:
(SEQ ID NO: 143)
DILLTQSPVILSVSPGERVSFSCRASQSIGTNIHWYQQRTNGSPRLLIK
YASESISGIPSRFSGSGSGTDFTLSINSVESEDIADYYCQQNNNWPTTF
GAGTKLELK.
Panitumumab VH:
(SEQ ID NO: 144)
QVQLQESGPGLVKPSETLSLTCTVSGGSVSSGDYYWTWIRQSPGKGLEW
IGHIYYSGNTNYNPSLKSRLTISIDTSKTQFSLKLSSVTAADTAIYYCV
RDRVTGAFDIWGQGTMVTVSS.
Panitumumab VL:
(SEQ ID NO: 145)
DIQMTQSPSSLSASVGDRVTITCQASQDISNYLNWYQQKPGKAPKLLIY
DASNLETGVPSRFSGSGSGTDFTFTISSLQPEDIATYFCQHFDHLPLAF
GGGTKVEIK.
Nimotuzumab VH:
(SEQ ID NO: 146)
QVQLQQSGAEVKKPGSSVKVSCKASGYTFTNYYIYWVRQAPGQGLEWIG
GINPTSGGSNFNEKFKTRVTITADESSTTAYMELSSLRSEDTAFYFCTR
QGLWFDSDGRGFDFWGQGTTVTVSS.
Nimotuzumab VL:
(SEQ ID NO: 147)
DIQMTQSPSSLSASVGDRVTITCRSSQNIVHSNGNTYLDWYQQTPGKAP
KLLIYKVSNRFSGVPSRFSGSGSGTDFTFTISSLQPEDIATYYCFQYSH
VPWTFGQGTKLQI.
Necitumumab VH:
(SEQ ID NO: 148)
QVQLQESGPGLVKPSQTLSLTCTVSGGSISSGDYYWSWIRQPPGKGLEW
IGYIYYSGSTDYNPSLKSRVTMSVDTSKNQFSLKVNSVTAADTAVYYCA
RVSIFGVGTFDYWGQGTLVTVSS.
Necitumumab VL:
(SEQ ID NO: 149)
EIVMTQSPATLSLSPGERATLSCRASQSVSSYLAWYQQKPGQAPRLLIY
DASNRATGIPARFSGSGSGTDFTLTISSLEPEDFAVYYCHQYGSTPLTF
GGGTKAEIK.
Depatuxizumab VH:
(SEQ ID NO: 150)
QVQLQESGPGLVKPSQTLSLTCTVSGYSISSDFAWNWIRQPPGKGLEWM
GYISYSGNTRYQPSLKSRITISRDTSKNQFFLKLNSVTAADTATYYCVT
AGRGFPYWGQGTLVTVSS.
Depatuxizumab VL:
(SEQ ID NO: 151)
DIQMTQSPSSMSVSVGDRVTITCHSSQDINSNIGWLQQKPGKSFKGLIY
HGTNLDDGVPSRFSGSGSGTDYTLTISSLQPEDFATYYCVQYAQFPWTF
GGGTKLEIK.

In another example, when the ABD that binds an antigen of interest binds to a BCMA polypeptide, exemplary VH and VL pairs can be selected from the BCMA-binding VL and VL pair from belantamab, teclistamab, elranatamab or pavurutamab:

Belantamab VH:
(SEQ ID NO: 152)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSS.
Belantamab VL:
(SEQ ID NO: 153)
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIK.
Pavurutamab VH:
(SEQ ID NO: 154)
QVQLVQSGAEVKKPGASVKVSCKASGYTFTNHIIHWVRQAPGQCLEWMG
YINPYPGYHAYNEKFQGRATMTSDTSTSTVYMELSSLRSEDTAVYYCAR
DGYYRDTDVLDYWGQGTLVTVSS.
Pavurutamab VL:
(SEQ ID NO: 155)
DIQMTQSPSSLSASVGDRVTITCQASQDISNYLNWYQQKPGKAPKLLIY
YTSRLHTGVPSRFSGSGSGTDFTFTISSLEPEDIATYYCQQGNTLPWTF
GCGTKVEIK.

In another example, when the ABD that binds an antigen of interest binds to a PD-L1 polypeptide, exemplary VH and VL pairs can be selected from the PD-L1-binding VH and VL pair from antibodies 3G10, 12A4, 10A5, 5F8, 10H10, 1B12, 7H1, 11E6, 12B7, and 13G4 shown in U.S. Pat. No. 7,943,743, the disclosure of which is incorporated herein by reference, or of any of the antibodies MPDL3280A (atezolizumab, Tecentriq™, see, e.g., U.S. Pat. No. 8,217,149, anti-PD-L1 from Roche/Genentech), MDX-1105 (anti-PD-L1 from Bristol-Myers Squibb), MSB0010718C (avelumab; anti-PD-L1 from Pfizer) and MED14736 (durvalumab; anti-PD-L1 from AstraZeneca). In another example, when the ABD that binds an antigen of interest binds to a B7-H3 polypeptide, exemplary VH and VL pairs can be selected from the B7-H3-binding VH and VL pairs of enoblituzumab, TRL4542 shown in PCT publication no. WO2018/129090, 8H9 shown in PCT publication no. WO2018/209346, or any of the antibodies of PCT publication nos. WO2016/106004, WO2017/180813, WO2019/024911, WO2019/225787, WO2020/063673, WO2020/094120, WO2020/102779, WO2020/140094 and WO2020/151384. Examples of single domain B7H3 ABDs include Affibody™ formats described in PCT publication WO2020/041626 and single domain antibodies (sdAb) of PCT publication nos. WO2020/076970 and WO2021/247794. In another example, when the ABD that binds an antigen of interest binds to a B7-H6 polypeptide, exemplary VH and VL pairs can be selected from the B7-H6-binding VH and VL pairs shown in U.S. Pat. Nos. 11,034,766, 8,822,652, 9,676,855, 11,034,766, 11,034,767 or in PCT publication nos. WO2013/037727 or WO2021/064137. In another example, when the ABD that binds an antigen of interest binds to a B7-H4polypeptide, exemplary VH and VL pairs can be selected from the B7-H4-binding VH and VL of alsevalimab or the VH and VL pairs shown in U.S. Pat. Nos. 10,626,176 9,676,854, 9,574,000, 10,150,813, 10,814,011 or in PCT publication nos. WO2009/073533, WO2019/165077 WO2019/169212, WO2019/147670, WO2021/155307, WO2022/039490, WO2019/154315 or WO2021/185934. The disclosures of VH, VL and CDRs sequences of the above are incorporated herein by reference.

In one embodiment, the ABD that binds an antigen of interest binds to a cancer antigen, a viral antigen, a microbial antigen, or an antigen present on an infected cell (e.g. virally infected) or on a pro-inflammatory immune cell. In one embodiment, said antigen is a polypeptide selectively expressed or overexpressed on a tumor cell, and infected cell or a pro-inflammatory cell. In one embodiment, said antigen is a polypeptide that when inhibited, decreases the proliferation and/or survival of a tumor cell, an infected cell or a pro-inflammatory cell.

The ABDs which are incorporated into the polypeptides can be tested for any desired activity prior to inclusion in a multispecific NKp46-binding protein, for example the ABD can be tested in a suitable format (e.g. as conventional IgG antibody, fab, Fab′2 or scFv) for binding to (e.g. binding affinity) for its binding partner.

An ABD derived from an antibody will generally comprise at minimum a hypervariable region sufficient to confer binding activity. It will be appreciated that an ABD may comprise other amino acids or functional domains as may be desired, including but not limited to linker elements (e.g. linker peptides, CH1, Cκ or Cλ domains, hinges, or fragments thereof). In one example an ABD comprises an scFv, a V_{H} domain and a V_{L} domain, or a single domain antibody (nanobody or dAb) such as a V-NAR domain, Darpln or a V_{H}H domain. ABDs can be made of a V_{H} and a V_{L} domain that associate with one another to form the ABD.

In one embodiment, one or both of the V_{H} and V_{L} pairs that form an ABD for NKp46 and antigen of interest are within a tandem variable region such as an scFv (a V_{H} fused to a V_{L} domain via a flexible polypeptide linker).

In one embodiment, one or both ABDs for NKp46 and antigen of interest can have a conventional or non-conventional Fab structure. A Fab structure can be characterized as a VH or VL variable domain linked to a CH1 domain and a complementary variable domain (VL or VH, respectively) linked to a complementary Cκ (or Cλ) constant domain, wherein the CH1 and Cκ (or Cλ) constant domains associate (dimerize). For example a Fab can be formed from a VH-CH1 unit (VH fused to a CH1) on a first polypeptide chain that dimerizes with a VL-Cκ unit (VL fused to a CK) on a second chain. Alternatively, a Fab can be formed from a VH-Cκ unit (VH fused to a CK) on a first polypeptide chain that dimerizes with a VL-CH1 unit (VL fused to a CH1) on a second chain.

In some embodiments, one of the ABDs for NKp46 and antigen of interest comprises a Fab structure, in which a variable domain is linked to a CH1 domain and a complementary variable domain is linked to a complementary Cκ (or Cλ) constant domain, wherein the CH1 and Cκ (or Cλ) constant domains associate to form a heterodimeric protein, and the other ABD comprise or consists of an scFv or a single binding domain (e.g. VhH domain, DARPin). The scFv or a single binding domain can optionally be fused to a Cκ or Cλ domain or hinge domain.

The CH1 and/or Cκ domains can then be linked to a CH2 domain, optionally in each case via a hinge region (or a suitable domain linker). The CH2 domain(s) is/are then linked to a CH3 domain. The CH2-CH3 domains can thus optionally be embodied as a full-length Fc domain (optionally a full-length Fc domain, except that the CH3 domain that lacks the C-terminal lysine).

The Fc domain dimer that is capable of binding to human FcRn can be specified to be capable or binding to CD16A and optionally other Fcγ receptors (e.g., CD16B, CD32A, CD32B and/or CD64), or to have reduced (e.g. compared to a wild-type Fc domain) or abolished binding to CD16A and optionally other Fcγ receptors. In one embodiment, an Fc moiety may be obtained by production of the polypeptide in a host cell or by a process that yields N297-linked glycosylation, e.g. a mammalian cell. In one embodiment, an Fc moiety comprises a human gamma isotype constant region comprising one or more amino acid modifications, e.g. in the CH2 domain, that increases binding to CD16 or CD16A.

The cytokine receptor antigen binding domain can readily be embodied as a cytokine, (e.g. a type 1 cytokine such as an IL-2, IL-15, IL-21, IL-7, IL-27 or IL-12 cytokine, an IL-18 cytokine or a type 1 interferon such as IFN-α or IFN-β). Exemplary cytokine receptor ABDs and modified cytokines are further described herein.

Once appropriate antigen binding domains having desired specificity and/or activity are identified, nucleic acids encoding each of the or ABD can be separately placed, in suitable arrangements, in an appropriate expression vector or set of vectors, together with DNA encoding any elements such as CH1, CK, CH2 and CH3 domains or portions thereof, mutant IL2 polypeptides and any other optional elements (e.g. DNA encoding a hinge-derived or linker elements) for transfection into an appropriate host. ABDs will be arranged in an expression vector, or in separate vectors as a function of which type of polypeptide is to be produced, so as to produce the polypeptide chains having the desired domains operably linked to one another. The host is then used for the recombinant production of the multispecific polypeptide.

For example, a polypeptide fusion product can be produced from a vector in which one ABD or a part thereof (e.g. a VH, VL or a VH/VL pair) is operably linked (e.g. directly, or via a CH1, Cκ or Cλ constant region and/or hinge region) to the N-terminus of a CH2 domain, and the CH2 domain is operably linked at its C-terminus to the N-terminus a CH3 domain. Another ABD or part thereof can be on a second polypeptide chain that forms a dimer, e.g. heterodimer, with the polypeptide comprising the first ABD.

The multispecific polypeptide can then be produced in an appropriate host cell or by any suitable synthetic process. A host cell chosen for expression of the multispecific polypeptide is an important contributor to the final composition, including, without limitation, the variation in composition of the oligosaccharide moieties decorating the protein in the immunoglobulin CH2 domain. Thus, one aspect of the invention involves the selection of appropriate host cells for use and/or development of a production cell expressing the desired therapeutic protein such that the multispecific polypeptide retains FcRn and CD16 binding. The host cell may be of mammalian origin or may be selected from COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. The host cell may be any suitable species or organism capable of producing N-linked glycosylated polypeptides, e.g. a mammalian host cell capable of producing human or rodent IgG type N-linked glycosylation.

Multimeric, multispecific proteins such as heterodimers, heterotrimers and hetero-tetramers can be produced according to a variety of domain arrangements in which the antigen of interest binding domain and NKp46 binding domain can each independently be a Fab (e.g. a conventional or non-conventional Fab structure), scFv or single domain antibody (nanobody or dAb, such as a V-NAR domain, Darpln™ or a VHH domain). Different domains onto different polypeptide chain that associate to form a multimeric protein. Accordingly, different proteins can be constructed based on Fc domain dimers that are capable of binding to human FcRn polypeptide (neonatal Fc receptor), with or without additionally binding to CD16 or CD16A and optionally other Fcγ receptors, e.g., CD16B, CD32A, CD32B and/or CD64). The greatest potentiation of NK cell cytotoxicity can be obtained through use of Fc moieties that have substantial binding to the activating human CD16 receptor (CD16A) binding; such CD16 binding can be obtained through the use of suitable CH2 and/or CH3 domains, as further described herein. In one embodiment, an Fc moiety is derived from a human IgG1 isotype constant region. Use of modified CH3 domains also contributes to the possibility of use a wide range of heteromultimeric protein structures. Accordingly, a protein comprises a first and a second polypeptide chain each comprising a variable domain fused to a human Fc domain monomer, optionally a Fc domain monomer comprising a CH3 domain capable of undergoing preferential CH3-CH3 hetero-dimerization, wherein the first and second chain associate via CH3-CH3 dimerization and the protein consequently comprises a Fc domain dimer. The variable domains of each chain can be part of the same or different antigen binding domains.

Multispecific proteins can thus be conveniently constructed using VH and VL pairs arranged as scFv or Fab structures, together with CH1 domains, CL domain, Fc domains and cytokines, and domain linkers. Preferably, the proteins will use minimal non-natural sequences, e.g. minimal use of non-Ig linkers, optionally no more than 5, 4, 3, 2 or 1 domain linker(s) that is not an antibody-derived sequence, optionally wherein domain linker(s) are no more than 15, 10 or 5 amino acid residues in length. In one embodiment, the CD16 ABD is a Fc domain dimer.

In some embodiment, the multispecific proteins (e.g. dimers, trimers, tetramers) may comprise a domain arrangement of any of the following in which domains can be placed on the 2, 3 or 4 polypeptide chains, wherein the Fc domain is interposed between the NKp46 ABD and the antigen of interest (Antigen) ABD on the topological N-terminal side of the Fc domain dimer and the cytokine receptor ABD on the topological C-terminal end of the Fc domain dimer (e.g. the protein has (i) a terminal or distal cytokine receptor ABD at the C-terminal end and (ii) a terminal or distal antigen of interest (Antigen) ABD and NKp46 ABD at the topological N-terminal end), wherein the ABD that binds the cytokine receptor is connected to one of the Fc domain monomers of the Fc dimer via a flexible linker (e.g. a linker comprising G and S residues):
(NKp46 ABD)
   (Fc domain dimer) (cytokine receptor ABD)
(Antigen ABD)

The cytokine receptor ABD can be an IL2, IL15, IL18, IL21 or IFN-α polypeptide. The Fc domain dimer can be specified to bind human FcRn and optionally further one or more human Fcγ receptors (e.g. CD16A). The variable regions that associate to form a particular ABD can be on the same polypeptide chain or on different polypeptide chains. In one embodiment, one or both of the antigen of interest IL2, IL15, IL18, IL21 or IFN-α polypeptide (e.g. cancer antigen) ABD and the NKp46 ABD is formed from two variable regions present within a tandem variable region (e.g. an scFv), wherein In one embodiment, one or both of the antigen of interest ABD and NKp46 ABD comprises a tandem variable region (e.g. an scFv) and the other comprises a Fab structure. In another embodiment, both of the antigen of interest and NKp46 ABD comprises a Fab structure. In another embodiment one of the antigen of interest and NKp46 ABD comprises a Fab structure and the other comprises an scFv structure. In one embodiment, the IL2, IL15, IL18, IL21 or IFN-α polypeptide is fused, via a domain linker, to the C-terminus of a Fc domain monomer, which Fc domain in in turn fused at its N-terminus to an NKp46 ABD or to a portion thereof (e.g. a V-(CH1 or CL) segment).

The present disclosure provides advantageous approaches of making multimeric multispecific proteins which bind to the antigen of interest (monovalently or bivalently) and monovalently to each of NKp46, CD16A and cytokine receptor. The approaches readily allow domain configurations where the Fc domain is positioned between the NKp46 ABD and a cytokine polypeptide.

In certain examples, multimeric proteins may be composed of a central (first) polypeptide chain comprising one or two immunoglobulin variable domains, connected or fused, optionally via a CH1 or CL constant region or via a linker, to the N-terminus of an Fc domain, wherein the Fc domain is connected at its C-terminus to a cytokine polypeptide (e.g. to the C-terminus of the cytokine polypeptide. When only one variable domain is present on the central chain, an additional polypeptide chain (e.g. a light chain comprising a V and C domain, optionally a VK and a CK domain) will provide the complementary variable region to form and ABD with the variable domain of the central chain. When two variable domains are present on the central chain they can be arranged as an scFv to form an ABD. A further one or two additional chains (i.e. the third and fourth chain) can then provide at least one further ABD.

Thus, in addition to the first/central chain, a further one, two or three polypeptide chains will provide the complementary Fc domain and variable domains depending on whether the ABDs are configured as scFv or Fab. In this configuration, the Fc domain is interposed between the NKp46 binding ABD and the cytokine polypeptide in the multispecific protein. In this way, heterodimeric, heterotrimeric or heterotetrameric multispecific proteins can be constructed.

Examples of the domain arrangements (N- to C-termini, left to right) of central polypeptide chains include any of the following, wherein each V is a variable domain:

(Vₐ₋₁-(CH1 or C_{L})-(hinge or linker)-CH2-CH3-linker-Cyt (first/central chain)

or

(Vₐ₋₂-V_{b-2})-(hinge or linker)-CH2-CH3-linker-Cyt (first/central chain)

In these domain arrangements of the first/central chain, Vₐ₋₁ is a light chain or heavy chain variable domain which will form an ABD together with a variable region V_{b-1} on a further polypeptide chain (e.g. the further chain comprising the variable region V_{b-1} fused to a constant region); Vₐ₋₂ and V_{b-2} together form an scFv (one of Vₐ₋₂ and V_{b-2} is a light chain variable domain and the other is a heavy chain variable domain and they are separated by a flexible polypeptide linker; in any embodiment, a V-V can accordingly be specified as comprising a linker placed between the two V regions); and the CH3 domain is connected or fused to the cytokine via a domain linker (e.g. a flexible chemical or polypeptide linker). Optionally, when the ABD is a single domain such as a VHH, anticalin or Darpin™ type structure, Vₐ₋₂-V_{b-2} can be replaced by the respective single domain.

A second polypeptide chain can then be configured to comprise one or two immunoglobulin variable domains, optionally a constant region, and an Fc domain suitable to undergo CH3-CH3 dimerization with the first/central polypeptide chain. When the second polypeptide chain has one immunoglobulin variable domain it can conveniently be fused to a CH1 or CL domain which is in turn fused to the CH2 domain via a hinge region. When the second polypeptide chain has two immunoglobulin variable domains, the two variable domains can together form an scFv and be fused to the CH2 domain via a polypeptide linker.

A second polypeptide chain can comprise a domain arrangement:

(Vₐ₋₄-V_{b-4})-(hinge or linker)-CH2-CH3 (second chain)

or

(Vₐ₋₃-(CH1 or C_{K})-(hinge or linker)-CH2-CH3 (second chain)

In these domain arrangements of the second chain, Vₐ₋₃ is a light chain or heavy chain variable domain which will form an ABD together with a variable region V_{b-3} on a further polypeptide chain (e.g. a chain comprising the variable region V_{b-3} fused to a light chain constant region); Vₐ₋₄ and V_{b-4} together form an scFv (one of Vₐ₋₄ and V_{b-4} is a light chain variable domain and the other is a heavy chain variable domain and they are separated by a flexible polypeptide linker).

When dimeric proteins are desired, the following heterodimer can thus be configured:

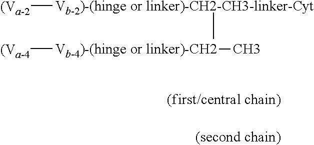

For heterotrimeric multispecific proteins, one of the first and second chains can be selected to comprise two variable domains (e.g., as an scFv), and a third polypeptide chain can then be provided, comprising the following domain arrangement:

(V-(CH1 or C_{κ}) (third chain)

In the domain arrangement of the third chain, V, which can be also be designated V_{b-1} or V_{b-3}, is a light chain or heavy chain variable domain which will form an ABD together with the variable region Vₐ₋₁ or Vₐ₋₃ on the first or second polypeptide chain. The CH1 or CK domain in the third chain will be selected so as to undergo CH1-CK dimerization with the respective first or second polypeptide chain with which the third chain is selected to associate (one of the associating chains has CH1 and the other has CK).

The resulting heterotrimer proteins can thus be constructed for example molecules having the following domain arrangement:

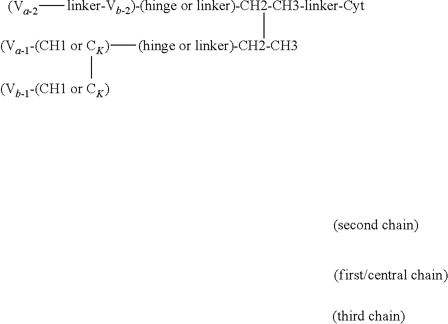

wherein the first/central chain and the second chain associate by CH3-CH3 dimerization and the first/central chain and the third chain associate by the CH1 or Cκ dimerization, wherein the domains of the first/central chain and the third chain are selected to be complementary to permit the first and third chains to associate by CH1-Cκ dimerization, and wherein Vₐ₋₁, V_{b-1}, Vₐ₋₂ and V_{b-2} are each a V_{H} domain or a V_{L} domain, and wherein one of Vₐ₋₁ and V_{b-1} is a V_{H} and the other is a V_{L} such that Vₐ₋₁ and V_{b-1} form a first antigen binding domain (ABD), wherein one of Vₐ₋₂ and V_{b-2} is a VH and the other is a V_{L} such that Vₐ₋₂ and V_{b-2} form a second antigen binding domain (e.g. an scFv wherein Vₐ₋₂ and V_{b-2} are separated by a linker), wherein one of the ABD binds NKp46 and the other binds an antigen of interest. In one specific embodiment, Vₐ₋₂ and V_{b-2} form an ABD that binds NKp46 and Vₐ₋₁ and V_{b-1} form an ABD that binds the antigen of interest. In another embodiment, Vₐ₋₂ and V_{b-2} form an ABD that binds the antigen of interest and Vₐ₋₁ and V_{b-1} form an ABD that binds NKp46.

Another exemplary structure has following domain arrangements:

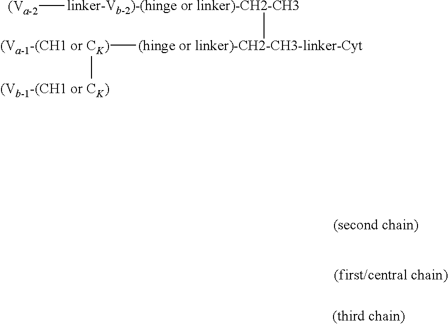

In one specific embodiment, Vₐ₋₁ and V_{b-1} form an ABD that binds NKp46 and Vₐ₋₂ and V_{b-2} form an ABD that binds the antigen of interest. In another embodiment, Vₐ₋₁ and V_{b-1} form an ABD that binds the antigen of interest and Vₐ₋₂ and V_{b-2} form an ABD that binds NKp46.

In heterotetrameric multispecific proteins, both of the first and second chains can be selected to comprise one variable domain fused to a CH1 or CK constant domain, and a third polypeptide chain as shown above can be provided, as well as a fourth polypeptide chain comprising the following domain arrangement:

(V-(CH1 or C_{κ}) (fourth chain)

In the domain arrangement of the fourth chain, V, which can be also be designated V_{b-1} or V_{b-3}, is a light chain or heavy chain variable domain which will form an ABD together with the variable region Vₐ₋₁ or Vₐ₋₃ on the first or second polypeptide chain. If the V of the third chain is designated V_{b-1} and the third chain associates with the first chain such that V_{b-1} and Vₐ₋₁ form an ABD, then the V of the fourth chain will be V_{b-3} and the fourth chain will associate with the second chain such that V_{b-3} associates with Vₐ₋₃ to form an ABD. The CH1 or CK domain in the fourth chain will be selected so as to undergo CH1-CK dimerization with the respective first or second polypeptide chain with which the fourth chain is selected to associate (one of the associating chains has CH1 and the other has CK).

The resulting heterotetramer proteins can then be constructed, for example molecules having the following domain arrangements:

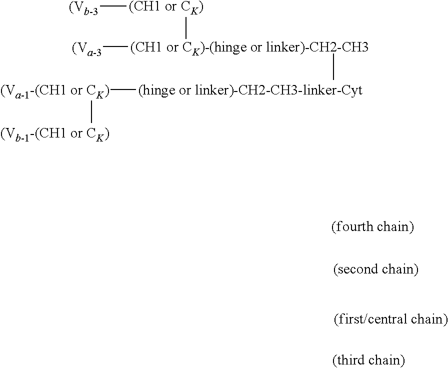

wherein the first/central chain and the second chain associate by CH3-CH3 dimerization and the first/central chain and the third chain associate by the CH1 or Cκ dimerization, wherein the domains of the first/central chain and the third chain are selected to be complementary to permit the first and third chains to associate by CH1-Cκ dimerization, wherein the domains of the second chain and the fourth chain are selected to be complementary to permit the second and fourth chains to associate by CH1-Cκ dimerization, and wherein Vₐ₋₁, V_{b-1}, Vₐ₋₃ and V_{b-3} are each a V_{H} domain or a V_{L} domain, and wherein one of Vₐ₋₁ and V_{b-1} is a V_{H} and the other is a V_{L} such that Vₐ₋₁ and V_{b-1} form a first antigen binding domain (ABD), wherein one of Vₐ₋₃ and V_{b-3} is a VH and the other is a V_{L} such that Vₐ₋₃ and V_{b-3} form a second antigen binding domain, wherein one of the ABD binds NKp46 and the other binds an antigen of interest. In one specific embodiment, Vₐ₋₃ and V_{b-3} an ABD that binds NKp46 and Vₐ₋₁ and V_{b-1} form an ABD that binds the antigen of interest. In another specific embodiment, Vₐ₋₃ and V_{b-3} an ABD that binds the antigen of interest and Vₐ₋₁ and V_{b-1} form an ABD that binds NKp46.

In this way, a range of different domain arrangements can be constructed from the variable domains, constant domains and (L2v polypeptides. Example of domain arrangements of resulting heterodimer, heterotrimer and heterotetramer proteins are shown in Table 1 below (domain linkers not shown).

| | |
|---|---|
| 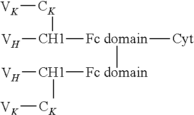 | (third polypeptide) (first polypeptide) (second polypeptide) (fourth polypeptide) |
| 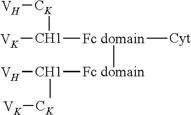 | (third polypeptide) (first polypeptide) (second polypeptide) (fourth polypeptide) |
| 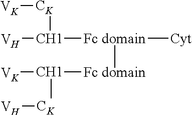 | (third polypeptide) (first polypeptide) (second polypeptide) (fourth polypeptide) |
| 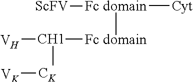 | (first polypeptide) (second polypeptide) (third polypeptide) |
| 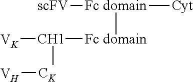 | (first polypeptide) (second polypeptide) (third polypeptide) |
| 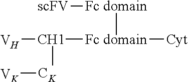 | (first polypeptide) (second polypeptide) (third polypeptide) |
| 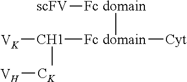 | (first polypeptide) (second polypeptide) (third polypeptide) |

As illustrated in Table 1, in some embodiments, multispecific heterotetramer proteins can be generated on the natural immunoglobulin architecture containing two pairs of heavy chain and light chain combination with each pair having distinct binding specificity, with the cytokine polypeptide bound to the C-terminus of the Fc domain of one of the two heavy chains (one of the first and second chains), e.g. via a domain linker. Other multispecific heterotrimer and heterotetramer proteins can be constructed in which VH and VK domains are substituted by one another and/or in which CH1 and CK are domains are substituted by one another compared to the natural immunoglobulin architecture. If desired, CH1 and/or CL domains can be engineered to promote or enhance the desired heterodimerization through steric repulsion, or charge steering interaction. The correct dimerization of the light chains (third and where present fourth chains) can be enhanced through the introduction of amino acid substitutions that give rise to attractive/repulsive charge pairs into the CH1 and CK domains. Homodimerization of the two heavy chains is mediated by the CH3 interaction. To promote heterodimeric formation, amino acid substitutions can be introduced to the two respective CH3 regions.

In one embodiment, a multispecific protein can be generated by post-production assembly from structures based on half-antibodies, wherein one of the heavy chains bears at its C-terminus a cytokine fused via a domain linker (e.g., a (-linker-Cyt) moiety fused to the C-terminus of the Fc domain monomer), thereby solving the issues of heavy and light chain mispairing. Such multispecific protein will advantageously contain modification to favor heterodimerization of the half-antibodies, for example comprising a F405L mutation in one Fc monomer and a K409R mutation in the other Fc monomer, see, e.g., Labrijn et al., (2013) PNAS 110 (13) 5145-515. Each half-antibody-type structure is individually produced in separate cell line and purified. The purified antibodies are then subjected to mild reduction to obtain half-antibodies, which were then assembled into multispecific proteins and purified from the mixture using conventional purifications methods.

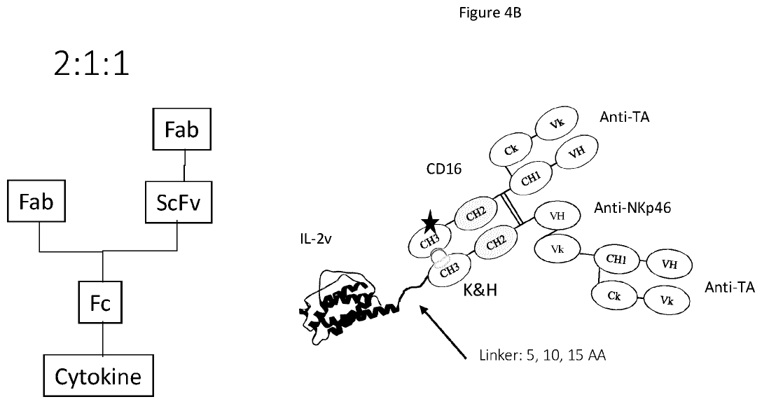

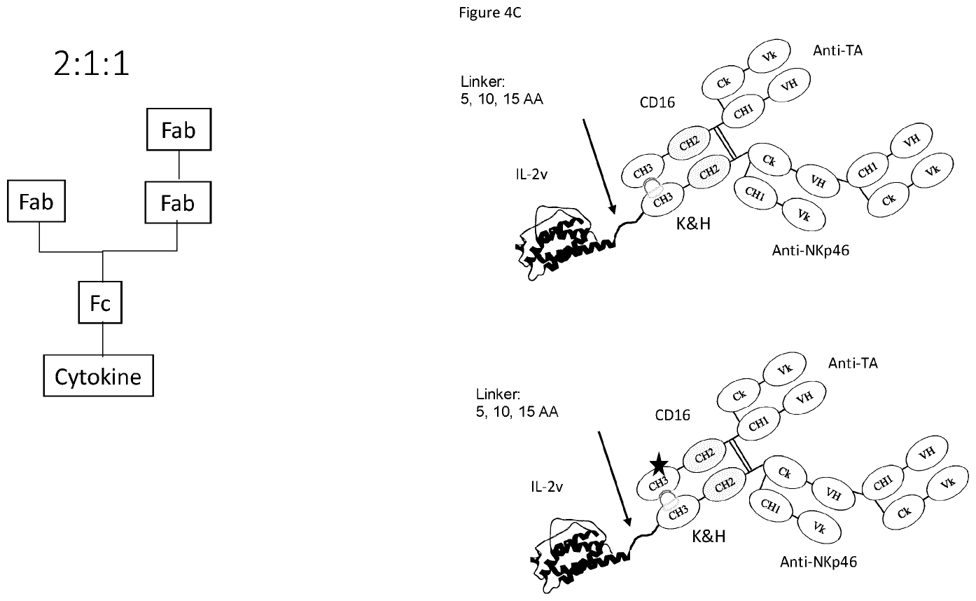

Yet further multispecific proteins can be produced using similar architecture that has two binding sites for antigen of interest (e.g. binds the antigen(s) of interest bivalently or binds to each of two different antigens of interest monovalently), binds NKp46 monovalently and binds the cytokine receptor monovalently, i.e., the multispecific protein has a 2:1:1 configuration. Examples are shown in FIG. 4.

One example is heterotetramer protein made from three different polypeptides which has two Fab structures and one scFv, where the two Fabs bind to the antigen(s) of interest. The protein can be constructed in which the Fc domain is interposed between the NKp46 ABD and the cytokine, having the following domain arrangements:

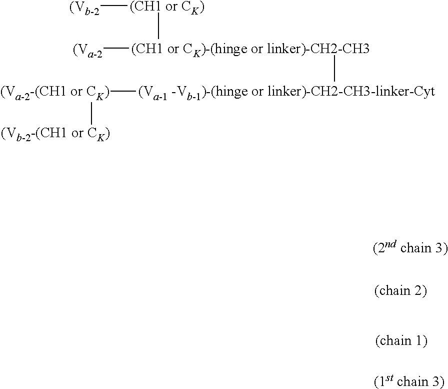

wherein the chain 1 and the chain 2 associate by CH3-CH3 dimerization, the chain 1 and the chain 3 (1^{st} of two identical chain 3 polypeptides) associate by CH1 or Cκ dimerization, and the chain 2 and the chain 3 (2^{nd} of two identical chain 3 polypeptides) associate by CH1 or Cκ dimerization, wherein the domains of the chains 1, 2 and the chain 3 are selected to be complementary to permit the chains 1 and 2 to associate with chain 3 by CH1-Cκ dimerization, and wherein Vₐ₋₁, V_{b-1}, Vₐ₋₂ and V_{b-2} are each a V_{H} domain or a V_{L} domain, and wherein one of Vₐ₋₁ and V_{b-1} is a V_{H} and the other is a V_{L} such that Vₐ₋₁ and V_{b-1} form a first antigen binding domain (ABD), wherein one of Vₐ₋₂ and V_{b-2} is a VH and the other is a V_{L} such that Vₐ₋₂ and V_{b-2} form a second antigen binding domain, wherein Vₐ₋₁ and V_{b-1} an ABD that binds NKp46 and Vₐ₋₂ and V_{b-2} form the ABDs that binds the antigen(s) of interest.

Another example is heteropentamer protein constructed from four different chains which has three Fab structures, two of which bind to the antigen of interest. The protein can be constructed in which the Fc domain is interposed between the NKp46 ABD and the cytokine, having the following domain arrangements:

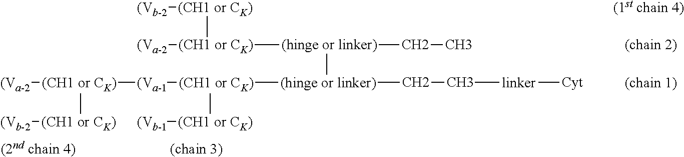

wherein the chain 1 and the chain 2 associate by CH3-CH3 dimerization, the chain 1 and the chain 3 associate by CH1 or Cκ dimerization, and the chain 1 and the chain 4 (2^{nd} of two identical chain 4 polypeptides) associate by CH1 or Cκ dimerization, wherein the domains of the chain 1 and the chain 3 are selected to be complementary to permit the chains 1 and 3 to associate by CH1-Cκ dimerization, wherein the domains of the chains 1 and 2 and 4 are selected to be complementary to permit the chains 1 and 2 and chain 4 (each of the two identical chain 4 polypeptides) to associate by CH1-Cκ dimerization, and wherein Vₐ₋₁, V_{b-1}, Vₐ₋₂ and V_{b-2} are each a V_{H} domain or a V_{L} domain, and wherein one of Vₐ₋₁ and V_{b-1} is a V_{H} and the other is a V_{L} such that Vₐ₋₁ and V_{b-1} form a first antigen binding domain (ABD), wherein one of Vₐ₋₂ and V_{b-2} is a VH and the other is a V_{L} such that Vₐ₋₂ and V_{b-2} form a second antigen binding domain, wherein Vₐ₋₁ and V_{b-1} an ABD that binds NKp46 and Vₐ₋₂ and V_{b-2} form the ABDs that binds the antigen(s) of interest.

In any embodiment, it may be specified that the protein has a Fc domain dimer comprised of a first and second Fc domain monomer placed on separate chains that dimerize via CH3-CH3 association, wherein one of the Fc domain monomers is connected to the both the anti-NKp46 ABD and the cytokine, and the other (second) Fc domain monomer has a free C-terminus (e.g., no anti-NKp46 ABD or cytokine fused to its C-terminus).

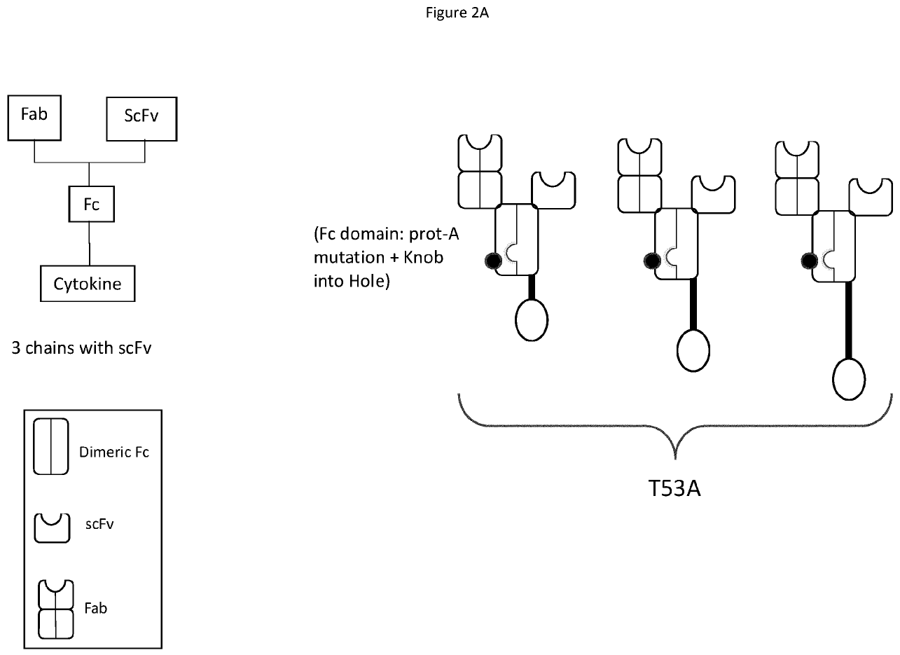

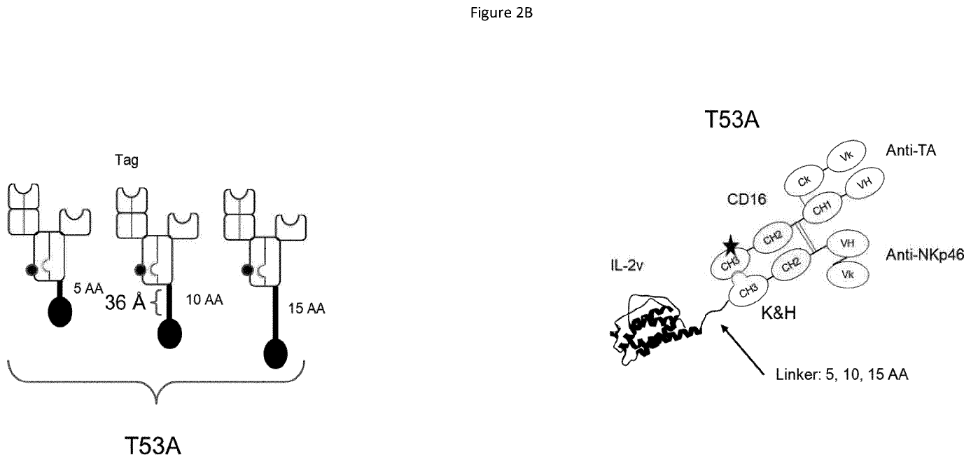

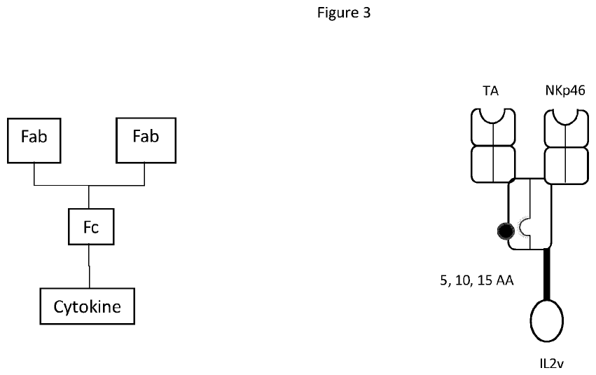

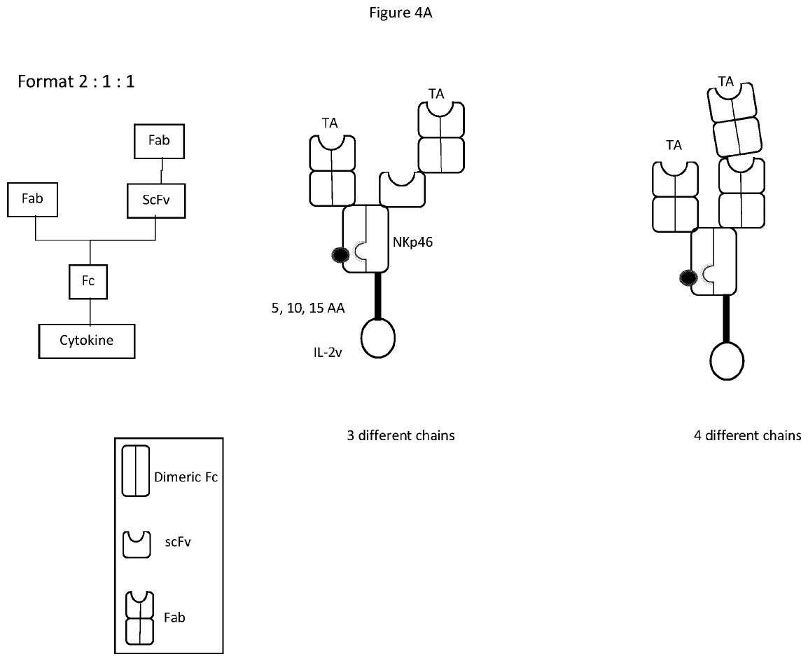

Optionally in any embodiment herein, fusions or linkages on the same polypeptide chain between different domains, particularly where the domains are not naturally directly fused to one another (e.g., between two V domains placed in tandem, between V domains and Fc domain monomers, between CH1 or Cκ domains and Fc domains, between Fc domain monomers and cytokine) may occur via intervening amino acid sequences, for example via a hinge region or linker peptide. In some domain arrangements or structures herein that are depicted without showing domain linkers, it will be appreciated that the domain arrangements can be specified as having domain linkers between specified domains. For example, the cytokine can be specified as being fused to an adjacent domain via a domain linker, and a domain linker can be inserted in the relevant domain arrangement or structure. In another example, tandem variable domains (e.g. in an scFv) can be specified as being fused to one another via a domain linker, and a domain linker can be inserted between the two V regions in the relevant domain arrangement or structure. In another example, a CH1 or CL (or CK) constant region can be fused to an Fc domain or CH2 domain thereof via a domain linker or hinge domain or portion thereof, and accordingly a domain linker or hinge domain or portion thereof can be inserted between CH1 or CL domain and the Fc domain or CH2 domain in the relevant domain arrangement or structure. An example of the domain arrangement of a multispecific protein with linkers shown is shown in FIG. 2B for the representative heterotrimer in format “T53A”, shows domain linkers such as hinge and glycine-serine linkers, and interchain disulfide bridges.

In any embodiment herein, a polypeptide chain (e.g., chain 1, 2, 3 or 4) can be specified as having a free N and/or C terminus (no other protein domains at the terminus of the polypeptide chain).

In any embodiment herein, the proteins domains described herein can optionally be specified as being indicated from N- to C-termini. Protein arrangements of the disclosure for purposes of illustration are shown from N-terminus (on the left) to C-terminus (on the right). Adjacent domains on a polypeptide chain can be referred to as being fused to one another (e.g. a domain can be said to be fused to the C-terminus of the domain on its left, and/or a domain can be said to be fused to the N-terminus of the domain on its right). The proteins domains described herein can be fused to one another directly (e.g. V domains fused directly to CH1 or CL domains) or via linkers or short intervening amino acid sequences that serve to connect the domains on a polypeptide chain (e.g. they may optionally be specified to lack other predetermined functionality, or to lack specific binding to a predetermined ligand). Two polypeptide chains will be bound to one another (indicated by “i”), by non-covalent interactions, and optionally can further be attached via interchain disulfide bonds, formed between cysteine residues within complementary CH1 and Cκ domains.

### Connections and Linkers

Generally, there are a number of suitable linkers that can be used in the multispecific proteins, including peptide bonds, generated by recombinant techniques. In some embodiments, the linker is a “domain linker”, used to link any two domains as outlined herein together. Adjacent protein domains can be specified as being connected or fused to one another by a domain linker. An exemplary domain linker is a (poly)peptide linker, optionally a flexible (poly)peptide linker. Amino acid-based linkers such as peptide linkers or polypeptide linkers, used interchangeably herein, may have a subsequence derived from a particular domain such as a hinge, CH1 or CL domain, or may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 2 to 30 amino acids in length. In one embodiment, linkers of 4 to 20 amino acids in length may be used, with from about 5 to about 15 amino acids finding use in some embodiments. While any suitable linker can be used, many embodiments, linkers (e.g. flexible linkers) can utilize a glycine-serine polypeptide or polymer, including for example comprising (GS)ₙ, (GSG₂S)ₙ, (G₄S)ₙ, (GSSS)ₙ, (GSSSS)ₙ (SEQ ID NO: 171) and (GGGS)ₙ, where n is an integer of at least one (optionally n is 1, 2, 3 or 4), glycine-alanine polypeptide, alanine-serine polypeptide, and other flexible linkers. Linkers comprising glycine and serine residues generally provides protease resistance. One example of a (GS), linker is a linker having the amino acid sequence STGS; such a linker can be useful to fuse a domain to the C-terminus of an Fc domain (or a CH3 domain thereof). In some embodiments peptide linkers comprising (G₂S)ₙ are used, wherein, for example, n=1-20, e.g., (G₂S), (G₂S)₂, (G₂S)₃, (G₂S)₄, (G₂S)₅, (G₂S)₆, (G₂S)₇ or (G₂S)₈, or (G₃S)ₙ, wherein, for example, n is an integer from 1-15. In one embodiment, a domain linker comprises a (G₄S)ₙ peptide, wherein, for example, n is an integer from 1-10, optionally 1-6, optionally 1-4. In some embodiments peptide linkers comprising (GS₂)ₙ (GS₃)ₙ or (GS₄)ₙ are used, wherein, for example, n=1-20, e.g., (GS₂), (GS₂)₂, (GS₂)₃, (GS₃)₁, (GS₃)₂, (GS₃)₃, (GS₄)₁, (GS₄)₂, (GS₄)₃, wherein, for example, n is an integer from 1-15. In one embodiment, a domain linker comprises a (GS₄)ₙ peptide, wherein, for example, n is an integer from 1-10, optionally 1-6, optionally 1-4. In one embodiment, a domain linker comprises a C-terminal GS dipeptide, e.g., the linker comprises (GS₄) and has the amino acid sequence a GSSSS (SEQ ID NO: 171), GSSSSGSSSS (SEQ ID NO: 172), GSSSSGSSSSGS (SEQ ID NO: 173) or GSSSSGSSSSGSSSS (SEQ ID NO: 174).

Any of the peptide or domain linkers may be specified to comprise a length of at least 3 residues, at least 4 residues, at least 5 residues, at least 10 residues, at least 15 residues, or more. In other embodiments, the linkers comprise a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, between 2-30 residues, between 2 and 50 residues, between 5-15 residues or between 10 and 50 residues.

Examples of polypeptide linkers may include sequence fragments from CH1 or CL domains; for example the first 4-12 or 5-12 amino acid residues of the CL/CH1 domains are particularly useful for use in linkages of scFv moieties. Linkers can be derived from immunoglobulin light chains, for example CK or Cλ. Linkers can be derived from immunoglobulin heavy chains of any isotype, including for example Cy1, Cy2, Cy3, Cy4 and Cμ. Linker sequences may also be derived from other proteins such as Ig-like proteins (e.g. TCR, FcR, KIR), hinge region-derived sequences, and other natural sequences from other proteins. In certain domain arrangements, V_{H} and V_{L} domains are linked to another in tandem separated by a linker peptide (e.g. an scFv) and in turn be fused to the N- or C-terminus of an Fc domain (or CH2 domain thereof). Such tandem variable regions or scFv can be connected to the Fc domain via a hinge region or a portion thereof, an N-terminal fragment of a CH1 or CL domain, or a glycine- and serine-containing flexible polypeptide linker.

Fc domains can be connected to other domains via immunoglobulin-derived sequence or via non-immunoglobulin sequences, including any suitable linking amino acid sequence. Advantageously, immunoglobulin-derived sequences can be readily used between CH1 or CL domains and Fc domains, in particular, where a CH1 or CL domain is fused at its C-terminus to the N-terminus of an Fc domain (or CH2 domain). An immunoglobulin hinge region or portion of a hinge region can and generally will be present on a polypeptide chain between a CH1 domain and a CH2 domain. A hinge or portion thereof can also be placed on a polypeptide chain between a CL (e.g. CK) domain and the CH2 domain of an Fc domain when a CL is adjacent to an Fc domain on the polypeptide chain. However, it will be appreciated that a hinge region can optionally be replaced for example by a suitable linker peptide, e.g. a flexible polypeptide linker.

In tandem variable regions (e.g. scFv), two V domains (e.g. a V_{H} domain and V_{L} domains are generally linked together by a linker of sufficient length to enable the ABD to fold in such a way as to permit binding to the antigen for which the ABD is intended to bind. Examples of linkers include linkers comprising glycine and serine residues, e.g., the amino acid sequence GEGTSTGSGGSGGSGGAD (SEQ ID NO: 388). In another specific embodiment, the V_{H} domain and V_{L} domains of an scFv are linked together by the amino acid sequence (G₄S)₃.

In one embodiment, a (poly)peptide linker used to link a VH or VL domain of an scFv to a CH2 domain of an Fc domain comprises a fragment of a CH1 domain or CL domain and/or hinge region. For example, an N-terminal amino acid sequence of CH1 can be fused to a variable domain in order to mimic as closely as possible the natural structure of a wild-type antibody. In one embodiment, the linker comprises an amino acid sequence from a hinge domain or an N-terminal CH1 amino acid. In one embodiment, the linker peptide mimics the regular VK-CK elbow junction, e.g., the linker comprises or consists of the amino acid sequence RTVA.

In one embodiment, the hinge region used to connect the C-terminal end of a CH1 or CK domain (e.g. of a Fab) with the N-terminal end of a CH2 domain will be a fragment of a hinge region (e.g. a truncated hinge region without cysteine residues) or may comprise one or more amino acid modifications which remove (e.g. substitute by another amino acid, or delete) a cysteine residue, optionally both cysteine residues in a hinge region. Removing cysteines can be useful to prevent undesired disulfide bond formation, e.g., the formation of disulfide bridges in a monomeric polypeptide.

A “hinge” or “hinge region” or “antibody hinge region” herein refers to the flexible polypeptide or linker between the first and second constant domains of an antibody. Structurally, the IgG CH1 domain ends at EU position 220, and the IgG CH2 domain begins at residue EU position 237. Thus for an IgG the hinge generally includes positions 221 (D221 in IgG1) to 236 (G236 in IgG1), wherein the numbering is according to the EU index. References to specific amino acid residues within constant region domains found within the polypeptides shall be, unless otherwise indicated or as otherwise dictated by context, be defined according to Kabat, in the context of an IgG antibody.

In one embodiment, the hinge region (or fragment thereof) is derived form a hinge domain of a human IgG1 antibody. For example a hinge domain may comprise the amino acid sequence: THTCPPCPAPELL (SEQ ID NO: 166), or an amino acid sequence at least 60%, 70%, 80% or 90% identical thereto, optionally wherein one or both cysteines are deleted or substituted by a different amino acid residue.

In one embodiment, the hinge region (or fragment thereof) is derived from a Cμ2-C Cμ3 hinge domain of a human IgM antibody. For example a hinge domain may comprise the amino acid sequence: NASSMCVPSPAPELL (SEQ ID NO: 167), or an amino acid sequence at least 60%, 70%, 80% or 90% identical thereto, optionally wherein one or both cysteines are deleted or substituted by a different amino acid residue.

Polypeptide chains that dimerize and associate with one another via non-covalent bonds or interactions may or may not additionally be bound by an interchain disulfide bond formed between respective CH1 and Cκ domains, and/or between respective hinge domains on the chains. CH1, Cκ and/or hinge domains (or other suitable linking amino acid sequences) can optionally be configured such that interchain disulfide bonds are formed between chains such that the desired pairing of chains is favored and undesired or incorrect disulfide bond formation is avoided. For example, when two polypeptide chains to be paired each possess a CH1 or Cκ adjacent to a hinge domain, the polypeptide chains can be configured such that the number of available cysteines for interchain disulfide bond formation between respective CH1/Cκ-hinge segments is reduced (or is entirely eliminated). For example, the amino acid sequences of respective CH1, Cκ and/or hinge domains can be modified to remove cysteine residues in both the CH1/Cκ and the hinge domain of a polypeptide; thereby the CH1 and Cκ domains of the two chains that dimerize will associate via non-covalent interaction(s).

In another example, the CH1 or Cκ domain adjacent to (e.g., N-terminal to) a hinge domain comprises a cysteine capable of interchain disulfide bond formation, and the hinge domain which is placed at the C-terminus of the CH1 or Cκ comprises a deletion or substitution of one or both cysteines of the hinge (e.g. Cys 239 and Cys 242, as numbered for human IgG1 hinge according to Kabat). In one embodiment, the hinge region (or fragment thereof) comprise the amino acid sequence: THTSPPSPAPELL (SEQ ID NO: 168), or an amino acid sequence at least 60%, 70%, 80% or 90% identical thereto.

In another example, the CH1 or Cκ domain adjacent (e.g., N-terminal to) a hinge domain comprises a deletion or substitution at a cysteine residue capable of interchain disulfide bond formation, and the hinge domain placed at the C-terminus of the CH1 or Cκ comprises one or both cysteines of the hinge (e.g. Cys 239 and Cys 242, as numbered for human IgG1 hinge according to Kabat). In one embodiment, the hinge region (or fragment thereof) comprises the amino acid sequence: THTCSSCPAPELL (SEQ ID NO: 169), or an amino acid sequence at least 60%, 70%, 80% or 90% identical thereto.

In another example, a hinge region is derived from an IgM antibody. In such embodiments, the CH1/Cκ pairing mimics the Cμ2 domain homodimerization in IgM antibodies. For example, the CH1 or Cκ domain adjacent (e.g., N-terminal to) a hinge domain comprises a deletion or substitution at a cysteine capable of interchain disulfide bond formation, and an IgM hinge domain which is placed at the C-terminus of the CH1 or Cκ comprises one or both cysteines of the hinge. In one embodiment, the hinge region (or fragment thereof) comprises the amino acid sequence: THTCSSCPAPELL (SEQ ID NO: 170), or an amino acid sequence at least 60%, 70%, 80% or 90% identical thereto.

Alternatively to the polypeptide linkers, a variety of nonproteinaceous polymer or chemical linkers may find use in the multispecific proteins. For example nonproteinaceous polymers including but not limited to polyethylene glycol (PEG), polypropylene glycol, polyoxyalkylenes, or copolymers of polyethylene glycol and polypropylene glycol, may find use as linkers. In some examples, an amino acid sequence in a polypeptide chain of a multispecific protein may be modified to introduce a reactive group, optionally a protected reactive group, and the so-modified protein or chain is then reacted with a linker or a polypeptide comprising a complementary reactive group. In some examples, an amino acid residue in a polypeptide chain of a multispecific protein can be bound to a linker comprising a reactive group (for further reaction with a second polypeptide functionalized with a linker with a complementary reactive group) or directly a second polypeptide via an enzyme catalyzed reaction. For example, a polypeptide comprising an acceptor glutamine or lysine can reacted with linker comprising a primary amine in the presence of a transglutamine enzyme (e.g. Bacterial Transglutaminase, BTG) such that the transglutaminase enzyme catalyzes the conjugation of the linker to an acceptor glutamine residue within the primary structure of the polypeptide, for example within an immunoglobulin constant domain or within a TGase recognition tag inserted or appended to (e.g., fused to) a constant region. A second polypeptide can also be functionalized with a linker in a similar manner, and when the conjugated linkers each bear complementary reactive groups (e.g. R on the linker of one polypeptide and R′ on the linker of the other polypeptide), the two functionalized polypeptides can be reacted such that they are bound via the linker comprising the residue of the reaction or R with R′. Examples of reactive group pairs R and R′ include a range of groups capable of biorthogonal reaction, for example 1,3-dipolar cycloaddition between azides and cyclooctynes (copper-free click chemistry), between nitrones and cyclooctynes, oxime/hydrazone formation from aldehydes and ketones and the tetrazine ligation (see also WO2013/092983). The resulting linker and functionalized antibody, or the Y element thereof, can thus comprise a RR′ group resulting from the reaction of R and R′, for example a triazole. Methods and linkers for use in BTG-mediated conjugation to antibodies is described in PCT publication no. WO2014/202773, the disclosure of which is incorporated by reference. “Transglutaminase”, used interchangeably with “TGase” or “TG”, refers to an enzyme capable of cross-linking proteins through an acyl-transfer reaction between the γ-carboxamide group of peptide-bound glutamine and the ε-amino group of a lysine or a structurally related primary amine such as amino pentyl group, e.g. a peptide-bound lysine, resulting in a ε-(γ-glutamyl)lysine isopeptide bond. TGases include, inter alia, bacterial transglutaminase (BTG) such as the enzyme having EC reference EC 2.3.2.13 (protein-glutamine-γ-glutamyltransferase). The term “acceptor glutamine” residue, when referring to a glutamine residue of an antibody, means a glutamine residue that is recognized by a TGase and can be cross-linked by a TGase through a reaction between the glutamine and a lysine or a structurally related primary amine such as amino pentyl group. Preferably the acceptor glutamine residue is a surface-exposed glutamine residue. The term “TGase recognition tag” refers to a sequence of amino acids comprising an acceptor glutamine residue and that when incorporated into (e.g. appended to) a polypeptide sequence, under suitable conditions, is recognized by a TGase and leads to cross-linking by the TGase through a reaction between an amino acid side chain within the sequence of amino acids and a reaction partner. The recognition tag may be a peptide sequence that is not naturally present in the polypeptide comprising the enzyme recognition tag. Examples of TGase recognition tags include the amino acid sequences disclosed in WO2012/059882 and WO2014/072482, the disclosure of which sequences are incorporated herein by reference

### Constant Regions

Constant region domains can be derived from any suitable human antibody, particularly human antibodies of gamma isotype, including, the constant heavy (CH1) and light (CL, Cκ or Cλ) domains, hinge domains, CH2 and CH3 domains.

With respect to heavy chain constant domains, “CH1” generally refers to positions 118-220 according to the EU index. Depending on the context, a CH1 domain (e.g. as shown in the domain arrangements), can optionally comprise residues that extend into the hinge region such that the CH1 comprises at least part of a hinge region. For example, when positioned C-terminal on a polypeptide chain and/or or C-terminal to the Fc domain, and/or within a Fab structure that is or C-terminal to the Fc domain, the CH1 domain can optionally comprise at least part of a hinge region, for example CH1 domains can comprise at least an upper hinge region, for example an upper hinge region of a human IgG1 hinge, optionally further in which the terminal threonine of the upper hinge can be replaced by a serine. Such a CH2 domain can therefore comprise at its C-terminus the amino acid sequence: EPKSCDKTHS (SEQ ID NO: 389).

Exemplary human CH1 domain amino acid sequences include:

(SEQ ID NO: 156)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKR
V
or
(SEQ ID NO: 157)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHS.
or
(SEQ ID NO: 158)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHT.

Exemplary human Cκ domain amino acid sequences include:

(SEQ ID NO: 159)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In some exemplary configurations, the multispecific protein can be a heterodimer, a heterotrimer or a heterotetramer comprising one or two Fabs (e.g. one Fab binding NKp46 and the other binding the antigen of interest), in which variable regions, CH1 and/or CL domains are engineered by introducing amino acid substitutions in a knob-into-holes or electrostatic steering approach to promote the desired chain pairings of CH1 domains with CK domains. In some exemplary configurations, the multispecific protein can be a heterodimer, a heterotrimer or a heterotetramer comprising one or two Fabs (e.g. one Fab binding NKp46 and the other binding the antigen of interest), wherein a Fab has a VH/VL crossover (VH and VL replace one another) or a CH1/CL crossover (the CH1 and CL replace one another), and wherein the CH1 and/or CL domains comprise amino acid substitutions to promote correct chain association by knob-into-holes or electrostatic steering.

“CH2” generally refers to positions 237-340 according to the EU index, and “CH3” generally refers to positions 341-447 according to the EU index. CH2 and CH3 domains can be derived from any suitable antibody. Such CH2 and CH3 domains can be used as wild-type domains or may serve as the basis for a modified CH2 or CH3 domain. Optionally the CH2 and/or CH3 domain is of human origin or may comprise that of another species (e.g., rodent, rabbit, non-human primate) or may comprise a modified or chimeric CH2 and/or CH3 domain, e.g., one comprising portions or residues from different CH2 or CH3 domains, e.g., from different antibody isotypes or species antibodies.

In any of the domain arrangements, the Fc domain monomer may comprise a CH2-CH3 unit (a full length CH2 and CH3 domain or a fragment thereof). In heterodimers or heterotrimers comprising two chains with Fc domain monomers (i.e. the heterodimers or heterotrimers comprise a Fc domain dimer), the CH3 domain will be capable of CH3-CH3 dimerization (e.g. it will comprise a wild-type CH3 domain or a CH3 domain with a wild-type sequence at the CH3 interface, or it will comprise a CH3 domain with modifications to promote a desired CH3-CH3 dimerization).

Exemplary human IgG1 CH2-CH3 Fc domain amino acid sequences include:

(SEQ ID NO: 160)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCS
VMHEALHNHYTQKSLSLSPG.

An Fc domain may optionally further comprise a C-terminal lysine (K). In some exemplary configurations, the multispecific protein can be a heterodimer, a heterotrimer or a heterotetramer, wherein the polypeptide chains are engineered for heterodimerization among each other so as to produce the desired protein. In embodiments where the desired chain pairings are not driven by CH1-Cκ dimerization or where enhancement of pairing is desired, the chains may comprise constant or Fc domains with amino acid modifications (e.g., substitutions) that favor the preferential hetero-dimerization of the two different chains over the homo-dimerization of two identical chains.

In some embodiments, a “knob-into-holes” approach is used in which the domain interfaces (e.g. CH3 domain interface of the antibody Fc region) are mutated so that the antibodies preferentially heterodimerize. These mutations create altered charge polarity across the interface (e.g. Fc dimer interface) such that co-expression of electrostatically matched chains (e.g. Fc-containing chains) support favorable attractive interactions thereby promoting desired heterodimer (e.g. Fc heterodimer) formation, whereas unfavorable repulsive charge interactions suppress unwanted heterodimer (e.g., Fc homodimer) formation. See for example mutations and approaches reviewed in Brinkmann and Kontermann, 2017 MAbs, 9(2): 182-212, the disclosure of which is incorporated herein by reference. For example the “Hole” mutations on a first Fc monomer can comprise Y349C/T366S/L368A/Y407V and the complementary “Knob” mutations on the second Fc monomer can comprise S354C/T366W (Kabat EU numbering). For example one heavy chain comprises a T366W substitution and the second heavy chain comprises a T366S, L368A and Y407V substitution, see, e.g. Ridgway et al (1996) Protein Eng., 9, pp. 617-621; Atwell (1997) J. Mol. Biol., 270, pp. 26-35; and WO2009/089004, the disclosures of which are incorporated herein by reference. In another approach, one heavy chain comprises a F405L substitution and the second heavy chain comprises a K409R substitution, see, e.g., Labrijn et al. (2013) Proc. Natl. Acad. Sci. U.S.A., 110, pp. 5145-5150. In another approach, one heavy chain comprises T350V, L351Y, F405A, and Y407V substitutions and the second heavy chain comprises T350V, T366S, K392L, and T394W substitutions, see, e.g. Von Kreudenstein et al., (2013) mAbs 5:646-654. In another approach, one heavy chain comprises both K409D and K392D substitutions and the second heavy chain comprises both D399K and E356K substitutions, see, e.g. Gunasekaran et al., (2010) J. Biol. Chem. 285:19637-19646. In another approach, one heavy chain comprises D221E, P228E and L368E substitutions and the second heavy chain comprises D221R, P228R, and K409R substitutions, see, e.g. Strop et al., (2012) J. Mol. Biol. 420: 204-219. In another approach, one heavy chain comprises S364H and F405A substitutions and the second heavy chain comprises Y349T and, T394F substitutions, see, e.g. Moore et al., (2011) mAbs 3: 546-557. In another approach, one heavy chain comprises a H435R substitution and the second heavy chain optionally may or may not comprise a substitution, see, e.g. U.S. Pat. No. 8,586,713. When such hetero-multimeric antibodies have Fc regions derived from a human IgG2 or IgG4, the Fc regions of these antibodies can be engineered to contain amino acid modifications that permit CD16 binding. In some embodiments, the antibody may comprise mammalian antibody-type N-linked glycosylation at residue N297 (Kabat EU numbering).

In some embodiments, one or more pairs of disulfide bonds such as A287C and L306C, V259C and L306C, R292C and V302C, and V323C and I332C (Kabat numbering) are introduced into the Fc region to increase stability, for example further to a loss of stability caused by other Fc modifications. Additional example includes introducing K3381, A339K, and K340S mutations to enhance Fc stability and aggregation resistance (Gao et al, 2019 Mol Pharm. 2019; 16:3647).

In some embodiments, where a multispecific protein is intended to have reduced binding to a human Fc gamma receptor. In some embodiments, where a multispecific protein is intended to have reduced binding to a human CD16A polypeptide (and optionally further reduced binding to CD32A, CD32B and/or CD64), the Fc domain is a human IgG4 Fc domain, optionally further wherein the Fc domain comprises a S228P mutation to stabilize the hinge disulfide. In one embodiment, the Fc domain has an amino acid sequence at least 90%, 95% or 99% identical to a human IgG4 Fc domain, optionally further comprising a Kabat S228P mutation.

In embodiments, where a multispecific protein is intended to have reduced binding to human CD16A polypeptide (and optionally further reduced binding to CD32A, CD32B and/or CD64), a CH2 and/or CH3 domain (or Fc domain comprising same) may comprise a modification to decrease or abolish binding to FcγRIIIA (CD16). For example, CH2 mutations in a Fc domain dimer proteins at reside N297 (Kabat numbering) can substantially eliminate CD16A binding. However the person of skill in the art will appreciate that other configurations can be implemented. For example, substitutions into human IgG1 or IgG2 residues at positions 233-236 and/or residues at positions 327, 330 and 331 were shown to greatly reduce binding to Fcγ receptors and thus ADCC and CDC. Furthermore, Idusogie et al. (2000) J. Immunol. 164(8):4178-84 demonstrated that alanine substitution at different positions, including K322, significantly reduced complement activation.

In one embodiment, the asparagine (N) at Kabat heavy chain residue 297 can be substituted by a residue other than an asparagine (e.g. a glutamine, a residue other than glutamine, for example a serine).

In one embodiment, an Fc domain modified to reduce binding to CD16A comprises a substitution in the Fc domain at Kabat residues 234, 235 and 322. In one embodiment, the protein comprises a substitution in the Fc domain at Kabat residues 234, 235 and 331. In one embodiment, the protein comprises a substitution in the Fc domain at Kabat residues 234, 235, 237 and 331. In one embodiment, the protein comprises a substitution in the Fc domain at Kabat residues 234, 235, 237, 330 and 331. In one embodiment, the Fc domain is of human IgG1 subtype. Amino acid residues are indicated according to EU numbering according to Kabat.

In one embodiment, an Fc domain modified to reduce binding to CD16A comprises an amino acid modification (e.g. substitution) at one or more of Kabat residue(s) 233-236, optionally one or more of residues 233-237, or at one, two or three of residues 234, 235 and/or 237, and an amino acid modification (e.g. substitution) at Kabat residue(s) 330 and/or 331. One example of such an Fc domain comprises substitutions at Kabat residues L234, L235 and P331 (e.g., L234A/L235E/P331S or (L234F/L235E/P331S). Another example of such an Fc domain comprises substitutions at Kabat residues L234, L235, G237 and P331 (e.g., L234A/L235E/G237A/P331S). Another example of such an Fc domain comprises substitutions at Kabat residues L234, L235, G237, A330 and P331 (e.g., L234A/L235E/G237A/A330S/P331S). In one embodiment, an antibody comprises an human IgG1 Fc domain comprising L234A/L235E/N297X/P331S substitutions, L234F/L235E/N297X/P331S substitutions, L234A/L235E/G237A/N297X/P331S substitutions, or L234A/L235E/G237A/N297X/A330S/P331S substitutions, wherein X can be any amino acid other than an asparagine. In one embodiment, X is a glutamine; in another embodiment, X is a residue other than a glutamine (e.g. a serine).

In one embodiment, an Fc domain that has low or reduced binding to CD16A comprises a human IgG4 Fc domain, wherein the Fc domain has the amino acid sequence below (human IgG4 with S228P substitution), or an amino acid sequence at least 90%, 95% or 99% identical thereto.

(SEQ ID NO: 161)
ASTKG PSVFPLAPCS RSTSESTAAL GCLVKDYFPE PVTVSWNSGA LTSGVHTFPA
VLQSSGLYSL SSVVTVPSSS LGTKTYTCNV DHKPSNTKVD KRVESKYGPP
CPPCPAPEFL GGPSVFLFPP KPKDTLMISR TPEVTCVVVD VSQEDPEVQF
NWYVDGVEVH NAKTKPREEQ FNSTYRVVSV LTVLHQDWLN GKEYKCKVSN
KGLPSSIEKT ISKAKGQPRE PQVYTLPPSQ EEMTKNQVSL TCLVKGFYPS
DIAVEWESNG QPENNYKTTP PVLDSDGSFF LYSRLTVDKS RWQEGNVFSC
SVMHEALHNH YTQKSLSLSL.

In one embodiment, an Fc domain modified to reduce binding to CD16A comprises the amino acid sequence below, or an amino acid sequence at least 90%, 95% or 99% identical thereto but retaining the amino acid residues at Kabat positions 234, 235 and 331 (underlined):

(SEQ ID NO: 162)
A S T K G P S V F P L A P S S K S T S G G T A A L G C L V K D
Y F P E P V T V S W N S G A L T S G V H T F P A V L Q S S G L
Y S L S S V V T V P S S S L G T Q T Y I C N V N H K P S N T K
V D K R V E P K S C D K T H T C P P C P A P E A E G G P S V F
L F P P K P K D T L M I S R T P E V T C V V V D V S H E D P E
V K F N W Y V D G V E V H N A K T K P R E E Q Y N S T Y R V V
S V L T V L H Q D W L N G K E Y K C K V S N K A L P A S I E K
T I S K A K G Q P R E P Q V Y T L P P S R E E M T K N Q V S L
T C L V K G F Y P S D I A V E W E S N G Q P E N N Y K T T P P
V L D S D G S F F L Y S K L T V D K S R W Q Q G N V F S C S V
M H E A L H N H Y T Q K S L S L S P G.

In one embodiment, an Fc domain modified to reduce binding to CD16A comprises the amino acid sequence below, or an amino acid sequence at least 90%, 95% or 99% identical thereto but retaining the amino acid residues at Kabat positions 234, 235 and 331 (underlined):

(SEQ ID NO: 163)
A S T K G P S V F P L A P S S K S T S G G T A A L G C L V K D
Y F P E P V T V S W N S G A L T S G V H T F P A V L Q S S G L
Y S L S S V V T V P S S S L G T Q T Y I C N V N H K P S N T K
V D K R V E P K S C D K T H T C P P C P A P E F E G G P S V F
L F P P K P K D T L M I S R T P E V T C V V V D V S H E D P E
V K F N W Y V D G V E V H N A K T K P R E E Q Y N S T Y R V V
S V L T V L H Q D W L N G K E Y K C K V S N K A L P A S I E K
T I S K A K G Q P R E P Q V Y T L P P S R E E M T K N Q V S L
T C L V K G F Y P S D I A V E W E S N G Q P E N N Y K T T P P
V L D S D G S F F L Y S K L T V D K S R W Q Q G N V F S C S V
M H E A L H N H Y T Q K S L S L S P G.

In one embodiment, an Fc domain modified to reduce binding to CD16A comprises the amino acid sequence below, or an amino acid sequence at least 90%, 95% or 99% identical thereto but retaining the amino acid residues at Kabat positions 234, 235, 237, 330 and 331 (underlined):

(SEQ ID NO: 164)
A S T K G P S V F P L A P S S K S T S G G T A A L G C L V K D
Y F P E P V T V S W N S G A L T S G V H T F P A V L Q S S G L
Y S L S S V V T V P S S S L G T Q T Y I C N V N H K P S N T K
V D K R V E P K S C D K T H T C P P C P A P E A E G A P S V F
L F P P K P K D T L M I S R T P E V T C V V V D V S H E D P E
V K F N W Y V D G V E V H N A K T K P R E E Q Y N S T Y R V V
S V L T V L H Q D W L N G K E Y K C K V S N K A L P S S I E K
T I S K A K G Q P R E P Q V Y T L P P S R E E M T K N Q V S L
T C L V K G F Y P S D I A V E W E S N G Q P E N N Y K T T P P
V L D S D G S F F L Y S K L T V D K S R W Q Q G N V F S C S V
M H E A L H N H Y T Q K S L S L S P G.

In one embodiment, an Fc domain modified to reduce binding to CD16A comprises the amino acid sequence below, or a sequence at least 90%, 95% or 99% identical thereto but retaining the amino acid residues at Kabat positions 234, 235, 237 and 331 (underlined):

(SEQ ID NO: 165)
A S T K G P S V F P L A P S S K S T S G G T A A L G C L V K D
Y F P E P V T V S W N S G A L T S G V H T F P A V L Q S S G L
Y S L S S V V T V P S S S L G T Q T Y I C N V N H K P S N T K
V D K R V E P K S C D K T H T C P P C P A P E A E G A P S V F
L F P P K P K D T L M I S R T P E V T C V V V D V S H E D P E
V K F N W Y V D G V E V H N A K T K P R E E Q Y N S T Y R V V
S V L T V L H Q D W L N G K E Y K C K V S N K A L P A S I E K
T I S K A K G Q P R E P Q V Y T L P P S R E E M T K N Q V S L
T C L V K G F Y P S D I A V E W E S N G Q P E N N Y K T T P P
V L D S D G S F F L Y S K L T V D K S R W Q Q G N V F S C S V
M H E A L H N H Y T Q K S L S L S P G.

Any of the above Fc domain sequences can optionally further comprise a C-terminal lysine (K), i.e. as in the naturally occurring sequence.

In certain embodiments herein where binding to CD16 (CD16A) is desired, a CH2 and/or CH3 domain (or Fc domain comprising same) may have wild-type/unmodified Fc gamma receptor binding sites (e.g. a wild-type Fc domain) or may comprise one or more amino acid modifications (e.g. amino acid substitutions) which increase binding to human CD16 and optionally another receptor such as FcRn. Optionally, the modifications will not substantially decrease or abolish the ability of the Fc-derived polypeptide to bind to neonatal Fc receptor (FcRn), e.g. human FcRn. Typical modifications include modified human IgG1-derived constant regions comprising at least one amino acid modification (e.g. substitution, deletions, insertions), and/or altered types of glycosylation, e.g., hypofucosylation. Such modifications can affect interaction with Fc receptors: FcγRI (CD64), FcγRII (CD32), and FcγRIII (CD16). FcγRI (CD64), FcγRIIA (CD32A) and FcγRIII (CD 16) are activating (i.e., immune system enhancing) receptors while FcγRIIB (CD32B) is an inhibiting (i.e., immune system dampening) receptor. A modification may, for example, increase binding of the Fc domain to FcγRIIIa on effector (e.g. NK) cells and/or decrease binding to FcγRIIB. Examples of modifications are provided in PCT publication no. WO2014/044686, the disclosure of which is incorporated herein by reference. Specific mutations (in IgG1 Fc domains) which affect (enhance) FcγRIIIa or FcRn binding are also set forth below.

| | | | Effector | Effect of |
|---|---|---|---|---|
| Isotype | Species | Modification | Function | Modification |
| IgG1 | Human | T250Q/M428L | Increased | Increased |
| | | | binding to FcRn | half-life |
| IgG1 | Human | 1M252Y/S254T/ | Increased | Increased |
| | | T256E + | binding to FcRn | half-life |
| | | H433K/N434F | | |
| IgG1 | Human | E333A | Increased | Increased |
| | | | binding to | ADCC and |
| | | | FcγRIIIa | CDC |
| IgG1 | Human | S239D/I332E or | Increased | Increased |
| | | S239D/A330L/ | binding to | ADCC |
| | | I332E | FcγRIIIa | |
| IgG1 | Human | P257I/Q311 | Increased | Unchanged |
| | | | binding to FcRn | half-life |
| IgG1 | Human | S239D/I332E/ | Increased | Increased |
| | | G236A | FcγRIIa/FcγRIIb | macrophage |
| | | | ratio | phagocytosis |

In some embodiments, the multispecific protein comprises a variant Fc region comprise at least one amino acid modification (for example, possessing 1, 2, 3, 4, 5, 6, 7, 8, 9, or more amino acid modifications) in the CH2 and/or CH3 domain of the Fc region, wherein the modification enhances binding to a human CD16 polypeptide. In other embodiments, the multispecific protein comprises at least one amino acid modification (for example, 1, 2, 3, 4, 5, 6, 7, 8, 9, or more amino acid modifications) in the CH2 domain of the Fc region from amino acids 237-341, or within the lower hinge-CH2 region that comprises residues 231-341. In some embodiments, the multispecific protein comprises at least two amino acid modifications (for example, 2, 3, 4, 5, 6, 7, 8, 9, or more amino acid modifications), wherein at least one of such modifications is within the CH3 region and at least one such modifications is within the CH2 region. Encompassed also are amino acid modifications in the hinge region. In one embodiment, encompassed are amino acid modifications in the CH1 domain, optionally in the upper hinge region that comprises residues 216-230 (Kabat EU numbering). Any suitable functional combination of Fc modifications can be made, for example any combination of the different Fc modifications which are disclosed in any of U.S. Pat. Nos. 7,632,497; 7,521,542; 7,425,619; 7,416,727; 7,371,826; 7,355,008; 7,335,742; 7,332,581; 7,183,387; 7,122,637; 6,821,505 and 6,737,056; and/or in PCT Publications Nos. WO2011/109400; WO 2008/105886; WO 2008/002933; WO 2007/021841; WO 2007/106707; WO 06/088494; WO 05/115452; WO 05/110474; WO 04/1032269; WO 00/42072; WO 06/088494; WO 07/024249; WO 05/047327; WO 04/099249 and WO 04/063351; and/or in Lazar et al. (2006) Proc. Nat. Acad. Sci. USA 103(11): 405-410; Presta, L. G. et al. (2002) Biochem. Soc. Trans. 30(4):487-490; Shields, R. L. et al. (2002) J. Biol. Chem. 26; 277(30):26733-26740 and Shields, R. L. et al. (2001) J. Biol. Chem. 276(9):6591-6604).

In some embodiments, the multispecific protein comprises an Fc domain comprising at least one amino acid modification (for example, 1, 2, 3, 4, 5, 6, 7, 8, 9, or more amino acid modifications) relative to a wild-type Fc region, such that the molecule has an enhanced binding affinity for human CD16 relative to the same molecule comprising a wild-type Fc region, optionally wherein the variant Fc region comprises a substitution at any one or more of positions 221, 239, 243, 247, 255, 256, 258, 267, 268, 269, 270, 272, 276, 278, 280, 283, 285, 286, 289, 290, 292, 293, 294, 295, 296, 298, 300, 301, 303, 305, 307, 308, 309, 310, 311, 312, 316, 320, 322, 326, 329, 330, 332, 331, 332, 333, 334, 335, 337, 338, 339, 340, 359, 360, 370, 373, 376, 378, 392, 396, 399, 402,404, 416, 419, 421, 430,434, 435, 437, 438 and/or 439 (Kabat EU numbering).

In one embodiment, the multispecific protein comprises an Fc domain comprising at least one amino acid modification (for example, 1, 2, 3, 4, 5, 6, 7, 8, 9, or more amino acid modifications) relative to a wild-type Fc region, such that the molecule has enhanced binding affinity for human CD16 relative to a molecule comprising a wild-type Fc region, optionally wherein the variant Fc region comprises a substitution at any one or more of positions 239, 298, 330, 332, 333 and/or 334 (e.g. S239D, S298A, A330L, I332E, E333A and/or K334A substitutions), optionally wherein the variant Fc region comprises a substitution at residues S239 and I332, e.g. a S239D and I332E substitution (Kabat EU numbering).

In some embodiments, the multispecific protein comprises an Fc domain comprising N-linked glycosylation at Kabat residue N297. In some embodiments, the multispecific protein comprises an Fc domain comprising altered glycosylation patterns that increase binding affinity for human CD16. Such carbohydrate modifications can be accomplished by, for example, by expressing a nucleic acid encoding the multispecific protein in a host cell with altered glycosylation machinery. Cells with altered glycosylation machinery are known in the art and can be used as host cells in which to express recombinant antibodies to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1176195; PCT Publications WO 06/133148; WO 03/035835; WO 99/54342, each of which is incorporated herein by reference in its entirety. In one aspect, the multispecific protein contains one or more hypofucosylated constant regions. Such multispecific protein may comprise an amino acid alteration or may not comprise an amino acid alteration and/or may be expressed or synthesized or treated under conditions that result in hypofucosylation. In one aspect, a multispecific protein composition comprises a multispecific protein described herein, wherein at least 20, 30, 40, 50, 60, 75, 85, 90, 95% or substantially all of the antibody species in the composition have a constant region comprising a core carbohydrate structure (e.g. complex, hybrid and high mannose structures) which lacks fucose. In one embodiment, provided is a multispecific protein composition which is free of N-linked glycans comprising a core carbohydrate structure having fucose. The core carbohydrate will preferably be a sugar chain at Asn297.

Optionally, a multispecific protein comprising a Fc domain dimer can be characterized by having a binding affinity to a human CD16A polypeptide that is within 1-log of that of a conventional human IgG1 antibody, e.g., as assessed by surface plasmon resonance.

In one embodiment, the multispecific protein comprising a Fc domain dimer in which an Fc domain is engineered to enhance Fc receptor binding can be characterized by having a binding affinity to a human CD16A polypeptide that is at least 1-log greater than that of a conventional or wild-type human IgG1 antibody, e.g., as assessed by surface plasmon resonance.

In one embodiment, a multispecific protein comprising a Fc domain dimer can be characterized by having a binding affinity to a human FcRn (neonatal Fc receptor) polypeptide that is within 1-log of that of a conventional human IgG1 antibody, e.g., as assessed by surface plasmon resonance.

Optionally a multispecific protein comprising a Fc domain dimer can be characterized by a Kd for binding (monovalent) to a human Fc receptor polypeptide (e.g., CD16A) of less than 10⁻⁵ M (10 μmolar), optionally less than 10⁻⁶ M (1 μmolar), as assessed by surface plasmon resonance (e.g. as in the Examples herein, SPR measurements performed on a Biacore T100 apparatus (Biacore GE Healthcare), with bispecific antibodies immobilized on a Sensor Chip CM5 and serial dilutions of soluble CD16 polypeptide injected over the immobilized bispecific antibodies.

### Cytokine Receptor ABD

The antigen binding domain that binds to a cytokine receptor on NK cells (cytokine receptor ABD) can advantageously comprise a suitable cytokine polypeptide or polypeptide fragment such that the cytokine receptor ABD binds the cytokine receptor on the surface of an NK cell. The cytokine can for example be a full length wild-type IL-2, IL-15, IL-21, IL-7, IL-27, IL-12, IL-18, IFN-α or IFN-β polypeptide, a fragment thereof sufficient to bind to the NK cell receptor for such cytokine, or a variant of any of the foregoing. The cytokine molecule can be a fragment comprising at least 20, 30, 40, 50, 60, 70, 80 or 100 contiguous amino acids of a human cytokine, wherein the cytokine retains the ability to bind its cytokine receptor present on the surface of an NK cell. In certain embodiments, the cytokine is a variant of a human cytokine comprising one or more amino acid modifications (e.g. amino acid substitutions) compared to the wild-type human cytokine, for example to decrease binding affinity to a receptor present on non-NK cells, for example Treg cells, CD4 T cells, CD8 T cells. The cytokine can for example be a type I cytokine and a member of the common cytokine receptor gamma-chain (cg-chain) cytokine family, that signals via a heteromultimeric or heterdimeric receptor complex comprised of a receptor subunit (e.g., IL-2Rβ/IL-15RP or IL-21R) subunit that associates with the common gamma-chain (CD132).

In one embodiment, the multispecific proteins that binds to NKp46 and optionally further CD16A permits the incorporation of a wild-type cytokine (or fragment of variant thereof) that retain substantially full activity and/or binding affinity at the cytokine receptor expressed on NK cells, in comparison to the human wild-type cytokine counterpart. In one embodiment, the cytokine is a wild-type cytokine or fragment thereof, or is a modified cytokine, wherein the cytokine is does not have a substantially reduced ability to induce signaling and/or does not have a substantially reduced binding affinity at its receptor on NK cells (e.g. CD122, IL-21R, IL-7Ra, IL-27Ra, IL-12R, IL-18R). In one embodiment, the cytokine does not comprise a modification (e.g., substitution, deletion, etc.) that substantially reduces its ability to induce signaling through its receptor on NK cells (e.g. CD122, IL-21R, IL-7Ra, IL-27Ra, IL-12R, IL-18R). In one embodiment, the cytokine retains at least 80%, 90% of the ability of a wild-type cytokine counterpart to induce signaling through its receptor on NK cells (e.g. CD122, IL-21R, IL-7Ra, IL-27Ra, IL-12R, IL-18R). Optionally, signaling is assessed by bringing the cytokine (e.g. as a recombinant protein domain or within a multispecific protein of the disclosure) into contact with an NK cell and measuring signaling, e.g. measuring STAT phosphorylation in the NK cells.

In some embodiments, when the exemplary anti-NKp46 VH/VL pairs disclosed herein having a KD for NKp46 in the range of about 15 nM, or functionally conservative variants thereof, are used in the multispecific proteins, the cytokine or cytokine receptor ABD (either as a free cytokine or as incorporated into a multispecific protein) can be specified as binding its receptor, as determined by SPR, with a binding affinity (KD) of 200 nM or less, 100 nM or less, 50 nM or less or 25 nM or less. In one embodiment, the cytokine or cytokine receptor ABD binds its receptor, as determined by SPR, with a binding affinity (KD) that is 1 nM or higher than 1 nM, optionally that is higher than 10 nM optionally that is higher than 15 nM. In one embodiment, the cytokine or cytokine receptor ABD binds its receptor, as determined by SPR, with a binding affinity (KD) between about 1 nm and about 200 nm, optionally) between about 1 nm and about 100 nm optionally between about 10 nM and about 200 nM, optionally between about 10 nM and about 100 nM optionally between about 15 nM and about 100 nM.

When the cytokine-binding ABD is a CD122-binding ABD, the ABD can be or comprise a suitable interleukin-2 (IL-2) polypeptide such that the CD122 ABD binds CD122. As exemplified herein, the ABD is advantageously a variant or modified IL-2 polypeptide that has reduced binding to CD25 (IL-2Rα) (e.g. reduced or abolished binding affinity, for example as determined by SPR) compared to a wild-type human interleukin-2. Such a variant or modified IL-2 polypeptide is also referred herein to as an “IL2v” or a “not-alpha IL-2”. The CD122-binding ABD can optionally be specified to have a binding affinity for human CD122 that is substantially equivalent to that of wild-type human IL-2, or that is reduced (attenuated) compared to wild-type human IL-2. The CD122-binding ABD can optionally be specified to have an ability to induce CD122 signaling and/or binding affinity for CD122 that is substantially equivalent to that of wild-type human IL-2. In one embodiment, the CD122-binding ABD has a reduction in binding affinity for CD25 that is greater than the reduction in binding affinity for CD122, for example a reduction of at least 1-log, 2-log or 3-log in binding affinity for CD25 and a reduction in binding affinity for CD122 that is less than 1-log.

IL-2 is believed to bind IL-2Rβ (CD122) in its form as a monomeric IL-2 receptor (IL-2R), followed by recruitment of the IL-2Rγ (CD132; also termed common γ chain) subunit. In cells that do not express CD25 at their surface, binding (e.g. reduced binding) to CD122 can therefore optionally be specified as being binding in or to a CD122:CD132 complex. The CD122 (or CD122:CD132 complex) can optionally be specified as being present at the surface of an NK cell. In cells that express CD25 at their surface, IL-2 is believed to bind CD25 (IL-2Rα) in its form as a monomeric IL-2 receptor, followed by association of the subunits IL-2Rβ and IL-2Rγ. Binding (e.g. reduced binding, partially reduced binding) to CD25 can therefore optionally be specified as being binding in or to a CD25:CD122 complex or a CD25:CD122:CD132 complex.

In a multispecific protein herein, the multispecific protein can optionally be specified as being configured and/or in a conformation (or capable of adopting a conformation) in which the CD122 ABD (e.g. IL2v) is capable of binding to CD122 at the surface of a cell (e.g. an NK cell, a CD122+CD25− cell) when the multispecific protein is bound to NKp46 (and optionally further to CD16) at the surface of said cell. Optionally further, the multispecific protein:CD122 complex is capable of binding to CD132 at the surface of said cell.

The CD122 ABD or IL2v can be a modified IL-2 polypeptide, for example a monomeric IL-2 polypeptide modified by introducing one more amino acid substitutions, insertions or deletions that decrease binding to CD25.

In some embodiments, where binding to CD25 is sought to be selectively decreased, a IL-2 polypeptide can be modified by binding or associating it with one or more other additional molecules such as polymers or (poly)peptides that result in a further decrease of or abolished binding to CD25. For example a wild-type or mutated IL-2 polypeptide can be modified or further modified by binding to it another moiety that shields, masks, binds or interacts with CD25-binding site of human IL-2, thereby decreasing binding to CD25. In some examples, molecules such as polymers (e.g. PEG polymers) are conjugated to an IL-2 polypeptide to shield or mask the epitope on IL-2 that is bound by CD25, for example by introduction (e.g. substitution) to install an amino acid containing a dedicated chemical hook at a specific site on the IL-2 polypeptide. In other examples, a wild-type or variant IL-2 polypeptide is bound to anti-IL-2 monoclonal antibody or antibody fragment that binds or interacts with CD25-binding site of human IL-2, thereby decreasing binding to CD25.

In any embodiment, an IL2 polypeptide can be a full-length IL-2 polypeptide or it can be an IL-2 polypeptide fragment, so long as the fragment or IL2v that comprises it retains the specified activity (e.g. retaining at least partial CD122 binding, compared to wild-type IL-2 polypeptide).

As shown herein, an IL2v polypeptide can advantageously comprise an IL-2 polypeptide comprising one or more amino acid mutations designed to reduce its ability to bind to human CD25 (IL-2Rα), while retaining at least at least some, or optionally substantially full, ability to bind human CD122.

Various IL2v or not-alpha IL-2 moieties have been described which reduce the activation bias of IL-2 on CD25⁺ cells. Such IL2v reduce binding to IL-2Rα and maintain at least partial binding to IL-2Rβ. Several IL2v polypeptides have been described, many having mutations in amino acid residue regions 35-72 and/or 79-92 of the IL-2 polypeptide. For example, decreased affinity to IL-2Rα may be obtained by substituting one or more of the following residues in the sequence of a wild-type IL-2 polypeptide: R38, F42, K43, Y45, E62, P65, E68, V69, and L72 (amino acid residue numbering is with reference to the mature IL-2 polypeptide shown in SEQ ID NO: 352).

The wild-type mature human IL-2 protein and a wild-type mature IL-2p protein fragment lacking the three first residues APT are shown below in SEQ ID NOS: 352 and 353, respectively:

Wild-type mature human IL-2
(SEQ ID NO: 352)
APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTEMCEYADETATIVEFLNRWITFCQSIISTLT.
Wild-type mature IL-2p:
(SEQ ID NO: 353)
SSSTKKTQLQLEHLLLDLQMILNGINNYKNPKLTRMLTFKFYMPKKATE
LKHLQCLEEELKPLEEVLNLAQSKNFHLRPRDLISNINVIVLELKGSET
TEMCEYADETATIVEFLNRWITFCQSIISTLT.

An exemplary IL2v (also referred to herein as IL2v in the Examples) can have the amino acid of wild-type IL-2 with the five amino acid substitutions T3A, F42A, Y45A, L72G and C125A, as shown below, optionally further with deletion of the three N-terminal residues APA:

(SEQ ID NO: 354)
APASSSTKKTQLQLEHLLLDLQMILNGINNYKNPKLTRMLTAKFAMPKK
ATELKHLQCLEEELKPLEEVLNGAQSKNFHLRPRDLISNINVIVLELKG
SETTEMCEYADETATIVEFLNRWITFAQSIISTLT.

As few as one or two mutations can reduce binding to IL-2Rα and L-2Rβ. For example, as exemplified in the multispecific proteins herein, the IL2v polypeptide having two amino acid substitutions R38A and F42K in the wild-type IL-2p amino acid sequence displayed suitable reduced binding to IL-2Rα, with retention of binding to IL-2Rβ resulting in highly active multispecific proteins, referred to herein as IL2v2.

IL2v2 (R38A/F42K substitutions):
(SEQ ID NO: 355)
SSSTKKTQLQLEHLLLDLQMILNGINNYKNPKLTAMLTKKFYMPKKATE
LKHLQCLEEELKPLEEVLNLAQSKNFHLRPRDLISNINVIVLELKGSET
TFMCEYADETATIVEFLNRWITFCQSIISTLT.

In one embodiment, an IL2v polypeptide has the wild-type IL-2p amino acid sequence with the three amino acid substitutions R38A, F42K and T41A, as shown below, referred to herein as IL2v3:

IL2v3 (R38A/T41A/F42K substitutions):
(SEQ ID NO: 356)
SSSTKKTQLQLEHLLLDLQMILNGINNYKNPKLTAMLAKKFYMPKKATE
LKHLQCLEEELKPLEEVLNLAQSKNFHLRPRDLISNINVIVLELKGSET
TFMCEYADETATIVEFLNRWITFCQSIISTLT.

Thus, in one embodiment, an IL2 variant comprises at least one or at least two amino acid modifications (e.g. substitution, insertion, deletion) compared to a human wild type IL-2 polypeptide. In one embodiment, an IL2v comprises a R38 substitution (e.g. R38A) and an F42 substitution (e.g., F42K), compared to a human wild type IL-2 polypeptide. In one embodiment, an IL2v comprises a R38 substitution (e.g. R38A), an F42 substitution (e.g., F42K) and a T41 substitution (e.g. T41A), compared to a human wild type IL-2 polypeptide. In one embodiment, an IL2v comprises a T3 substitution (e.g. T3A), an F42 substitution (e.g., F42A), a Y45 substitution (e.g. Y45A), a L72 substitution (e.g. L72G) and a C125 substitution (e.g. C125A), compared to a human wild type IL-2 polypeptide. Optionally the IL2v comprises an amino acid sequence identical to or at least 70%, 80%, 90%, 95%, 98% or 99% identical to the polypeptide of SEQ ID NOS: 352-356. Optionally the IL2v comprises a fragment of a human IL-2 polypeptide, wherein the fragment has an amino sequence identical to or at least 70%, 80%, 90%, 95%, 98% or 99% identical to a contiguous sequence of 40, 50, 60, 70 or 80 amino acids of the polypeptide of SEQ ID NOS: 352-356.

Any combination of the positions can be modified. In some embodiments, the IL-2 variant comprises two or more modification. In some embodiments, the IL-2 variant comprises three or more modification. In some embodiments, the IL-2 variant comprises four, five, or six or more modifications.

IL2 variant polypeptides can for example comprise two, three, four, five, six or seven amino acid modifications (e.g. substitutions). For example, U.S. Pat. No. 5,229,109, the disclosure of which is incorporated herein by reference, provides a human IL2 polypeptide having a R38A and F42K substitution. U.S. Pat. No. 9,447,159, the disclosure of which is incorporated herein by reference, describes human IL2 polypeptides having substitutions T3A, F42A, Y45A, and L72G substitutions. U.S. Pat. No. 9,266,938, the disclosure of which is incorporated herein by reference, describes human IL2 polypeptides having substitutions at residue L72 (e.g. L72G, L72A, L72S, L72T, L72Q, L72E, L72N, L72D, L72R, and L72K), residue F42 (e.g. F42A, F42G, F42S, F42T, F42Q, F42E, F42N, F42D, F42R, and F42K); and at residue Y45 (e.g., Y45A, Y45G, Y45S, Y45T, Y45Q, Y45E, Y45N, Y45D, Y45R and Y45K), including for example the triple mutation F42A/Y45A/L72G to reduce or abolish the affinity for IL-2Rα receptor. Yet further WO2020/057646, the disclosure of which is incorporated herein by reference, relates to amino acid sequence of IL-2v polypeptides comprising amino acid substitutions in various combinations among amino acid residues K35, T37, R38, F42, Y45, E61 and E68. Yet further, WO2020252418, the disclosure of which is incorporated herein by reference, relates to amino acid sequence of IL-2v polypeptides having at least one amino acid residues position R38, T41, F42, F44, E62, P65, E68, Y107, or C125 substituted with another amino acid, for example wherein the amino acid substitution is selected from the group consisting of: the substitution of L19D, L19H, L19N, L19P, L19Q, L19R, L19S, L19Y at position 19, the substitution of R38A, R38F, R38G at position 38, the substitution of T41A, T41G, and T41V at position 41, the substitution of F42A at position 42, the substitution of F44G and F44V at position 44, the substitution of E62A, E62F, E62H, and E62L at position 62, the substitution of P65A, P65E, P65G, P65H, P65K, P65N, P65Q, P65R at position 65, the substitution of E68E, E68F, E68H, E68L, and E68P at position 68, the substitution of Y107G, Y107H, Y107L and Y107V at position 107, and the substitution of C1251 at position 125, the substitution of Q126E at position 126. Numbering of positions is with respect to Wild-type mature human IL-2.

A modified IL-2 can optionally be specified as exhibiting a KD for binding to CD25 or to a CD25:CD122:CD132 complex that is decreased by at least 1-log, optionally at least 2-log, optionally at least 3-log, compared to a wild-type human IL-2 polypeptide (e.g. comprising the amino acid sequence of SEQ ID NO: 352). A modified IL-2 can optionally be specified as exhibiting less than 20%, 30%, 40% or 50% of binding affinity to CD25 or to a CD25:CD122:CD132 complex compared to a wild-type human IL-2 polypeptide. An IL2 can optionally be specified as exhibiting at least 50%, 70%, 80% or 90% of binding affinity to CD122 or to a CD122:CD132 complex compared to a wild-type human IL-2 polypeptide. In some embodiments, an IL2 exhibits at least 50%, 60%, 70% or 80% but less than 100% of binding affinity to CD122 or to a CD122:CD132 complex compared to a wild-type human IL-2 polypeptide. In some embodiments, an IL2v exhibits less than 50% of binding affinity to CD25 and at least 50%, 60%, 70% or 80% of binding affinity to CD122, compared to wild-type IL-2 polypeptide.

Differences in binding affinity of wild-type and disclosed mutant polypeptide for CD25 and CD122 and complexes thereof can be measured, e.g., in standard surface plasmon resonance (SPR) assays that measure affinity of protein-protein interactions familiar to those skilled in the art.

Exemplary IL2 variant polypeptides have one or more, two or more, or three or more CD25-affinity-reducing amino acid substitutions relative to the wild-type mature IL-2 polypeptide having an amino acid sequence of SEQ ID NO: 352. In one embodiment, the exemplary IL2v polypeptides comprise one or more, two or more, or three or more substituted residues selected from the following group: Q11, H16, L18, L19, D20, D84, S87, Q22, R38, T41, F42, K43, Y45, E62, P65, E68, V69, L72, D84, S87, N88, V91, I92, T123, Q126, S127, I129, and S130.

In one embodiment, the exemplary IL2 variant polypeptide has one, two, three, four, five or more of amino acid residues position R38, T41, F42, F44, E62, P65, E68, Y107, or C125 substituted with another amino acid.

In one embodiment, decreased affinity to CD25 or a protein complex comprising such (e.g., a CD25:CD122:CD132 complex) may be obtained by substituting one or more of the following residues in the sequence of the wild-type mature IL-2 polypeptide: R38, F42, K43, Y45, E62, P65, E68, V69, and L72.

In one embodiment, a CD122 ABD or IL-2 polypeptide is an IL-2 mimetic polypeptide. Synthetic IL-2/IL-15 polypeptide mimics can be computationally designed to bind to CD122, but not to CD25, for example as described in Silva et al, (2019) Nature 565(7738): 186-191 and WO2020/005819, the disclosures of which are incorporated herein by reference, also provides IL-2 and IL-15 mimetic polypeptides that bind CD122 but not CD25.

For example, an IL-2 mimetic polypeptide can be characterized as a non-naturally occurring polypeptide comprising domains X₁, X₂, X₃, and X₄, wherein:
(a) X₁ is a peptide comprising the amino acid sequence at least 85% identical to EHALYDAL (SEQ ID NO: 357);
(b) X₂ is a helical-peptide of at least 8 amino acids in length;
(c) X₃ is a peptide comprising the amino acid sequence at least 85% identical to YAFNFELI (SEQ ID NO: 358);
(d) X₄ is a peptide comprising the amino acid sequence at least 85% identical to ITILQSWIF (SEQ ID NO: 359);
wherein X₁, X₂, X₃, and X₄ may be in any order in the polypeptide,
wherein amino acid linkers may be present between any of the domains, and wherein the polypeptide binds to CD122 (or to the CD122:CD132 heterodimer). Optionally, the polypeptides bind the CD122:CD132 heterodimer with a binding affinity of 200 nM or less, 100 nM or less, 50 nM or less or 25 nM or less.

In one aspect, the invention provides non-naturally occurring polypeptides comprising domains X₁, X₂, X₃, and X₄, wherein:
(a) X₁ is a peptide comprising the ammo acid sequence EHALYDAL (SEQ ID NO: 357);
(b) X₂ is a helical-peptide of at least 8 amino acids in length;
(c) X₃ is a peptide comprising the amino acid sequence YAFNFELI (SEQ ID NO: 358);
(d) X₄ is a peptide comprising the amino acid sequence ITILQSWIF (SEQ ID NO:359);
wherein X₁, X₂, X₃, and X₄ may be in any order in the polypeptide,
wherein amino acid linkers may be present between any of the domains, and wherein the polypeptide binds to CD122 (or to the CD122:CD132 heterodimer). Optionally, the polypeptides bind the CD122:CD132 heterodimer with a binding affinity of 200 nM or less, 100 nM or less, 50 nM or less or 25 nM or less, optionally between about 1 nm and about 100 nm, optionally between about 10 nM and about 200 nM, optionally between about 10 nM and about 100 nM optionally between about 15 nM and about 100 nM.

In one example, X₁, X₃, and X₄ may be any suitable length, meaning each domain may contain any suitable number of additional amino acids other than the peptides of SEQ ID NOS: 357, 358, and 359, respectively. In one embodiment, X, is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide PKKKIQLHAEHALYDALMILNI (SEQ ID NO: 360); X₃ is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical along its length the amino acid sequence LEDYAFNFELILEEIARLFESG (SEQ ID NO: 361); and X₄ is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical along its length to the amino acid sequence EDEQEEMANAIITILQSWIFS (SEQ ID NO: 362).

In one example, a computationally designed synthetic IL-2 polypeptide or mimetic (or CD122 ABD) comprises the amino acid sequence (neoleukin) shown below (with or without a (GS₄)₃ domain linker:

(SEQ ID NO: 363)
PKKKIQLHAEHALYDALMILNIVKTNSPPAEEKLEDYAFNF
ELILEEIARLFESGDQKDEAEKAKRMKEWMKRIKTTASEDE
QEEMANAIITILQSWIFS.
or
(SEQ ID NO: 364)
GSSSSGSSSSGSSSSPKKKIQLHAEHALYDALMILNIVKTN
SPPAEEKLEDYAFNFELILEEIARLFESGDQKDEAEKAKRM
KEWMKRIKTTASEDEQEEMANAIITILQSWIFS.

In yet other examples, an IL-2 polypeptide is modified by connecting, fusing, binding or associating it with one or more other additional compounds, chemical compounds, polymer (e.g. PEG), or polypeptides or polypeptide chains that result in a decrease of binding to CD25. For example a wild-type IL-2 polypeptide or fragment thereof can be modified by binding to it a CD25 binding peptide or polypeptide, including but not limited to an anti-IL-2 monoclonal antibody or antibody fragment thereof that binds or interacts with CD25-binding site of human IL-2, thereby decreasing binding to CD25.

In one example, an IL-2 further comprises a receptor domain, e.g., a cytokine receptor domain. In one embodiment, the cytokine molecule comprises an IL-2 receptor, or a fragment thereof (e.g., an IL-2 binding domain of an IL-2 receptor alpha). In one example, a CD25-derived polypeptide is fused to an IL-2 polypeptide, as described in Lopes et al, J Immunother Cancer. 2020; 8(1), the disclosure of which is incorporated herein by reference. In one example, the IL-2 is a variant fusion protein comprising a circularly permuted (cp) IL-2 fused to a CD25 polypeptide (see e.g., PCT publication no. WO2020/249693, the disclosure of which is incorporated herein by reference). Where the CD122 ABD comprises a circularly permuted (cp) IL-2 fused to a CD25 polypeptide, the ABD can comprise a cpIL-2:IL-2Rα polypeptide or protein of described in PCT publication no. WO2013/184942, the disclosure of which is incorporated herein by reference. For example, the permuted (cp) IL-2 variant fused to a CD25 polypeptide can have the amino acid sequence: SKNFHLRPRDLISNINVIVLELKGSETTFMCEYADETATIVEFLNRWITFSQSIISTLTGGSS STKKTQLQLEHLLLDLQMILNGINNYKNPKLTRMLTFKFYMPKKATELKHLQCLEEELK PLEEVLNLAQGSGGGSELCDDDPPEIPHATFKAMAYKEGTMLNCECKRGFRRIKSGSLY MLCTGNSSHSSWDNQCQCTSSATRNTTKQVTPQPEEQKERKTTEMQSPMQPVDQASLP GHCREPPPWENEATERIYHFWGQMVYYQCVQGYRALHRGPAESVCKMTHGKTRWT QPQLICTG (SEQ ID NO: 365), or an amino acid sequence having sequence identity that is about 75%, 80%, 85%, 90%, 91%, 92%, 93%, 94%, 95%, 96%, 97%, 98%, 99% or higher over a contiguous stretch of about 20 amino acids up to the full length of SEQ ID NO: 365.

In one example, IL-2 is associated with a specific anti-IL-2 monoclonal antibody (mAb), thus forming an IL-2/anti-IL-2 mAb complex (IL-2cx). Such complexes have been shown to overcome the CD25 binding (Boyman et al., Science 311, 1924-1927 (2006). An exemplary anti-IL2 antibody is antibody NARA1. PCT publication no. WO2017/122130, the disclosure of which is incorporated herein by reference, describes fusion proteins in which flexible linkers are used to connect IL-2 to the variable region of the light or heavy chain of NARA1. Sahin et al. (Nature Communications volume 11, Article number: 6440 (2020)) describe an improved construct in which IL-2 is brought into contact with and bound to the complementarity-determining region 1 of the light chain (L-CDR1) of NARA1, which resulted in a protein complex in which the IL-2 is bound to its antigen-binding groove on the antibody fragment (or the polypeptide chain(s) of the fragment).

In other examples, an IL-2 polypeptide or fragment thereof can be modified by binding to it a moiety of interest (e.g. a compound, chemical compounds, polymer, linear or branched PEG polymer), covalently attached to a natural amino acid or to an unnatural amino acid installed at a selected position. Such a modified interleukin 2 (IL-2) polypeptide can comprise at least one unnatural amino acid at a position on the polypeptide that reduces binding between the modified IL-2 polypeptide and CD25 but retains significant binding to the CD122:CD132 signaling complex, wherein the reduced binding to CD25 is compared to binding between a wild-type IL-2 polypeptide and CD25. An unnatural amino acid can be positioned at any one or more of residues K35, T37, R38, T41, F42, K43, F44, Y45, E60, E61, E62, K64, P65, E68, V69, N71, L72, M104, C105, and Y107 of IL-2. As disclosed in PCT publication nos. WO2019/028419 and WO2019/014267, the disclosures of which are incorporated herein by reference, the unnatural amino acid can be incorporated into the modified IL-2 polypeptide by an orthogonal tRNA synthetase/tRNA pair. The unnatural amino acid can for example comprise a lysine analogue, an aromatic side chain, an azido group, an alkyne group, or an aldehyde or ketone group. The modified IL-2 polypeptide can then be covalently attached to a water-soluble polymer, a lipid, a protein, or a peptide through the unnatural amino acid. Examples of suitable polymers include polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof, or a polysaccharide such as dextran, polysialic acid (PSA), hyaluronic acid (HA), amylose, heparin, heparan sulfate (HS), dextrin, or hydroxyethyl-starch (HES).

In some examples, an exemplary IL2v/not-alpha IL-2 conjugate can comprise a full-length or fragment of an IL-2 polypeptide in which an amino acid residue in the IL-2 polypeptide (e.g. a residue at position selected from K35, F42, F44, K43, E62, P65, R38, T41, E68, Y45, V69, and L72) is replaced by a natural or non-natural amino acid residue attached to a polymer via a chemical linker. The polymer can be a PEG polymer, e.g. a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa.

A modified IL2 polypeptide can comprising at least one unnatural amino acid at a position on the polypeptide that reduces binding between the modified IL-2 polypeptide and CD25 but retains significant binding with CD122:CD132 signaling complex to form a CD122:CD132 complex, wherein the reduced binding to CD25 is compared to binding between a wild-type IL-2 polypeptide and CD25 An exemplary 112v/not-alpha IL-2 conjugate is THOR-707 (Synthorx inc).

For example, as described in PCT publication no. WO2020/163532, the disclosure of which is incorporated herein by reference, an amino acid residue in the IL-2 conjugate is replaced by the structure of Formula (I):

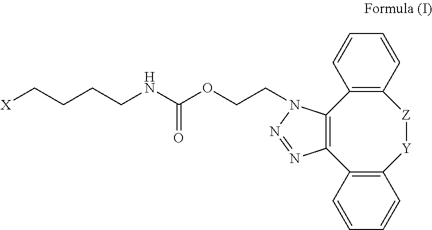

wherein:
Z is CH2 and Y is

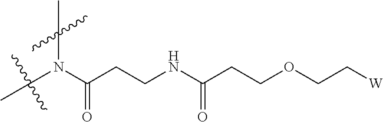

Y is CH2 and Z is

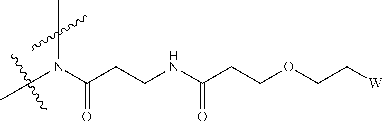

Z is CH2 and Y is

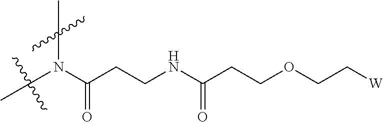

or,
Y is CH2 and Z is

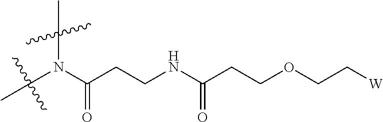

and wherein, W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

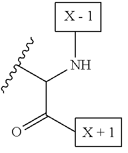 [SMILES: *C(NC)C(C)=O]

In one embodiment, an IL-2 comprises a releasable polymer (e.g. a releasable PEG polymer), e.g. the IL-2 is conjugated, linked or bound to a releasable polymer that results in a decrease in CD25 binding in vivo and/or in vitro. Example of such modified IL-2 include bempegaldesleukin or RSLAIL-2 (Nektar Therapeutics inc.), which exhibits about a 60-fold decrease in affinity to CD25 relative to IL-2, but only about a 5-fold decrease in affinity CD122 relative to IL-2. “Bempegaldesleukin” (CAS No. 1939126-74-5) is an IL-2 in which human interleukin-2 (des-1-alanine, 125-serine), is N-substituted with an average of six [(2,7-bis{[methylpoly(oxyethylene)iokD]carbamoyl}-9H-fluoren-9-yl)methoxy]carbonyl moieties at its amino residues. As disclosed in PCT publication no. WO2020/095183, the disclosure of which is incorporated herein by reference, the releasable PEG comprised can be based upon a 2,7,9-substituted fluorene, with poly(ethylene glycol) chains extending from the 2- and 7-positions on the fluorene ring via amide linkages (fluorene-C(0)-NH˜), and having releasable covalent attachment to IL-2 via attachment to a carbamate nitrogen atom attached via a methylene group (—CH2-) to the 9-position of the fluorene ring.

In another embodiment where the cytokine-binding ABD is a CD122-binding ABD, the ABD can be or comprise a suitable interleukin-15 (IL-15) polypeptide such that the CD122 ABD binds CD122. In some embodiments, the cytokine molecule is an IL-15 molecule, e.g., a full length, a fragment or a variant (IL-15v) of IL-15, e.g., human IL-15. In some embodiments, the IL-15 molecule comprises a wild-type human IL-15 amino acid sequence, e.g., having the amino acid sequence of SEQ ID NO: 366. In some embodiments, the IL-15 molecule comprises an amino sequence at least 70%, 80%, 90%, 95%, 98% or 99% identical to a mature wild-type human IL-15 amino acid sequence of SEQ ID NO: 366. In other embodiments, the IL-15 molecule is a variant of human IL-15, e.g., having one or more amino acid modifications. Optionally the IL-15 comprises a fragment of a human IL-15 polypeptide, wherein the fragment has an amino sequence is identical to or at least 70%, 80%, 90%, 95%, 98% or 99% identical to a contiguous sequence of 40, 50, 60, 70 or 80 amino acids of the wild-type mature human IL-15 polypeptide of SEQ ID NO: 366.

Wild-type mature human IL-15:
(SEQ ID NO: 366)
NW VNVISDLKKI EDLIQSMHID ATLYTESDVH
PSCKVTAMKC FLLELQVISL ESGDASIHDT
VENLIILANN SLSSNGNVTE SGCKECEELE
EKNIKEFLQS FVHIVQMFIN TS.

In some embodiments, an IL-15 variant comprises a modification (e.g. substitution) at position 45, 51, 52, or 72 (with reference to the sequence of human IL-15, SEQ ID NO: 366), e.g., as described in US 2016/0184399. In some embodiments, the IL-15 variant comprises four, five, or six or more modifications. In some embodiments, the IL-15 variant comprises one or more modification at amino acid position 8, 10, 61, 64, 65, 72, 101, or 108 (with reference to the sequence of human IL-15, SEQ ID NO: 366). In some embodiments the IL-15 variant possesses increased affinity for CD122 as compared with wild-type IL-15. In some embodiments the IL-15 variant possesses decreased affinity for CD122 as compared with wild-type IL-15. In some embodiments, the mutation is chosen from D8N, K10Q, D61N, D61H, E64H, N65H, N72A, N72H, Q101N, Q108N, or Q108H (with reference to the sequence of human IL-15, SEQ ID NO: 366). Any combination of the positions can be mutated. In some embodiments, the IL-15 variant comprises two or more mutations. In some embodiments, the IL-15 variant comprises three or more mutations. In some embodiments, the IL-15 variant comprises four, five, or six or more mutations. In some embodiments the IL-15 variant comprises mutations at positions 61 and 64. In some embodiments the mutations at positions 61 and 64 are D61N or D61H and E64Q or E64H. In some embodiments the IL-15 variant comprises mutations at positions 61 and 108. In some embodiments the mutations at positions 61 and 108 are D61N or D61H and Q108N or Q108H.

The extracellular domain of IL-15Rα comprises a domain referred to as the sushi domain, which binds IL-15. The general sushi domain, also referred to as complement control protein (CCP) modules or short consensus repeats (SCR), is a protein domain found in several proteins, including multiple members of the complement system. The sushi domain adopts a beta-sandwich fold, which is bounded by the first and fourth cysteine of four highly conserved cysteine residues, comprising a sequence stretch of approximately 60 amino acids (Norman, Barlow, et al. J Mol Biol. 1991; 219(4):717-25). The amino acid residues bounded by the first and fourth cysteines of the sushi domain IL-15Rα comprise a 62 amino acid polypeptide referred to as the minimal domain. Including additional amino acids of IL-15Rα at the N- and C-terminus of the minimal sushi domain, such as inclusion of N-terminal lie and Thr and C-terminal lie and Arg residues result in a 65 amino acid extended sushi domain.

The CD122 ABD can further comprise a receptor domain, e.g., a cytokine receptor domain. In one embodiment, the cytokine molecule comprises an IL-15 receptor, or a fragment thereof (e.g., an IL-15 binding domain of an IL-15 receptor alpha).

In some embodiments, the CD122 ABD binds an IL-15 receptor alpha (IL-15Rα) sushi domain, a first domain linker, and an IL-15 polypeptide, e.g. from N- to C-terminus, a IL-15Rα sushi domain fused to a domain linker, in turn fused to an IL-15 polypeptide. Optionally the IL-15 polypeptide is a variant IL-15 polypeptide, e.g., comprising one or more amino acid substitutions. In other embodiments, the variant IL-15 domain comprises the amino acid sequence of SEQ ID NO: 366 and amino acid substitutions selected from the group consisting of N4D/N65D, D30N/N65D, and D30N/E64Q/N65D.

A sushi domain as described herein may comprise one or more mutations relative to a wild-type sushi domain. In some embodiments, the IL-15Rα sushi domain comprises the amino acid sequence:

(SEQ ID NO: 367)
ITCPPPMSVEHADIWVKSYSLYSRERYICNSGF
KRKAGTSSLTECVLNKATNVAHWTTPSLKCIR

An IL-15 polypeptide can be modified by connecting, fusing, binding or associating it with one or more other additional compounds using any of several known techniques, for example by conjugation or binding to chemical compounds, polymer (e.g. PEG), or polypeptides or polypeptide chains that result in a decrease of binding to IL-15Rα. In one example, a wild-type IL-15 polypeptide or fragment thereof can be modified by binding to it a IL-15Rα binding peptide or polypeptide, including but not limited to an anti-IL-15 monoclonal antibody or antibody fragment thereof that binds or interacts with IL-15Rα-binding site of human IL-15, thereby decreasing binding to IL-15Rα.

In another example, an IL-15 polypeptide or fragment thereof can be modified by binding to it a moiety of interest (e.g. a compound, chemical compounds, polymer, linear or branched PEG polymer), covalently attached to a novel amino acid installed at a selected position. Such a modified IL-15 polypeptide can comprise at least one unnatural amino acid at a position on the polypeptide that reduces binding between the modified IL-15 polypeptide and IL-15Rα but retains significant binding with CD122:CD132 signaling complex to form an CD122:CD132 complex, wherein the reduced binding to IL-15Rα is compared to binding between a wild-type IL-15 polypeptide and IL-15Rα. The unnatural amino acid can be positioned at any one or more of residues N1, W2, V3, N4, 16, S7, D8, K10, K11, E13, D14, L15, Q17, S18, M19, H20, 121, D22, A23, T24, L25, Y26, E28, S29, D30, V31, H32, P33, S34, C35, K36, V37, T38, K41, L44, E46, Q48, V49, S51, L52, E53, S54, G55, D56, A57, S58, H60, D61, T62, V63, E64, N65, 167, 168, L69, N71, N72, S73, L74, S75, S76, N77, G78, N79, V80, T81, E82, S83, G84, C85, K86, E87, C88, E89, E90, L91, E92, E93, K94, N95, 196, K97, E98, L100, Q101, S102, V104, H105, Q108, M109, F110, I111, N112, T113, and S114 of IL-15. As disclosed in WO2019165453, WO2019/028419 and WO2019/014267, the disclosures of which are incorporated herein by reference, the unnatural amino acid can be incorporated into the modified IL-2 polypeptide by an orthogonal tRNA synthetase/tRNA pair. The unnatural amino acid can for example comprise a lysine analogue, an aromatic side chain, an azido group, an alkyne group, or an aldehyde or ketone group. The modified IL-15 polypeptide can then be covalently attached to a water-soluble polymer, a lipid, a protein, or a peptide through the unnatural amino acid. Examples of suitable polymers include polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof, or a polysaccharide such as dextran, polysialic acid (PSA), hyaluronic acid (HA), amylose, heparin, heparan sulfate (HS), dextrin, or hydroxyethyl-starch (HES).

For example, as described in WO2020/163532, the disclosure of which is incorporated herein by reference, an amino acid residue in the IL-15 conjugate is replaced by the structure of Formula (I):

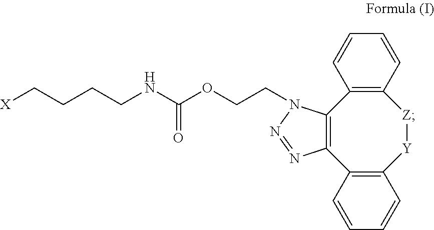

wherein:
Z is CH2 and Y is

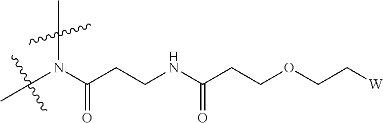

Y is CH2 and Z is

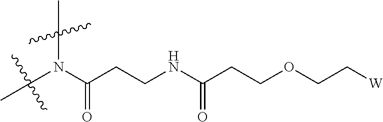

Z is CH2 and Y is

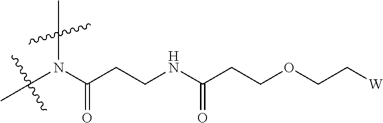

or,
Y is CH2 and Z is

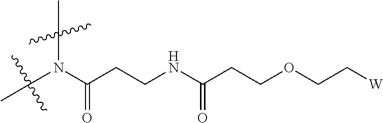

and wherein, W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

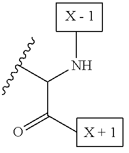 [SMILES: *C(NC)C(C)=O]

In one embodiment, an IL-15 comprises a releasable polymer (e.g. a releasable PEG polymer), e.g. the IL-15 is conjugated, linked or bound to a releasable polymer that results in a decrease in IL-15R binding in vivo and/or in vitro. Examples include compounds disclosed in PCT publication no. WO2020/097556, the disclosure of which is incorporated herein by reference. For example, the modified IL-15 can comprise comprising compound having the structure:

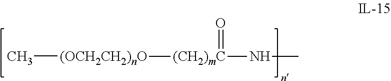

wherein (n) is an integer from about 150 to about 3,000, (m) is an integer selected from 2, 3, 4, and 5, (n′) is 1, and ˜NH˜ represents an amino group of the IL-15 polypeptide.

In another embodiment where the cytokine-binding ABD binds an IL-21 receptor (IL-21R)-binding ABD, the ABD can be or comprise a suitable interleukin-21 (IL-21) polypeptide such that the IL-21R ABD binds IL-21R on the surface of NK cells. IL-21R is similar in structure to the IL-2 receptor and the IL-15 receptor, in that each of these cytokine receptors comprises a common gamma chain (γc). In some embodiments, the cytokine molecule is an IL-21 molecule, e.g., a full length, a fragment or a variant of IL-21, e.g., human IL-21. In embodiments, the IL-21 molecule is a wild-type, human IL-21, e.g., having the amino acid sequence of SEQ ID NO: 368. In some embodiments, the IL-15 molecule comprises an amino sequence at least 70%, 80%, 90%, 95%, 98% or 99% identical to a mature wild-type human IL-21 amino acid sequence of SEQ ID NO: 368. In other embodiments, the IL-21 molecule is a variant of human IL-21, e.g., having one or more amino acid modifications. Optionally the IL-21 comprises a fragment of a human IL-21 polypeptide, wherein the fragment has an amino sequence is identical to or at least 70%, 80%, 90%, 95%, 98% or 99% identical to a contiguous sequence of 40, 50, 60, 70 or 80 amino acids of the polypeptide of SEQ ID NO: 368.

Wild-type mature human IL-21:
(SEQ ID NO: 368)
HKSSSQ GQDRHMIRMR QLIDIVDQLK NYVNDLVPEF
LPAPEDVETN CEWSAFSCFQ KAQLKSANTG NNERIINVSI
KKLKRKPPST NAGRRQKHRL TCPSCDSYEK KPPKEFLERE
KSLLOKMIHQ HLSSRTHGSE DS

In some embodiments, the IL-21 variant can comprise an IL-21 polypeptide comprising one or more amino acid mutations designed to reduce its ability to bind to human IL-21R, while retaining substantial ability to bind human IL-21R. For example, the IL-21 can be characterized as binding to human IL-21R with a KD that is greater than or is about 0.04 nM, as determined by SPR.

Examples of such IL-21 variants are provided in PCT publication no. WO2019028316, the disclosure of which is incorporated herein by reference. In exemplary aspects, the amino acid substitutions are located at two amino acid positions selected from the group consisting of 10, 14, 20, 75, 76, 77, 78 and 81 according to numbering of SEQ ID NO: 368 or at two amino acid positions selected from the group consisting of 5, 9, 15, 70, 71, 72, 73, and 76, according to the amino acid position numbering of SEQ ID NO: 369. In exemplary aspects, the IL-21 variant comprises the amino acid sequence:

(SEQ ID NO: 369)
QGQDX HMXXM XXXXX XVDXL KNXVN
DLVPE FLPAP EDVET NCEWS AFSCF
QKAQL KSANT GNNEX XIXXX XXXLX
XXXXX TNAGR RQKHR LTCPS CDSYE
KKPPK EFLXX FXXLL XXMXX QHXSS
RTHGS EDS,

wherein X represents any amino acid, and wherein the IL-21 variant amino acid sequence differs from the amino acid sequence of human IL-21 (SEQ ID NO: 368) by at least 1 amino acid.

In exemplary aspects, the IL-21 variant comprises the sequence of SEQ ID NO: 369, wherein SEQ ID NO: 369 differs from SEQ ID NO: 368 by at least one amino acid at a position designated by X in SEQ ID NO: 369. In exemplary aspects, the IL-21 variant has at least about 30%, at least about 40%, at least about 50%, at least about 60%, at least about 70%, at least about 80%, at least about 85%, at least about 90%, or has greater than about 90% (e.g., about 91%, about 92%, about 93%, about 94%, about 95%, about 96%, about 97%, about 98%, or about 99%) sequence identity to SEQ ID NO: 368.

In exemplary embodiments, the IL-21 variant comprises an amino acid substitution relative to the wild-type IL-21 amino acid sequence within the N-terminal half of the amino acid sequence, e.g. at a position within positions 10-30 or 13-28 (both inclusive), according to the amino acid position numbering of SEQ ID NO: 368. In other exemplary embodiments, the IL-21 variant comprises an amino acid substitution relative to the wild-type IL-21 amino acid sequence within the C-terminal half of the amino acid sequence, e.g., at a position within positions 105-138 or 114-128 (both inclusive), according to the amino acid position numbering of SEQ ID NO: 368. In other exemplary embodiments, the IL-21 variant comprises an amino acid substitution relative to the wild-type IL-21 amino acid sequence in the middle third of the amino acid sequence, e.g., at a position within positions 60-90 or 70-85 (both inclusive), according to the amino acid position numbering of SEQ ID NO: 368.

Optionally, the IL-21 variant comprises only one amino acid substitution, relative to the wild-type IL-21 amino acid sequence. Optionally, the amino acid substitution is located at an amino acid position selected from the group consisting of: 10, 13, 14, 16, 17, 18, 19, 20, 21, 24, 28, 70, 71, 73, 74, 75, 76, 77, 78, 80, 81, 82, 83, 84, 85, 114, 115, 117, 118, 121, 122, 124, 125, or 128, according to the amino acid position numbering of SEQ ID NO: 368.

In another embodiment where the cytokine-binding ABD binds an IL-18 receptor (IL-18Ra)-binding ABD, the ABD can be or comprise a suitable interleukin-18 (IL-18) polypeptide such that the IL-18R ABD binds IL-18Ra on the surface of NK cells. In some embodiments, the cytokine molecule is an IL-18 molecule, e.g., a full length, a fragment or a variant of IL-18, e.g., human IL-18. In embodiments, the IL-18 molecule is a wild-type, human IL-18, e.g., having the amino acid sequence of SEQ ID NO: 370. In some embodiments, the IL-18 molecule comprises an amino sequence at least 70%, 80%, 90%, 95%, 98% or 99% identical to a mature wild-type human IL-18 amino acid sequence of SEQ ID NO: 370. In other embodiments, the IL-18 molecule is a variant of human IL-18, e.g., having one or more amino acid modifications. Optionally the IL-18 comprises a fragment of a human IL-18 polypeptide, wherein the fragment has an amino sequence is identical to or at least 70%, 80%, 90%, 95%, 98% or 99% identical to a contiguous sequence of 40, 50, 60, 70 or 80 amino acids of the polypeptide of SEQ ID NO: 370.

Wild-type mature human IL-18:
(SEQ ID NO: 370)
YFGKLESKLSVIRNLNDQVLFIDQGNRPLFEDMT
DSDCRDNAPRTIFIISMYKDSQPRGMAVTISVKC
EKISTLSCENKIISFKEMNPPDNIKDTKSDIIFF
QRSVPGHDNKMQFESSSYEGYFLACEKERDLEKL
ILKKEDELGDRSIMFTVQNED.

In one embodiment, an IL-18 is modified to decrease its binding affinity for IL-18BP while not substantially decreasing affinity for IL-18Ra. For example, an IL-18 may comprise of a modification such as amino acid substitutions at positions M51, S55, R104 and/or N110 that are not involved in IL-18Ra binding, optionally further in combination with a substitution at K53 and/or M60 (positions are with reference to the wild-type mature IL-18 amino acid sequence). In one embodiment, the IL-18 has a M51S, S55A, R104Q, R104K or R104S and/or N110A substitution. In one embodiment, the IL-18 comprises a K53S or K53A substitution. In one embodiment, the IL-18 comprises a M60S or M60K substitution.

In another embodiment, the cytokine-binding ABD binds a type I interferon receptor, for example interferon-α receptor (IFN-αR). The ABD can be or comprise a suitable type I interferon, for example an interferon-α (IFN-α) or interferon-β (IFN-β) polypeptide such that the IFN-α ABD binds IFN-αR on the surface of NK cells. The interferon-α receptor is also known as the interferon α/β receptor (IFNAR), a heterodimeric transmembrane receptor that is composed of the two subunits IFNAR1 and IFNAR2. For type I IFNs, the main STAT signaling complex is formed by IFN-stimulated gene factor 3 consisting of STAT1, STAT2, and IFN regulatory factor (IRF)-9. In some embodiments, the cytokine molecule is an IFN-α or IFN-β molecule, e.g., a full length, a fragment or a variant of IFN-α or IFN-β, e.g., human IFN-α or IFN-β, for example a human IFN-α1, IFN-α2, IFN-α4, IFN-α5, IFN-α6, IFN-α7, IFN-α8, IFN-α10, IFN-α12, IFN-α14, IFN-α16 or IFN-α17 polypeptide. In some embodiments, the IFN-α or IFN-β molecule is a wild-type, human IFN-α or IFN-β, e.g., having the amino acid sequence of any of SEQ ID NOS: 371-382. In other embodiments, the IFN-α or IFN-β molecule is a variant of human IFN-α or IFN-β, e.g., having one or more amino acid modifications. In some embodiments, the IFN-α or IFN-β molecule comprises an amino sequence at least 70%, 80%, 90%, 95%, 98% or 99% identical to a mature wild-type human IFN-α or IFN-β amino acid sequence of SEQ ID NOS: 371-382, respectively. In other embodiments, the IFN-α or IFN-β molecule is a variant of human IFN-α or IFN-β, e.g., having one or more amino acid modifications. Optionally the IFN-α or IFN-β comprises a fragment of a human IFN-α or IFN-β polypeptide, wherein the fragment has an amino sequence is identical to or at least 70%, 80%, 90%, 95%, 98% or 99% identical to a contiguous sequence of 40, 50, 60, 70 or 80 amino acids of the polypeptide of SEQ ID NOS: 371-382.

### Wild-Type Human IFN-α Mature Proteins:

IFNα2
(SEQ ID NO: 371)
CDLPQTHSLGSRRTLMLLAQMRRISLESCLKDRHDFGFPQEEFGN
QFQKAETIPVLHEMIQQIFNLESTKDSSAAWDETLLDKFYTELYQ
QLNDLEACVIQGVGVTETPLMKEDSILAVRKYFQRITLYLKEKKY
SPCAWEVVRAEIMRSESLSTNLQESLRSKE.
IFNα1
(SEQ ID NO: 372)
CDLPETHSLDNRRTLMLLAQMSRISPSSCLMDRHDFGFPQEEFDG
NQFQKAPAISVLHELIQQIFENLETTKDSSAAWDEDLLDKFCTEL
YQQLNDLEACVMQEERVGETPLMNADSILAVKKYFRRITLYLTEK
KYSPCAWEVVRAEIMRSLSLSTNLQERLRRKE.
IFNα4
(SEQ ID NO: 373)
CDLPQTHSLGNRRALILLAQMGRISHFSCLKDRHDFGFPEEEFDG
HQFQKTQAISVLHEMIQQTENLESTEDSSAAWEQSLLEKFSTELY
QQLNDLEACVIQEVGVEETPLMNEDSILAVRKYFQRITLYLTEKK
YSPCAWEVVRAEIMRSLSFSTNLQKRLRRKD.
IFNα5
(SEQ ID NO: 374)
CDLPQTHSLSNRRTLMIMAQMGRISPFSCLKDRHDFGFPQEEFDG
NQFQKAQAISVLHEMIQQTENLESTKDSSATWDETLLDKFYTELY
QQLNDLEACMMQEVGVEDTPLMNVDSILTVRKYFORITLYLTEKK
YSPCAWEVVRAEIMRSESLSANLQERLRRKE.
IFNα6
(SEQ ID NO: 375)
CDLPQTHSLGHRRTMMLLAQMRRISLFSCLKDRHDFRFPQEEFDG
NQFQKAEAISVLHEVIQQTENLESTKDSSVAWDERLLDKLYTELY
QQLNDLEACVMQEVWVGGTPLMNEDSILAVRKYFQRITLYLTEKK
YSPCAWEVVRAEIMRSFSSSRNLQERLRRKE.
IFNα7
(SEQ ID NO: 376)
CDLPQTHSLRNRRALILLAQMGRISPFSCLKDRHEFRFPEEEFDG
HQFQKTQAISVLHEMIQQTENLESTEDSSAAWEQSLLEKFSTELY
QQLNDLEACVIQEVGVEETPLMNEDFILAVRKYFQRITLYLMEKK
YSPCAWEVVRAEIMRSFSFSTNLKKGLRRKD.
IFNα8
(SEQ ID NO: 377)
CDLPQTHSLGNRRALILLAQMRRISPFSCLKDRHDFEFPQEEFDD
KQFQKAQAISVLHEMIQQTENLESTKDSSAALDETLLDEFYIELD
QQLNDLESCVMQEVGVIESPLMYEDSILAVRKYFQRITLYLTEKK
YSSCAWEVVRAEIMRSFSLSINLQKRLKSKE.
IFNα10
(SEQ ID NO: 378)
CDLPQTHSLGNRRALILLGQMGRISPFSCLKDRHDFRIPQEEFDG
NQFQKAQAISVLHEMIQQTENLFSTEDSSAAWEQSLLEKESTELY
QQLNDLEACVIQEVGVEETPLMNEDSILAVRKYFQRITLYLIERK
YSPCAWEVVRAEIMRSLSFSTNLQKRLRRKD.
IFNα14
(SEQ ID NO: 379)
CNLSQTHSLNNRRTLMLMAQMRRISPFSCLKDRHDFEFPQEEFDG
NQFQKAQAISVLHEMMQQTENLFSTKNSSAAWDETLLEKFYIELF
QQMNDLEACVIQEVGVEETPLMNEDSILAVKKYFQRITLYLMEKK
YSPCAWEVVRAEIMRSLSESTNLQKRLRRKD.
IFNα16
(SEQ ID NO: 380)
CDLPQTHSLGNRRALILLAQMGRISHFSCLKDRYDFGFPQEVEDG
NQFQKAQAISAFHEMIQQTENLESTKDSSAAWDETLLDKFYIELF
QQLNDLEACVTQEVGVEEIALMNEDSILAVRKYFQRITLYLMGKK
YSPCAWEVVRAEIMRSFSESTNLQKGLRRKD.
IFNα17
(SEQ ID NO: 381)
CDLPQTHSLGNRRALILLAQMGRISPFSCLKDRHDFGLPQEEFDG
NQFQKTQAISVLHEMIQQTENLESTEDSSAAWEQSLLEKESTELY
QQLNNLEACVIQEVGMEETPLMNEDSILAVRKYFQRITLYLTEKK
YSPCAWEVVRAEIMRSLSESTNLQKILRRKD.

In certain aspects, the IFN-α or IFN-β variant polypeptide has an amino acid sequence that has at least about 30%, at least about 40%, at least about 50%, at least about 60%, at least about 70%, at least about 80%, at least about 85%, at least about 90%, or has greater than about 90% (e.g., about 91%, about 92%, about 93%, about 94%, about 95%, about 96%, about 97%, about 98%, or about 99%) sequence identity to SEQ ID NOS: 371-482, respectively.

In some embodiments, the wild type or modified signaling agent is a modified interferon-α having decreased binding affinity for its receptor, particularly IFNAR2. In such embodiments, the modified IFNα1, IFNα2, IFNα4, IFNα5, IFNα6, IFNα7, IFNα8, IFNα10, IFNα12, IFNα14, IFNα16 or IFNα17 agent has reduced affinity for and/or induction of signaling at IFNAR (IFNAR1 and/or IFNAR2 chains).

With the exception of wild-type IFNα1, wild-type IFNs bind to IFNAR2 at affinities (KD), e.g., as determined by microcal or SPR, between 0.1 nM and 5 nM and to IFNAR1 at an affinity of about 1 μM. IFNα1 binds to IFNAR2 with a KD of about 200 nM. In some embodiments, an IFN is modified so as to have an affinity for IFNAR1 and/or IFNAR2 that is equal or less than that of the NKp46 ABD for NKp46. In some embodiments, an IFN is modified so as to have an affinity for IFNAR1 and/or IFNAR2 that is at least 1-log less than that of the NKp46 AD for NKp46.

For example, in the exemplary NKp46 ABDs shown herein, the NKp46 ABD has a KID for NKp46 binding of about 15 nM. An IFN can thus be modified by introduction of a modification that causes a reduction of binding affinity of between 10-fold (1-log) and 1000-fold (3-log) (an increase in KID of between 1-log and 3-log). An IFN can include any of the amino acid substitutions shown in the table below. The table below shows exemplary single amino acid substitutions that decrease binding affinity of IFN-α polypeptides to IFNAR2, with a cut-off of a decrease in affinity (higher KID) of at least 1 log compared to the wild-type counterpart and no more than 3-log compared to the wild-type counterpart. The table below shows the relative affinity based on KID values for IFNAR2 of the mutated cytokine compared to the wild-type cytokine.

| | IFNAR2 | | | | | | | | | | |
|---|---|---|---|---|---|---|---|---|---|---|---|
| | relative | | | | | | | | | | |
| IFNα2 | affinity | IFNa6 | IFNα4 | IFNα17 | IFNα10 | IFNα7 | IFNα5 | IFNα14 | IFNα16 | IFNα1 | IFNα8 |
| L15A | 0.1 | M15A | L15A | L15A | L15A | L15A | L15A | L15A | L15A | L15A | L15A |
| L30A | 0.0013 | L30A | L30A | L30A | L30A | L30A | L30A | L30A | L30A | L30A | L30A |
| L30V | 0.023 | L30V | L30V | L30V | L30V | L30V | L30V | L30V | L30V | L30V | L30V |
| R144A | 0.03 | R145A | R145A | R145A | R145A | R145A | R145A | R145A | R145A | R145A | R145A |
| A145M | 0.16 | A146M | A146M | A146M | A146M | A146M | A146M | A146M | A146M | A146M | A146M |
| A145G | 0.03 | A146G | A146G | A146G | A146G | A146G | A146G | A146G | A146G | A146G | A146G |
| M148A | 0.03 | M149A | M149A | M149A | M149A | M149A | M149A | M149A | M149A | M149A | M149A |
| R149A | 0.01 | R150A | R150A | R150A | R150A | R150A | R150A | R150A | R150A | R150A | R150A |
| S152A | 0.1 | S153A | S153A | S153A | S153A | S153A | S153A | S153A | S153A | S153A | S153A |
| L153A | 0.1 | S154A | F154A | F154A | F154A | F154A | L154A | F154A | F154A | L154A | L154A |

The table below shows exemplary single amino acid substitutions decreasing binding affinity of IFN-α polypeptides to IFNAR1, with an at least 2-fold decrease in affinity. The table shows the relative affinity based on KID values for IFNAR1 of the mutated cytokine compared to the wild-type cytokine.

| | IFNAR1 | | | | | | | | | | |
|---|---|---|---|---|---|---|---|---|---|---|---|
| | relative | | | | | | | | | | |
| IFNα2 | affinity | IFNα | IFNα4 | IFNα17 | IFNα10 | IFNα7 | IFNα5 | IFNα14 | IFNα16 | IFNα1 | IFNα8 |
| F64A | 0.44 | F65A | F65A | F65A | F65A | F65A | F65A | F65A | F65A | F65A | F65A |
| N65A | 0.29 | N66A | N66A | N66A | N66A | N66A | N66A | N66A | N66A | N66A | N66A |
| T69A | 0.4 | T70A | T70A | T70A | T70A | T70A | T70A | T70A | T70A | T70A | T70A |
| S73A | 0.5 | S74A | S74A | S74A | S74A | S74A | S74A | S74A | S74A | S74A | S74A |
| L80A | 0.17 | L81A | L81A | L81A | L81A | L81A | L81A | L81A | L81A | L81A | L81A |
| Y85A | 0.35 | Y86A | Y86A | S86A | S86A | S86A | S86A | Y86A | Y86A | C86A | Y86A |
| Y89A | 0.25 | Y90A | Y90A | Y90A | Y90A | Y90A | Y90A | F90A | F90A | Y90A | D90A |
| N93A | 0.46 | N94A | N94A | N94A | N94A | N94A | N94A | N94A | N94A | N94A | N94A |
| E96A | 0.51 | E97A | E97A | E97A | E97A | E97A | E97A | E97A | E97A | E97A | E97A |
| L117A | 0.45 | L118A | L118A | L118A | L118A | L118A | L118A | L118A | L118A | L118A | L118A |
| R120A | <0.05 | R121A | R121A | R121A | R121A | R121A | R121A | K121A | R121A | K121A | R121A |

Mutant forms of IFNα2 are also described for example in PCT publication nos. WO2008/124086, WO2010/030671, WO2013/059885, WO2013/107791, WO2015/007520 and WO2020/198661, the disclosures of which are incorporated hereby by reference.

In some embodiments, said IFNα2 mutant (IFNα2a or IFNα2b) is mutated at one or more amino acids at positions 144-154, such as amino acid positions 148, 149 and/or 153. In some embodiments, the IFNα2 mutant comprises one or more mutations selected from L153A, R149A, and M148A, described in WO2013/107791. In some embodiments, the IFNα2 mutants have reduced affinity and/or activity for IFNAR1. In some embodiments, the IFNα2 mutant comprises one or more mutations selected from F64A, N65A, T69A, L80A, Y85A, and Y89A, as described in WO2010/030671. In some embodiments, the IFNα2 mutant comprises one or more mutations selected from K133A, R144A, R149A, and L153A as described in WO2008/124086. In some embodiments, the IFNα2 mutant comprises one or more mutations selected from R120E and R120E/K121E, as described in WO2015/007520 and WO2010/030671. In one embodiment, the mutant human IFNα2 comprises an amino acid sequence having at least 95% identity with SEQ ID NO: 371, wherein the mutant IFNα2 has one or more mutations at positions L15, A19, R22, R23, L26, F27, L30, L30, K31, D32, R33, H34, D35, Q40, H57, E58, Q61, F64, N65, T69, L80, Y85, Y89, D 114, L117, R120, R125, K 133, K 134, R144, A145, M 148, R149, S 152, L153, and N156 with respect to SEQ ID NO: 371. In some embodiments, the human IFNα2 mutant comprises one or more mutations selected from, L15A, A19W, R22A, R23A, L26A, F27A, L30A, L30V, K31A, D32A, R33K, R33A, R33Q, H34A, D35A, Q40A, T106A, T106E, D114R, L117A, R120A, R125A, K134A, R144A, A145G, A145M, M148A, R149A, S152A, L153A, and N156A as disclosed in WO 2013/059885, for example in some embodiments, the human IFNα2 mutant comprises the mutations H57Y, E58N, Q61S, and/or L30A; the mutations H57Y, E58N, Q61S, and/or R33A; the mutations H57Y, E58N, Q61S, and/or M148A; the mutations H57Y, E58N, Q61S, and/or L153A; the mutations N65A, L80A, Y85A, and/or Y89A; or the mutations N65A, L80A, Y85A, Y89A, and/or D114A.

In embodiments, the wild type or modified signaling agent is a modified interferon-α having decreased binding affinity for its receptor, particularly IFNAR2. In such embodiments, the modified IFNα2 agent has reduced affinity for and/or induction of signaling at IFNAR (IFNAR1 and/or IFNAR2 chains).

In some embodiments, the IFNα1 interferon is modified to have a mutation at one or more amino acids at positions L15, A19, R23, S25, L30, D32, R33, H34, Q40, C86, D115, L118, K121, R126, E133, K134, K135, R145, A146, M149, R150, S153, L154, and N157 with reference to SEQ ID NO: 372. The mutations can optionally be a hydrophobic mutation and can be, e.g., selected from alanine, valine, leucine, and isoleucine. In some embodiments, the FNα1 interferon is modified to have a one or more mutations selected from L15A, A19W, R23A, S25A, L30A, L30V, D32A, R33K, R33A, R33Q, H34A, Q40A, C86S, C86A, D115R, L118A, K121A, K121 E, R126A, R126E, E133A, K134A, K135A, R145A, R145D, R145E, R145G, R145H, R1451, R145K, R145L, R145N, R145Q, R145S, R145T, R145V, R145Y, A146D, A146E, A146G, A146H, A1461, A146K, A146L, A146M, A146N, A146Q, A146R, A146S, A146T, A146V, A146Y, M149A, M149V, R150A, S153A, L154A, and N157A with reference to SEQ ID NO: 372. In some embodiments, the FNα1 mutant comprises one or more multiple mutations selected from L30A/H58Y/E59N/Q62S, R33A/H58Y/E59N/Q62S, M149A/H58Y/E59N/Q62S, L154A/H58Y/E59N/Q62S, R145A/H58Y/E59N/Q62S, D115A/R121A, L118A/R121A, L118A/R121 A/K122A, R121A/K122A, and R121 E/K122E with reference to SEQ ID NO: 372. In some embodiments, the IFN-α1 is a variant that comprises one or more mutations which reduce undesired disulphide pairings wherein the one or more mutations are, e.g., at amino acid positions C1, C29, C86, C99, or C139 with reference to SEQ ID NO: 372. In some embodiments, the mutation at position C86 can be, e.g., C86S or C86A or C86Y.

In embodiments, the wild type or modified signaling agent is IFN-β. In some embodiments, the IFN-β is human having a sequence as shown below:

(SEQ ID NO: 382)
MSYNLLGFLQRSSNFQCQKLLWQLNGRLEYCLKDRMNFDIPEEIK
QLQQFQKEDAALTIYEMLQNIFAIFRQDSSSTGWNETIVENLLAN
VYHQINHLKTVLEEKLEKEDFTRGKLMSSLHLKRYYGRILHYLKA
KEYSHCAWTIVRVEILRNFYFINRLTGYLRN.

In some embodiments, the human IFN-β is a non-glycosylated form of human IFN-β that has a Met-1 deletion and a Cys-17 to Ser mutation. In various embodiments, the modified IFN-β has one or more mutations that reduce its binding to or its affinity for the IFNAR1 subunit of IFNAR. In one embodiment, the modified IFN-β has reduced affinity and/or activity at IFNAR1. In various embodiments, the modified IFN-β is human IFN-β and has one or more mutations at positions F67, R71, L88, Y92, 195, N96, K123, and R124. In some embodiments, the one or more mutations are substitutions selected from F67G, F67S, R71A, L88G, L88S, Y92G, Y92S, 195A, N96G, K123G, and R124G.

In some embodiments, the modified IFN-β has one or more mutations that reduce its binding to or its affinity for the IFNAR2 subunit of IFNAR. In one embodiment, the modified IFN-β has reduced affinity and/or activity at IFNAR2. In various embodiments, the modified IFN-β is human IFN-β and has one or more mutations at positions W22, R27, L32, R35, V148, L151, R152, and Y155. In some embodiments, the one or more mutations are substitutions selected from W22G, R27G, L32A, L32G, R35A, R35G, V148G, L151G, R152A, R152G, and Y155G.

Exemplary IFN-β mutations are described in PCT publication nos. WO2020/198661, WO2000/023114 and US20150011732 the disclosures of which are incorporated hereby by reference.

In another embodiment where the cytokine-binding ABD binds an IL-7 receptor (IL-7R)-binding ABD, the ABD can be or comprise a suitable interleukin-7 (IL-7) polypeptide such that the IL-7R ABD binds IL-7Rα on the surface of NK cells. In some embodiments, the cytokine molecule is an IL-7 molecule, e.g., a full length, a fragment or a variant of IL-7, e.g., human IL-7. In embodiments, the IL-7 molecule is a wild-type, human IL-7, e.g., having the amino acid sequence of SEQ ID NO: 383. In some embodiments, the IL-7 molecule comprises an amino sequence at least 70%, 80%, 90%, 95%, 98% or 99% identical to a mature wild-type human IL-7 amino acid sequence of SEQ ID NO: 383. In other embodiments, the IL-7 molecule is a variant of human IL-7, e.g., having one or more amino acid modifications. Optionally the IL-7 comprises a fragment of a human IL-7 polypeptide, wherein the fragment has an amino sequence is identical to or at least 70%, 80%, 90%, 95%, 98% or 99% identical to a contiguous sequence of 40, 50, 60, 70 or 80 amino acids of the polypeptide of SEQ ID NO: 383.

Wild-type mature human IL-7:
(SEQ ID NO: 383)
DCDIEGKDGKQYESVLMVSIDQLLDSMKEIGSNCLNNEFNFFKRH
ICDANKEGMFLFRAARKLRQFLKMNSTGDEDLHLLKVSEGTTILL
NCTGQVKGRKPAALGEAQPTKSLEENKSLKEQKKLNDLCFLKRLL
QEIKTCWNKILMGTKEH

Wild-type IL-7 bind to IL-7Rα with an affinity (KD), e.g., as determined by microcal or SPR, of between about 50-100 nM. In some embodiments, an IL-7 is modified so as to have an affinity for IL-7Rα that is equal or less than that of the NKp46 ABD for NKp46. In some embodiments, an IL-7 is modified so as to have an affinity for IL-7Rα that is at least 1-log less than that of the NKp46 ABD for NKp46. In some embodiments, an IL-7 is modified so as to have an affinity for IL-7Rα that is at least 1-log less than that of the NKp46 ABD for NKp46, but no more than 3-log, or optionally 2-log, less than that of the NKp46 ABD for NKp46. For example, in the exemplary NKp46 ABDs shown herein, the NKp46 ABD has a KD for NKp46 binding of about 15 nM. An IL-7 can thus be modified by introduction of a modification such as amino acid substitutions Q22A, D74A and/or K81A (with reference to the wild-type mature IL-7 amino acid sequence) that causes a reduction of affinity between IL-7 and IL-7Ra.

In another embodiment where the cytokine-binding ABD binds an IL-27 receptor (IL-27R)-binding ABD, the ABD can be or comprise a suitable interleukin-27 (IL-27) polypeptide such that the IL-27R ABD binds IL-27R (WSX-1 and/or gp130) on the surface of NK cells. In some embodiments, the cytokine molecule is an IL-27 molecule, e.g., a full length, a fragment or a variant comprising the P28 and EBI3 subunits, e.g., human single chain or heterodimeric IL-27 comprising the P28 and EBI3 subunits, optionally wherein the EBI3 and p28 subunits of IL-27 are linked by a domain linker (e.g., a flexible polypeptide linker, a linker containing glycine a serine residues, a (G₄S)₂ or (G₄S)₃ linker) into a single-chain format. Single-chain forms of IL-27 can be generated consisting of the p28 subunit linked to the EBI3 subunit by a flexible linker, either through the C-terminus of p28 linked to the N-terminus of EBI3 or vice versa. In embodiments, the IL-27 molecule is a wild-type, human IL-27, e.g., a single chain fusion product or a heterodimer comprising the amino acid sequences of SEQ ID NOS: 384 and 385 or a IL27R-binding fragment of any of the SEQ ID NOS: 384 and 385. In some embodiments, the IL-27 molecule comprises an amino sequence at least 70%, 80%, 90%, 95%, 98% or 99% identical to a mature wild-type human IL-27 p28 subunit amino acid sequence of SEQ ID NO: 384 and/or an amino sequence at least 70%, 80%, 90%, 95%, 98% or 99% identical to a mature wild-type human IL-27 EBI3 subunit amino acid sequence of SEQ ID NO: 385. In other embodiments, the IL-27 molecule is a variant of human IL-27, e.g., having one or more amino acid modifications. Optionally the IL-27 comprises a fragment of a human IL-27 p28 subunit polypeptide, wherein the fragment has an amino sequence that is identical to or at least 70%, 80%, 90%, 95%, 98% or 99% identical to a contiguous sequence of 40, 50, 60, 70 or 80 amino acids of the polypeptide of SEQ ID NO: 384, and/or a fragment of a human IL-27 EBI3 subunit polypeptide, wherein the fragment has an amino sequence that is identical to or at least 70%, 80%, 90%, 95%, 98% or 99% identical to a contiguous sequence of 40, 50, 60, 70 or 80 amino acids of the polypeptide of SEQ ID NO: 385. The p28 subunit can be specified as being linked at its N-terminus to the multispecific protein (or to the NKp46 ABD thereof). The EBI3 subunit can be specified as being linked, at its N-terminus, to the C-terminus of the p28 subunit, optionally via a domain linker, or can be specified as being placed on a separate polypeptide that associates with the p28 subunit.

Wild-type mature human IL-27 p28 subunit:
(SEQ ID NO: 384)
FPRPPGRPQLSLQELRREFTVSLHLARKLLSEVRGQAHRFAESHL
PGVNLYLLPLGEQLPDVSLTFQAWRRLSDPERLCFISTTLQPFHA
LLGGLGTQGRWTNMERMQLWAMRLDLRDLQRHLRFQVLAAGENLP
EEEEEEEEEEEEERKGLLPGALGSALQGPAQVSWPQLLSTYRLLH
SLELVLSRAVRELLLLSKAGHSVWPLGFPTLSPQP.
Wild-type mature human IL-27 EBI3 subunit:
(SEQ ID NO: 385)
RKGPPAALTLPRVQCRASRYPIAVDCSWTLPPAPNSTSPVSFIAT
YRLGMAARGHSWPCLQQTPTSTSCTITDVQLESMAPYVLNVTAVH
PWGSSSSFVPFITEHIIKPDPPEGVRLSPLAERQLQVQWEPPGSW
PFPEIFSLKYWIRYKRQGAARFHRVGPIEATSFILRAVRPRARYY
VQVAAQDLTDYGELSDWSLPATATMSLGK

In some embodiments, an IL-27 is modified so as to have an affinity for WSX-1 and/or gp130 that is equal or less than that of the NKp46 ABD for NKp46. In some embodiments, an IL-27 is modified so as to have an affinity for WSX-1 and/or gp130 that is at least 1-log less than that of the NKp46 ABD for NKp46. In some embodiments, an IL-27 is modified so as to have an affinity for WSX-1 and/or gp130 that is at least 1-log less than that of the NKp46 ABD for NKp46, but no more than 3-log, or optionally 2-log, less than that of the NKp46 ABD for NKp46.

In another embodiment where the cytokine-binding ABD binds an IL-12 receptor (IL-12R)-binding ABD, the ABD can be or comprise a suitable interleukin-12 (IL-12) polypeptide such that the IL-12R ABD binds IL-12R (IL-12Rβ1 and/or IL-12Rβ2) on the surface of NK cells. In some embodiments, the cytokine molecule is an IL-12 molecule, e.g., a full length, a fragment or a variant comprising the P35 and P40 subunits, e.g., human single chain or heterodimeric IL-12 comprising the P35 and P40 subunits, optionally wherein the p40 and p35 subunits of IL-12 are linked by a domain linker (e.g., a flexible polypeptide linker, a linker containing glycine a serine residues, a (G₄S)₂ or (G₄S)₃ linker) into a single-chain format. Single-chain forms of IL-12 can be generated consisting of the p35 subunit linked to the p40 subunit by a flexible linker, either through the C-terminus of p35 linked to the N-terminus of p40 or vice versa. In embodiments, the IL-12 molecule is a wild-type, human IL-12, e.g., a single chain fusion product or a heterodimer comprising the amino acid sequences of SEQ ID NOS: 386 and 387 or a IL12R-binding fragment of any of the SEQ ID NOS: 386 or 387. In some embodiments, the IL-12 molecule comprises an amino sequence at least 70%, 80%, 90%, 95%, 98% or 99% identical to a mature wild-type human IL-12 p35 subunit amino acid sequence of SEQ ID NO: 386 and/or an amino sequence at least 70%, 80%, 90%, 95%, 98% or 99% identical to a mature wild-type human IL-12 p40 subunit amino acid sequence of SEQ ID NO: 387. In other embodiments, the IL-12 molecule is a variant of human IL-12, e.g., having one or more amino acid modifications. Optionally the IL-12 comprises a fragment of a human IL-12 p35 subunit polypeptide, wherein the fragment has an amino sequence that is identical to or at least 70%, 80%, 90%, 95%, 98% or 99% identical to a contiguous sequence of 40, 50, 60, 70 or 80 amino acids of the polypeptide of SEQ ID NO: 386, and/or a fragment of a human IL-12 p40 subunit polypeptide, wherein the fragment has an amino sequence that is identical to or at least 70%, 80%, 90%, 95%, 98% or 99% identical to a contiguous sequence of 40, 50, 60, 70 or 80 amino acids of the polypeptide of SEQ ID NO: 387. The p35 (alpha) and P40 (beta) can be specified as being linked by a disulphide bridge between Cys74 of the P35 subunit and the Cys177 of the P40 subunit. The p35 subunit can be specified as being linked at its N-terminus to the multispecific protein (or to the NKp46 ABD thereof). The p40 subunit can be specified as being linked, at its N-terminus, to the C-terminus of the p35 subunit, optionally via a domain linker, or can be specified as being placed on a separate polypeptide that associates with the p35 subunit.

Wild-type mature human IL-12 p35 subunit:
(SEQ ID NO: 387)
RNLPVATPDPGMFPCLHHSQNLLRAVSNMLQKARQTLEFYPCTSE
EIDHEDITKDKTSTVEACLPLELTKNESCLNSRETSFITNGSCLA
SRKTSEMMALCLSSIYEDLKMYQVEFKTMNAKLLMDPKRQIELDO
NMLAVIDELMQALNENSETVPQKSSLEEPDFYKTKIKLCILLHAF
RIRAVTIDRVMSYLNAS.
Wild-type mature human IL-12 p40 subunit:
(SEQ ID NO: 386)
IWELKKDVYVVELDWYPDAPGEMVVLTCDTPEEDGITWTLDQSSE
VLGSGKTLTIQVKEFGDAGQYTCHKGGEVLSHSLLLLHKKEDGIW
STDILKDQKEPKNKTFLRCEAKNYSGRFTCWWLTTISTDLTESVK
SSRGSSDPQGVTCGAATLSAERVRGDNKEYEYSVECQEDSACPAA
EESLPIEVMVDAVHKLKYENYTSSFFIRDIIKPDPPKNLQLKPLK
NSRQVEVSWEYPDTWSTPHSYFSLTFCVQVQGKSKREKKDRVFTD
KTSATVICRKNASISVRAQDRYYSSSWSEWASVPCS

Wild-type IL-12 dimer binds to IL-12Rβ1 and IL-12Rβ2 with an affinity (KD), e.g., as determined by microcal or SPR, of about 5-7 nM and 5 nM, respectively, and IL-12 dimer binds to IL12Rβ1:IL-12Rβ2 dimers with a KD of about 50 μM. In some embodiments, an IL-12 is modified so as to have an affinity for IL-12Rβ1 and/or IL-12Rβ2 that is equal or less than that of the NKp46 ABD for NKp46. In some embodiments, an IL-12 is modified so as to have an affinity for IL-12Rβ1 and/or IL-12Rβ2 that is at least 1-log less than that of the NKp46 ABD for NKp46. In some embodiments, an IL-12 is modified so as to have an affinity for IL-12Rβ1 and/or IL-12Rβ2 that is at least 1-log lower (1-log higher KD) than that of the NKp46 ABD for NKp46, but no more than 3-log, or optionally 2-log, lower than that of the NKp46 ABD for NKp46.

### NKp46 Variable Region and CDR Sequences

In some embodiments, the multispecific protein or NKp46 ABD thereof (or the anti-NKp46 antibody from which the ABD is derived) binds the D1 domain of NKp46, the D2 domain of NKp46, or bind a region spanning the D1 and D2 domains (at the border of the D1 and D2 domains, the D1/D2 junction), of the NKp46 polypeptide of SEQ ID NO: 1. In some embodiments, the multispecific proteins comprise VH/VL pair from an anti-NKp46 antibody having an affinity for human NKp46, as a full-length IgG antibody, characterized by a K_{D} of less than 10⁻⁸ M, less than 10⁻⁹ M, or less than 10⁻¹⁰ M. In some embodiments, the multispecific proteins have an affinity (KD) for human NKp46 of between 1 and 100 nM, optionally between 1 and 50 nM, optionally between 1 and 20 nM, optionally about 10 or 15 nM, as determined by SPR.

In one embodiment, the multispecific protein (or a NKp46-binding ABD or VH/VL pair thereof, for example as when configured in the multispecific protein or as a conventional full-length antibody) binds NKp46 at substantially the same region, site or epitope on NKp46 as antibody NKp46-1, NKp46-2, NKp46-3, NKp46-4, NKp46-6 or NKp46-9. In another embodiment, the antibodies at least partially overlaps, or includes at least one residue in the segment or epitope bound by NKp46-1, NKp46-2, NKp46-3, NKp46-4, NKp46-6 or NKp46-9. In one embodiment, all key residues of the epitope are in a segment corresponding to domain D1 or D2. In one embodiment, the antibody or multispecific protein binds a residue present in the D1 domain as well as a residue present in in the D2 domain. In one embodiment, the antibodies bind an epitope comprising 1, 2, 3, 4, 5, 6, 7 or more residues in the segment corresponding to domain D1 or D2 of the NKp46 polypeptide of SEQ ID NO: 1. In one embodiment, the antibodies bind domain D1 and further bind an epitope comprising 1, 2, 3, or 4 of the residues R101, V102, E104 and/or L105.

In another embodiment, the antibodies or multispecific proteins bind NKp46 at the D1/D2 domain junction and bind an epitope comprising or consisting of 1, 2, 3, 4 or 5 of the residues K41, E42, E119, Y121 and/or Y194.

In another embodiment, the antibodies or multispecific proteins bind domain D2 and bind an epitope comprising 1, 2, 3, or 4 of the residues P132, E133, I135, and/or S136.

The Examples section provided describes a series of mutant human NKp46 polypeptides. In the examples, the binding of multispecific protein to cells transfected with the NKp46 mutants was measured and compared to the ability of anti-NKp46 antibody to bind wild-type NKp46 polypeptide (SEQ ID NO: 1). A reduction in binding between an anti-NKp46 antibody or NKp46 binding multispecific protein and a mutant NKp46 polypeptide as described herein means that there is a reduction in binding affinity (e.g., as measured by known methods such FACS testing of cells expressing a particular mutant, or by Biacore testing of binding to mutant polypeptides) and/or a reduction in the total binding capacity of the anti-NKp46 antibody (e.g., as evidenced by a decrease in Bmax in a plot of anti-NKp46 antibody concentration versus polypeptide concentration). A significant reduction in binding indicates that the mutated residue is directly involved in the binding to the anti-NKp46 antibody to NKp46 or is in close proximity to the binding protein when the anti-NKp46 antibody or NKp46 binding multispecific protein is bound to NKp46. An antibody epitope will thus preferably include such residue and may include additional residues adjacent to such residue.

In some embodiments, a significant reduction in binding means that the binding affinity and/or capacity between an NKp46 ABD or NKp46 binding multispecific protein and a mutant NKp46 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type NKp46 polypeptide (e.g., the polypeptide shown in SEQ ID NO: 1). In certain embodiments, binding is reduced below detectable limits. In some embodiments, a significant reduction in binding is evidenced when binding of an anti-NKp46 antibody to a mutant NKp46 polypeptide is less than 50% (e.g., less than 45%, 40%, 35%, 30%, 25%, 20%, 15% or 10%) of the binding observed between the anti-NKp46 antibody and a wild-type NKp46 polypeptide (e.g., the polypeptide shown in SEQ ID NO: 1 (or the extracellular domain thereof)). Such binding measurements can be made using a variety of binding assays known in the art. A specific example of one such assay is described in the Example section.

In some embodiments, NKp46 binding multispecific proteins exhibit significantly lower binding for a mutant NKp46 polypeptide in which a residue in a wild-type NKp46 polypeptide (e.g., SEQ ID NO: 1) is substituted. In the shorthand notation used here, the format is: Wild type residue: Position in polypeptide: Mutant residue, with the numbering of the residues as indicated in SEQ ID NO: 1.

In some embodiments, an NKp46 binding multispecific binds a wild-type NKp46 polypeptide but has decreased binding to a mutant NKp46 polypeptide having a mutation (e.g., an alanine substitution) any one or more of the residues R101, V102, E104 and/or L105 (with reference to SEQ ID NO: 1) compared to binding to the wild-type NKp46).

In some embodiments, a NKp46-binding multispecific protein binds a wild-type NKp46 polypeptide but has decreased binding to a mutant NKp46 polypeptide having a mutation (e.g., an alanine substitution) at one or more of residues K41, E42, E119, Y121 and/or Y194 (with reference to SEQ ID NO: 1) compared to binding to the wild-type NKp46).

In some embodiments, a NKp46-binding multispecific protein binds a wild-type NKp46 polypeptide but has decreased binding to a mutant NKp46 polypeptide having a mutation (e.g., an alanine substitution) at one or more of residues P132, E133, I135, and/or S136 (with reference to SEQ ID NO: 1) compared to binding to the wild-type NKp46).

The amino acid sequence of the heavy chain variable region of antibodies NKp46-1, NKp46-2, NKp46-3, NKp46-4, NKp46-6 and NKp46-9 are listed herein in Table B (SEQ ID NOS: 3, 5, 7, 9, 11 and 13 respectively), the amino acid sequence of the light chain variable region of antibodies NKp46-1, NKp46-2, NKp46-3, NKp46-4, NKp46-6 and NKp46-9 are also listed herein in Table B (SEQ ID NOS: 4, 6, 8, 10, 12 and 14 respectively).

A NKp46-binding multispecific protein that binds essentially the same epitope or determinant as monoclonal antibody NKp46-1, NKp46-2, NKp46-3, NKp46-4, NKp46-6 or NKp46-9; optionally the antibody comprises a hypervariable region of antibody NKp46-1, NKp46-2, NKp46-3, NKp46-4, NKp46-6 or NKp46-9. In any of the embodiments herein, antibody NKp46-1, NKp46-2, NKp46-3, NKp46-4, NKp46-6 or NKp46-9 can be characterized by its amino acid sequence and/or nucleic acid sequence encoding it. In one embodiment, the antibody comprises the Fab or F(ab′)₂ portion of NKp46-1, NKp46-2, NKp46-3, NKp46-4, NKp46-6 or NKp46-9. Also provided is an antibody that comprises the heavy chain variable region of NKp46-1, NKp46-2, NKp46-3, NKp46-4, NKp46-6 or NKp46-9. According to one embodiment, an antibody comprises the three CDRs of the heavy chain variable region of NKp46-1, NKp46-2, NKp46-3, NKp46-4, NKp46-6 or NKp46-9. Also provided is a polypeptide that further comprises one, two or three of the CDRs of the light chain variable region of NKp46-1, NKp46-2, NKp46-3, NKp46-4, NKp46-6 or NKp46-9. Optionally any one or more of said light or heavy chain CDRs may contain one, two, three, four or five or more amino acid modifications (e.g. substitutions, insertions or deletions).

A multispecific protein or NKp46-binding ABD can for example comprise:
(a) the heavy chain variable region of NKp46-1, NKp46-2, NKp46-3, NKp46-4, NKp46-6 or NKp46-9 as set forth in Table B, optionally wherein one, two, three or more amino acids may be substituted by a different amino acid;
(b) the light chain variable region NKp46-1, NKp46-2, NKp46-3, NKp46-4, NKp46-6 or NKp46-9 as set forth in Table B, optionally wherein one, two, three or more amino acids may be substituted by a different amino acid;
(c) the heavy chain variable region of NKp46-1, NKp46-2, NKp46-3, NKp46-4, NKp46-6 or NKp46-9 as set forth in Table B, optionally wherein one or more of these amino acids may be substituted by a different amino acid; and the respective light chain variable region of NKp46-1, NKp46-2, NKp46-3, NKp46-4, NKp46-6 or NKp46-9 as set forth in Table B, optionally wherein one, two, three or more amino acids may be substituted by a different amino acid;
(d) the heavy chain CDR 1, 2 and 3 (HCDR1, HCDR2) amino acid sequence of NKp46-1, NKp46-2, NKp46-3, NKp46-4, NKp46-6 or NKp46-9 as shown in Table A, optionally wherein one, two, three or more amino acids in a CDR may be substituted by a different amino acid;
(e) the light chain CDR 1, 2 and 3 (LCDR1, LCDR2, LCDR3) amino acid sequence of NKp46-1, NKp46-2, NKp46-3, NKp46-4, NKp46-6 or NKp46-9 as shown in Table A, optionally wherein one, two, three or more amino acids in a CDR may be substituted by a different amino acid; or
(f) the heavy chain CDR 1, 2 and 3 (HCDR1, HCDR2, HCDR3) amino acid sequence of NKp46-1, NKp46-2, NKp46-3, NKp46-4, NKp46-6 or NKp46-9 as shown in Table A, optionally wherein one, two, three or more amino acids in a CDR may be substituted by a different amino acid; and the light chain CDRs 1, 2 and 3 (LCDR1, LCDR2, LCDR3) amino acid sequence of the respective NKp46-1, NKp46-2, NKp46-3, NKp46-4, NKp46-6 or NKp46-9 antibody as shown in Table A, optionally wherein one, two, three or more amino acids in a CDR may be substituted by a different amino acid.

In one embodiment, the aforementioned CDRs are according to Kabat, e.g. as shown in Table A. In one embodiment, the aforementioned CDRs are according to Chothia numbering, e.g. as shown in Table A. In one embodiment, the aforementioned CDRs are according to IMGT numbering, e.g. as shown in Table A.

In another aspect of any of the embodiments herein, any of the CDR1, CDR2 and CDR3 of the heavy and light chains may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs listed in the corresponding SEQ ID NO or Table A.

In another aspect, a multispecific protein competes for binding to an epitope on NKp46 with a monoclonal antibody according to (a) to (f), above.

The sequences of the CDRs, according to IMGT, Kabat and Chothia definitions systems, are summarized in Table A below. The sequences of the variable chains of the antibodies according to the invention are listed in Table B below. In any embodiment herein, a V_{L} or V_{H} sequence can be specified or numbered so as to contain or lack a signal peptide or any part thereof.

TABLE A

| | | HCDR1 | | HCDR2 | | HCDR3 | |
|---|---|---|---|---|---|---|---|
| | CDR | SEQ | | SEQ | | SEQ | |
| mAb | definition | ID | Sequence | ID | Sequence | ID | Sequence |
| NKp46-1 | Kabat | 15 | DYVIN | 18 | EIYPGSGTNYYNEKFKA | 21 | RGRYGLYAMDY |
| | Chothia | 16 | GYTFTDY | 19 | PGSG | 22 | GRYGLYAMD |
| | IMGT | 17 | GYTFTDYV | 20 | GYTFTDYVIYPGSGTN | 23 | ARRGRYGLYAMD |
| | | | | | | | Y |
| NKp46-2 | Kabat | 31 | SDYAWN | 34 | YITYSGSTSYNPSLES | 36 | GGYYGSSWGVFA |
| | | | | | | | Y |
| | Chothia | 32 | GYSITSDY | | YSG | 37 | GYYGSSWGVFA |
| | IMGT | 33 | GYSITSDYA | 35 | ITYSGST | 38 | ARGGYYGSSWGV |
| | | | | | | | FAY |
| NKp46-3 | Kabat | 46 | EYTMH | 49 | GISPNIGGTSYNQKFKG | 51 | RGGSFDY |
| | Chothia | 47 | GYTFTEY | | PNIG | 52 | GGSFD |
| | IMGT | 48 | GYTFTEYT | 50 | ISPNIGGT | 53 | ARRGGSFDY |
| NKp46-4 | Kabat | 60 | SFTMH | 63 | YINPSSGYTEYNQKFKD | 65 | GSSRGFDY |
| | Chothia | 61 | GYTFTSF | | PSSG | 66 | SSRGFD |
| | IMGT | 62 | GYTFTSFT | 64 | INPSSGYT | 67 | VRGSSRGFDY |
| NKp46-6 | Kabat | 73 | SSWMH | 76 | HIHPNSGISNYNEKFKG | 78 | GGRFDD |
| | Chothia | 74 | GYTFTSS | | PNSG | | GRED |
| | IMGT | 75 | GYTFTSSW | 77 | IHPNSGIS | 79 | ARGGRFDD |
| NKp46-9 | Kabat | 85 | SDYAWN | 88 | YITYSGSTNYNPSLKS | 89 | CWDYALYAMDC |
| | Chothia | 86 | GYSITSDY | | YSG | 90 | WDYALYAMD |
| | IMGT | 87 | GYSITSDYA | 35 | ITYSGST | 91 | ARCWDYALYAMD |
| | | | | | | | C |
| Bab281 | Kabat | 97 | NYGMN | 100 | WINTNTGEPTYAEEFKG | 102 | DYLYYFDY |
| | Chothia | 98 | GYTFTNY | | TNTG | 103 | YLYYFD |
| | IMGT | 99 | GYTFTNYG | 101 | INTNTGEP | 104 | ARDYLYYFDY |
| NKp46-1 | Kabat | 24 | RASQDISNYLN | 27 | YTSRLHS | 28 | QQGNTRPWT |
| | Chothia | 25 | SQDISNY | | YTS | 29 | YTSGNTRPW |
| | IMGT | 26 | QDISNY | | YTS | 30 | YTSQQGNTRPW |
| | | | | | | | T |
| NKp46-2 | Kabat | 39 | RVSENIYSYLA | 42 | NAKTLAE | 43 | QHHYGTPWT |
| | Chothia | 40 | SENIYSY | | NAK | 44 | HYGTPW |
| | IMGT | 41 | ENIYSY | | NAK | 45 | QHHYGTPWT |
| NKp46-3 | Kabat | 54 | RASQSISDYLH | 57 | YASQSIS | 58 | QNGHSFPLT |
| | Chothia | 55 | SQSISDY | | YAS | 59 | GHSFPL |
| | IMGT | 56 | QSISDY | | YAS | | QNGHSFPLT |
| NKp46-4 | Kabat | 68 | RASENIYSNLA | 70 | AATNLAD | 71 | QHFWGTPRT |
| | Chothia | | SENIYSN | | AAT | 72 | FWGTPR |
| | IMGT | 69 | ENIYSN | | AAT | | QHFWGTPRT |
| NKp46-6 | Kabat | 80 | RASQDIGSSLN | 81 | ATSSLDS | 82 | LQYASSPWT |
| | Chothia | | SQDIGSS | | ATS | 83 | YASSPWT |
| | IMGT | | QDIGSS | | ATS | 84 | LQYASSPWT |
| NKp46-9 | Kabat | 92 | RTSENIYSYLA | 93 | NAKTLAE | 94 | QHHYDTPLT |
| | Chothia | | SENIYSY | | NAK | 95 | NAKHYDTPL |
| | IMGT | | ENIYSY | | NAK | 96 | QHHYDTPLT |
| Bab281 | Kabat | 105 | KASENVVTYVS | 108 | GASNRYT | 109 | GQGYSYPYT |
| | Chothia | 106 | SENVVTY | | GAS | 110 | GYSYPY |
| | IMGT | 107 | ENVVTY | | GAS | 111 | GQGYSYPYT |

TABLE B

| | SEQ ID | |
|---|---|---|
| Antibody | NO | Amino acid sequence |
| NKp46-1 VH | 3 | QVQLQQSGPELVKPGASVKMSCKASGYTFT |
| | | DYVINWGKQRSGQGLEWIGEIYPGSGTNYY |
| | | NEKFKAKATLTADKSSNIAYMQLSSLTSED |
| | | SAVYFCARRGRYGLYAMDYWGQGTSVTVSS |
| NKp46-1 VL | 4 | DIQMTQTTSSLSASLGDRVTISCRASQDIS |
| | | NYLNWYQQKPDGTVKLLIYYTSRLHSGVPS |
| | | RFSGSGSGTDYSLTINNLEQEDIATYFCQQ |
| | | GNTRPWTFGGGTKLEIK |
| NKp46-2 VH | 5 | EVQLQESGPGLVKPSQSLSLTCTVTGYSIT |
| | | SDYAWNWIRQFPGNKLEWMGYITYSGSTSY |
| | | NPSLESRISITRDTSTNQFFLQLNSVTTED |
| | | TATYYCARGGYYGSSWGVFAYWGQGTLVTV |
| | | SA |
| NKp46-2 VL | 6 | DIQMTQSPASLSASVGETVTITCRVSENIY |
| | | SYLAWYQQKQGKSPQLLVYNAKTLAEGVPS |
| | | RESGSGSGTQFSLKINSLQPEDFGSYYCQH |
| | | HYGTPWTFGGGTKLEIK |
| NKp46-3 VH | 7 | EVQLQQSGPELVKPGASVKISCKTSGYTFT |
| | | EYTMHWVKQSHGKSLEWIGGISPNIGGTSY |
| | | NQKFKGKATLTVDKSSSTAYMELRSLTSED |
| | | SAVYYCARRGGSFDYWGQGTTLTVSS |
| NKp46-3 VL | 8 | DIVMTQSPATLSVTPGDRVSLSCRASQSIS |
| | | DYLHWYQQKSHESPRLLIKYASQSISGIPS |
| | | RESGSGSGSDFTLSINSVEPEDVGVYYCQN |
| | | GHSFPLTFGAGTKLELK |
| NKp46-4 VH | 9 | QVQLQQSAVELARPGASVKMSCKASGYTFT |
| | | SFTMHWVKQRPGQGLEWIGYINPSSGYTEY |
| | | NQKFKDKTTLTADKSSSTAYMQLDSLTSDD |
| | | SAVYYCVRGSSRGFDYWGQGTLVTVSA |
| NKp46-4 VL | 10 | DIQMIQSPASLSVSVGETVTITCRASENIY |
| | | SNLAWFQQKQGKSPQLLVYAATNLADGVPS |
| | | RESGSGSGTQYSLKINSLQSEDFGIYYCQH |
| | | EWGTPRTFGGGTKLEIK |
| NKp46-6 VH | 11 | QVQLQQPGSVLVRPGASVKLSCKASGYTFT |
| | | SSWMHWAKQRPGQGLEWIGHIHPNSGISNY |
| | | NEKFKGKATLTVDTSSSTAYVDLSSLTSED |
| | | SAVYYCARGGREDDWGAGTTVTVSS |
| NKp46-6 VL | 12 | DIQMTQSPSSLSASLGERVSLTCRASQDIG |
| | | SSLNWLQQEPDGTIKRLIYATSSLDSGVPK |
| | | RFSGSRSGSDYSLTISSLESEDFVDYYCLQ |
| | | YASSPWTFGGGTKLEIK |
| NKp46-9 VH | 13 | DVQLQESGPGLVKPSQSLSLTCTVTGYSIT |
| | | SDYAWNWIRQFPGNKLEWMGYITYSGSTNY |
| | | NPSLKSRISITRDTSKNQFFLQLNSVTTED |
| | | TATYYCARCWDYALYAMDCWGQGTSVTVSS |
| NKp46-9 VL | 14 | DIQMTQSPASLSASVGETVTITCRTSENIY |
| | | SYLAWCQQKQGKSPQLLVYNAKTLAEGVPS |
| | | RESGSGSGTHESLKINSLQPEDFGIYYCQH |
| | | HYDTPLTFGAGTKLELK |

VH and VL pairs of an NKp46 ABD can optionally be function-conservative variants of the VH and VL of any of antibodies NKp46-1, NKp46-2, NKp46-3, NKp46-4, NKp46-6 or NKp46-9. “Function-conservative variants” are those in which a given amino acid residue in a protein (e.g. an antibody or antibody fragment) has been changed without altering the overall conformation and function of the protein, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity with the antibody capable of specifically binding to a KIR3DL2 polypeptide as defined hereinabove as determined by BLAST or FASTA algorithms, preferably at least 75%, more preferably at least 85%, still preferably at least 90%, and even more preferably at least 95%, and which has the same or substantially similar properties or functions as the antibodies capable of specifically binding to a KIR3DL2 polypeptide as defined hereinabove.

Exemplary humanized VH and VL domains can comprise all of an antigen binding region of antibody NKp46-1, NKp46-2, NKp46-3, NKp46-4, NKp46-6 or NKp46-9, for example having the amino acids of the SEQ ID NOS shown in Table 2.

A light chain variable region of a NKp46-1, NKp46-2, NKp46-3, NKp-46-4, NKp46-6 or NKp46-9 antibody may comprise, for the respective antibody: a human light chain FR1 framework region; a LCDR1 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human light chain FR2 framework region; a LCDR2 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human light chain FR3 framework region; and a LCDR3 region comprising an amino acid sequence as set forth in Table A, or a sequence of at least 4, 5, 6, 7, 8, 9 or 10 contiguous amino acids thereof, wherein one or more of these amino acids may be deleted or substituted by a different amino acid. Optionally, the variable region further comprises a human light chain FR4 framework region. Humanization of NKp46-1, NKp46-2, NKp46-3, NKp-46-4, and NKp46-9 VH/VL domains is described in PCT publication no. WO2017114694, the disclosure of which is incorporated herein by reference, amino acid sequence shown below.

NKp46-1: “H1” heavy chain variable region
(SEQ ID NO: 112)
QVQLVQSGAEVKKPGSSVKVSCKASGYTFSDYVINWVRQAPGQGL
EWMGEIYPGSGTNYYNEKFKAKATITADKSTSTAYMELSSLRSED
TAVYYCARRGRYGLYAMDYWGQGTTVTVSS.
NKp46-1: “H3” heavy chain variable region
(SEQ ID NO: 113)
QVQLVQSGAEVKKPGSSVKVSCKASGYTFTDYVINWGRQAPGQGL
EWIGEIYPGSGTNYYNEKFKAKATITADKSTSTAYMELSSLRSED
TAVYFCARRGRYGLYAMDYWGQGTTVTVSS.
NKp46-1: “L1” light chain variable region
(SEQ ID NO: 114)
DIQMTQSPSSLSASVGDRVTITCRASQDISNYLNWYQQKPGKAPK
LLIYYTSRLHSGVPSRFSGSGSGTDFTFTISSLQPEDIATYFCQQ
GNTRPWTFGGGTKVEIK.
NKp46-2: “H1” heavy chain variable region
(SEQ ID NO: 115)
QVQLQESGPGLVKPSQTLSLTCTVSGYSISSDYAWNWIRQPPGKG
LEWIGYITYSGSTSYNPSLESRVTISRDTSKNQFSLKLSSVTAAD
TAVYYCARGGYYGSSWGVFAYWGQGTLVTVSS
NKp46-2: “H2” heavy chain variable region
(SEQ ID NO: 116)
QVQLQESGPGLVKPSQTLSLTCTVSGYSISSDYAWNWIRQPPGKG
LEWMGYITYSGSTSYNPSLESRITISRDTSKNQFSLKLSSVTAAD
TAVYYCARGGYYGSSWGVFAYWGQGTLVTVSS.
NKp46-2: “H3” heavy chain variable region
(SEQ ID NO: 117)
QVQLQESGPGLVKPSQTLSLTCTVSGYSITSDYAWNWIRQPPGKG
LEWMGYITYSGSTSYNPSLESRITISRDTSKNQFSLKLSSVTAAD
TAVYYCARGGYYGSSWGVFAYWGQGTLVTVSS.
NKp46-2: “L1” light chain variable region
(SEQ ID NO: 118)
DIQMTQSPSSLSASVGDRVTITCRVSENIYSYLAWYQQKPGKAPK
LLVYNAKTLAEGVPSRFSGSGSGTDFTLTISSLQPEDFATYYCQH
HYGTPWTFGGGTKVEIK.
NKp46-3: “H1” heavy chain variable region
(SEQ ID NO: 119)
QVQLVQSGAEVKKPGSSVKVSCKASGYTFSEYTMHWVRQAPGQGL
EWMGGISPNIGGTSYNQKFKGRVTITADKSTSTAYMELSSLRSED
TAVYYCARRGGSFDYWGQGTTVTVSS.
NKp46-3: “H3” heavy chain variable region
(SEQ ID NO: 120)
QVQLVQSGAEVKKPGSSVKVSCKASGYTFSEYTMHWVRQAPGQGL
EWIGGISPNIGGTSYNQKFKGRATITADKSTSTAYMELSSLRSED
TAVYYCARRGGSFDYWGQGTTVTVSS.
NKp46-3: “H4” heavy chain variable region
(SEQ ID NO: 121)
QVQLVQSGAEVKKPGSSVKVSCKASGYTFSEYTMHWVRQAPGQGL
EWIGGISPNIGGTSYNQKFKGRATLTADKSTSTAYMELSSLRSED
TAVYYCARRGGSFDYWGQGTTVTVSS.
NKp46-3: “L1” light chain variable region
(SEQ ID NO: 122)
EIVMTQSPATLSVSPGERATLSCRASQSISDYLHWYQQKPGQAPRL
LIKYASQSISGIPARESGSGSGTDFTLTISSLEPEDFAVYYCQNG
HSFPLTFGQGTKLEIK.
NKp46-4: “H1” heavy chain variable region
(SEQ ID NO: 123)
QVQLVQSGAEVKKPGASVKVSCKASGYTFTSFTMHWVRQAPGQGL
EWIGYINPSSGYTEYNQKFKDRVTITADKSTSTAYMELSSLRSED
TAVYYCVRGSSRGFDYWGQGTLVTVSS.
NKp46-4: “H2” heavy chain variable region
(SEQ ID NO: 124)
QVQLVQSGAEVKKPGASVKVSCKASGYTFTSFTMHWVRQAPGQGL
EWIGYINPSSGYTEYNQKFKDRTTITADKSTSTAYMELSSLRSED
TAVYYCVRGSSRGFDYWGQGTLVTVSS.
NKp46-4: “H3” heavy chain variable region
(SEQ ID NO: 125)
QVQLVQSGAEVKKPGASVKVSCKASGYTFTSFTMHWVRQAPGQGL
EWIGYINPSSGYTEYNQKEKDRTTLTADKSTSTAYMELSSLRSED
TAVYYCVRGSSRGFDYWGQGTLVTVSS.
NKp46-4: “L2” light chain variable region
(SEQ ID NO: 126)
DIQMTQSPSSLSASVGDRVTITCRASENIYSNLAWFQQKPGKAPK
LLVYAATNLADGVPSRESGSGSGTDYTLTISSLQPEDFATYYCQH
FWGTPRTFGGGTKVEIK.
NKp46-9: “H1” heavy chain variable region
(SEQ ID NO: 127)
QVQLQESGPGLVKPSQTLSLTCTVSGGSISSDYAWNWIRQPPGKG
LEWIGYITYSGSTNYNPSLKSRVTISRDTSKNQFSLKLSSVTAAD
TAVYYCARCWDYALYAMDCWGQGTTVTVSS.
NKp46-9: “H2” heavy chain variable region
(SEQ ID NO: 128)
QVQLQESGPGLVKPSQTLSLTCTVSGYSISSDYAWNWIRQPPGKG
LEWIGYITYSGSTNYNPSLKSRVTISRDTSKNQFSLKLSSVTAAD
TAVYYCARCWDYALYAMDCWGQGTTVTVSS.
NKp46-9: “H3” heavy chain variable region
(SEQ ID NO: 129)
QVQLQESGPGLVKPSQTLSLTCTVSGYSISSDYAWNWIRQPPGKG
LEWMGYITYSGSTNYNPSLKSRITISRDTSKNQFSLKLSSVTAAD
TAVYYCARCWDYALYAMDCWGQGTTVTVSS.
NKp46-9: “L1” light chain variable region
(SEQ ID NO: 130)
DIQMTQSPSSLSASVGDRVTITCRTSENIYSYLAWCQQKPGKAPK
LLIYNAKTLAEGVPSRFSGSGSGTDFTLTISSLOPEDFATYYCQH
HYDTPLTFGQGTKLEIK.
NKp46-9: “L2” light chain variable region
(SEQ ID NO: 131)
DIQMTQSPSSLSASVGDRVTITCRTSENIYSYLAWCQQKPGKAPK
LLVYNAKTLAEGVPSRESGSGSGTDFTLTISSLOPEDFATYYCQH
HYDTPLTFGQGTKLEIK.

Examples of VH and VL combinations include:
(a) a VH comprising a CDR1, 2 and 3 of SEQ ID NO: 3 and a FR1, 2 and 3 of a human IGHV1-69 gene segment, and a VL comprising a CDR1, 2 and 3 of SEQ ID NO: 4 and a FR1, 2 and 3 of a human IGKV1-33 gene segment;
(b) a VH comprising a CDR1, 2 and 3 of SEQ ID NO: 5 and a FR1, 2 and 3 of a human IGHV4-30-4 gene segment, and a VL comprising a CDR1, 2 and 3 of SEQ ID NO: 6 and a FR1, 2 and 3 of a human IGKV1-39 gene segment;
(c) a VH comprising a CDR1, 2 and 3 of SEQ ID NO: 7 and a FR1, 2 and 3 of a human IGHV1-69 gene segment, and a VL comprising a CDR1, 2 and 3 of SEQ ID NO: 8 and a FR1, 2 and 3 of a human IGKV3-11 and/or IGKV3-15 gene segment;
(d) a VH comprising a CDR1, 2 and 3 of SEQ ID NO: 9 and a FR1, 2 and 3 of a human IGHV1-46 and/or a IGHV1-69 gene segment, and a VL comprising a CDR1, 2 and 3 of SEQ ID NO: 10 and a FR1, 2 and 3 of a human IGKV1-NL1 gene segment; or
(e) a VH comprising a CDR1, 2 and 3 of SEQ ID NO: 13 and a FR1, 2 and 3 of a human IGHV4-30-4 gene segment, and a VL comprising a CDR1, 2 and 3 of SEQ ID NO: 14 and a FR1, 2 and 3 of a human IGKV1-39 gene segment.

In another aspect, examples of humanized anti-NKp46 VH and VL combinations include:
(a) a VH comprising an amino acid sequence at least 70%, 80%, 90%, 95%, 98% or 100% identical to the amino acid sequence of the NKp46-1 H1 or H3 variable domain, and a VL comprising an amino acid sequence at least 70%, 80%, 90%, 95%, 98% or 100% identical to the amino acid sequence of the NKp46-1 L1 variable domain;
(b) a VH comprising an amino acid sequence at least 70%, 80%, 90%, 95%, 98% or 100% identical to the amino acid sequence of the NKp46-2 H1, H2 or H3 variable domain, and a VL comprising an amino acid sequence at least 70%, 80%, 90%, 95%, 98% or 100% identical to the amino acid sequence of the NKp46-2 L1 variable domain;
(c) a VH comprising an amino acid sequence at least 70%, 80%, 90%, 95%, 98% or 100% identical to the amino acid sequence of the NKp46-3 H1, H3 or H4 variable domain, and a VL comprising an amino acid sequence at least 70%, 80%, 90%, 95%, 98% or 100% identical to the amino acid sequence of the NKp46-3 L1 variable domain;
(d) a VH comprising an amino acid sequence at least 70%, 80%, 90%, 95%, 98% or 100% identical to the amino acid sequence of the NKp46-4 H1 variable domain, and a VL comprising an amino acid sequence at least 70%, 80%, 90%, 95%, 98% or 100% identical to the amino acid sequence of the NKp46-4 L2 variable domain;
(e) a VH comprising an amino acid sequence at least 70%, 80%, 90%, 95%, 98% or 100% identical to the amino acid sequence of the NKp46-9 H2 variable domain, and a VL comprising an amino acid sequence at least 70%, 80%, 90%, 95%, 98% or 100% identical to the amino acid sequence of the NKp46-9 L1 or L2 variable domain; or
(f) a VH comprising an amino acid sequence at least 70%, 80%, 90%, 95%, 98% or 100% identical to the amino acid sequence of the NKp46-9 H3 variable domain, and a VL comprising an amino acid sequence at least 70%, 80%, 90%, 95%, 98% or 100% identical to the amino acid sequence of the NKp46-9 L1 or L2 variable domain.

TABLE 2

| Antibody | VH (SEQ ID NO) | VL (SEQ ID NO) |
|---|---|---|
| NKp46-1 H1L1 | 112 | 114 |
| NKp46-1 H3L1 | 113 | 114 |
| NKp46-2 H1L1 | 115 | 118 |
| NKp46-2 H2L1 | 116 | 118 |
| NKp46-2 H3L1 | 117 | 118 |
| NKp46-3 H1L1 | 119 | 122 |
| NKp46-3 H3L1 | 120 | 122 |
| NKp46-3 H4L1 | 121 | 122 |
| NKp46-4 H1L2 | 123 | 126 |
| NKp46-4 H2L2 | 124 | 126 |
| NKp46-4 H3L2 | 125 | 126 |
| NKp46-9 H2L1 | 128 | 130 |
| NKp46-9 H2L2 | 128 | 131 |
| NKp46-9 H3L1 | 129 | 130 |
| NKp46-9 H3L2 | 129 | 131 |

### Activity Testing

A multispecific protein can be assessed for biological activity, e.g., antigen binding, ability to elicit proliferation of NK cells, ability to elicit target cell lysis by NK and/or elicit activation of NK cells, including any specific signaling activities elicited thereby, for example cytokine production or cell surface expression of markers of activation. In one embodiment, provided are methods of assessing the biological activity, e.g., antigen binding, ability to elicit target cell lysis and/or specific signaling activities elicited thereby, of a multispecific protein of the disclosure. It will be appreciated that when the specific contribution or activity of one of the components of the multispecific protein is to be assessed (e.g. an NKp46 binding ABD, antigen-of-interest binding ABD, an Fc domain, cytokine receptor ABD, etc.), the multispecific format can be produced in a suitable format which allows for assessment of the component (e.g. domain) of interest. The present disclosure also provides such methods, for use in testing, assessing, making and/or producing a multispecific protein. For example, where the contribution or activity of a cytokine is assessed, the multispecific protein can be produced as a protein having the cytokine and another protein in which the cytokine is modified to delete it or otherwise modulate its activity (e.g., wherein the two multispecific proteins otherwise have the same or comparable structure), and tested in an assay of interest. For example, where the contribution or activity of an anti-NKp46 ABD is assessed, the multispecific protein can be produced as a protein having the ABD and another protein in which the ABD is absent or is replaced by an ABD that does not bind NKp46 (e.g., an ABD that binds an antigen not present in the assay system), wherein the two multispecific proteins otherwise have the same or comparable structure, and the two multispecific proteins are tested in an assay of interest. In another example, where the contribution or activity of an anti-antigen of interest (e.g. tumor antigen) ABD is assessed, the multispecific protein can be produced as a protein having the ABD and another protein in which the ABD is absent or is replaced by an ABD that does not bind the tumor antigen or anti-antigen of interest (e.g., an ABD that binds an antigen not present in the assay system, an ABD that bind to a different tumor antigen), wherein the two multispecific proteins otherwise have the same or comparable structure, and the two multispecific proteins are tested in an assay of interest.

In one aspect of any embodiment described herein, the multispecific protein is capable of inducing activation of an NKp46-expressing cell (e.g. an NK cell, a reporter cell) when the protein is incubated in the presence of the NKp46-expressing cell (e.g. purified NK cells) and a target cell (e.g. tumor cell) that expresses the antigen of interest (e.g. tumor antigen).

In one aspect of any embodiment described herein, the multispecific protein is capable of inducing NKp46 signaling in an NKp46-expressing cell (e.g. an NK cell, a reporter cell) when the protein is incubated in the presence of an NKp46-expressing cell (e.g. purified NK cells) and a target cell that expresses the antigen of interest). In one aspect of any embodiment described herein, the multispecific protein is capable of inducing CD16A signaling in an CD16A and NKp46-expressing cell (e.g. an NK cell, a reporter cell) when the protein is incubated in the presence of a CD16A and NKp46-expressing cell (e.g. purified NK cells) and a target cell that expresses the antigen of interest).

Optionally, NK cell activation or signaling in characterized by the increased expression of a cell surface marker of activation, e.g. CD107, CD69, Sca-1 or Ly-6A/E, KLRG1, etc.

In one aspect of any embodiment described herein, the multispecific protein is capable of inducing an increase of CD137 present on the cell surface of an NKp46- and/or a CD16-expressing cell (e.g. an NK cell, a reporter cell) when the protein is incubated in the presence of the NKp46- and/or a CD16-expressing cell (e.g. purified NK cells), optionally in the absence of target cells.

In one aspect of any embodiment described herein, the multispecific protein is capable of activating or enhancing the proliferation of NK cells by at least 10-fold, at least 50-fold, or at least 100-fold compared to the same multispecific protein lacking the cytokine receptor ABD (e.g. the CD122 ABD). Optionally the multispecific protein displays an EC50 for activation or enhancing the proliferation of NK cells that is at least 10-fold, 50-fold or 100-fold lower than its EC50 for activation or enhancing the proliferation of CD25-expressing T cells.

In one aspect of any embodiment described herein, the multispecific protein is capable of activating or enhancing the proliferation of NK cells over CD25-expressing T cells, by at least 10-fold, at least 50-fold, or at least 100-fold. Optionally, the CD25 expressing T cells are CD4 T cells, optionally Treg cells, or CD8 T cells.

Activation or enhancement of proliferation via cytokine receptor in cells (e.g. NK cells, CD4 T cells, CD8 Tcells or Treg cells) by the cytokine receptor ABD-containing protein can be determined by measuring the expression of pSTAT or the cell proliferation markers (e.g. Ki67) in said cells following the treatment with the multispecific protein. Activation or enhancement of proliferation via the IL-2R pathway in cells (e.g. NK cells, CD4 T cells, CD8 Tcells or Treg cells) by the CD122 ABD-containing protein can be determined by measuring the expression of pSTAT5 or the cell proliferation marker Ki67 in said cells following the treatment with the multispecific protein. IL-2 and IL-15 lead to the phosphorylation of the STAT5 protein, which is involved in cell proliferation, survival, differentiation and apoptosis. Phosphorylated STAT5 (pSTAT5) translocates into the nucleus to regulate transcription of the target genes including the CD25. STAT5 is also required for NK cell survival and NK cells are tightly regulated by the JAK-STAT signaling pathway. In one aspect of any embodiment described herein, the multispecific protein is capable of inducing STAT5 signaling in an NKp46-expressing cell (e.g. an NK cell) when the protein is incubated in the presence of an NKp46-expressing cell (e.g. purified NK cells). In one aspect of any embodiment described herein, the multispecific protein is capable of causing an increase of expression of pSTAT5 in NK cells over CD25-expressing T cells, by at least 10-fold, at least 50-fold, or at least 100-fold. Optionally the multispecific protein displays an EC₅₀ for induction of expression of pSTAT5 in NK cells that is at least 10-fold, 50-fold or 100-fold lower than its EC50 for induction of expression of pSTAT5 in CD25-expressing T cells. Similarly, cytokine receptor signal transduction can also be assessed for other cytokine/cytokine receptor pairs, such as IL-15 (STAT5), IL-21 (STAT3), IL-27 (STAT1), IL-12 (STAT4), etc.

Activity can be measured for example by bringing NKp46-expressing cells (or CD25-expressing cells, depending on the assay) into contact with the multispecific polypeptide, optionally further in presence of target cells (e.g. tumor cells). In some embodiments, activity is measured for example by bringing target cells and NK cells (i.e. NKp46-expressing cells) into contact with one another, in presence of the multispecific polypeptide. The NKp46-expressing cells may be employed either as purified NK cells or NKp46-expressing cells, or as NKp46-expressing cells within a population of peripheral blood mononuclear cell (PBMC). The target cells can be cells expressing the antigen of interest, optionally tumor cells.

In one example, the multispecific protein can be assessed for the ability to cause a measurable increase in any property or activity known in the art as associated with NK cell activity, respectively, such as marker of cytotoxicity (CD107) or cytokine production (for example IFN-γ or TNF-α), increases in intracellular free calcium levels, the ability to lyse target cells, for example in a redirected killing assay, etc.

In the presence of target cells (target cells expressing the antigen of interest) and NK cells that express NKp46, the multispecific protein will be capable of causing an increase in a property or activity associated with NK cell activity (e.g. activation of NK cell cytotoxicity, CD107 expression, IFNγ production, killing of target cells) in vitro. For example, a multispecific protein according to the invention can be selected based on its ability to increase an NK cell activity by more than about 20%, preferably by least about 30%, at least about 40%, at least about 50%, or more compared to that achieved with the same effector: target cell ratio with the same NK cells and target cells that are not brought into contact with the multispecific protein, as measured by an assay that detects NK cell activity, e.g., an assay which detects the expression of an NK activation marker or which detects NK cell cytotoxicity, e.g., an assay that detects CD107 or CD69 expression, IFNγ production, or a classical in vitro chromium release test of cytotoxicity. Examples of protocols for detecting NK cell activation and cytotoxicity assays are described in the Examples herein, as well as for example, in Pessino et al, J. Exp. Med, 1998, 188 (5): 953-960; Sivori et al, Eur J Immunol, 1999. 29:1656-1666; Brando et al, (2005) J. Leukoc. Biol. 78:359-371; El-Sherbiny et al, (2007) Cancer Research 67(18):8444-9; and Nolte-'t Hoen et al, (2007) Blood 109:670-673). In a classical in vitro chromium release test of cytotoxicity, the target cells are labeled with ⁵¹Cr prior to addition of NK cells, and then the killing is estimated as proportional to the release of ⁵¹Cr from the cells to the medium, as a result of killing. Optionally, a multispecific protein according to the invention can be selected for or characterized by its ability to have greater ability to induce NK cell activity towards target cells, i.e., lysis of target cells compared to a conventional human IgG1 antibody that binds to the same antigen of interest, as measured by an assay of NK cell activity (e.g. an assay that detects NK cell-mediated lysis of target cells that express the antigen of interest).

As shown herein, a multispecific protein, the different ABDs contribute to the overall activity of the multispecific protein that ultimately manifests itself in potent anti-tumor activity in vivo. Testing methods exemplified herein allow the in vitro assessment of the activities of the different individual ABDs of the multispecific protein by making variants of the multispecific protein that lack a particular ABD and/or using cells that lack receptors for the particular ABD. As shown herein, a multispecific protein according to the disclosure, when it does not comprise the cytokine receptor ABD (e.g. the CD122 ABD) and when it possesses an Fc domain that does not bind CD16, does not substantially induce NKp46 signaling (and/or NK activation that results therefrom) of NK cells when the protein is not bound to the antigen of interest on target cells (e.g. in the absence of the target cells). Thus, the monovalent NKp46 binding component of the multispecific protein does not itself cause NKp46 signaling. Accordingly, in the case of multispecific proteins possessing an Fc domain that binds CD16, such multispecific protein can be produced in a configuration where the cytokine receptor ABD (e.g. CD122 ABD) is inactivated (e.g. modified, masked or deleted, thereby eliminating its ability to bind IL-2Rs) and the protein can be assessed for its ability to elicit NKp46 signaling or NKp46-mediated NK cell activation by testing the effect of this multispecific protein on NKp46 expression, by CD16-negative NK cells. The multispecific protein can optionally be characterized as not substantially causing (or increasing) NKp46 signaling by an NKp46-expressing, CD16-negative cell (e.g. a NKp46⁺CD16⁻ NK cell, a reporter cell) when the multispecific protein is incubated with such NKp46-expressing, CD16-negative cells (e.g., purified NK cells or purified reporter cells) in the absence of target cells.

In one aspect of any embodiment herein, a multispecific protein can for example be characterized by:
(a) being capable of inducing cytokine receptor (e.g. CD122) signaling (e.g., as determined by assessing STAT signaling, for example assessing STAT phosphoylation) in an NKp46-expressing cell (e.g. an NK cell) when the multispecific protein is incubated in the presence of an NKp46-expressing cell (e.g. purified NK cells);
(b) being capable of inducing NK cells that express NKp46 (and optionally further CD16) to lyse target cells, when incubated in the presence of the NK cells and target cells; and
(c) lack of NK cell activation or cytotoxicity and/or lack of agonist activity at NKp46 when incubated with NK cells (optionally CD16-negative NK cells, NKp46-expressing NK cells that do not express CD16), in the absence of target cells, optionally wherein the NK cells are purified NK cells, when the multispecific protein is modified to lack the cytokine receptor ABD (e.g. CD122 ABD) or comprises an inactivated cytokine receptor ABD.

### Uses of Compounds

In one aspect, provided is the use of any of the multispecific proteins and/or cells which express the proteins (or a polypeptide chain thereof) for the manufacture of a pharmaceutical preparation for the treatment, prevention or diagnosis of a disease in a mammal in need thereof. Provided also are the use any of the compounds defined above as a medicament or an active component or active substance in a medicament. In a further aspect the invention provides methods for preparing a pharmaceutical composition containing a compound as defined herein, to provide a solid or a liquid formulation for administration (e.g., by subcutaneous or intravenous injection). Such a method or process at least comprises the step of mixing the compound with a pharmaceutically acceptable carrier.

In one aspect, provided is a method to treat, prevent or more generally affect a predefined condition in an individual or to detect a certain condition by using or administering a multispecific protein or antibody described herein, or a (pharmaceutical) composition comprising same.

For example, in one aspect, the invention provides a method of restoring or potentiating the activity and/or proliferation of NKp46-expressing cells, particularly NKp46⁺ NK cells (e.g. NKp46⁺CD16⁺ NK cells, NKp46⁺CD16⁻ NK cells) in a patient in need thereof (e.g. a patient having a cancer, or a viral or bacterial infection), comprising the step of administering a multispecific protein described herein to said patient. In one aspect, the invention provides a method of selectively restoring or potentiating the activity and/or proliferation of NK cells of over CD25-expressing lymphocytes, e.g. CD4 T cells, CD8 T cells, Treg cells. In one embodiment, the method is directed at increasing the activity of NKp46⁺ lymphocytes (e.g. NKp46⁺CD16⁺ NK cells, NKp46⁺CD16⁻ NK cells) in patients having a disease in which increased lymphocyte (e.g. NK cell) activity is beneficial or which is caused or characterized by insufficient NK cell activity, such as a cancer, or a viral or microbial/bacterial infection.

In one aspect, the invention provides a method of restoring or potentiating the activity and/or proliferation of tumor-infiltrating NK cells or intra-tumoral NKp46-expressing cells, particularly NKp46⁺ NK cells (e.g. NKp46⁺CD16⁺ NK cells, NKp46⁺CD16⁻ NK cells) in a patient in need thereof (e.g. a patient having a solid tumor), comprising the step of administering a multispecific protein described herein to said patient.

In one aspect, the invention provides a method of increasing the number of tumor-infiltrating NK cells or intra-tumoral NKp46-expressing cells, particularly activated NKp46-expressing cells, particularly NKp46⁺ NK cells (e.g. NKp46⁺CD16⁺ NK cells, NKp46⁺CD16⁻ NK cells) in a patient in need thereof (e.g. a patient having a solid tumor), comprising the step of administering a multispecific protein described herein to said patient.

In another aspect, the invention provides a method of restoring or potentiating the activity and/or proliferation of NKp46⁺ NK cells (e.g. NKp46⁺CD16⁺ NK cells, NKp46⁺CD16⁻ NK cells) in a patient in need thereof (e.g. a patient having a cancer, or a viral, parasite or bacterial infection), comprising the step of contacting cells derived from the patient, e.g., immune cells and optionally target cells expressing an antigen of interest with a multispecific protein according to the invention and reinfusing the multispecific protein treated cells into the patient. In one embodiment, this method is directed at increasing the activity of NKp46+ lymphocytes (e.g. NKp46⁺CD16⁺ NK cells) in patients having a disease in which increased lymphocyte (e.g. NK cell) activity is beneficial or which is caused or characterized by insufficient NK cell activity, such as a cancer, or a viral or microbial, e.g., bacterial or parasite infection.

In another embodiment the subject multispecific proteins may be used or administered in combination with immune cells, particularly NK cells, derived from a patient who is to be treated or from a different donor, and these NK cells administered to a patient in need thereof such as a patient having a disease in which increased lymphocyte (e.g. NK cell) activity is beneficial or which is caused or characterized by insufficient NK cell activity, such as a cancer, or a viral or microbial, e.g., bacterial or parasite infection. As NK cells (unlike CAR-T cells) do not express TCRs, these NK cells, even those derived from different donors will not induce a GVHD reaction (see e.g., Glienke et al., “Advantages and applications of CAR-expressing natural killer cells”, Front. Pharmacol. 6, Art. 21:1-6 (2015); Hermanson and Kaufman, Front. Immunol. 6, Art. 195:1-6 (2015)).

In one embodiment, the multispecific protein disclosed herein that mediates NK cell activation, proliferation, tumor infiltration and/or target cell lysis via multiple activating receptors of effector cells, including NKp46, CD16 and CD122, can be used advantageously for treatment of individuals whose effector cells or tumor-infiltrating effector cells (e.g. NKp46⁺ NK cells) cells are hypoactive, exhausted or suppressed, for example a patient who has a significant population of effector cells characterized by the expression and/or upregulation of one or multiple inhibitory receptors (e.g. TIM-3, PD1, CD96, TIGIT, etc.), or the downregulation or low level of expression of CD16 (e.g., presence of elevated proportion of NKp46⁺CD16⁻ NK cells).

The multispecific polypeptides described herein can be used to prevent or treat disorders that can be treated with antibodies, such as cancers, solid and non-solid tumors, hematological malignancies, infections such as viral infections, and inflammatory or autoimmune disorders.

In one embodiment, the antigen of interest (the non-NKp46 antigen) is an antigen expressed on the surface of a malignant cell of a type of cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sézary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukemia (T-Lbly/T-ALL).

In one embodiment, a multispecific protein is used to prevent or treat a cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma. Other exemplary disorders that can be treated according to the invention include hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sézary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukaemia (T-Lbly/T-ALL).

In one example, the tumor antigen is an antigen expressed on the surface of a lymphoma cell or a leukemia cell, and the multispecific protein is administered to, and/or used for the treatment of, an individual having a lymphoma or a leukemia.

In one embodiment, the inventive multispecific polypeptides described herein can be used to prevent or treat a cancer characterized by tumor cells that express the antigen of interest (e.g. tumor antigen) to which the multispecific protein of the disclosure specifically binds.

In one aspect, the methods of treatment comprise administering to an individual a multispecific protein described herein in a therapeutically effective amount, e.g., for the treatment of a disease as disclosed herein, for example any of the cancers identified above. A therapeutically effective amount may be any amount that has a therapeutic effect in a patient having a disease or disorder (or promotes, enhances, and/or induces such an effect in at least a substantial proportion of patients with the disease or disorder and substantially similar characteristics as the patient).

The multispecific protein may be used with our without a prior step of detecting the expression of the antigen of interest (e.g. tumor antigen) on target cells in a biological sample obtained from an individual (e.g. a biological sample comprising cancer cells, cancer tissue or cancer-adjacent tissue). In another embodiment, the disclosure provides a method for the treatment or prevention of a cancer in an individual in need thereof, the method comprising:
a) detecting cells (e.g. tumor cells) in a sample from the individual that express an antigen of interest (e.g. the antigen of interest to which the multispecific protein specifically binds via its antigen of interest ABD), and
b) upon a determination that cells which express an antigen of interest are comprised in the sample, optionally at a level corresponding at least to a reference level (e.g. corresponding to an individual deriving substantial benefit from a multispecific protein, or optionally at a level that is increased compared to a reference level (e.g. corresponding to a healthy individual or an individual not deriving substantial benefit from a protein described herein), administering to the individual a multispecific protein of the disclosure that binds to an antigen of interest, to NKp46, to cytokine receptor (e.g. CD122), and optionally to CD16A (e.g., via its Fc domain).

In some embodiments, the multispecific proteins are used to treat a tumor characterized by low levels of surface expression of the antigen of interest. Accordingly, in, the tumor or cancer can be characterized by cells expressing a low level of tumor antigen. Optionally, the level of the tumor antigen is less than 100,000 tumor antigen copies per cancer cell. In some aspects, the level of the tumor antigen is less than 90,000, less than 75,000, less than 50,000, or less than 40,000 tumor antigen copies per cancer cell. The uses optionally further comprise detecting the level of tumor antigen of one or more cancer cells of the subject.

The multispecific protein may be used with our without a prior step of detecting or characterizing NK cells from an individual to be treated. Optionally, in one embodiment, the invention provides a method for the treatment or prevention of a cancer in an individual in need thereof, the method comprising:
a) detecting NK cells (e.g. tumor-infiltrating NK cells) in a tumor sample from an individual (or within the tumor and/or within adjacent tissue), and
b) upon a determination that the tumor or tumor sample is characterized by a low number or activity of NK cells, optionally at a level or number that is decreased compared to a reference level (e.g. at a level corresponding to an individual deriving no, low or insufficient benefit from a conventional IgG antibody therapy such a conventional IgG1 antibody that binds to the same cancer antigen), administering to the individual a multispecific protein that binds to a cancer antigen, to NKp46 (e.g., monovalently), to cytokine receptor (e.g., CD122) and optionally to CD16A.

In some embodiments, an individual has a tumor characterized by a CD16 (e.g. CD16A) deficient tumor microenvironment. Optionally, the methods of treatment using a multispecific protein comprise a step of detecting the expression level of CD16 in a sample (e.g. a tumor sample) from the individual. Detecting the CD16 optionally comprises detecting the level of CD16A or CD16B. In some aspects, the CD16 deficient microenvironment is assessed in a patient having undergone a hematopoietic stem cell transplantation. Optionally, the CD16 deficient microenvironment comprises a population of infiltrating NK cells, and the infiltrating NK cells have less than 50% expression of CD16 as compared to a control NK cell. In some aspects, the infiltrating NK cells have less than 30%, less than 20%, or less than 10% expression of CD16 as compared to a control NK cell. Optionally, the CD16 deficient microenvironment comprises a population of infiltrating NK cells, and at least 10% of the infiltrating NK cells have reduced expression of CD16 as compared to a control NK cell. In some aspects, at least 20%, at least 30%, or at least 40% of the infiltrating NK cells have reduced expression of CD16 as compared to a control NK cell.

Optionally, in one embodiment, provided is a method for the treatment or prevention of a cancer in an individual in need thereof, the method comprising:
a) detecting CD16 expression in cells (e.g. in tumor-infiltrating NK cells) from a tumor or tumor sample (e.g., tumor and/or within adjacent tissue) from an individual, and
b) upon a determination that the tumor or tumor sample is characterized by a CD16 deficient microenvironment, administering to the individual a multispecific protein that binds to a cancer antigen, to NKp46, and to the cytokine receptor (e.g. CD122) (and optionally further to CD16A).

Optionally, in one embodiment, provided is a method for the treatment or prevention of a cancer in an individual in need thereof, the method comprising:
a) detecting CD16 expression at the surface of NK cells (e.g. tumor-infiltrating NK cells) in a tumor sample from an individual (or within the tumor and/or within adjacent tissue), and
b) upon a determination that the tumor or tumor sample is characterized by an elevated proportion of CD16⁻ NK cells, optionally at a level or number that is increased compared to a reference level, administering to the individual a multispecific protein that binds to a cancer antigen, to NKp46, and to the cytokine receptor (e.g. CD122) (and optionally further to CD16A).

In one embodiment, the disclosure provides a method for the treatment or prevention of a disease (e.g. a cancer) in an individual in need thereof, the method comprising:
a) detecting cell surface expression of one or a plurality inhibitory receptors on immune effector cells (e.g. NK cells, T cells) or a ligand(s) thereof on a tumor cell in a sample from the individual (e.g. in circulation or in the tumor environment), and
b) upon a determination of cell surface expression of said one or a plurality inhibitory receptors on immune effector cells or ligand(s) thereof on a tumor cell, optionally at a level that is increased compared to a reference level (e.g. at a level that is increased compared to a healthy individual, an individual not suffering from immune exhaustion or suppression, or an individual not deriving substantial benefit from a protein described herein), administering to the individual a multispecific protein (e.g. a multispecific protein) that binds to an antigen of interest (e.g. a cancer antigen), to NKp46 (e.g., monovalently), and to the cytokine receptor (e.g. CD122) (and optionally further to CD16A).

In some embodiments, the multispecific proteins are used to treat an individual having a gastric cancer or a prostate cancer. Decreased cell surface expression of NKG2D on immune effector cells has been observed in gastric cancer and prostate cancer.

In some embodiments, an individual has NK cells and/or T cells characterized by decreased expression of NKG2D, e.g. decreased cell surface expression. The level of expression can be for example compared to a reference value, for example a reference value corresponding to NKG2D levels observed on NK and/or T cells in healthy individuals. In some embodiments, an individual has NK and/or T cell characterized by decreased expression of NKG2D on NK and/or T cells in the tumor microenvironment. In some embodiments, an individual has NK and/or T cell characterized by presence (e.g. at increased levels) of soluble ligands of NKG2D in the tumor microenvironment, for example soluble MICA, MICB or ULBP proteins.

In one embodiment, the disclosure provides a method for the treatment or prevention of a disease (e.g. a cancer) in an individual in need thereof, the method comprising:
a) detecting cell surface expression of NKG2D polypeptides on immune effector cells (e.g. NK cells, T cells) in a sample from the individual (e.g. in circulation or in the tumor environment), and
b) upon a determination of decreased cell surface expression of one or a plurality inhibitory receptors on immune effector cells, optionally at a level that is decreased compared to a reference level (e.g. at a level that is increased compared to a healthy individual, an individual not suffering from immune exhaustion or suppression, or an individual not deriving substantial benefit from a protein described herein), administering to the individual a multispecific protein (e.g. a multispecific protein) that binds to an antigen of interest (e.g. a cancer antigen), to NKp46 (e.g., monovalently), and to the cytokine receptor (e.g. CD122) (and optionally further to CD16A).

In one embodiment, a multispecific protein may be used as a monotherapy (without other therapeutic agents), or in combined treatments with one or more other therapeutic agents. In one embodiment, a multispecific protein is administered in the absence of combined treatment with an agent selected from IL-2, IL-15, IL-21, IL-7, IL-27, IL-12, IL-18, IFN-α and IFN-β polypeptide.

The multispecific proteins can also be included in kits. The kits may optionally further contain any number of polypeptides and/or other compounds, e.g., 1, 2, 3, 4, or any other number of multispecific proteins and/or other compounds. It will be appreciated that this description of the contents of the kits is not limiting in any way. For example, the kit may contain other types of therapeutic compounds. Optionally, the kits also include instructions for using the polypeptides, e.g., detailing the herein-described methods such as in the detection or treatment of specific disease conditions.

Also provided are pharmaceutical compositions comprising the subject multispecific proteins and optionally other compounds as defined above. A multispecific protein and optionally another compound may be administered in purified form together with a pharmaceutical carrier as a pharmaceutical composition. The form depends on the intended mode of administration and therapeutic or diagnostic application. The pharmaceutical carrier can be any compatible, nontoxic substance suitable to deliver the compounds to the patient. Pharmaceutically acceptable carriers are well known in the art and include, for example, aqueous solutions such as (sterile) water or physiologically buffered saline or other solvents or vehicles such as glycols, glycerol, oils such as olive oil or injectable organic esters, alcohol, fats, waxes, and inert solids. A pharmaceutically acceptable carrier may further contain physiologically acceptable compounds that act for example to stabilize or to increase the absorption of the compounds Such physiologically acceptable compounds include, for example, carbohydrates, such as glucose, sucrose or dextrans, antioxidants, such as ascorbic acid or glutathione, chelating agents, low molecular weight proteins or other stabilizers or excipients One skilled in the art would know that the choice of a pharmaceutically acceptable carrier, including a physiologically acceptable compound, depends, for example, on the route of administration of the composition Pharmaceutically acceptable adjuvants, buffering agents, dispersing agents, and the like, may also be incorporated into the pharmaceutical compositions. Non-limiting examples of such adjuvants include by way of example inorganic and organic adjuvants such as alum, aluminum phosphate and aluminum hydroxide, squalene, liposomes, lipopolysaccharides, double stranded (ds) RNAs, single stranded(s-s) DNAs, and TLR agonists such as unmethylated CpG's.

Multispecific proteins according to the invention can be administered parenterally. Preparations of the compounds for parenteral administration must be sterile. Sterilization is readily accomplished by filtration through sterile filtration membranes, optionally prior to or following lyophilization and reconstitution. The parenteral route for administration of compounds is in accord with known methods, e.g. injection or infusion by intravenous, intraperitoneal, intramuscular, intraarterial, or intralesional routes. The compounds may be administered continuously by infusion or by bolus injection. A typical composition for intravenous infusion could be made up to contain 100 to 500 ml of sterile 0.9% NaCl or 5% glucose optionally supplemented with a 20% albumin solution and 1 mg to 10 g of the compound, depending on the particular type of compound and its required dosing regimen. Methods for preparing parenterally administrable compositions are well known in the art.

### EXAMPLES

### Preparation of Multispecific Proteins

The amino acid sequences of the three chains of the GA101-T53A-NKp46-IL2v protein are shown in SEQ ID NOS: 175, 176 and 177. Variants of GA101-T53A-NKp46-IL2v were produced having the three polypeptide chains of SEQ ID NOS: 178, 179 and 180 and SEQ ID NOS: 181, 182 and 183, respectively. FIG. 2A shows the topology of the exemplary “T53A” format multispecific proteins used in the Examples. This format has one scFv and one Fab structure located topologically N-terminal, and a cytokine topologically C-terminal, with the dimeric Fc domain interposed between the scFv and Fab on the N-terminal side and the cytokine on the C-terminal side. In the protein of chains of SEQ ID NOS: 175, 176 and 177, the NKp46 binding domain has an scFv structure and the tumor antigen (TA or TAg) binding domain has a Fab structure comprising the VH-VL pair of the anti-CD20 antibody GA101. The domain structure of the exemplary “T53A” format protein used in the Examples is shown in FIG. 2B. FIG. 2B shows domain linkers (such as hinge and glycine-serine linkers) of different lengths, and interchain disulfide bridges. The Fc domain has Fc gamma receptor binding site amino acid sequences of wild-type human IgG1 and thus retains binding to CD16A. The Fc domains further contained “knob-into-holes” mutations to favor heterodimerization. The K&N (knobs into holes) mutations were: “Hole” (Y350C, T367S, L369A and Y408V) on the Fab-bearing fragment and “Knob” (S355C and T367W) on the ScFv-bearing fragment.

Additionally, one of the Fc monomers that make up the dimeric domain (the monomer indicated with the black circle) also contained mutations in the CH3 domain (H435R and Y436F, according to EU numbering, disclosed in Jendeberg et al. (1997) Journal of Immunological Methods 201: 25-34) to reduce binding to Protein A; these mutations which are optional do not affect the activity of the protein but can potentially improve efficiency of purification by permitting the elimination of homodimers.

The sequences encoding each polypeptide chain for each multispecific antigen-binding protein were inserted into the pTT-5 vector between the HindIII and BamHI restriction sites. The three vectors (prepared as endotoxin-free midipreps or maxipreps) were used to cotransfect EXPI-293F cells (Life Technologies) in the presence of PEI (37° C., 5% CO₂, 150 rpm). The cells were used to seed culture flasks at a density of 1×10⁶ cells per ml (EXP1293 medium, Gibco). Valproic Acid (final concentration 0.5 mM), glucose (4 g/L) and tryptone N1 (0.5%) were added. The supernatant was harvested after six days after and passed through a Stericup filter with 0.22 μm pores.

The multispecific antigen-binding proteins were purified from the supernatant following harvesting using rProtein A Sepharose Fast Flow (GE Healthcare, reference 17-1279-03). Size Exclusion Chromatography (SEC) purifications were then performed and the proteins eluted at the expected size were finally filtered on a 0.22 μm device.

The amino acid sequence of the polypeptide chains of the multispecific proteins produced are shown below in Table 3.

TABLE 3

| Sample | Frag M (chain 2) | Frag H (chain 1) | Frag L (chain 3) |
|---|---|---|---|
| GA101- | QVQLVQSGAEVKKPGSSVK | QVQLVQSGAEVKKPGSSVKVSCKASGYT | DIVMTQTPLSLPVTP |
| T53A- | VSCKASGYAFSYSWINWVR | FSDYVINWVRQAPGQGLEWMGEIYPGSG | GEPASISCRSSKSLL |
| NKp46- | QAPGQGLEWMGRIFPGDGD | TNYYNEKFKAKATITADKSTSTAYMELS | HSNGITYLYWYLQKP |
| 1-IL2v | TDYNGKFKGRVTITADKST | SLRSEDTAVYYCARRGRYGLYAMDYWGQ | GQSPQLLIYQMSNLV |
| 10aa | STAYMELSSLRSEDTAVYY | GTTVTVSSVEGGSGGSGGSGGSGGVDDI | SGVPDRESGSGSGTD |
| linker | CARNVFDGYWLVYWGQGTL | QMTQSPSSLSASVGDRVTITCRASQDIS | FTLKISRVEAEDVGV |
| | VTVSSASTKGPSVFPLAPS | NYLNWYQQKPGKAPKLLIYYTSRLHSGV | YYCAQNLELPYTFGG |
| | SKSTSGGTAALGCLVKDYF | PSRFSGSGSGTDFTFTISSLQPEDIATY | GTKVEIKRTVAAPSV |
| | PEPVTVSWNSGALTSGVHT | FCQQGNTRPWTFGGGTKVEIKTHTCPPC | FIFPPSDEQLKSGTA |
| | FPAVLQSSGLYSLSSVVTV | PAPELLGGPSVFLFPPKPKDTLMISRTP | SVVCLLNNFYPREAK |
| | PSSSLGTQTYICNVNHKPS | EVTCVVVDVSHEDPEVKFNWYVDGVEVH | VQWKVDNALQSGNSQ |
| | NTKVDKRVEPKSCDKTHTC | NAKTKPREEQYNSTYRVVSVLTVLHQDW | ESVTEQDSKDSTYSL |
| | PPCPAPELLGGPSVFLFPP | LNGKEYKCKVSNKALPAPIEKTISKAKG | SSTLTLSKADYEKHK |
| | KPKDTLMISRTPEVTCVVV | QPREPQVYTLPPCREEMTKNQVSLWCLV | VYACEVTHQGLSSPV |
| | DVSHEDPEVKFNWYVDGVE | KGFYPSDIAVEWESNGQPENNYKTTPPV | TKSENRGEC(SEQ |
| | VHNAKTKPREEQYNSTYRV | LDSDGSFFLYSKLTVDKSRWQQGNVFSC | ID NO: 177) |
| | VSVLTVLHQDWLNGKEYKC | SVMHEALHNHYTQKSLSLSPGGSSSSGS | |
| | KVSNKALPAPIEKTISKAK | SSSAPASSSTKKTQLQLEHLLLDLQMIL | |
| | GQPREPQVCTLPPSREEMT | NGINNYKNPKLTRMLTAKFAMPKKATEL | |
| | KNQVSLSCAVKGFYPSDIA | KHLQCLEEELKPLEEVLNGAQSKNFHLR | |
| | VEWESNGQPENNYKTTPPV | PRDLISNINVIVLELKGSETTFMCEYAD | |
| | LDSDGSFFLVSKLTVDKSR | ETATIVEFLNRWITFAQSIISTLT | |
| | WQQGNVFSCSVMHEALHNR | (SEQ ID NO: 176) | |
| | FTQKSLSLSPGK(SEQ ID | | |
| | NO: 175) | | |
| GA101- | QVQLVQSGAEVKKPGSSVK | QVQLVQSGAEVKKPGSSVKVSCKASGYT | DIVMTQTPLSLPVTP |
| T53A- | VSCKASGYAFSYSWINWVR | FSDYVINWVRQAPGQGLEWMGEIYPGSG | GEPASISCRSSKSLL |
| NKp46- | QAPGQGLEWMGRIFPGDGD | TNYYNEKFKAKATITADKSTSTAYMELS | HSNGITYLYWYLQKP |
| 1-IL2v- | TDYNGKFKGRVTITADKST | SLRSEDTAVYYCARRGRYGLYAMDYWGQ | GQSPQLLIYQMSNLV |
| short | STAYMELSSLRSEDTAVYY | GTTVTVSSVEGGSGGSGGSGGSGGVDDI | SGVPDRFSGSGSGTD |
| linker | CARNVFDGYWLVYWGQGTL | QMTQSPSSLSASVGDRVTITCRASQDIS | FTLKISRVEAEDVGV |
| | VTVSSASTKGPSVFPLAPS | NYLNWYQQKPGKAPKLLIYYTSRLHSGV | YYCAQNLELPYTFGG |
| | SKSTSGGTAALGCLVKDYF | PSRFSGSGSGTDFTFTISSLQPEDIATY | GTKVEIKRTVAAPSV |
| | PEPVTVSWNSGALTSGVHT | FCQQGNTRPWTFGGGTKVEIKTHTCPPC | FIFPPSDEQLKSGTA |
| | FPAVLQSSGLYSLSSVVTV | PAPELLGGPSVFLFPPKPKDTLMISRTP | SVVCLLNNFYPREAK |
| | PSSSLGTQTYICNVNHKPS | EVTCVVVDVSHEDPEVKFNWYVDGVEVH | VQWKVDNALQSGNSQ |
| | NTKVDKRVEPKSCDKTHTC | NAKTKPREEQYNSTYRVVSVLTVLHQDW | ESVTEQDSKDSTYSL |
| | PPCPAPELLGGPSVFLFPP | LNGKEYKCKVSNKALPAPIEKTISKAKG | SSTLTLSKADYEKHK |
| | KPKDTLMISRTPEVTCVVV | QPREPQVYTLPPCREEMTKNQVSLWCLV | VYACEVTHQGLSSPV |
| | DVSHEDPEVKFNWYVDGVE | KGFYPSDIAVEWESNGQPENNYKTTPPV | TKSENRGEC(SEQ |
| | VHNAKTKPREEQYNSTYRV | LDSDGSFFLYSKLTVDKSRWQQGNVESC | ID NO: 180) |
| | VSVLTVLHQDWLNGKEYKC | SVMHEALHNHYTQKSLSLSPGGSSSSAP | |
| | KVSNKALPAPIEKTISKAK | ASSSTKKTQLQLEHLLLDLQMILNGINN | |
| | GQPREPQVCTLPPSREEMT | YKNPKLTRMLTAKFAMPKKATELKHLQC | |
| | KNQVSLSCAVKGFYPSDIA | LEEELKPLEEVLNGAQSKNFHLRPRDLI | |
| | VEWESNGQPENNYKTTPPV | SNINVIVLELKGSETTFMCEYADETATI | |
| | LDSDGSFFLVSKLTVDKSR | VEFLNRWITFAQSIISTLT(SEQ ID | |
| | WQQGNVFSCSVMHEALHNR | NO: 179) | |
| | FTQKSLSLSPGK(SEQ ID | | |
| | NO: 178) | | |
| GA 101- | QVQLVQSGAEVKKPGSSVK | QVQLVQSGAEVKKPGSSVKVSCKASGYT | DIVMTQTPLSLPVTP |
| T53A- | VSCKASGYAFSYSWINWVR | FSDYVINWVRQAPGQGLEWMGEIYPGSG | GEPASISCRSSKSLL |
| NKp46- | QAPGQGLEWMGRIFPGDGD | TNYYNEKFKAKATITADKSTSTAYMELS | HSNGITYLYWYLQKP |
| 1-IL2v- | TDYNGKFKGRVTITADKST | SLRSEDTAVYYCARRGRYGLYAMDYWGQ | GQSPQLLIYQMSNLV |
| long | STAYMELSSLRSEDTAVYY | GTTVTVSSVEGGSGGSGGSGGSGGVDDI | SGVPDRFSGSGSGTD |
| linker | CARNVFDGYWLVYWGQGTL | QMTQSPSSLSASVGDRVTITCRASQDIS | FTLKISRVEAEDVGV |
| | VTVSSASTKGPSVFPLAPS | NYLNWYQQKPGKAPKLLIYYTSRLHSGV | YYCAQNLELPYTFGG |
| | SKSTSGGTAALGCLVKDYF | PSRFSGSGSGTDFTFTISSLQPEDIATY | GTKVEIKRTVAAPSV |
| | PEPVTVSWNSGALTSGVHT | FCQQGNTRPWTFGGGTKVEIKTHTCPPC | FIFPPSDEQLKSGTA |
| | FPAVLQSSGLYSLSSVVTV | PAPELLGGPSVFLFPPKPKDTLMISRTP | SVVCLLNNFYPREAK |
| | PSSSLGTQTYICNVNHKPS | EVTCVVVDVSHEDPEVKFNWYVDGVEVH | VQWKVDNALQSGNSQ |
| | NTKVDKRVEPKSCDKTHTC | NAKTKPREEQYNSTYRVVSVLTVLHQDW | ESVTEQDSKDSTYSL |
| | PPCPAPELLGGPSVFLFPP | LNGKEYKCKVSNKALPAPIEKTISKAKG | SSTLTLSKADYEKHK |
| | KPKDTLMISRTPEVTCVVV | QPREPQVYTLPPCREEMTKNQVSLWCLV | VYACEVTHQGLSSPV |
| | DVSHEDPEVKENWYVDGVE | KGFYPSDIAVEWESNGQPENNYKTTPPV | TKSFNRGEC(SEQ |
| | VHNAKTKPREEQYNSTYRV | LDSDGSFFLYSKLTVDKSRWQQGNVFSC | ID NO: 183) |
| | VSVLTVLHQDWLNGKEYKC | SVMHEALHNHYTQKSLSLSPGSSSSGSS | |
| | KVSNKALPAPIEKTISKAK | SSGSSSSAPASSSTKKTQLQLEHLLLDL | |
| | GQPREPQVCTLPPSREEMT | QMILNGINNYKNPKLTRMLTAKFAMPKK | |
| | KNQVSLSCAVKGFYPSDIA | ATELKHLQCLEEELKPLEEVLNGAQSKN | |
| | VEWESNGQPENNYKTTPPV | FHLRPRDLISNINVIVLELKGSETTEMC | |
| | LDSDGSFFLVSKLTVDKSR | EYADETATIVEFLNRWITFAQSIISTLT | |
| | WQQGNVFSCSVMHEALHNR | (SEQ ID NO: 182) | |
| | FTQKSLSLSPGK(SEQ ID | | |
| | NO: 181) | | |

### Example 1: IL2v Limits IL2R Activation on Treg

A heterotrimeric Fc-domain-containing protein containing one C-terminal moiety of mutant IL-2 was assessed for its ability to activate Treg cells. The protein incorporates a variant IL-2 polypeptide (IL-2v) in which a human IL-2 polypeptide is modified by introducing the mutations T3A, C125A, F42A, Y45A and L72G, conferring decreased binding affinity for 0025 compared to wild-type human IL-2.

The heterotrimeric had an IL2v fused to the C-terminus of one of the chains of an isotype control (IC) Fab, which in turn was fused to the C-terminus of an Fc domain mutated to substantially eliminate CD16A binding, in turn fused to another IC Fab. The IC Fabs have a VHNVL pair which does not bind to any protein in the test system. This heterotrimeric protein (IL2v immunoconjugate) was compared to an identical heterotrimeric protein in which IL2v was replaced by a wild-type human IL-2 polypeptide (IL2pWT immunoconjugate), and to recombinantly produced full-length wild-type IL-2 (rec huIL-2).

Briefly, 1M/well of purified PBMC were seeded in 96-well plate and treated with increasing doses of recombinant huIL-2, IC-T6-IC-IL2 (IL2pWT immunoconjugate) or IC-T6-IC-IL2v (IL2v immunoconjugate) (dose from 133 nM to 0,0000013 nM) for 20 min at 37° C., 5.5% CO2 in incubator. STAT5 phosphorylation was then analysed by flow cytometry on Tregs (gated on CD3+CD4+CD25+FoxP3+).

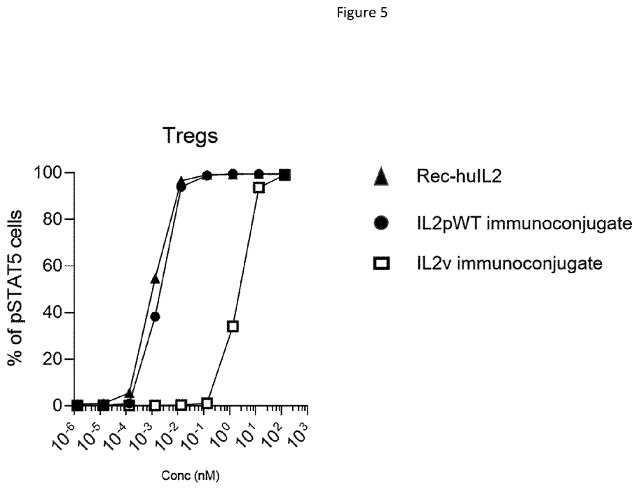

Results are shown in FIG. 5. The IL2v immunoconjugate resulted in an approximately 3-log decrease in percent of pSTAT5+ cells among the Treg, compared to IL2pWT immunoconjugate and rec huIL-2. The IL2v immunoconjugate protein incorporating a mutated human IL-2 therefore displays a strongly decreased ability to activate Treg cells compared to wild-type IL-2.

### Example 2: NKCE-IL2v with Topologically N-Terminal NKp46 and Tumor Antigen Binding Sites Promotes Potent NK Cell-Mediated Cytotoxicity

Three-dimensional modelling of NKp46, CD16A and IL2 receptor proteins at the cell surface suggested that in a setting where a dimeric Fc domain was positioned adjacent and N-terminal to the IL2v, the Fc-CD16A interaction may be close to the cell membrane and could lead to the IL2v moiety being positioned too close to the cell membrane to permit efficient interaction with CD122 whose IL-2 binding site is positioned about 70 Angstroms from the cell surface.

A series of different heterotrimeric proteins were compared that all bound tumor antigen (CD20, indicated interchangeably also as “GA101” referring to the anti-CD20 VH/VL pair), CD16A (by inclusion of a dimeric Fc domain with wild-type Fc gamma receptor binding sites), NKp46 and CD122 (by inclusion of an IL2v molecule). The structure of the GA101-T53A-NKp46-IL2v proteins is shown in FIGS. 2A and 2B, with the NKp46-binding domain (as an scFv) was placed at the N-terminus of one of the monomers that make up the dimeric Fc domain, and the IL2v placed at the C-terminus of the same Fc domain (domain arrangement of the NKp46, Fc and IL2v moieties from N- to C-terminus: anti-NKp46 scFv-dimeric Fc-IL2v). The length of the flexible domain linker separating IL2v from the Fc domain was varied, including a short linker of 5 amino acid residues, the 10 amino acid residue linker of GA101-T53A-NKp46-IL2v, and a long linker of 15 amino acid residues. The domain structure of the GA101-T53A-NKp46-IL2v proteins are shown in FIG. 2B.

The proteins tested were:
GA101-T53A-NKp46-IL2v (10 residue linker)
GA101-T53A-NKp46-IL2v Short Linker (5 residue linker)
GA101-T53A-NKp46-IL2v Long Linker (15 residue linker).

In this experiment, NK cell engager proteins were assessed for their ability to induce killing of RAJI tumor cells (CD20+) by NK cells from two human donors at effector:target ratio of 10:1 in a standard 4-hour cytotoxicity assay using calcein release as readout. Briefly, purified NK cells were rested overnight in complete medium. Resting NK cells were then cocultured with Raji tumor cells previously loaded with calcein release, in a 10 to 1 ratio. Cells were incubated with test proteins described above (doses from 20 μg/ml to 0,0001 ug/ml) for 4 h at 37° C., 5.5% C02 in incubator.

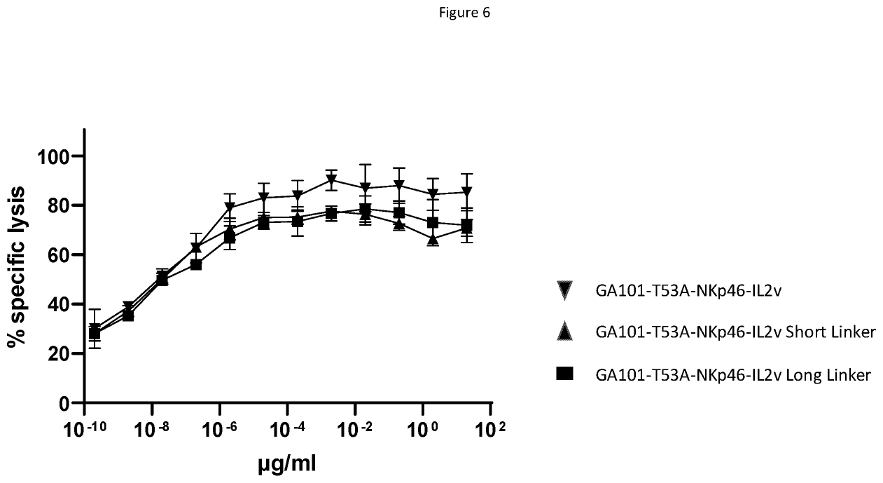

Results are shown in FIG. 6, showing % specific lysis induced by NK cells on the y-axis and concentration of test protein on the x-axis. All GA101-T53A-NKp46-IL2v proteins, including 5, 10 or 15 residue linkers, were highly potent in ability to mediate NK cell cytotoxicity toward tumor target cells.

### Example 3: NKCE-IL2v Promotes IL2R Activation Selectively in NK Cells

The heterotrimeric Fc-domain-containing GA101-T53A-NKp46-IL2v protein shown in FIG. 2B were assessed for its ability to activate Treg cells, NK cells, CD4 T cells and CD8 T cells.

Briefly, 1M/well of purified PBMC were seeded in 96-well plate and treated with increasing doses of NKCE-IL2v or IL2v immunoconjugate (dose from 133 nM to 0,0000013 nM) for 20 min at 37° C., 5.5% CO₂ in incubator. STAT5 phosphorylation was then analysed by flow cytometry on NK cells (CD3-CD56+), CD8 T cells (CD3+CD8+), CD4 T cells (CD3+CD4+FoxP3−) and Tregs (gated on CD3+CD4+CD25+FoxP3+).

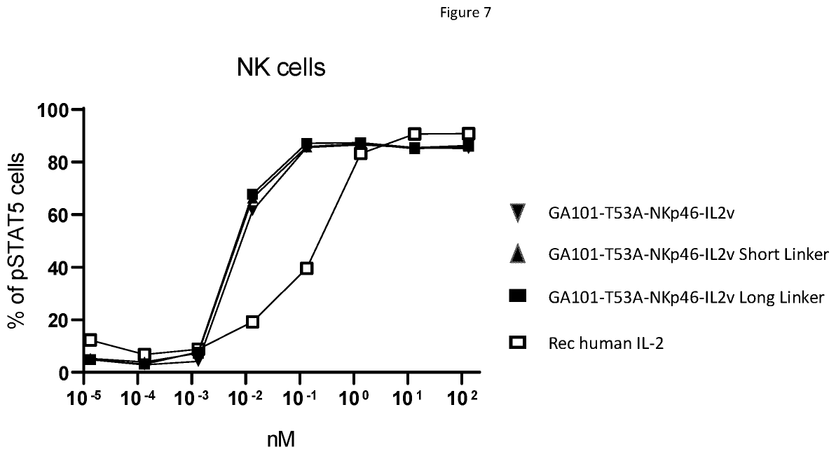

Results are shown in FIG. 7 showing % of pSTAT5 cells among NK cells on the y-axis and concentration of test protein on the x-axis. The GA101-T53A-NKp46-IL2v proteins were more potent than recombinant human IL-2 in the ability to activate NK cells.

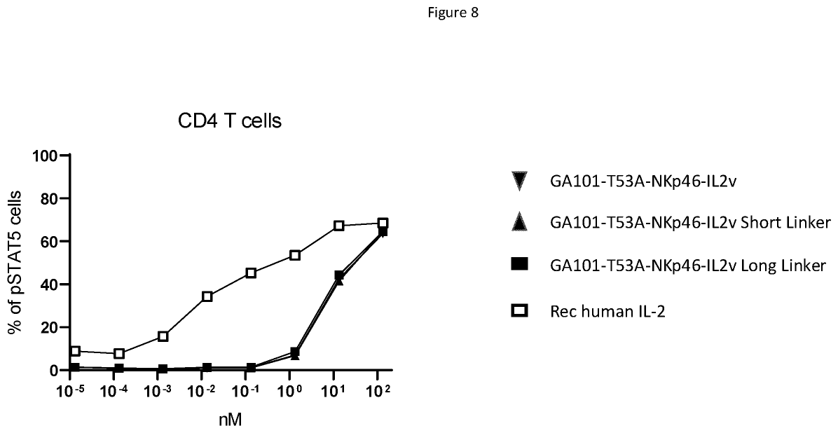

Results are shown in FIG. 8 showing % of pSTAT5 cells among CD4 T cells on the y-axis and concentration of test protein on the x-axis. The GA101-T53A-NKp46-IL2v proteins were less potent than recombinant human IL-2 in the ability to activate CD4 T cells.

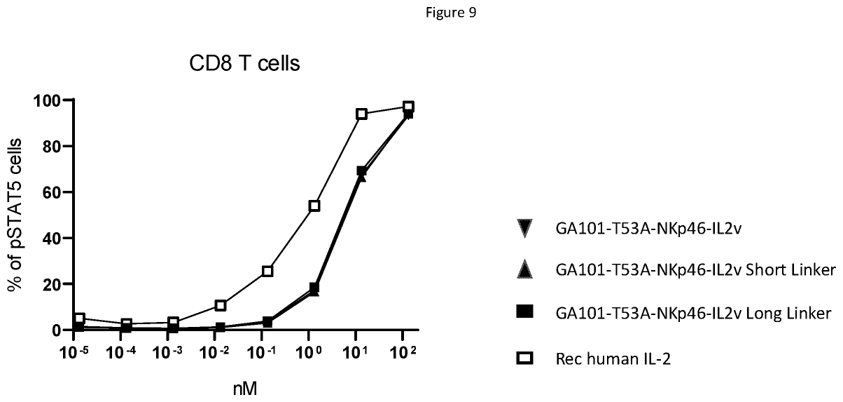

Results are shown in FIG. 9 showing % of pSTAT5 cells among CD8 T cells on the y-axis and concentration of test protein on the x-axis. The GA101-T53A-NKp46-IL2v proteins were less potent than recombinant human IL-2 in the ability to activate CD8 T cells.

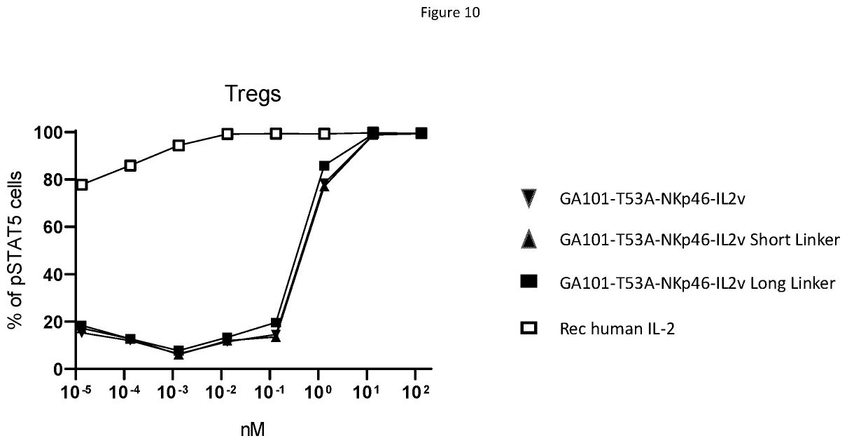

Results are shown in FIG. 10 showing % of pSTAT5 cells among Treg cells on the y-axis and concentration of test protein on the x-axis. The GA101-T53A-NKp46-IL2v proteins were less potent than recombinant human IL-2 in the ability to activate Treg cells.

Overall, the GA101-T53A-NKp46-IL2v displayed lower level of activation of Treg cells, CD4 T cells and CD8 T cells than recombinant IL-2. However, the GA101-T53A-NKp46-IL2v was far more potent (lower EC50) in increasing pSTAT5+ cells among the NK cells, compared recombinant human IL-2 that did not bind NKp46 or CD16A. The GA101-T53A-NKp46-IL2v proteins permitted a potent and selective activation of NK cells over Treg cells, CD4 T cells and CD8 T cells.

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e. g., all exact exemplary values provided with respect to a particular factor or measurement can be considered to also provide a corresponding approximate measurement, modified by “about,” where appropriate). All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as reference to an element or elements is intended to provide support for a similar aspect or embodiment of the invention that “consists of’,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1-45. (canceled)

46. A multispecific protein comprising: an antigen binding domain (ABD) that binds to an antigen of interest; an ABD that binds to a human NKp46 polypeptide; an Fc domain dimer capable of binding FcRn, optionally wherein the Fc domain dimer is further capable of binding to CD16A; and an ABD that binds a human cytokine receptor present on NK cells; wherein
(i) the multispecific protein comprises polypeptide chains 1, 2 and 3: 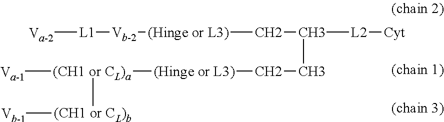
(ii) Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD), wherein one of Vₐ₋₂ and V_{b-2} is a V_{H} and the other is a V_{L} and wherein Vₐ₋₂ and V_{b-2} form a second ABD, wherein the first ABD binds the antigen of interest and the second ABD binds NKp46;
(iii) CH1 is a heavy chain constant domain 1 and CL is a light chain constant domain;
one of (CH1 or CL)ₐ and (CH1 or CL)_{b} is a CH1 and the other is a CL such that a (CH1/CL) pair is formed;
(iv) the hinge is an immunoglobulin hinge region or portion thereof;
(v) L1, L2 and L3 are each an amino acid domain linker, wherein L1, L2 and L3 can be different or the same;
(vi) CH2 and CH3 are human immunoglobulin CH2 and CH3 domains, respectively; and
(vii) Cyt is an ABD that binds a human cytokine receptor present on NK cells, optionally wherein Cyt is a cytokine polypeptide or portion thereof that binds to a cytokine receptor present on NK cells, wherein Cyt is a wild-type or variant human IL-2, IL-15, IL-21, IL-7, IL-27, IL-12, IL-18, IFN-α or IFN-β polypeptide.

47. A multispecific protein comprising: an antigen binding domain (ABD) that binds to an antigen of interest; an ABD that binds to a human NKp46 polypeptide; an Fc domain dimer capable of binding FcRn, optionally wherein the Fc domain dimer is further capable of binding to CD16A; and an ABD that binds a human cytokine receptor present on NK cells; wherein
(i) the multispecific protein comprises polypeptide chains 1, 2 and 3: 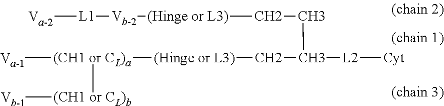
(ii) Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD), wherein one of Vₐ₋₂ and V_{b-2} is a V_{H} and the other is a V_{L} and wherein Vₐ₋₂ and V_{b-2} form a second ABD, wherein the first ABD binds NKp46 and the second ABD binds the antigen of interest;
(iii) CH1 is a heavy chain constant domain 1 and CL is a light chain constant domain;
one of (CH1 or CL)ₐ and (CH1 or CL)_{b} is a CH1 and the other is a CL such that a (CH1/CL) pair is formed;
(iv) the hinge is an immunoglobulin hinge region or portion thereof;
(v) L1, L2 and L3 are each an amino acid domain linker, wherein L1, L2 and L3 can be different or the same;
(vi) CH2 and CH3 are human immunoglobulin CH2 and CH3 domains, respectively; and
(vii) Cyt is an ABD that binds a human cytokine receptor present on NK cells, optionally wherein Cyt is a cytokine polypeptide or portion thereof that binds to a cytokine receptor present on NK cells, wherein Cyt is a wild-type or variant human IL-2, IL-15, IL-21, IL-7, IL-27, IL-12, IL-18, IFN-α or IFN-β polypeptide.

48. A multispecific protein comprising: an antigen binding domain (ABD) that binds to an antigen of interest; an ABD that binds to a human NKp46 polypeptide; an Fc domain dimer capable of binding FcRn, optionally wherein the Fc domain dimer is further capable of binding to CD16A; and an ABD that binds a human cytokine receptor present on NK cells, wherein
(i) the multispecific protein comprises a polypeptide chains 1, 2, 3 and 4 wherein: 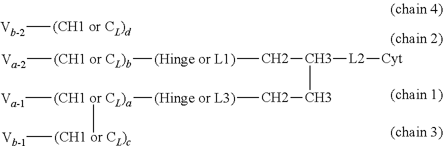
(ii) Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD), wherein one of Vₐ₋₂ and V_{b-2} is a V_{H} and the other is a V_{L} and wherein Vₐ₋₂ and V_{b-2} form a second ABD, wherein the first ABD binds the antigen of interest and the second ABD binds NKp46;
(iii) CH1 is a heavy chain constant domain 1 and CL is a light chain constant domain;
one of (CH1 or CL)ₐ and (CH1 or CL)_{c} is a CH1 and the other is a CL such that a (CH1/CL) pair is formed;
(iv) one of (CH1 or CL)_{b} and (CH1 or CL)_{d} is a CH1 and the other is a CL such that a (CH1/CL) pair is formed;
(v) the hinge is an immunoglobulin hinge region or portion thereof;
(vi) L1, L2 and L3 are each an amino acid domain linker, wherein L1, L2 and L3 can be different or the same;
(vii) CH2 and CH3 are human immunoglobulin CH2 and CH3 domains, respectively; and
Cyt is an ABD that binds a human cytokine receptor present on NK cells, optionally wherein
(viii) Cyt is a cytokine polypeptide or portion thereof that binds to a cytokine receptor present on NK cells, wherein Cyt is a wild-type or variant human IL-2, IL-15, IL-21, IL-7, IL-27, IL-12, IL-18, IFN-α or IFN-β polypeptide.

49. A multispecific protein capable of potentiating NK cell cytotoxicity toward a target cell expressing an antigen of interest, the multispecific protein comprising:
(a) an antigen binding domain (ABD) that binds to the antigen of interest;
(b) an ABD that binds to a human NKp46 polypeptide;
(c) an Fc domain dimer capable of binding FcRn, optionally wherein the Fc domain dimer is further capable of binding to CD16A; and
(d) an ABD that binds a human cytokine receptor present on NK cells, wherein the ABD Is a wild-type or variant human IL-2, IL-15, IL-21, IL-7, IL-27, IL-12, IL-18, IFN-α or IFN-β polypeptide;
wherein the ABD that binds to a human NKp46 polypeptide is connected at its C-terminus to the N-terminus of Fc domain, optionally via an Ig-derived or non-Ig-derived polypeptide linker, and the Fc domain is connected at its C-terminus to the N-terminus of the ABD that binds a human cytokine receptor via a polypeptide linker, and wherein the protein has a single ABD that binds to a human NKp46 polypeptide and a single ABD that binds a human cytokine receptor such that the protein exhibits monovalent binding to each of NKp46 and the human cytokine receptor.

50. The multispecific protein of claim 49, wherein the multispecific protein is capable of co-engaging NKp46 and the cytokine receptor, and further CD16A, on the surface of an NK cell.

51. The multispecific protein of claim 49, wherein the ABD that binds to a human NKp46 polypeptide and the cytokine polypeptide are configured to be capable of adopting a membrane planar binding conformation.

52. The multispecific protein of claim 49, wherein the multispecific protein is capable of directing an NKp46-expressing NK cell to lyse a target cell expressing the antigen of interest, wherein said lysis of the target cell is mediated by NKp46-signaling.

53. The multispecific protein of claim 46, wherein the ABD that binds to a human NKp46 polypeptide comprises a V_{H} and/or V_{L} domain that binds to the D1/D2 junction of the human NKp46 polypeptide, and wherein the cytokine is a modified IL-2 polypeptide.

54. The multispecific protein of claim 46, wherein the multispecific protein causes an increase in NK cell cytotoxicity toward a target cell expressing an antigen of interest, compared to
(i) a multispecific protein that is identical thereto except that the ABD that binds a human cytokine receptor is replaced by a control ABD without binding to any antigen present in the assay, and/or
(ii) a multispecific protein that is identical thereto except that ABD that binds NKp46 is replaced by a control ABD without binding to any antigen present in the assay.

55. The multispecific protein of claim 46, wherein the cytokine or the ABD that binds a cytokine receptor comprises an IL-2 polypeptide displaying reduced binding to CD25 compared to a wild-type human IL-2 polypeptide.

56. The multispecific protein of claim 46, wherein the target cell is a tumor cell.

57. The multispecific protein of claim 46, wherein the antigen of interest is a cancer antigen.

58. The multispecific protein of claim 46, wherein the antigen binding domain that binds NKp46 comprises:
(a) a VH comprising the same CDR 1, 2 and 3 peptides as the VH of SEQ ID NO: 3 and a VL comprising the same CDR 1, 2 and 3 peptides as the VL of SEQ ID NO: 4;
(b) a VH comprising the same CDR 1, 2 and 3 peptides as the VH of SEQ ID NO: 5 and a VL comprising the same CDR 1, 2 and 3 peptides of the VL of SEQ ID NO: 6;
(c) a VH comprising the same CDR 1, 2 and 3 peptides as the VH of SEQ ID NO: 7 and a VL comprising the same CDR 1, 2 and 3 peptides as the VL of SEQ ID NO: 8;
(d) a VH comprising the same CDR 1, 2 and 3 peptides as the VH of SEQ ID NO: 9 and a VL comprising the same CDR 1, 2 and 3 peptides as the VL of SEQ ID NO: 10;
(e) a VH comprising the same CDR 1, 2 and 3 peptides as the VH of SEQ ID NO: 11 and a VL comprising CDR 1, 2 and 3 of the VL of SEQ ID NO: 12; or
(f) a VH comprising the same CDR 1, 2 and 3 peptides as the VH of SEQ ID NO: 13 and a VL comprising the same CDR 1, 2 and 3 peptides as the VL of SEQ ID NO: 14.

59. A pharmaceutical composition comprising the multispecific protein of claim 46, and a pharmaceutically acceptable carrier or adjuvant.

60. A recombinant cell which expresses the multispecific protein of claim 46.

61. A nucleic acid or combination of nucleic acids which when expressed separately or in combination encode the multispecific protein of claim 46.

62. A method of potentiating NK cell activation, cytotoxicity and/or proliferation of NKp46⁺CD16⁺ NK cells and/or NKp46⁺CD16⁻ NK cells in a subject having a disease, the method comprising administering to the subject the multispecific protein of claim 46.

63. A method of treating a cancer, in a subject, the method comprising administering to the subject the multispecific protein of claim 46.

64. A method of making a heteromultimeric protein, comprising:
(a) providing a first nucleic acid encoding the first polypeptide chain of claim 46;
(b) providing a second nucleic acid encoding the second polypeptide chain of claim 46;
(c) providing a third nucleic acid comprising the third polypeptide chain of claim 46; and
(d) expressing said first, second and third nucleic acids in a single host cell or expressing said first, second and third nucleic acids separately in different host cells and combining the resultant polypeptide chains to produce a heteromultimeric protein comprising said first, second and third polypeptide chains; loading the resultant heteromultimeric protein onto an affinity purification support, optionally a Protein-A support, and recovering the heteromultimeric protein.

65. A method of making a heteromultimeric protein, comprising:
(a) providing a first nucleic acid encoding the first polypeptide chain of claim 48;
(b) providing a second nucleic acid encoding the second polypeptide chain of claim 48;
(c) providing a third nucleic acid comprising the third polypeptide chain of claim 48;
(d) providing a fourth nucleic acid encoding the fourth polypeptide chain of claim 48; and
(e) expressing said first, second, third and fourth nucleic acids in a single host cell or expressing said first, second, third and fourth nucleic acids separately in different host cells and combining the resultant polypeptide chains to produce a heteromultimeric protein comprising said first, second, third and fourth polypeptide chains; and loading the resultant heteromultimeric protein onto an affinity purification support, optionally a Protein-A support, and recovering the heteromultimeric protein.

66. A method for identifying or evaluating a multispecific polypeptide, comprising the steps of:
(a) providing nucleic acid(s) encoding the polypeptide chains of the multispecific protein of claim 46;
(b) expressing said nucleic acids in a host cell to produce said multispecific protein;
(c) recovering said multispecific protein; and
(c) evaluating the resultant multispecific protein to determine whether it elicits a biological activity of interest.
